(19) United States
(12) Patent Application Publication
(10) Pub. No.: US 2023/0337446 A1
(43) Pub. Date: Oct. 19, 2023

(54) LIGHT EMITTING ELEMENT AND AMINE COMPOUND FOR THE SAME

(71) Applicant: Samsung Display Co., Ltd., Yongin-si (KR)

(72) Inventor: Akinori YAMATANI, Yokohama (JP)

(21) Appl. No.: 18/300,959

(22) Filed: Apr. 14, 2023

(30) Foreign Application Priority Data
Apr. 15, 2022 (KR) 10-2022-0046641
Mar. 29, 2023 (KR) 10-2023-0041393

(51) Int. Cl. H10K 50/11; H10K 85/60; C07D 307/91; C07D 333/76; C07D 405/12; C07D 409/14; C07D 409/12; C07D 405/14

(52) U.S. Cl. CPC H10K 50/11; H10K 85/636; H10K 85/6574; H10K 85/626; H10K 85/6576; H10K 85/6572; C07D 307/91; C07D 333/76; C07D 405/12; C07D 409/14; C07D 409/12; C07D 405/14; C07B 2200/05

(57) ABSTRACT

Provided are a light emitting element and an amine compound for a light emitting element, and the light emitting element of an embodiment includes a first electrode, a second electrode on the first electrode, and at least one functional layer between the first electrode and the second electrode, wherein an amine compound represented by a set chemical formula is included in the functional layer, thereby improving the emission efficiency and element lifetime of the light emitting element.

### CROSS-REFERENCE TO RELATED APPLICATIONS

This application claims priority to and the benefit of Korean Patent Application Nos. 10-2022-0046641, filed on Apr. 15, 2022, and 10-2023-0041393, filed on Mar. 29, 2023, in the Korean Intellectual Property Office, the entire contents of which are hereby incorporated by reference.

### BACKGROUND

### 1. Field

Embodiments of the present disclosure herein relate to a light emitting element and an amine compound for a light emitting element, and, for example, to a light emitting element including a novel amine compound in a functional layer.

### 2. Description of the Related Art

Recently, the development of an organic electroluminescence display device as an image display device is being actively conducted. The organic electroluminescence display device is a so-called display device including a self-luminescent-type light emitting element in which holes and electrons injected from a first electrode and a second electrode recombine in an emission layer so that a light emitting material in the emission layer emits light to achieve display.

In the application of a light emitting element to a display device, the decrease of a driving voltage and the increase of emission efficiency and lifetime are desired, and development of materials for a light emitting element, stably achieving the requirements is being consistently researched.

In addition, in order to accomplish a light emitting element having a long lifetime, development of materials for a hole transport region having excellent hole transport properties and stability is being conducted.

### SUMMARY

Embodiments of the present disclosure provide a light emitting element that exhibits long-life characteristics.

The present disclosure also provides an amine compound which is a material for a light emitting element for improving element lifetime.

An embodiment provides a light emitting element including: a first electrode; a second electrode on the first electrode; and at least one functional layer between the first electrode and the second electrode, and including an amine compound represented by Formula 1 below.

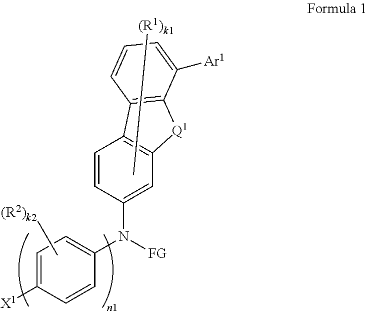

In Formula 1, Q¹ is O or S, Ar¹ is a substituted or unsubstituted phenyl group, R¹ and R² are each independently a hydrogen atom, a deuterium atom, a halogen atom, a substituted or unsubstituted silyl group, a substituted or unsubstituted aryl group of 6 to 30 ring-forming carbon atoms, or a substituted or unsubstituted heteroaryl group of 2 to 30 ring-forming carbon atoms, X¹ is a substituted or unsubstituted naphthyl group, or a substituted or unsubstituted heteroaryl group of 2 to ring-forming carbon atoms, n1 is an integer of 1 to 3, k1 is an integer of 0 to 6, k2 is an integer of 0 to 4, and FG is represented by Formula 2-1 or Formula 2-2 below.

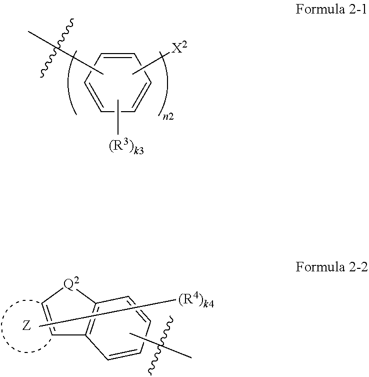

In Formula 2-1 and Formula 2-2, X² is a substituted or unsubstituted naphthyl group, or a substituted or unsubstituted heteroaryl group of 2 to 30 ring-forming carbon atoms, R³ and R⁴ are each independently a hydrogen atom, a deuterium atom, a halogen atom, a substituted or unsubstituted silyl group, a substituted or unsubstituted aryl group of 6 to 30 ring-forming carbon atoms, or a substituted or unsubstituted heteroaryl group of 2 to 30 ring-forming carbon atoms, Q² is O, S, NR⁵, or CR⁶R⁷, R⁵ to R⁷ are each independently a substituted or unsubstituted alkyl group of 1 to 20 carbon atoms, a substituted or unsubstituted aryl group of 6 to 50 ring-forming carbon atoms, or a substituted or unsubstituted heteroaryl group of 2 to 50 ring-forming carbon atoms, Z is a substituted or unsubstituted aromatic hydrocarbon ring of 6 to 30 ring-forming carbon atoms, or a substituted or unsubstituted aromatic heterocycle of 2 to 30 ring-forming carbon atoms, k3 is an integer of 0 to 4, k4 is an integer of 0 to 7 and n2 is an integer of 1 to 3, and a case where X¹ and X² are

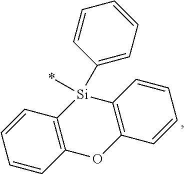 [SMILES: *[Si]1(c2ccccc2)c2ccccc2Oc2ccccc21]

is excluded.

In some embodiments, the at least one functional layer may include an emission layer, a hole transport region between the first electrode and the emission layer, and an electron transport region between the emission layer and the second electrode, and the hole transport region may include the amine compound represented by Formula 1.

In some embodiments, the hole transport region may include a hole injection layer on the first electrode, and an electron blocking layer on the hole injection layer, and the electron blocking layer may include the amine compound represented by Formula 1.

In some embodiments, Formula 2-1 may be represented by Formula 2-1a or Formula 2-1 b below.

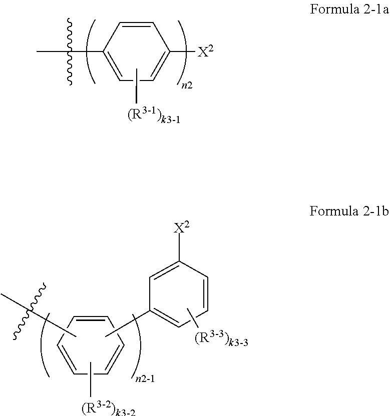

In Formula 2-1a and Formula 2-1b, R³⁻¹ to R³⁻³ are each independently a hydrogen atom, a deuterium atom, a halogen atom, a substituted or unsubstituted aryl group or 6 to 15 ring-forming carbon atoms, or a substituted or unsubstituted heteroaryl group of 2 to 15 ring-forming carbon atoms, k3-1 to k3-3 are each independently an integer of 0 to 4, n2-1 is an integer of 0 to 2, and X² and n2 are the same as defined with respect to Formula 2-1 and Formula 2-2.

In some embodiments, Formula 2-2 may be represented by any one among Formula 2-2a to Formula 2-2c below.

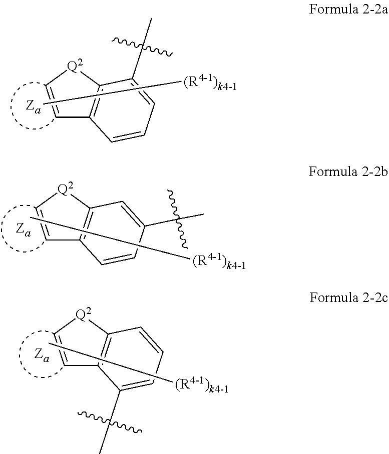

In Formula 2-2a to Formula 2-2c, Zₐ is a substituted or unsubstituted aromatic hydrocarbon ring of 6 to 10 ring-forming carbon atoms, R⁴⁻¹ is a hydrogen atom, a deuterium atom, a halogen atom, or a substituted or unsubstituted aryl group of 6 to 15 ring-forming carbon atoms, k4-1 is an integer of 0 to 7, and Q² is the same as defined with respect to Formula 2-2.

In some embodiments, FG may be represented by any one among Formula FG-1 to Formula FG-6 below.

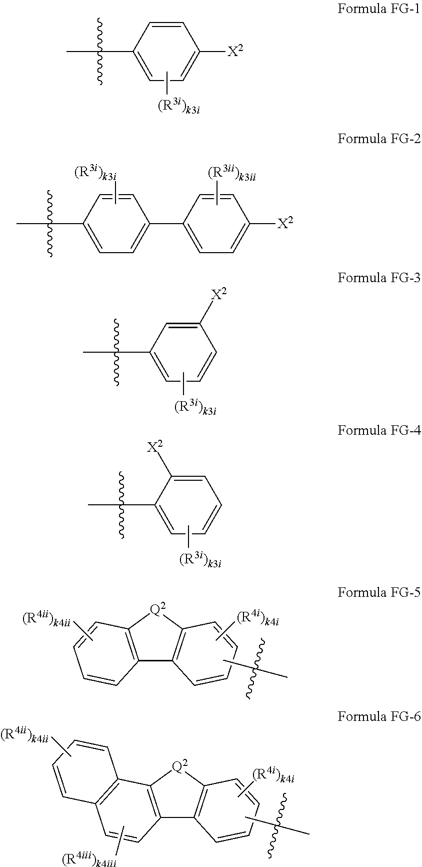

In Formula FG-1 to Formula FG-6, R³ⁱ, R³ⁱⁱ, and R⁴ⁱ to R⁴ⁱⁱⁱ are each independently a hydrogen atom, a deuterium atom, a halogen atom, or a substituted or unsubstituted phenyl group, k3i, k3ii, and k4ii are each independently an integer of 0 to 4, k4i is an integer of 0 to 3, k4iii is an integer of 0 to 2, and X² and Q² are the same as defined with respect to Formula 2-1 and Formula 2-2.

In some embodiments, Formula 1 may be represented by Formula 3-1 or Formula 3-2 below.

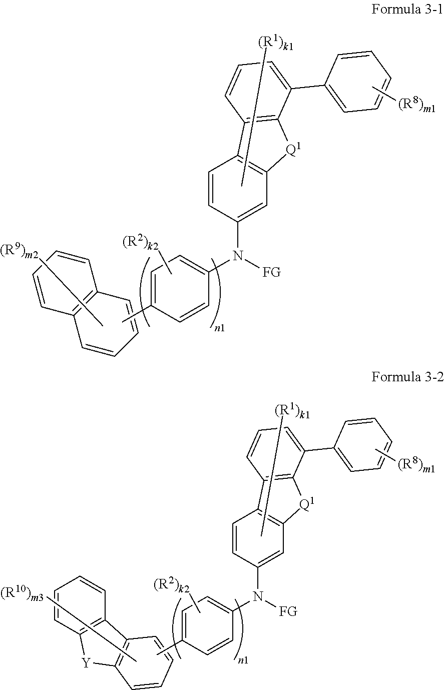

In Formula 3-1 and Formula 3-2, Y is O or NR¹¹, R⁸ to R¹¹ are each independently a hydrogen atom, a deuterium atom, a halogen atom, or a substituted or unsubstituted aryl group of 6 to 20 ring-forming carbon atoms, m1 is an integer of 0 to 5, m2 and m3 are each independently an integer of 0 to 7, and Q¹, R¹, R², n1, k1, k2, and FG are the same as defined with respect to Formula 1.

In some embodiments, Formula 1 may be represented by Formula 4-1 or Formula 4-2 below.

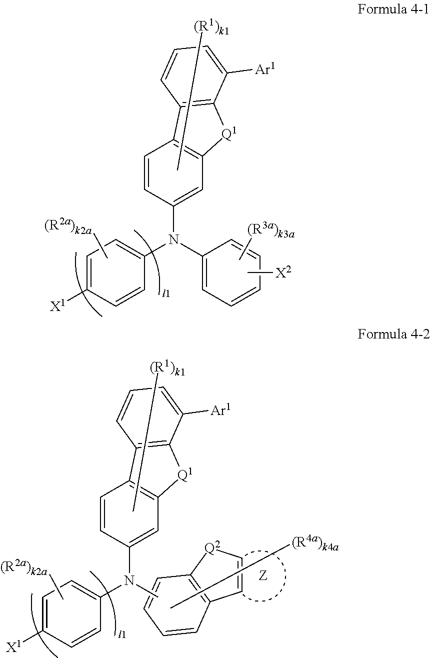

In Formula 4-1 and Formula 4-2, R^{2a} to R^{4a} are each independently a hydrogen atom, a deuterium atom, a halogen atom, or a substituted or unsubstituted phenyl group, k2a and k3a are each independently an integer of 0 to 4, k4a is an integer of 0 to 7, l1 is 1 or 2, and Ar¹, R¹, X¹, X², Q¹, Q², Z, and k1 are the same as defined with respect to Formula 1, Formula 2-1 and Formula 2-2.

In some embodiments, if FG is represented by Formula 2-1, at least one among X¹ and X² may be a substituted or unsubstituted naphthyl group, and if FG is represented by Formula 2-2, X¹ may be a substituted or unsubstituted naphthyl group.

In some embodiments, X¹ and X² may be each independently represented by any one among XS-1 to XS-6 below.

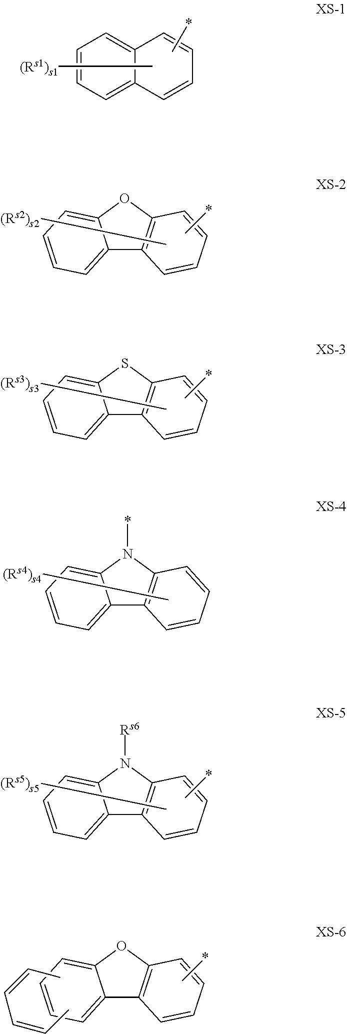

In XS-1 to XS-6, R^{s1} to R^{s6} are each independently a hydrogen atom, a deuterium atom, a halogen atom, or a substituted or unsubstituted aryl group of 6 to 20 ring-forming carbon atoms, s1 to s3, and s5 are each independently an integer of 0 to 7, and s4 is an integer of 0 to 8.

In some embodiments, the emission layer may include a compound represented by Formula E-1 below.

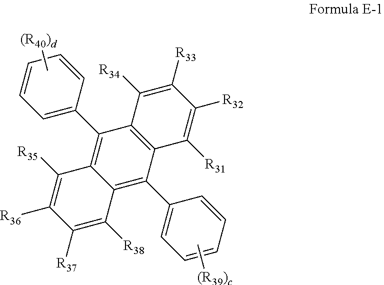

In Formula E-1, R₃₁ to R₄₀ are each independently a hydrogen atom, a deuterium atom, a halogen atom, a substituted or unsubstituted silyl group, a substituted or unsubstituted thio group, a substituted or unsubstituted oxy group, a substituted or unsubstituted alkyl group of 1 to 10 carbon atoms, a substituted or unsubstituted alkenyl group of 2 to 10 carbon atoms, a substituted or unsubstituted aryl group of 6 to 30 ring-forming carbon atoms, or a substituted or unsubstituted heteroaryl group of 2 to 30 ring-forming carbon atoms, or combined with an adjacent group to form a ring, and “c” and “d” are each independently an integer of 0 to 5.

In some embodiments, the amine compound may be represented by any one among the compounds of Compound Group 1, as described herein below.

### BRIEF DESCRIPTION OF THE DRAWINGS

The accompanying drawings are included to provide a further understanding of the subject matter of the present disclosure and are incorporated in and constitute a part of this specification. The drawings illustrate embodiments of the present disclosure and, together with the description, serve to explain principles of the subject matter of the present disclosure. In the drawings:

FIG. 1 is a plan view showing a display device according to an embodiment;

FIG. 2 is a cross-sectional view of a display device according to an embodiment;

FIG. 3 is a cross-sectional view schematically showing a light emitting element of an embodiment;

FIG. 4 is a cross-sectional view schematically showing a light emitting element of an embodiment;

FIG. 5 is a cross-sectional view schematically showing a light emitting element of an embodiment;

FIG. 6 is a cross-sectional view schematically showing a light emitting element of an embodiment;

FIG. 7 is a cross-sectional view of a display device according to an embodiment;

FIG. 8 is a cross-sectional view of a display device according to an embodiment;

FIG. 9 is a cross-sectional view showing a display device according to an embodiment; and

FIG. 10 is a cross-sectional view showing a display device according to an embodiment.

### DETAILED DESCRIPTION

The subject matter of the present disclosure may have various modifications and may be embodied in different forms, and example embodiments will be explained in more detail with reference to the accompany drawings. The subject matter of the present disclosure may, however, be embodied in different forms and should not be construed as limited to the embodiments set forth herein. Rather, all modifications, equivalents, and substituents which are included in the spirit and technical scope of the present disclosure should be available for the subject matter of the present disclosure.

Like reference numerals refer to like elements throughout. In the drawings, the dimensions of structures may be exaggerated for clarity of illustration. It will be understood that, although the terms first, second, etc. may be used herein to describe various elements, these elements should not be limited by these terms. These terms are only used to distinguish one element from another element. Thus, a first element could be termed a second element without departing from the spirit and scope of the present disclosure. Similarly, a second element could be termed a first element. As used herein, the singular forms are intended to include the plural forms as well, unless the context clearly indicates otherwise.

In the description, it will be further understood that the terms “comprises” and/or “comprising,” when used in this specification, specify the presence of stated features, numerals, steps, operations, elements, parts, or the combination thereof, but do not preclude the presence or addition of one or more other features, numerals, steps, operations, elements, parts, or the combination thereof.

In the description, when a layer, a film, a region, a plate, etc. is referred to as being “on” or “above” another part, it can be “directly on” the other part, or intervening layers may also be present. On the contrary, when a layer, a film, a region, a plate, etc. is referred to as being “under” or “below” another part, it can be “directly under” the other part, or intervening layers may also be present. Also, when an element is referred to as being “on” another element, it can be under the other element.

In the description, the term “substituted or unsubstituted” corresponds to substituted or unsubstituted with at least one substituent selected from the group consisting of a deuterium atom, a halogen atom, a cyano group, a nitro group, an amino group, a silyl group, an oxy group, a thio group, a sulfinyl group, a sulfonyl group, a carbonyl group, a boron group, a phosphine oxide group, a phosphine sulfide group, an alkyl group, an alkenyl group, an alkynyl group, a hydrocarbon ring group, an aryl group, and a heterocyclic group. In addition, each of the described substituents may be substituted or unsubstituted. For example, a biphenyl group may be interpreted as an aryl group or a phenyl group substituted with a phenyl group.

In the description, the term “forming a ring via the combination with an adjacent group” may mean forming a substituted or unsubstituted hydrocarbon ring, or a substituted or unsubstituted heterocycle via the combination with an adjacent group. The hydrocarbon ring includes an aliphatic hydrocarbon ring and an aromatic hydrocarbon ring. The heterocycle includes an aliphatic heterocycle and an aromatic heterocycle. The hydrocarbon ring and the heterocycle may be monocycles or polycycles. In addition, the ring formed via the combination with an adjacent group may be combined with another ring to form a spiro structure.

In the description, the term “adjacent group” may mean a substituent substituted for an atom which is directly combined with an atom substituted with a corresponding substituent, another substituent substituted for an atom which is substituted with a corresponding substituent, or a substituent sterically positioned at the nearest position to a corresponding substituent. For example, in 1,2-dimethylbenzene, two methyl groups may be interpreted as “adjacent groups” to each other, and in 1,1-diethylcyclopentene, two ethyl groups may be interpreted as “adjacent groups” to each other. In addition, in 4,5-dimethylphenanthrene, two methyl groups may be interpreted as “adjacent groups” to each other.

In the description, a halogen atom may be a fluorine atom, a chlorine atom, a bromine atom or an iodine atom.

In the description, an alkyl group may be a linear, branched, or cyclic type (e.g., a linear alkyl group, a branched alkyl group, and/or a cyclic alkyl group). The carbon number of the alkyl group may be 1 to 50, 1 to 30, 1 to 20, 1 to 10, or 1 to 6. Examples of the alkyl group may include methyl group, ethyl group, n-propyl group, isopropyl group, n-butyl group, s-butyl group, t-butyl group, i-butyl group, 2-ethylbutyl group, 3,3-dimethylbutyl group, n-pentyl group, i-pentyl group, neopentyl group, t-pentyl group, cyclopentyl group, 1-methylpentyl group, 3-methylpentyl group, 2-ethylpentyl group, 4-methyl-2-pentyl group, n-hexyl group, 1-methylhexyl group, 2-ethylhexyl group, 2-butylhexyl group, cyclohexyl group, 4-methylcyclohexyl group, 4-t-butylcyclohexyl group, n-heptyl group, 1-methylheptyl group, 2,2-dimethylheptyl group, 2-ethylheptyl group, 2-butylheptyl group, n-octyl group, t-octyl group, 2-ethyloctyl group, 2-butyloctyl group, 2-hexyloctyl group, 3,7-dimethyloctyl group, cyclooctyl group, n-nonyl group, n-decyl group, adamantyl group, 2-ethyldecyl group, 2-butyldecyl group, 2-hexyldecyl group, 2-octyldecyl group, n-undecyl group, n-dodecyl group, 2-ethyldodecyl group, 2-butyldodecyl group, 2-hexyldocecyl group, 2-octyldodecyl group, n-tridecyl group, n-tetradecyl group, n-pentadecyl group, n-hexadecyl group, 2-ethylhexadecyl group, 2-butylhexadecyl group, 2-hexylhexadecyl group, 2-octylhexadecyl group, n-heptadecyl group, n-octadecyl group, n-nonadecyl group, n-eicosyl group, 2-ethyleicosyl group, 2-butyleicosyl group, 2-hexyleicosyl group, 2-octyleicosyl group, n-henicosyl group, n-docosyl group, n-tricosyl group, n-tetracosyl group, n-pentacosyl group, n-hexacosyl group, n-heptacosyl group, n-octacosyl group, n-nonacosyl group, n-triacontyl group, etc., without limitation.

In the description, an alkenyl group means a hydrocarbon group including one or more carbon double bonds at a main chain (e.g., in the middle) or at a terminal end (e.g., the terminus) of an alkyl group having a carbon number of 2 or more. The alkenyl group may be a linear chain or a branched chain. The carbon number is not specifically limited, but may be 2 to 30, 2 to 20, or 2 to 10. Examples of the alkenyl group include a vinyl group, a 1-butenyl group, a 1-pentenyl group, a 1,3-butadienyl aryl group, a styrenyl group, a styrylvinyl group, etc., without limitation.

In the description, an alkynyl group means a hydrocarbon group including one or more carbon triple bonds at a main chain (e.g., in the middle) or at a terminal end (e.g., the terminus) of an alkyl group having a carbon number of 2 or more. The alkynyl group may be a linear chain or a branched chain. The carbon number is not specifically limited, but may be 2 to 30, 2 to 20, or 2 to 10. Examples of the alkynyl group include an ethynyl group, a propynyl group, etc., without limitation.

In the description, a hydrocarbon ring group means an optional functional group or substituent derived from an aliphatic hydrocarbon ring. The hydrocarbon ring group may be a saturated hydrocarbon ring group of 5 to 20 ring-forming carbon atoms.

In the description, an aryl group means an optional functional group or substituent derived from an aromatic hydrocarbon ring. The aryl group may be a monocyclic aryl group or a polycyclic aryl group. The carbon number for forming rings in the aryl group may be 6 to 50, 6 to 30, 6 to 20, or 6 to 15. Examples of the aryl group may include phenyl group, naphthyl group, fluorenyl group, anthracenyl group, phenanthryl group, biphenyl group, terphenyl group, quaterphenyl group, quinquephenyl group, sexiphenyl group, triphenylenyl group, pyrenyl group, benzofluoranthenyl group, chrysenyl group, etc., without limitation.

In the description, a fluorenyl group may be substituted, and two substituents may be combined with each other to form a spiro structure. Examples of a substituted fluorenyl group are as follows, but embodiments of the present disclosure are not limited thereto.

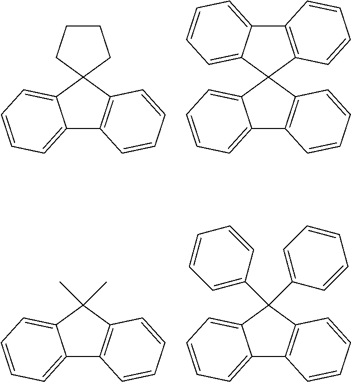

In the description, a heterocyclic group means an optional functional group or substituent derived from a ring including one or more among B, O, N, P, Si, and S as heteroatoms. The heterocyclic group includes an aliphatic heterocyclic group and an aromatic heterocyclic group. The aromatic heterocyclic group may be a heteroaryl group. The aliphatic heterocyclic group and the aromatic heterocyclic group may be a monocycle or a polycycle.

In the description, a heterocyclic group may include one or more among B, O, N, P, Si and S as heteroatoms. If the heterocyclic group includes two or more heteroatoms, two or more heteroatoms may be the same or different. In the description, the heterocyclic group may be a monocyclic heterocyclic group or polycyclic heterocyclic group and has concept including a heteroaryl group. The carbon number for forming rings of the heterocyclic group may be 2 to 50, 2 to 30, 2 to 20, or 2 to 10.

In the description, an aliphatic heterocyclic group may include one or more among B, O, N, P, Si, and S as heteroatoms. The number of ring-forming carbon atoms of the aliphatic heterocyclic group may be 2 to 30, 2 to 20, or 2 to 10. Examples of the aliphatic heterocyclic group may include an oxirane group, a thiirane group, a pyrrolidine group, a piperidine group, a tetrahydrofuran group, a tetrahydrothiophene group, a thiane group, a tetrahydropyran group, a 1,4-dioxane group, etc., without limitation.

In the description, a heteroaryl group may include one or more among B, O, N, P, Si, and S as heteroatoms. If the heteroaryl group includes two or more heteroatoms, two or more heteroatoms may be the same or different. The heteroaryl group may be a monocyclic heterocyclic group or polycyclic heterocyclic group. The carbon number for forming rings of the heteroaryl group may be 2 to 50, 2 to 30, 2 to 20, or 2 to 10. Examples of the heteroaryl group may include thiophene group, furan group, pyrrole group, imidazole group, triazole group, pyridine group, bipyridine group, pyrimidine group, triazine group, acridyl group, pyridazine group, pyrazinyl group, quinoline group, quinazoline group, quinoxaline group, phenoxazine group, phthalazine group, pyrido group pyrimidine group, pyrido pyrazine group, pyrazino pyrazine group, isoquinoline group, indole group, carbazole group, N-arylcarbazole group, N-heteroarylcarbazole group, N-alkylcarbazole group, benzoxazole group, benzoimidazole group, benzothiazole group, benzocarbazole group, benzothiophene group, dibenzothiophene group, thienothiophene group, benzofuran group, phenanthroline group, thiazole group, isoxazole group, oxazole group, oxadiazole group, thiadiazole group, phenothiazine group, dibenzosilole group, dibenzofuran group, etc., without limitation.

In the description, the same explanation of the above-described aryl group may be applied to an arylene group except that the arylene group is a divalent group. The same explanation of the above-described heteroaryl group may be applied to a heteroarylene group except that the heteroarylene group is a divalent group.

In the description, a boron group may mean the above-defined alkyl group or aryl group bonded to a boron atom. The boron group may include an alkyl boron group and an aryl boron group. Examples of the boron group include a dimethylboron group, a diethylboron group, a t-butylmethylboron group, a diphenylboron group, a phenylboron group, and/or the like, without limitation.

In the description, a silyl group includes an alkyl silyl group and an aryl silyl group. Examples of the silyl group include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, etc., without limitation.

In the description, the carbon number of a carbonyl group is not specifically limited, but the carbon number may be 1 to 40, 1 to 30, or 1 to 20. For example, the carbonyl group may have the structures below, but is not limited thereto.

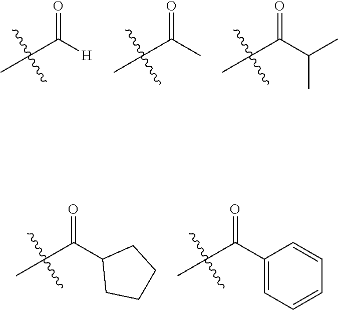

In the description, the carbon number of a sulfinyl group and sulfonyl group is not specifically limited, but may be 1 to 30. The sulfinyl group may include an alkyl sulfinyl group and an aryl sulfinyl group. The sulfonyl group may include an alkyl sulfonyl group and an aryl sulfonyl group.

In the description, a thio group may include an alkyl thio group and an aryl thio group. The thio group may mean the above-defined alkyl group or aryl group combined with a sulfur atom. Examples of the thio group include a methylthio group, an ethylthio group, a propylthio group, a pentylthio group, a hexylthio group, an octylthio group, a dodecylthio group, a cyclopentylthio group, a cyclohexylthio group, a phenylthio group, a naphthylthio group, etc., without limitation.

In the description, an oxy group may mean the above-defined alkyl group or aryl group which is combined with an oxygen atom. The oxy group may include an alkoxy group and an aryl oxy group. The alkoxy group may be a linear, branched or cyclic chain. The carbon number of the alkoxy group is not specifically limited but may be, for example, 1 to 20 or 1 to 10. Examples of the oxy group may include methoxy, ethoxy, n-propoxy, isopropoxy, butoxy, pentyloxy, hexyloxy, octyloxy, nonyloxy, decyloxy, benzyloxy, etc. However, embodiments of the present disclosure are not limited thereto.

In the description, the carbon number of an amine group is not specifically limited, but may be 1 to 30. The amine group may include an alkyl amine group and an aryl amine group. Examples of the amine group include a methylamine group, a dimethylamine group, a phenylamine group, a diphenylamine group, a naphthylamine group, a 9-methyl-anthracenylamine group, etc., without limitation.

In the description, alkyl groups in an alkylthio group, alkylsulfoxy group, alkylaryl group, alkylamino group, alkylboron group, alkyl silyl group, and alkyl amine group may be the same as the examples of the above-described alkyl group.

In the description, aryl groups in an aryloxy group, arylthio group, arylsulfoxy group, aryl amino group, arylboron group, and aryl silyl group may be the same as the examples of the above-described aryl group.

In the present description, a direct linkage may mean a single bond (e.g., a single covalent bond or the like).

In the present description,

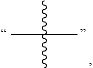

or “—*” means a position to be connected.

Hereinafter, the light emitting element of an embodiment will be explained referring to the drawings.

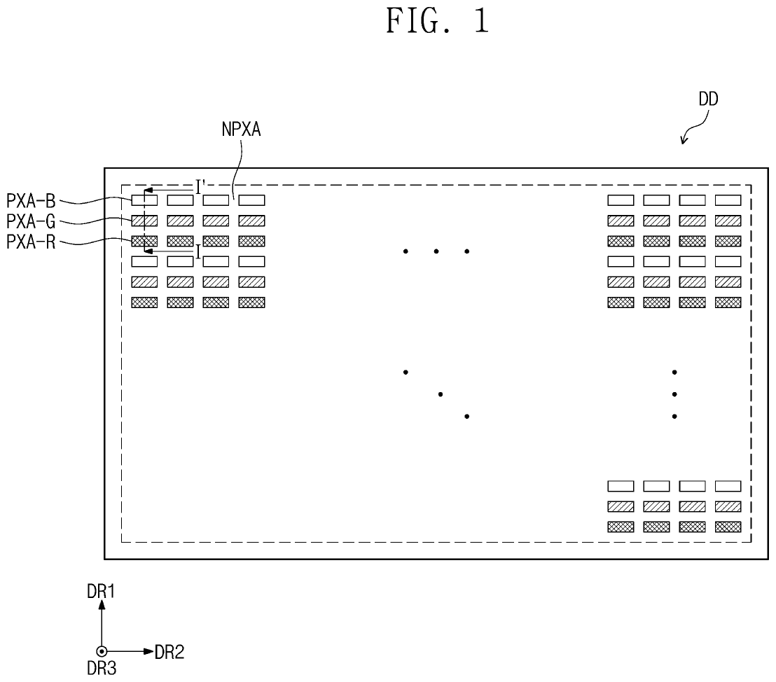

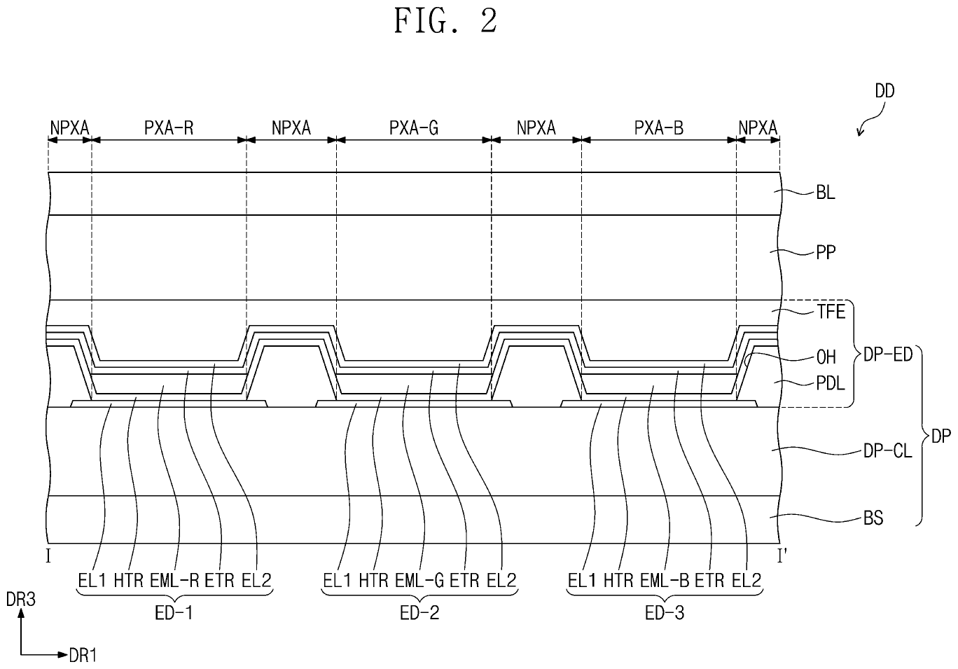

FIG. 1 is a plan view showing an embodiment of a display device DD. FIG. 2 is a cross-sectional view of a display device DD of an embodiment. FIG. 2 is a cross-sectional view showing a part corresponding to line I-I′ of FIG. 1.

The display device DD may include a display panel DP and an optical layer PP on the display panel DP. The display panel DP may include light emitting elements ED-1, ED-2 and ED-3. The display device DD may include a plurality of the light emitting elements ED-1, ED-2 and ED-3. The optical layer PP may be on the display panel DP and control reflected light by external light at the display panel DP. The optical layer PP may include, for example, a polarization layer and/or a color filter layer. Different from the drawings, the optical layer PP may be omitted in the display device DD of some embodiments.

A base substrate BL may be on the optical layer PP. The base substrate BL may be a member providing a base surface that the optical layer PP is on. The base substrate BL may be a glass substrate, a metal substrate, a plastic substrate, etc. However, embodiments of the present disclosure are not limited thereto, and the base substrate BL may be an inorganic layer, an organic layer or a composite material layer. In addition, different from the drawings, the base substrate BL may be omitted in some embodiments.

The display device DD according to an embodiment may further include a plugging layer. The plugging layer may be between a display element layer DP-ED and the base substrate BL. The plugging layer may be an organic layer. The plugging layer may include at least one among an acrylic resin, a silicon-based resin and an epoxy-based resin.

The display panel DP may include a base layer BS, a circuit layer DP-CL provided on the base layer BS and a display element layer DP-ED. The display element layer DP-ED may include a pixel definition layer PDL, the light emitting elements ED-1, ED-2 and ED-3 in the pixel definition layer PDL, and an encapsulating layer TFE on the light emitting elements ED-1, ED-2 and ED-3.

The base layer BS may be a member providing a base surface that the display element layer DP-ED is on. The base layer BS may be a glass substrate, a metal substrate, a plastic substrate, etc. However, embodiments of the present disclosure are not limited thereto, and the base layer BS may be an inorganic layer, an organic layer or a composite material layer.

In some embodiments, the circuit layer DP-CL is on the base layer BS, and the circuit layer DP-CL may include a plurality of transistors. Each of the transistors may include a control electrode, an input electrode, and an output electrode. For example, the circuit layer DP-CL may include switching transistors and driving transistors for driving the light emitting elements ED-1, ED-2 and ED-3 of the display element layer DP-ED.

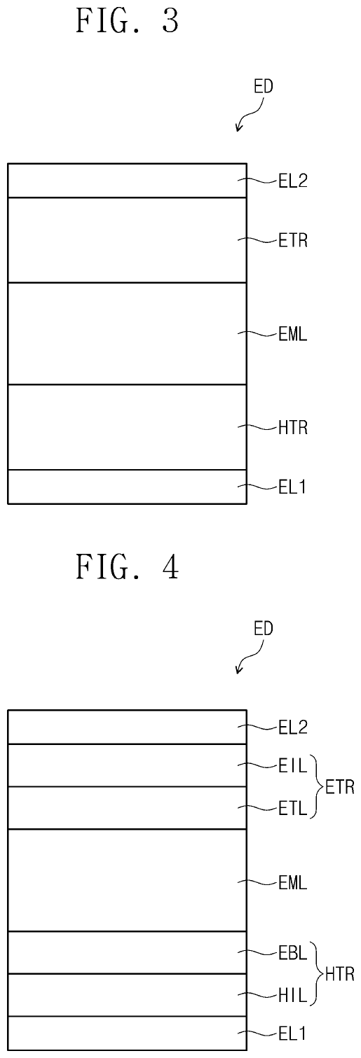

The light emitting elements ED-1, ED-2 and ED-3 may have the structures of the light emitting elements ED of embodiments according to FIG. 3 to FIG. 6, which will be further explained herein below. The light emitting elements ED-1, ED-2 and ED-3 may include a first electrode EL1, a hole transport region HTR, emission layers EML-R, EML-G and EML-B, an electron transport region ETR and a second electrode EL2.

In FIG. 2, shown is an embodiment where the emission layers EML-R, EML-G and EML-B of light emitting elements ED-1, ED-2 and ED-3 are in opening portions OH defined in a pixel definition layer PDL, and a hole transport region HTR, an electron transport region ETR and a second electrode EL2 are provided as common layers in all light emitting elements ED-1, ED-2 and ED-3. However, embodiments of the present disclosure are not limited thereto. Different from FIG. 2, in some embodiments, the hole transport region HTR and the electron transport region ETR may be patterned and provided in the opening portions OH defined in the pixel definition layer PDL. For example, in some embodiments, the hole transport region HTR, the emission layers EML-R, EML-G and EML-B, and the electron transport region ETR of the light emitting elements ED-1, ED-2 and ED-3 may be patterned by an ink jet printing method and provided.

The encapsulating layer TFE may cover the light emitting elements ED-1, ED-2 and ED-3. The encapsulating layer TFE may encapsulate the display element layer DP-ED. The encapsulating layer TFE may be a thin film encapsulating layer. The encapsulating layer TFE may be one layer or a stacked layer of a plurality of layers. The encapsulating layer TFE includes at least one insulating layer. The encapsulating layer TFE according to an embodiment may include at least one inorganic layer (hereinafter, encapsulating inorganic layer). In addition, the encapsulating layer TFE according to an embodiment may include at least one organic layer (hereinafter, encapsulating organic layer) and at least one encapsulating inorganic layer.

The encapsulating inorganic layer protects the display element layer DP-ED from moisture/oxygen, and the encapsulating organic layer protects the display element layer DP-ED from foreign materials such as dust particles. The encapsulating inorganic layer may include silicon nitride, silicon oxy nitride, silicon oxide, titanium oxide, and/or aluminum oxide, without specific limitation. The encapsulating organic layer may include an acrylic compound, an epoxy-based compound, etc. The encapsulating organic layer may include a photopolymerizable organic material, without specific limitation.

The encapsulating layer TFE may be on the second electrode EL2 and may fill the opening portion OH.

Referring to FIG. 1 and FIG. 2, the display device DD may include a non-luminous area NPXA and luminous areas PXA-R, PXA-G and PXA-B. The luminous areas PXA-R, PXA-G and PXA-B may be areas that emit light produced from the light emitting elements ED-1, ED-2 and ED-3, respectively. The luminous areas PXA-R, PXA-G and PXA-B may be separated (e.g., spaced apart) from each other on a plane.

The luminous areas PXA-R, PXA-G and PXA-B may be areas separated (e.g., spaced apart) by the pixel definition layer PDL. The non-luminous areas NPXA may be areas between neighboring luminous areas PXA-R, PXA-G and PXA-B and may be areas corresponding to the pixel definition layer PDL. In some embodiments, each of the luminous areas PXA-R, PXA-G and PXA-B may correspond to each pixel. The pixel definition layer PDL may divide the light emitting elements ED-1, ED-2 and ED-3. The emission layers EML-R, EML-G and EML-B of the light emitting elements ED-1, ED-2 and ED-3 may be provided and divided in the opening portions OH defined in the pixel definition layer PDL.

The luminous areas PXA-R, PXA-G and PXA-B may be divided into a plurality of groups according to the color of light produced from the light emitting elements ED-1, ED-2 and ED-3. In the display device DD of an embodiment, shown in FIG. 1 and FIG. 2, three luminous areas PXA-R, PXA-G and PXA-B emitting red light, green light and blue light are illustrated as an embodiment. For example, the display device DD of an embodiment may include a red luminous area PXA-R, a green luminous area PXA-G and a blue luminous area PXA-B, which are separated (e.g., spaced apart) from each other.

In the display device DD according to an embodiment, the plurality of light emitting elements ED-1, ED-2 and ED-3 may emit light having different wavelength regions. For example, in some embodiments, the display device DD may include a first light emitting element ED-1 that emits red light, a second light emitting element ED-2 that emits green light, and a third light emitting element ED-3 that emits blue light. For example, each of the red luminous area PXA-R, the green luminous area PXA-G, and the blue luminous area PXA-B of the display device DD may correspond to the first light emitting element ED-1, the second light emitting element ED-2, and the third light emitting element ED-3.

However, embodiments of the present disclosure are not limited thereto, and the first to third light emitting elements ED-1, ED-2 and ED-3 may emit light in the same wavelength region, or at least one thereof may emit light in a different wavelength region. For example, all the first to third light emitting elements ED-1, ED-2 and ED-3 may emit blue light.

The luminous areas PXA-R, PXA-G and PXA-B in the display device DD according to an embodiment may be arranged in a stripe shape. Referring to FIG. 1, a plurality of red luminous areas PXA-R, a plurality of green luminous areas PXA-G and a plurality of blue luminous areas PXA-B may be arranged along a second directional axis DR2. In addition, the red luminous area PXA-R, the green luminous area PXA-G and the blue luminous area PXA-B may be arranged by turns along a first directional axis DR1.

In FIG. 1 and FIG. 2, the areas of the luminous areas PXA-R, PXA-G and PXA-B are shown to be similar, but embodiments of the present disclosure are not limited thereto. The areas of the luminous areas PXA-R, PXA-G and PXA-B may be different from each other according to the wavelength region of light emitted. In some embodiments, the areas of the luminous areas PXA-R, PXA-G and PXA-B may mean areas on a plane defined by the first directional axis DR1 and the second directional axis DR2.

the arrangement type (or kind) of the luminous areas PXA-R, PXA-G and PXA-B is not limited to the configuration shown in FIG. 1, and the arrangement order of the red luminous areas PXA-R, the green luminous areas PXA-G and the blue luminous areas PXA-B may be provided in various combinations according to the properties of display quality desired or required for the display device DD. For example, the arrangement type (or kind) of the luminous areas PXA-R, PXA-G and PXA-B may be a pentile (PENTILE®) arrangement type (e.g., an RGBG matrix, RGBG structure, or RGBG matrix structure), or a diamond (Diamond Pixel™) arrangement type. PENTILE® is a duly registered trademark of Samsung Display Co., Ltd.

In addition, the areas of the luminous areas PXA-R, PXA-G and PXA-B may be different from each other. For example, in some embodiments, the area of the green luminous area PXA-G may be smaller than the area of the blue luminous area PXA-B, but embodiments of the present disclosure are not limited thereto.

Hereinafter, FIG. 3 to FIG. 6 are cross-sectional views schematically showing light emitting elements according to embodiments. The light emitting element ED according to an embodiment may include a first electrode EL1, a second electrode EL2 opposite to the first electrode EL1, and at least one functional layer between the first electrode EL1 and the second electrode EL2. The light emitting element ED of an embodiment may include an amine compound of an embodiment, which will be further explained herein below, in the at least one functional layer.

The light emitting element ED may include a hole transport region HTR, an emission layer EML, an electron transport region ETR, and/or the like, stacked in order, as the at least one functional layer. Referring to FIG. 3, the light emitting element ED of an embodiment may include a first electrode EL1, a hole transport region HTR, an emission layer EML, an electron transport region ETR, and a second electrode EL2, stacked in order.

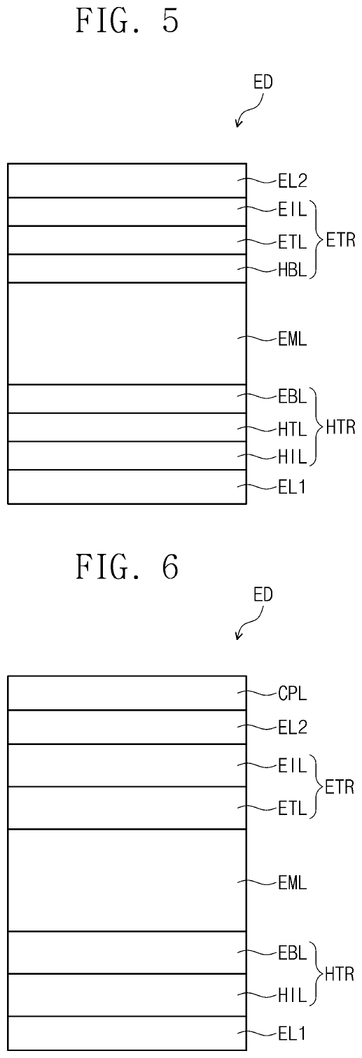

When compared with FIG. 3, FIG. 4 shows the cross-sectional view of a light emitting element ED of an embodiment, wherein a hole transport region HTR includes a hole injection layer HIL and an electron blocking layer EBL, and an electron transport region ETR includes an electron injection layer EIL and an electron transport layer ETL. In addition, when compared with FIG. 3, FIG. 5 shows the cross-sectional view of a light emitting element ED of an embodiment, wherein a hole transport region HTR includes a hole injection layer HIL, a hole transport layer HTL, and an electron blocking layer EBL, and an electron transport region ETR includes an electron injection layer EIL, an electron transport layer ETL, and a hole blocking layer HBL. When compared with FIG. 4, FIG. 6 shows the cross-sectional view of a light emitting element ED of an embodiment, including a capping layer CPL on the second electrode EL2.

The light emitting element ED of an embodiment may include an amine compound of an embodiment, which will be further explained herein below, in a hole transport region HTR. The light emitting element ED of an embodiment may include an amine compound of an embodiment in at least one among the hole injection layer HIL, hole transport layer HTL and electron blocking layer EBL of the hole transport region HTR. For example, in the light emitting element ED of an embodiment, the electron blocking layer EBL may include the amine compound of an embodiment. The electron blocking layer EBL is a layer playing the role of preventing or reducing electron injection from the electron transport region ETR to the hole transport region HTR.

In the light emitting element ED according to an embodiment, the first electrode EL1 has conductivity (e.g., electrical conductivity). The first electrode EL1 may be formed using a metal material, a metal alloy or a conductive compound. The first electrode EL1 may be an anode or a cathode. However, embodiments of the present disclosure are not limited thereto. In addition, the first electrode EL1 may be a pixel electrode. The first electrode EL1 may be a transmissive electrode, a transflective electrode, or a reflective electrode. The first electrode EL1 may include at least one selected among Ag, Mg, Cu, Al, Pt, Pd, Au, Ni, Nd, Ir, Cr, Li, Ca, LiF, Mo, Ti, W, In, Sn and Zn, compounds of two or more selected therefrom, mixtures of two or more selected therefrom, or oxides thereof.

If the first electrode EL1 is the transmissive electrode, the first electrode EL1 may include a transparent metal oxide such as indium tin oxide (ITO), indium zinc oxide (IZO), zinc oxide (ZnO), and indium tin zinc oxide (ITZO). If the first electrode EL1 is the transflective electrode or the reflective electrode, the first electrode EL1 may include Ag, Mg, Cu, Al, Pt, Pd, Au, Ni, Nd, Ir, Cr, Li, Ca, LiF/Ca (a stacked structure of LiF and Ca), LiF/Al (a stacked structure of LiF and Al), Mo, Ti, W, compounds thereof, or mixtures thereof (for example, a mixture of Ag and Mg). Also, the first electrode EL1 may have a structure including a plurality of layers including a reflective layer or a transflective layer formed using the above materials, and a transmissive conductive layer formed using ITO, IZO, ZnO, and/or ITZO. For example, the first electrode EL1 may have a three-layer structure of ITO/Ag/ITO. However, embodiments of the present disclosure are not limited thereto. The first electrode EL1 may include the above-described metal materials, combinations of two or more metal materials selected from the above-described metal materials, and/or oxides of the above-described metal materials. The thickness of the first electrode EL1 may be from about 700 Å to about 10,000 Å. For example, the thickness of the first electrode EL1 may be from about 1,000 Å to about 3,000 Å.

The hole transport region HTR is provided on the first electrode EL1. The hole transport region HTR may have a single layer formed using a single material, a single layer formed using a plurality of different materials, or a multilayer structure including a plurality of layers formed using a plurality of different materials.

The hole transport region HTR may include at least one selected from a hole injection layer HIL, a hole transport layer HTL, and an electron blocking layer EBL. In addition, in some embodiments, the hole transport region HTR may include a plurality of hole transport layers stacked.

In addition, otherwise, the hole transport region HTR may have the structure of a single layer of a hole injection layer HIL or a hole transport layer HTL, and may have a structure of a single layer formed using a hole injection material and a hole transport material. In some embodiments, the hole transport region HTR may have a structure of a single layer formed using a plurality of different materials, or a structure stacked from the first electrode EL1 of hole injection layer HIL/hole transport layer HTL, hole injection layer HIL/hole transport layer HTL/buffer layer, hole injection layer HIL/buffer layer, or hole transport layer HTL/buffer layer, without limitation.

The thickness of the hole transport region HTR may be, for example, about 50 Å to about 15,000 Å. The hole transport region HTR may be formed using various suitable methods such as a vacuum deposition method, a spin coating method, a cast method, a Langmuir-Blodgett (LB) method, an inkjet printing method, a laser printing method, and/or a laser induced thermal imaging (LITI) method.

The light emitting element ED of an embodiment may include the amine compound of an embodiment in a hole transport region HTR. In the light emitting element ED of an embodiment, the hole transport region HTR may include an electron injection layer EIL, a hole transport layer HTL, and an electron blocking layer EBL. For example, the electron blocking layer EBL may include the amine compound of an embodiment. In some embodiments, the amine compound may be represented by Formula 1.

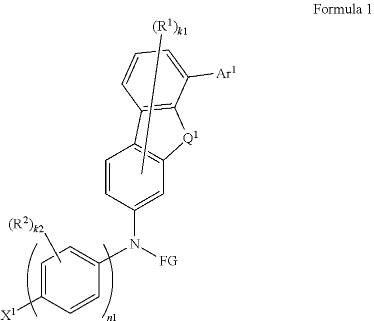

In Formula 1, Q¹ may be O or S, and Ar¹ may be a substituted or unsubstituted phenyl group. For example, if Q¹ is O, a substituted or unsubstituted phenyl group may be bonded to position 6 of a dibenzofuran group, and the nitrogen atom (N) of an amine compound may be connected at position 3 of the dibenzofuran group. In addition, if Q¹ is S, a substituted or unsubstituted phenyl group may be bonded to position 6 of a dibenzothiophene group, and the nitrogen atom (N) of an amine compound may be connected at position 3 of the dibenzothiophene group.

For example, the amine compound of an embodiment, represented by Formula 1 may include a substituent of a dibenzoheterole skeleton that is connected with a substituted or unsubstituted phenyl group at position 6 and is combined with the nitrogen atom (N) of an amine compound at position 3. Accordingly, the amine compound of an embodiment has high stability, and if used as the hole transport material of a light emitting element, may contribute to the improvement of the lifetime of the light emitting element, and may be suitably applied in the electron blocking layer of the light emitting element after suitably controlling a LUMO level.

In Formula 1, R¹ and R² may be each independently a hydrogen atom, a deuterium atom, a halogen atom, a substituted or unsubstituted silyl group, a substituted or unsubstituted aryl group of 6 to 30 ring-forming carbon atoms, or a substituted or unsubstituted heteroaryl group of 2 to 30 ring-forming carbon atoms. For example, R¹ and R² may be each independently a hydrogen atom, a deuterium atom, or a halogen atom. If each of R¹ and R² is a halogen atom, the amine compound of an embodiment may include a fluorine atom (F) as the halogen atom.

In Formula 1, n1 may be an integer of 0 to 3. For example, n1 may be 1 or 2. k1 may be an integer of 0 to 6, and k2 may be an integer of 0 to 4. A case where k1 is 0, may be the same as a case where k1 is 6, and R¹ is a hydrogen atom. A case where k2 is 0, may be the same as a case where k2 is 4, and R² is a hydrogen atom.

In some embodiments, if k1 is an integer of 2 or more, a plurality of R¹ may be all the same, or at least one may be different from the remainder. In some embodiments, if k1 is 0, the substituent of the dibenzoheterole skeleton in Formula 1 may be unsubstituted with R¹. If k2 is an integer of 2 or more, a plurality of R² may be all the same, or at least one may be different from the remainder. In some embodiments, if k2 is 0, the phenylene group in Formula 1 may be unsubstituted with R².

In Formula 1, X¹ may be a substituted or unsubstituted naphthyl group, or a substituted or unsubstituted heteroaryl group of 2 to 30 ring-forming carbon atoms. For example, X¹ may be a naphthyl group substituted with a deuterium atom, a halogen atom, or a substituted or unsubstituted aryl group of 6 to 20 ring-forming carbon atoms, an unsubstituted naphthyl group, or a substituted or unsubstituted heteroaryl group of 2 to 20 ring-forming carbon atoms, containing an oxygen atom (O) or a nitrogen atom (N) as a ring-forming atom. However, because

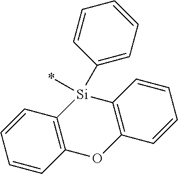 [SMILES: *[Si]1(c2ccccc2)c2ccccc2Oc2ccccc21]

is low in stability against holes and electrons, in the amine compound of an embodiment, a case where X¹ is

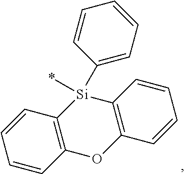 [SMILES: *[Si]1(c2ccccc2)c2ccccc2Oc2ccccc21]

may be excluded.

In some embodiments, X¹ may be represented by any one among XS-1 to XS-6 below.

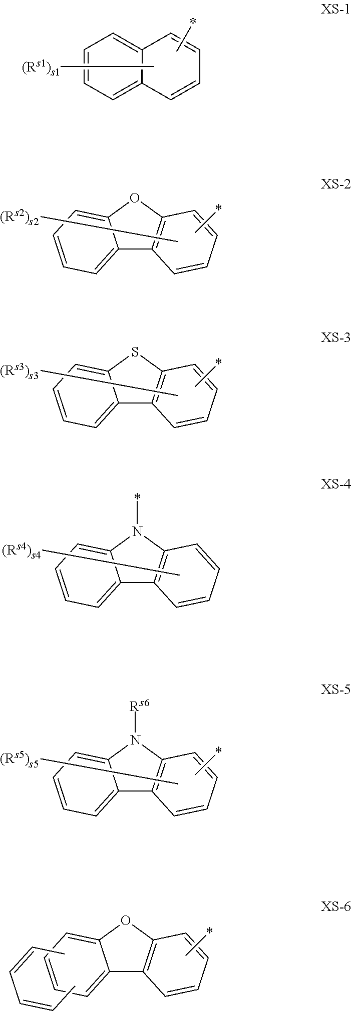

In XS-1 to XS-6, s1 to s3, and s5 may be each independently an integer of 0 to 7, and s4 may be an integer of 0 to 8. R^{s1} to R^{s6} may be each independently a hydrogen atom, a deuterium atom, a halogen atom, or a substituted or unsubstituted aryl group of 6 to 20 ring-forming carbon atoms. For example, each of R^{s1} to R^{s6} may be a hydrogen atom, a deuterium atom, a halogen atom, or a substituted or unsubstituted phenyl group. If each of R^{s1} to R^{s6} is a halogen atom, the amine compound of an embodiment may include a fluorine atom (F) as the halogen atom.

In some embodiments, a case where s1 is 0, may be the same as a case where s1 is 7, and R^{s1} is a hydrogen atom. A case where s2 is 0, may be the same as a case where s2 is 7, and R^{s2} is a hydrogen atom, and a case where s3 is 0, may be the same as a case where s3 is 7, and R^{s3} is a hydrogen atom. In addition, a case where s4 is 0, may be the same as a case where s4 is 8, and R^{s4} is a hydrogen atom, and a case where s5 is 0, may be the same as a case where s5 is 7, and R^{s5} is a hydrogen atom.

In some embodiments, if s1 is an integer of 2 or more, a plurality of R^{s1} may be all the same, or at least one may be different from the remainder. In some embodiments, if s1 is 0, XS-1 may be unsubstituted with R^{s1}. If s2 is an integer of 2 or more, a plurality of R^{s2} may be all the same, or at least one may be different from the remainder. In some embodiments, if s2 is 0, XS-2 may be unsubstituted with R^{s2}. If s3 is an integer of 2 or more, a plurality of R^{s3} may be all the same, or at least one may be different from the remainder. In some embodiments, if s3 is 0, XS-3 may be unsubstituted with R^{s3}. If s4 is an integer of 2 or more, a plurality of R^{s4} may be all the same, or at least one may be different from the remainder. In some embodiments, if s4 is 0, XS-4 may be unsubstituted with R^{s4}.

In Formula 1, FG may be represented by Formula 2-1 or Formula 2-2.

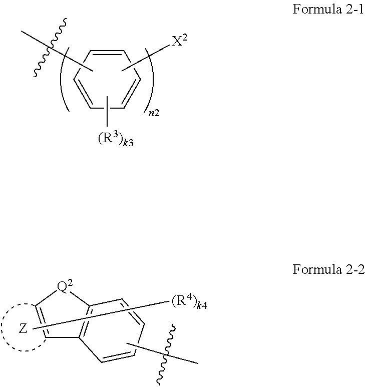

In Formula 2-1, n2 may be an integer of 1 to 3. For example, n2 may be 1 or 2. X² may be a substituted or unsubstituted naphthyl group, or a substituted or unsubstituted heteroaryl group of 2 to 30 ring-forming carbon atoms. For example, X² may be a naphthyl group substituted with a deuterium atom, a halogen atom, or a substituted or unsubstituted aryl group of 6 to 20 ring-forming carbon atoms, an unsubstituted naphthyl group, or a substituted or unsubstituted heteroaryl group of 2 to ring-forming carbon atoms, containing an oxygen atom (O) or a nitrogen atom (N) as a ring-forming atom. However, because

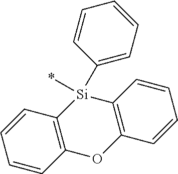 [SMILES: *[Si]1(c2ccccc2)c2ccccc2Oc2ccccc21]

is low in stability against holes and electrons, in the amine compound of an embodiment, a case where X² is

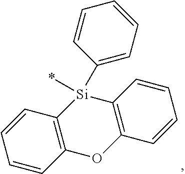 [SMILES: *[Si]1(c2ccccc2)c2ccccc2Oc2ccccc21]

may be excluded.

In some embodiments, X² may be represented by any one among XS-1 to XS-6. If X² is represented by any one among XS-1 to XS-6, the same contents explained referring to XS-1 to XS-6 may be applied for X².

In Formula 2-1 and Formula 2-2, R³ and R⁴ may be each independently a hydrogen atom, a deuterium atom, a halogen atom, a substituted or unsubstituted silyl group, a substituted or unsubstituted aryl group of 6 to 30 ring-forming carbon atoms, or a substituted or unsubstituted heteroaryl group of 2 to 30 ring-forming carbon atoms. In some embodiments, R³ and R⁴ may be each independently a hydrogen atom, a deuterium atom, a halogen atom, a substituted or unsubstituted aryl group of 6 to 15 ring-forming carbon atoms, or a substituted or unsubstituted heteroaryl group of 2 to 15 ring-forming carbon atoms. For example, R³ and R⁴ may be each independently a hydrogen atom, a deuterium atom, a halogen atom, a substituted or unsubstituted phenyl group, or a substituted or unsubstituted dibenzofuran group. In some embodiments, if each of R³ and R⁴ is a halogen atom, the amine compound of an embodiment may include a fluorine atom (F) as the halogen atom.

In Formula 2-1 and Formula 2-2, k3 may be an integer of 0 to 4, and k4 may be an integer of 0 to 7. A case where k3 is 0, may be the same as a case where k3 is 4, and R³ is a hydrogen atom. A case where k4 is 0, may be the same as a case where k4 is 7, and R⁴ is a hydrogen atom.

In some embodiments, if k3 is an integer of 2 or more, a plurality of R³ may be all the same, or at least one may be different from the remainder. In some embodiments, if k3 is 0, a phenylene group in Formula 2-1 may be unsubstituted with R³. If k4 is an integer of 2 or more, a plurality of R⁴ may be all the same, or at least one may be different from the remainder. In some embodiments, if k4 is 0, a fused ring substituent in Formula 2-2 may be unsubstituted with R⁴.

In Formula 2-2, Q² may be O, S, NR⁵, or CR⁶R⁷. R⁵ to R⁷ may be each independently a substituted or unsubstituted alkyl group of 1 to 20 carbon atoms, a substituted or unsubstituted aryl group of 6 to 50 ring-forming carbon atoms, or a substituted or unsubstituted heteroaryl group of 2 to 50 ring-forming carbon atoms. For example, each of R⁵ to R⁷ may be a substituted or unsubstituted phenyl group.

In Formula 2-2, Z may be a substituted or unsubstituted aromatic hydrocarbon ring of 6 to 30 ring-forming carbon atoms, or a substituted or unsubstituted aromatic heterocycle of 2 to 30 ring-forming carbon atoms. In some embodiments, Z may be a substituted or unsubstituted aromatic hydrocarbon ring of 6 to 10 ring-forming carbon atoms.

In Formula 2-2, the number of rings represented by Z may be 1 or 2. For example, in Formula 2-2, if the number of Z is 1, one ring at a portion represented by Z may form a fused ring, and if the number of Z is 2, two rings at a portion represented by Z may form a fused ring. In some embodiments, if the number of Z is 1, the fused ring represented by Formula 2-2 may be a substituent having three rings. In addition, if the number of Z is 2, the fused ring represented by Formula 2-2 may be a substituent having four rings.

The amine compound of an embodiment may include at least one substituted or unsubstituted naphthyl group as a substituent. For example, in Formula 1, if FG is represented by Formula 2-1, at least one among X¹ and X² may be a substituted or unsubstituted naphthyl group. In addition, if FG is represented by Formula 2-2, X¹ may be a substituted or unsubstituted naphthyl group.

In some embodiments, at least one among Ar¹, R¹, R², X¹, and FG in Formula 1 may include a deuterium atom, or a substituent including a deuterium atom. For example, the amine compound of an embodiment may include at least one deuterium atom as a substituent.

In some embodiments, Formula 2-1 may be represented by Formula 2-1a or Formula 2-1b. For example, the amine compound of an embodiment may be Formula 1 where FG is represented by Formula 2-1a or Formula 2-1b. In Formula 2-1a, the same contents explained referring to Formula 2-1 may be applied for n2. In Formula 2-1a and Formula 2-1b, the same contents explained referring to Formula 2-1 and Formula 2-2 may be applied for X².

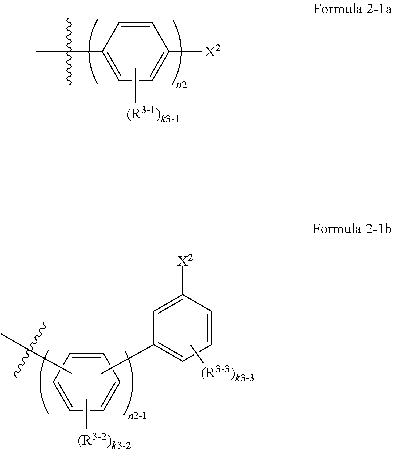

In Formula 2-1a and Formula 2-1b, R³⁻¹ to R³⁻³ may be each independently a hydrogen atom, a deuterium atom, a halogen atom, a substituted or unsubstituted aryl group or 6 to 15 ring-forming carbon atoms, or a substituted or unsubstituted heteroaryl group of 2 to 15 ring-forming carbon atoms. For example, each of R³⁻¹ to R³⁻³ may be a hydrogen atom, a deuterium atom, a halogen atom, or a substituted or unsubstituted phenyl group. In some embodiments, if each of R³⁻¹ to R³⁻³ is a halogen atom, the amine compound of an embodiment may include a fluorine atom (F) as the halogen atom.

In Formula 2-1b, n2-1 may be an integer of 0 to 2. For example, n2-1 may be 0 or 1. If n2-1 is 0, a phenyl group combined with X² and substituted or unsubstituted with R³⁻³ in Formula 2-1b may be directly bonded to the nitrogen atom of Formula 1.

In Formula 2-1a and Formula 2-1b, k3-1 to k3-3 may be each independently an integer of 0 to 4. A case where k3-1 is 0, may be the same as a case where k3-1 is 4, and R³⁻¹ is a hydrogen atom. A case where k3-2 is 0, may be the same as a case where k3-2 is 4, and R³⁻² is a hydrogen atom. A case where k3-3 is 0, may be the same as a case where k3-3 is 4, and R³⁻³ is a hydrogen atom.

In some embodiments, if k3-1 is an integer of 2 or more, a plurality of R³⁻¹ may be all the same, or at least one may be different from the remainder. In some embodiments, if k3-1 is 0, a phenylene group in Formula 2-1a may be unsubstituted with R³⁻¹. If k3-2 is an integer of 2 or more, a plurality of R³⁻² may be all the same, or at least one may be different from the remainder. In some embodiments, if k3-2 is 0, a phenylene group in Formula 2-1b may be unsubstituted with R³⁻². If k3-3 is an integer of 2 or more, a plurality of R³⁻³ may be all the same, or at least one may be different from the remainder. In some embodiments, if k3-3 is 0, a phenylene group in Formula 2-1b may be unsubstituted with R³⁻³.

In some embodiments, Formula 2-2 may be represented by Formula 2-2a to Formula 2-2c below. For example, the amine compound of an embodiment may be Formula 1 where FG is represented by any one among Formula 2-2a to Formula 2-2c. In Formula 2-2a to Formula 2-2c, the same contents explained referring to Formula 2-2 may be applied for Q².

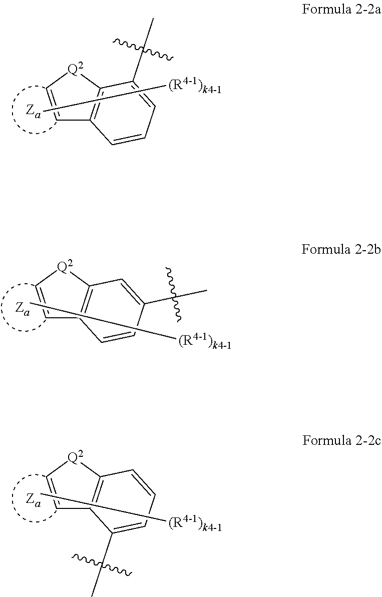

In Formula 2-2a to Formula 2-2c, Zₐ may be a substituted or unsubstituted aromatic hydrocarbon ring of 6 to 10 ring-forming carbon atoms. In some embodiments, the number of rings represented by Zₐ may be 1 or 2. For example, if the number of Zₐ is 1, one ring at a portion represented by Zₐ may form a fused ring, and if the number of Zₐ is 2, two rings at a portion represented by Zₐ may form a fused ring. In some embodiments, if the number of Zₐ is 1, the fused rings represented by Formula 2-2a and Formula 2-2b may be substituents having three rings. In addition, if the number of Zₐ is 2, the fused rings represented by Formula 2-2a to Formula 2-2c may be substituents having four rings.

In Formula 2-2a to Formula 2-2c, each R⁴⁻¹ may be independently a hydrogen atom, a deuterium atom, a halogen atom, or a substituted or unsubstituted aryl group of 6 to 15 ring-forming carbon atoms. For example, R⁴⁻¹ may be a hydrogen atom, a deuterium atom, a halogen atom, or a substituted or unsubstituted phenyl group. In some embodiments, if R⁴⁻¹ is a halogen atom, the amine compound of an embodiment may include a fluorine atom (F) as the halogen atom.

In Formula 2-2a to Formula 2-2c, k4-1 may be an integer of 0 to 7. A case where k4-1 is 0, may be the same as a case where k4-1 is 7, and R⁴⁻¹ is a hydrogen atom. In some embodiments, if k4-1 is an integer of 2 or more, a plurality of R⁴⁻¹ may be all the same, or at least one may be different from the remainder. In some embodiments, if k4-1 is 0, the fused ring substituents of Formula 2-2a to Formula 2-2c, may be unsubstituted with R⁴⁻¹.

In some embodiments, in the amine compound represented by Formula 1, FG may be represented by any one among Formula FG-1 to Formula FG-6 below. In Formula FG-1 to Formula FG-6, the same explanation referring to Formula 2-1 and Formula 2-2 may be applied for X² and Q². For example, in Formula FG-1 to Formula FG-4, the same explanation referring to Formula 2-1 may be applied for X², and in Formula FG-5 and Formula FG-6, the same explanation referring to Formula 2-2 may be applied for Q².

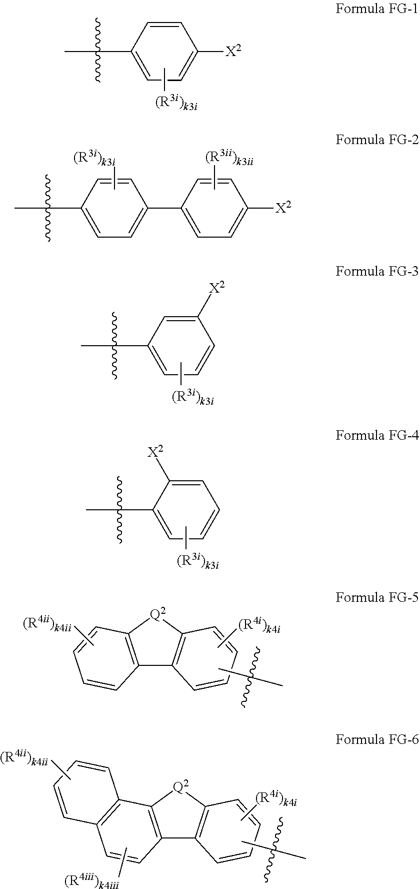

In Formula FG-1 to Formula FG-6, R³ⁱ, R³ⁱⁱ, and R¹ to R⁴ⁱⁱⁱ may be each independently a hydrogen atom, a deuterium atom, a halogen atom, or a substituted or unsubstituted phenyl group. For example, R³ⁱ and R³ⁱⁱ may be each independently a hydrogen atom, a deuterium atom, or a halogen atom, and R⁴ⁱ to R⁴ⁱⁱⁱ may be each independently a hydrogen atom, a deuterium atom, a halogen atom, or a substituted or unsubstituted phenyl group. In some embodiments, if each of R³ⁱ, R³ⁱⁱ, and R⁴ⁱ to R⁴ⁱⁱⁱ is a halogen atom, the amine compound of an embodiment may include a fluorine atom (F) as the halogen atom.

In Formula FG-1 to Formula FG-6, k3i, k3ii, and k4ii may be each independently an integer of 0 to 4, k4i may be an integer of 0 to 3, and k4iii may be an integer of 0 to 2. A case where k3i is 0, may be the same as a case where k3i is 4, and R³ⁱ is a hydrogen atom. Such explanation may be applied for cases where each of k3ii, and k4i to k4iii is 0.

In some embodiments, if each of k3i, k3ii, and k4i to k4iii is an integer of 2 or more, each of a plurality of R³ⁱ, R³ⁱⁱ, R⁴ⁱ, to R⁴ⁱⁱⁱ may be the same, or at least one may be different from the remainder. In some embodiments, if k3i, k3ii, and k4i to k4iii are 0, the amine compounds of embodiments may not include the substituents of R³ⁱ, R³ⁱⁱ, R⁴ⁱ, R⁴ⁱⁱ and/or R⁴ⁱⁱⁱ, respectively.

In some embodiments, the amine compound of an embodiment, represented by Formula 1 may be represented by Formula 3-1 or Formula 3-2. Formula 3-1 and Formula 3-2 may correspond to Formula 1 where Ar¹ and X¹ are embodied. In Formula 3-1 and Formula 3-2, the same contents explained referring to Formula 1 may be applied for O¹, R¹, R², n1, k1, k2, and FG.

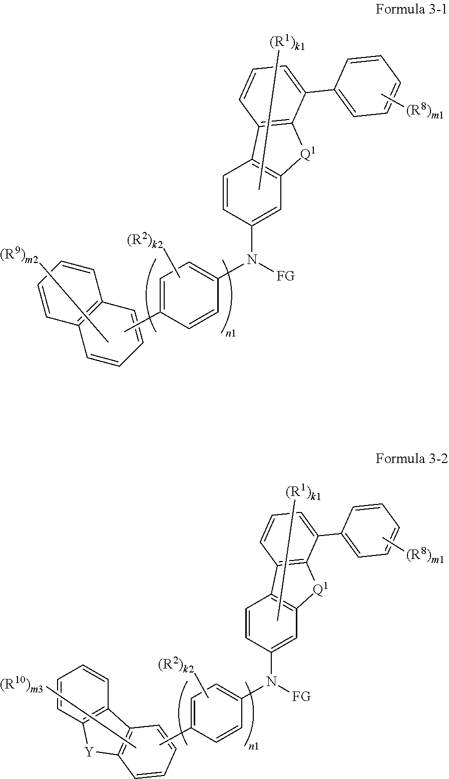

In Formula 3-2, Y may be O or NR¹¹. For example, if Y is O, the amine compound of an embodiment may include a substituted or unsubstituted dibenzofuran group as a substituent. In addition, if Y is NR¹¹, the amine compound of an embodiment may include a carbazole group substituted with R¹¹, or an unsubstituted carbazole group, as a substituent.

In Formula 3-1 and Formula 3-2, R⁸ to R¹¹ may be each independently a hydrogen atom, a deuterium atom, a halogen atom, or a substituted or unsubstituted aryl group of 6 to 20 ring-forming carbon atoms. For example, each of R⁸ to R¹¹ may be a hydrogen atom, a deuterium atom, a halogen atom, or a substituted or unsubstituted phenyl group. In some embodiments, if each of R⁸ to R¹¹ is a halogen atom, the amine compounds of embodiments, represented by Formula 3-1 and Formula 3-2 may include a fluorine atom (F) as the halogen atom.

In Formula 3-1 and Formula 3-2, m1 may be an integer of 0 to 5, m2 and m3 may be each independently an integer of 0 to 7. A case where m1 is 0, may be the same as a case where m1 is 5, and R⁸ is a hydrogen atom. A case where m2 is 0, may be the same as a case where m2 is 7, and R⁹ is a hydrogen atom. A case where m3 is 0, may be the same as a case where m3 is 7, and R¹⁰ is a hydrogen atom.

In some embodiments, if m1 is an integer of 2 or more, a plurality of R⁸ may be all the same, or at least one may be different from the remainder. In some embodiments, if m1 is 0, the amine compound represented by Formula 3-1 or Formula 3-2 may be unsubstituted with R⁸. If m2 is an integer of 2 or more, a plurality of R⁹ may be all the same, or at least one may be different from the remainder. In some embodiments, if m2 is 0, the amine compound represented by Formula 3-1 may be unsubstituted with R⁹. If m3 is an integer of 2 or more, a plurality of R¹⁰ may be all the same, or at least one may be different from the remainder. In some embodiments, if m3 is 0, the amine compound represented by Formula 3-2 may be unsubstituted with R¹⁰.

In some embodiments, the amine compound represented by Formula 1 may be represented by Formula 4-1 or Formula 4-2 below. Formula 4-1 and Formula 4-2 correspond to Formula 1 where n1, R² and FG are embodied. In Formula 4-1 and Formula 4-2, the same contents explained in Formula 1, Formula 2-1 and Formula 2-2 may be applied for Ar¹, R¹, X¹, X², Q¹, Q², Z, and k1.

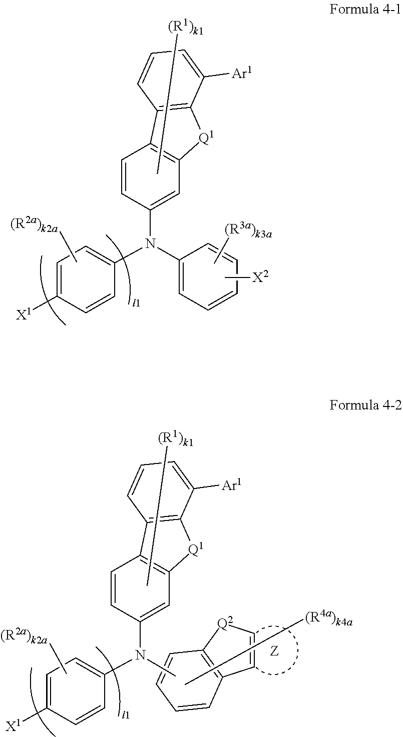

In Formula 4-1 and Formula 4-2, l1 may be 1 or 2. R^{2a} to R^{4a} may be each independently a hydrogen atom, a deuterium atom, a halogen atom, or a substituted or unsubstituted phenyl group. For example, R^{2a} and R^{3a} may be each independently a hydrogen atom, a deuterium atom, or a halogen atom, and R^{4a} may be a hydrogen atom, a deuterium atom, a halogen atom, or a substituted or unsubstituted phenyl group.

In Formula 4-1 and Formula 4-2, k2a and k3a may be each independently an integer of 0 to 4, and k4a may be an integer of 0 to 7. A case where k2a is 0, may be the same as a case where k2a is 4, and R^{2a} is a hydrogen atom. Such explanation may be applied for cases where each of k3a and k4a is 0.

In some embodiments, if each of k2a to k4a is an integer of 2 or more, each of a plurality of R^{2a}, R^{3a} and R^{4a} may be the same, or at least one may be different from the remainder. In some embodiments, if k2a to k4a are 0, the amine compounds of embodiments may not include the substituents of R^{2a}, R^{3a} and R^{4a}, respectively.

The amine compound of an embodiment, represented by Formula 1 may be represented by any one among the compounds in Compound Group 1 below. The hole transport region HTR of the light emitting element ED of an embodiment may include at least one among the amine compounds disclosed in Compound Group 1. For example, the electron blocking layer EBL of the light emitting element ED may include at least one among the amine compounds disclosed in Compound Group 1. In Compound Group 1, “D” is a deuterium atom.

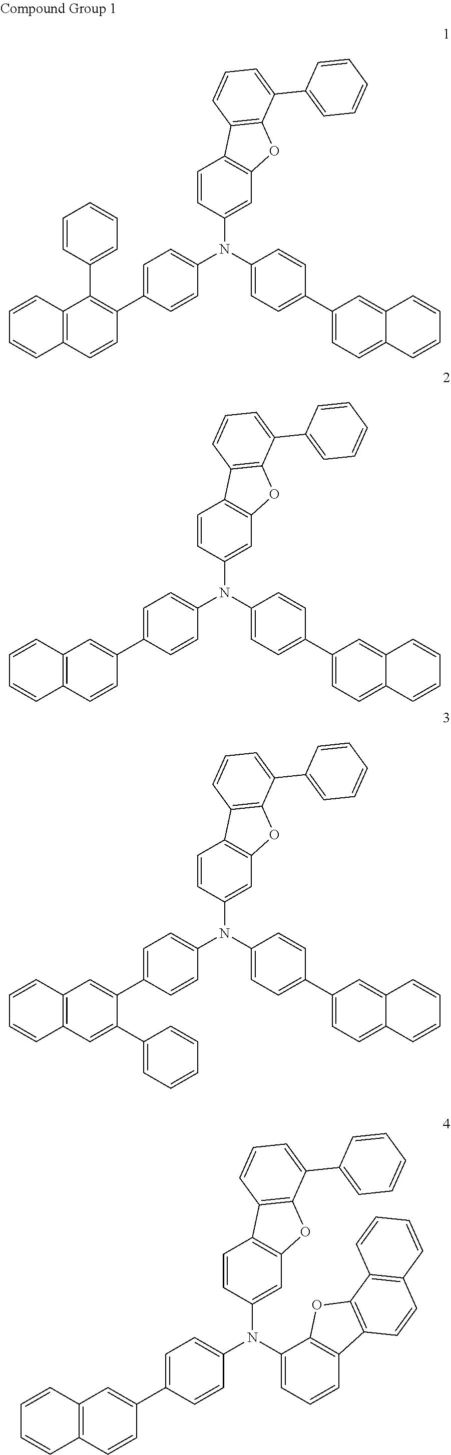

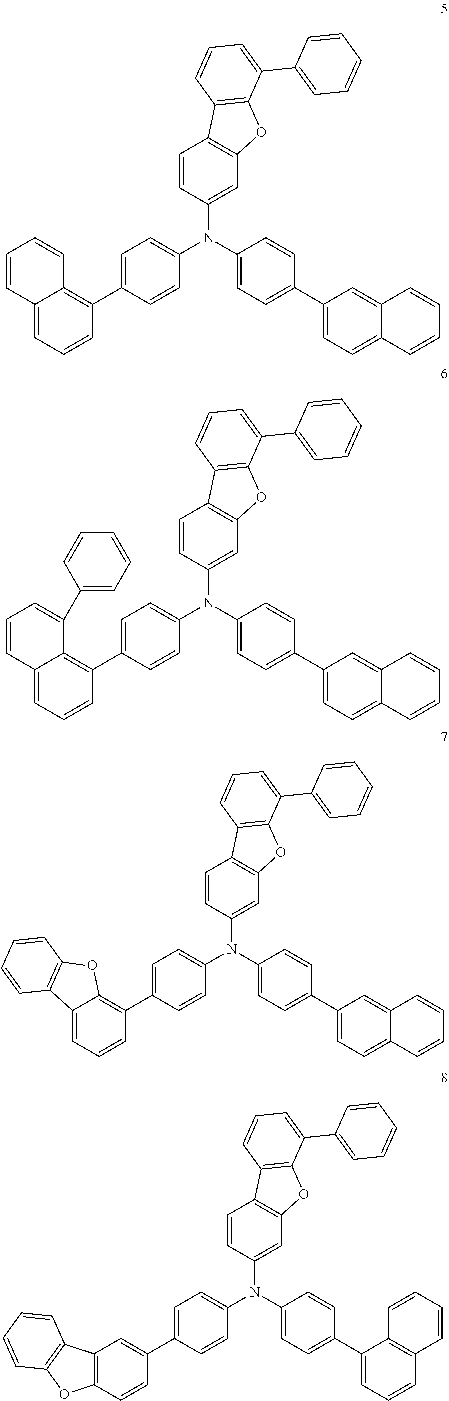

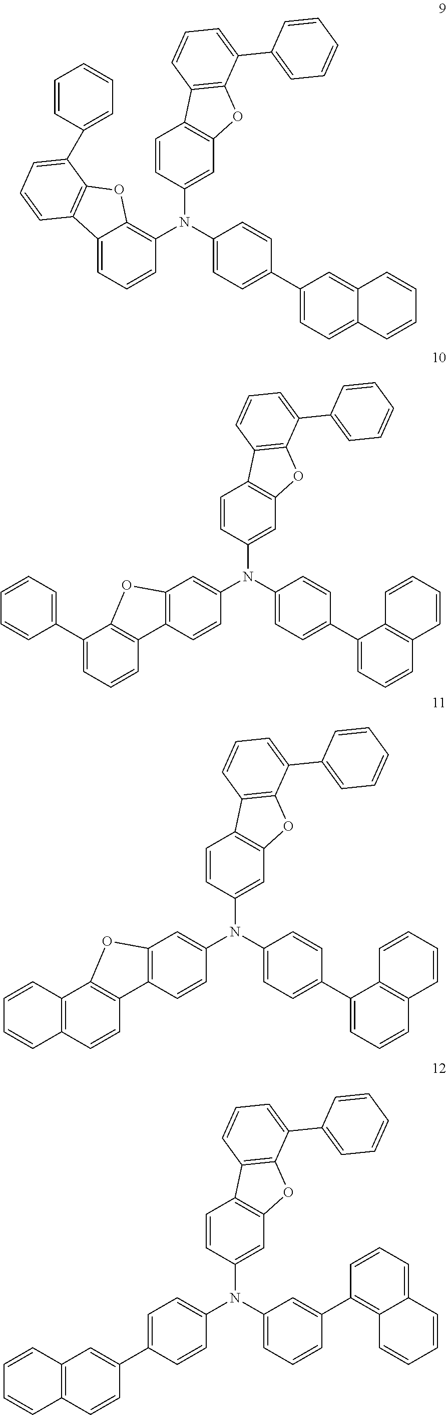

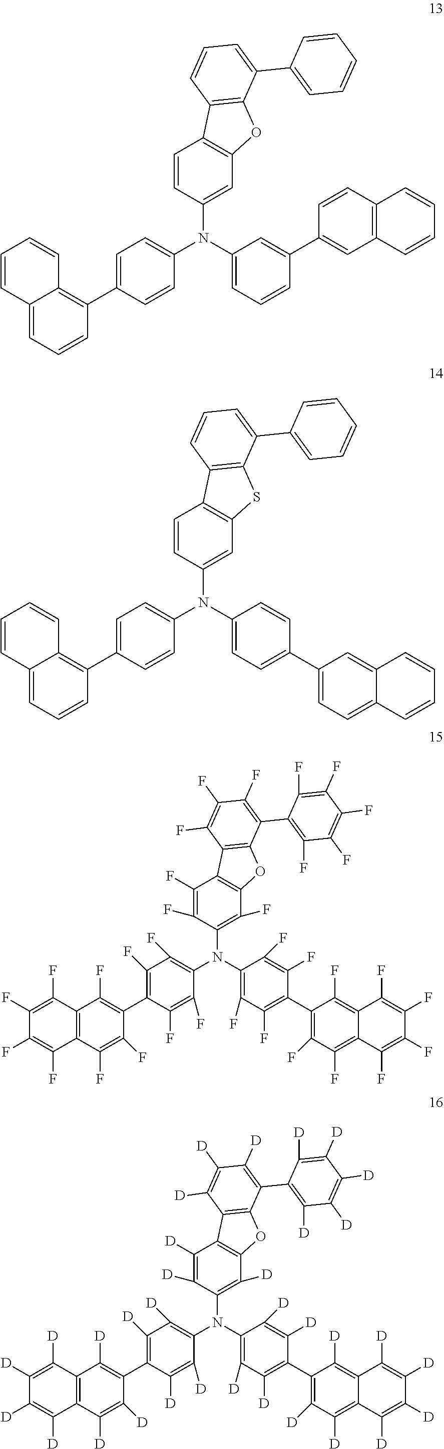

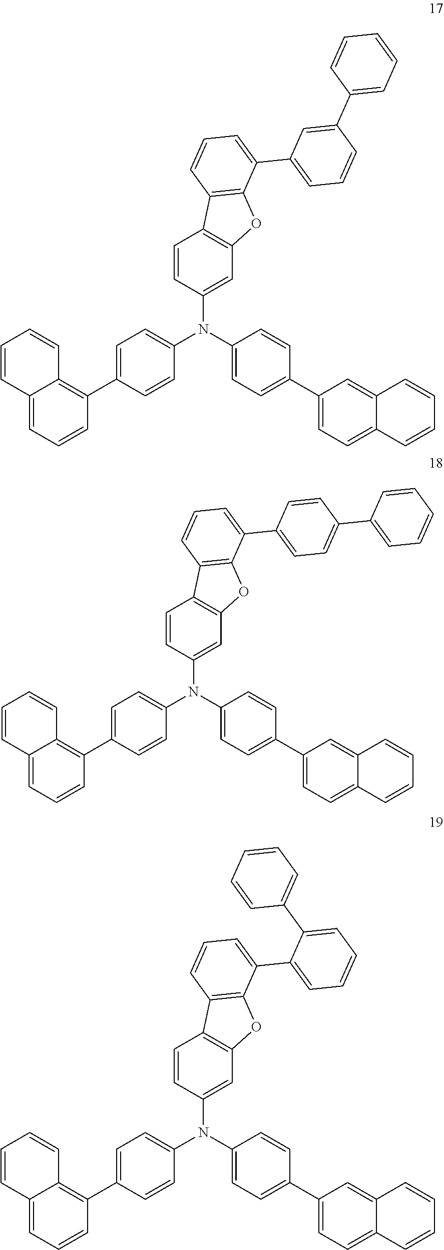

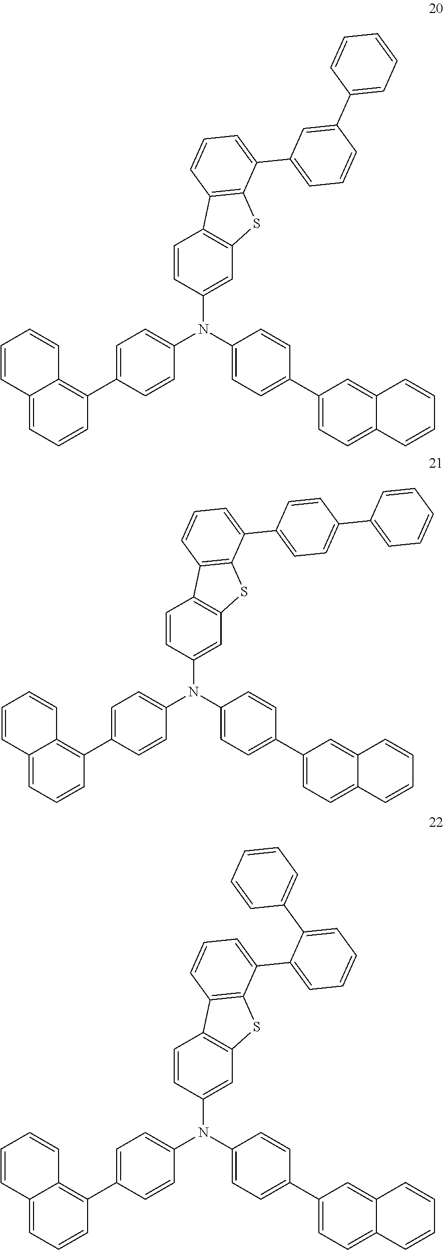

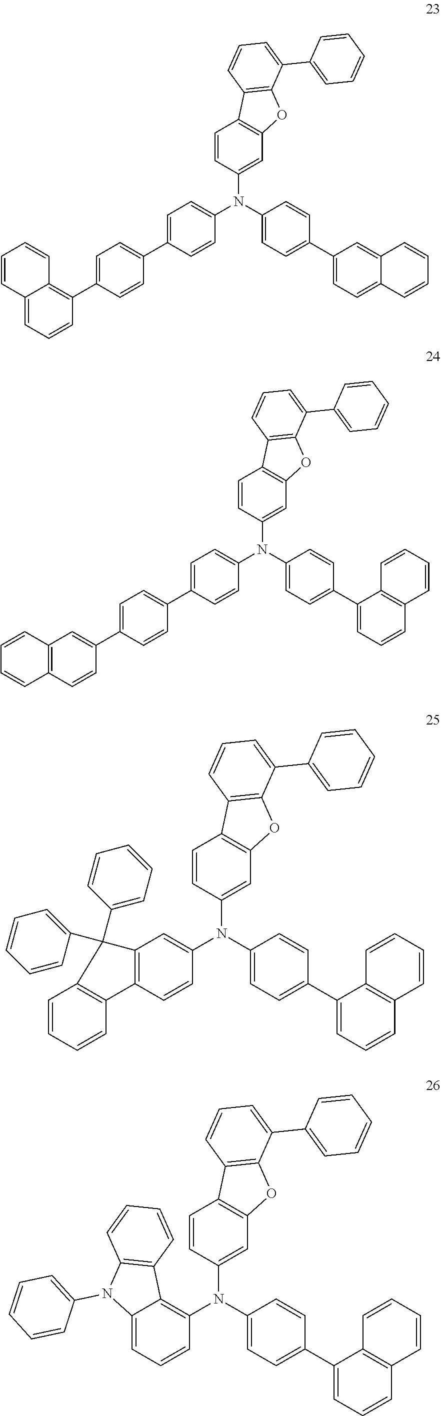 [SMILES: c1ccc(-c2cccc3c2oc2cc(N(c4ccc(-c5ccc(-c6cccc7ccccc67)cc5)cc4)c4ccc(-c5ccc6ccccc6c5)cc4)ccc23)cc1 ; c1ccc(-c2cccc3c2oc2cc(N(c4ccc(-c5ccc(-c6ccc7ccccc7c6)cc5)cc4)c4ccc(-c5cccc6ccccc56)cc4)ccc23)cc1 ; c1ccc(-c2cccc3c2oc2cc(N(c4ccc(-c5cccc6ccccc56)cc4)c4ccc5c(c4)C(c4ccccc4)(c4ccccc4)c4ccccc4-5)ccc23)cc1 ; c1ccc(-c2cccc3c2oc2cc(N(c4ccc(-c5cccc6ccccc56)cc4)c4cccc5c4c4ccccc4n5-c4ccccc4)ccc23)cc1]

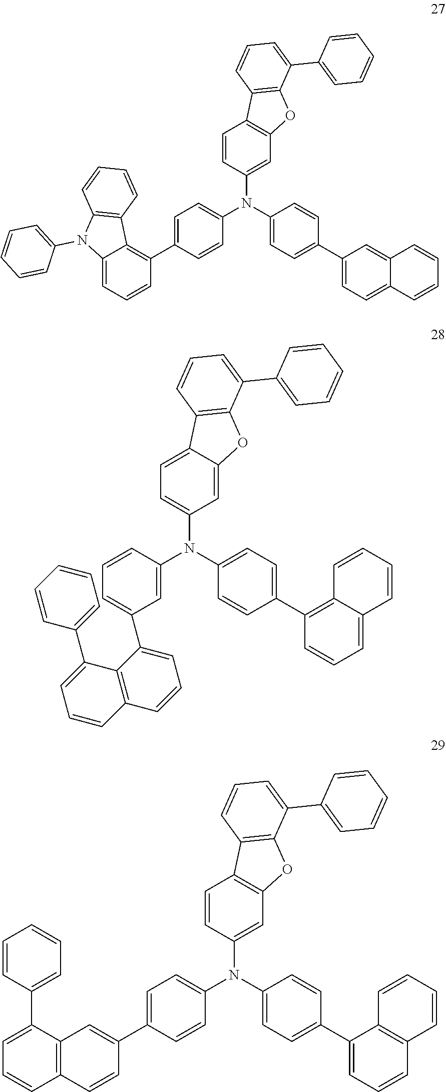 [SMILES: c1ccc(-c2cccc3c2oc2cc(N(c4ccc(-c5ccc6ccccc6c5)cc4)c4ccc(-c5cccc6c5c5ccccc5n6-c5ccccc5)cc4)ccc23)cc1 ; c1ccc(-c2cccc3c2oc2cc(N(c4ccc(-c5cccc6ccccc56)cc4)c4cccc(-c5cccc6cccc(-c7ccccc7)c56)c4)ccc23)cc1 ; c1ccc(-c2cccc3ccc(-c4ccc(N(c5ccc(-c6cccc7ccccc67)cc5)c5ccc6c(c5)oc5c(-c7ccccc7)cccc56)cc4)cc23)cc1]

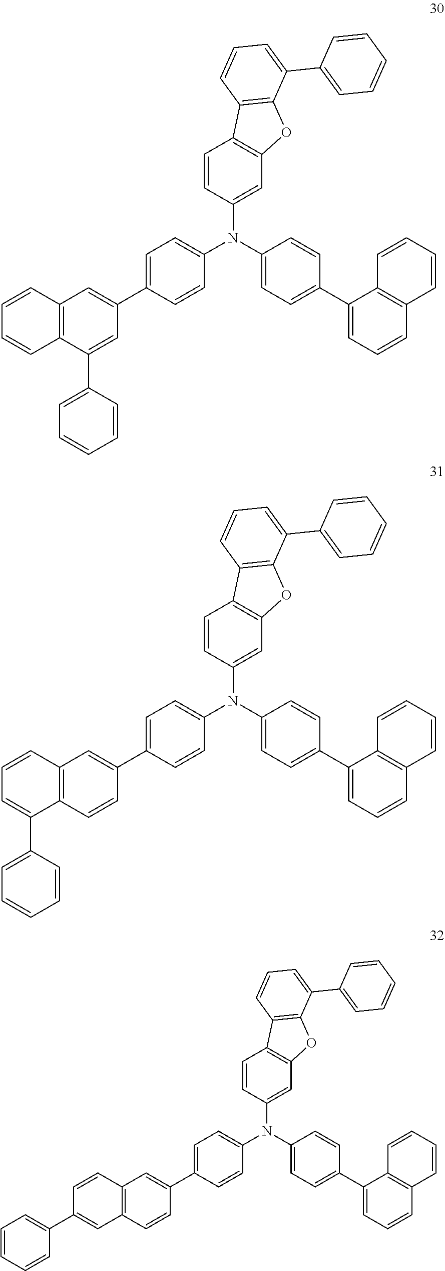 [SMILES: c1ccc(-c2cc(-c3ccc(N(c4ccc(-c5cccc6ccccc56)cc4)c4ccc5c(c4)oc4c(-c6ccccc6)cccc45)cc3)cc3ccccc23)cc1 ; c1ccc(-c2cccc3cc(-c4ccc(N(c5ccc(-c6cccc7ccccc67)cc5)c5ccc6c(c5)oc5c(-c7ccccc7)cccc56)cc4)ccc23)cc1 ; c1ccc(-c2ccc3cc(-c4ccc(N(c5ccc(-c6cccc7ccccc67)cc5)c5ccc6c(c5)oc5c(-c7ccccc7)cccc56)cc4)ccc3c2)cc1]

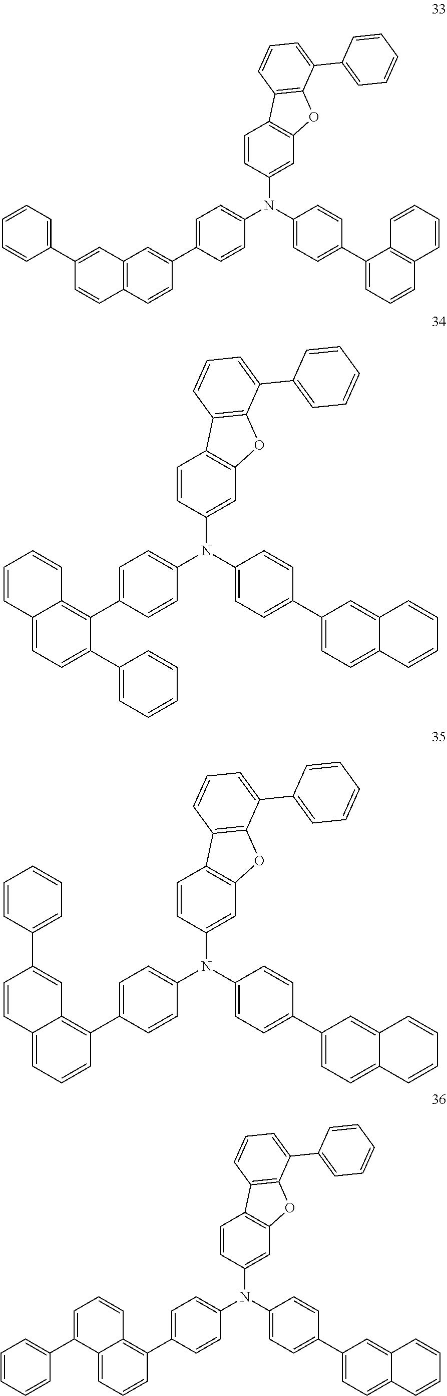

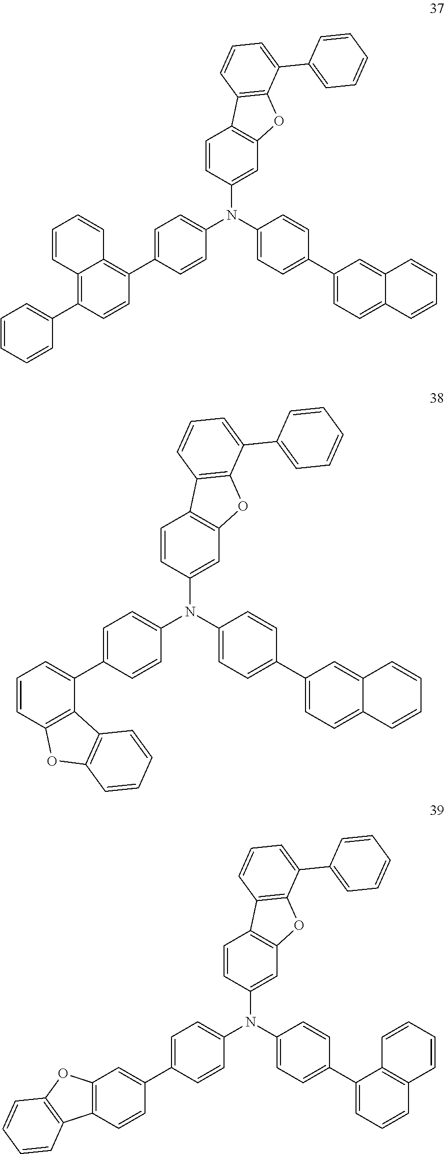 [SMILES: c1ccc(-c2ccc(-c3ccc(N(c4ccc(-c5ccc6ccccc6c5)cc4)c4ccc5c(c4)oc4c(-c6ccccc6)cccc45)cc3)c3ccccc23)cc1 ; c1ccc(-c2cccc3c2oc2cc(N(c4ccc(-c5ccc6ccccc6c5)cc4)c4ccc(-c5cccc6oc7ccccc7c56)cc4)ccc23)cc1 ; c1ccc(-c2cccc3c2oc2cc(N(c4ccc(-c5ccc6c(c5)oc5ccccc56)cc4)c4ccc(-c5cccc6ccccc56)cc4)ccc23)cc1]

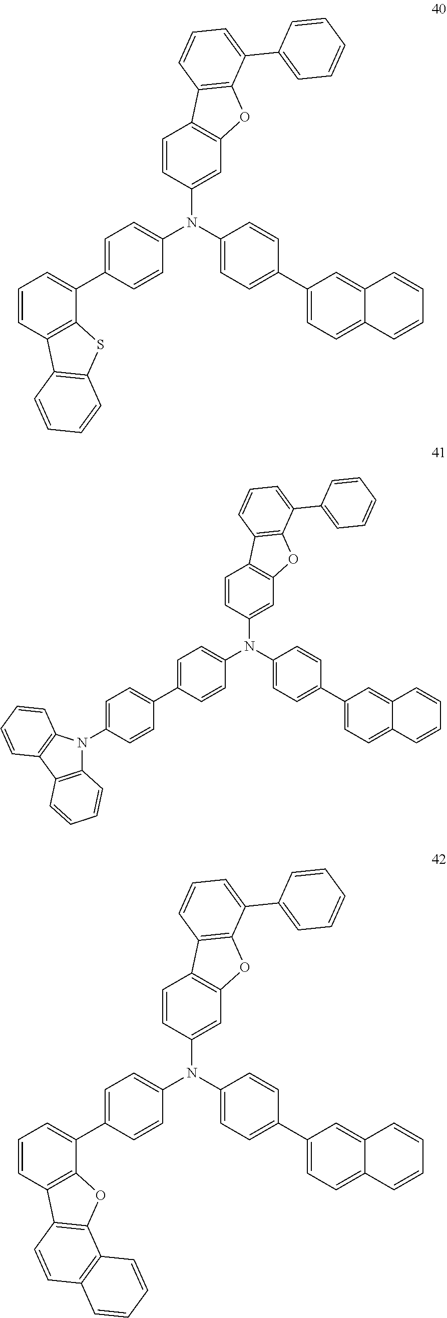 [SMILES: c1ccc(-c2cccc3c2oc2cc(N(c4ccc(-c5ccc6ccccc6c5)cc4)c4ccc(-c5cccc6c5sc5ccccc56)cc4)ccc23)cc1 ; c1ccc(-c2cccc3c2oc2cc(N(c4ccc(-c5ccc(-n6c7ccccc7c7ccccc76)cc5)cc4)c4ccc(-c5ccc6ccccc6c5)cc4)ccc23)cc1 ; c1ccc(-c2cccc3c2oc2cc(N(c4ccc(-c5ccc6ccccc6c5)cc4)c4ccc(-c5cccc6c5oc5c7ccccc7ccc65)cc4)ccc23)cc1]

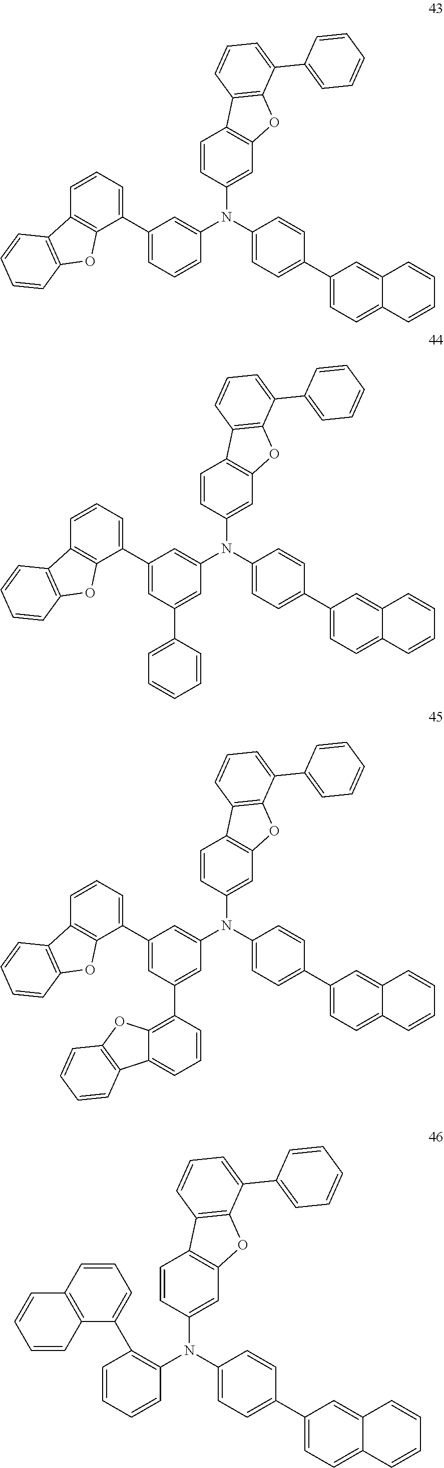 [SMILES: c1ccc(-c2cccc3c2oc2cc(N(c4ccc(-c5ccc6ccccc6c5)cc4)c4cccc(-c5cccc6c5oc5ccccc56)c4)ccc23)cc1 ; c1ccc(-c2cc(-c3cccc4c3oc3ccccc34)cc(N(c3ccc(-c4ccc5ccccc5c4)cc3)c3ccc4c(c3)oc3c(-c5ccccc5)cccc34)c2)cc1 ; c1ccc(-c2cccc3c2oc2cc(N(c4ccc(-c5ccc6ccccc6c5)cc4)c4cc(-c5cccc6c5oc5ccccc56)cc(-c5cccc6c5oc5ccccc56)c4)ccc23)cc1 ; c1ccc(-c2cccc3c2oc2cc(N(c4ccc(-c5ccc6ccccc6c5)cc4)c4ccccc4-c4cccc5ccccc45)ccc23)cc1]

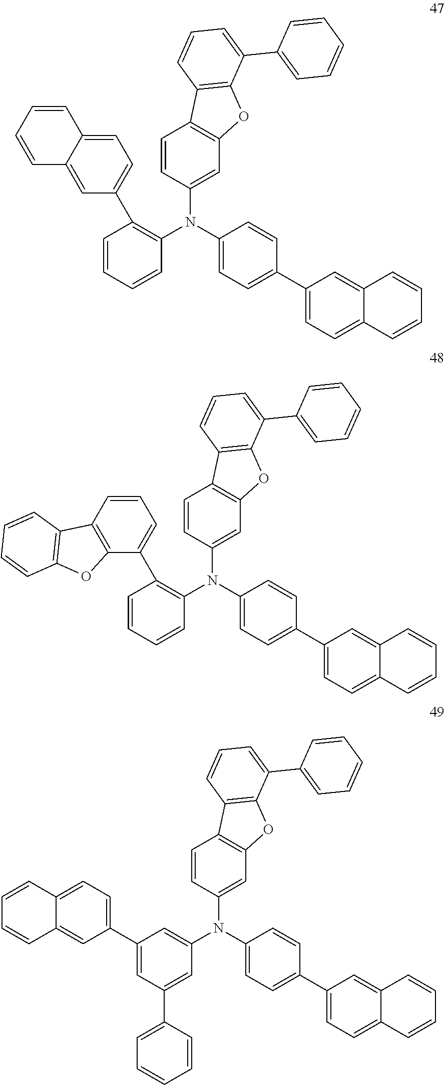

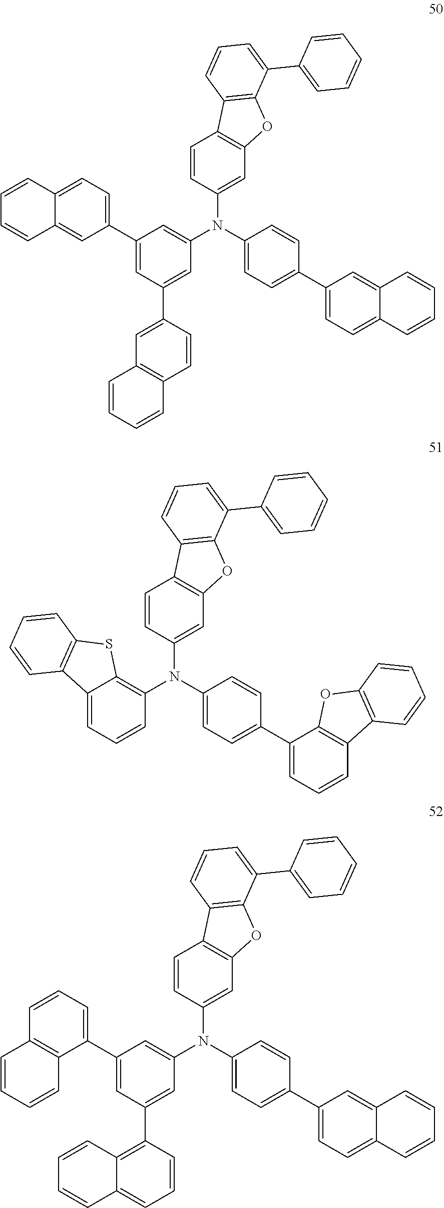 [SMILES: c1ccc(-c2cccc3c2oc2cc(N(c4ccc(-c5ccc6ccccc6c5)cc4)c4cc(-c5ccc6ccccc6c5)cc(-c5ccc6ccccc6c5)c4)ccc23)cc1 ; c1ccc(-c2cccc3c2oc2cc(N(c4ccc(-c5cccc6c5oc5ccccc56)cc4)c4cccc5c4sc4ccccc45)ccc23)cc1 ; c1ccc(-c2cccc3c2oc2cc(N(c4ccc(-c5ccc6ccccc6c5)cc4)c4cc(-c5cccc6ccccc56)cc(-c5cccc6ccccc56)c4)ccc23)cc1]

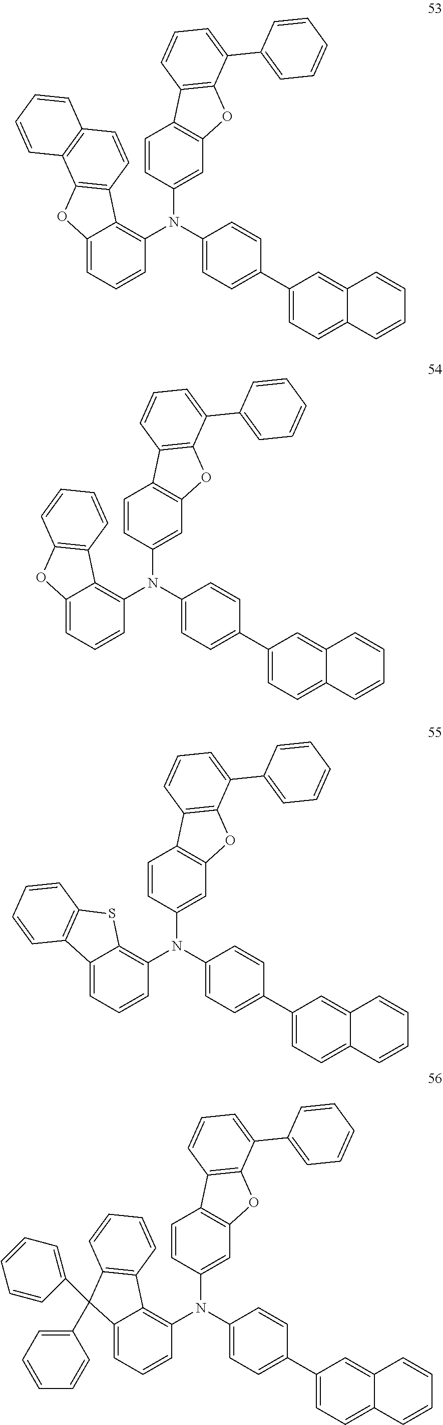

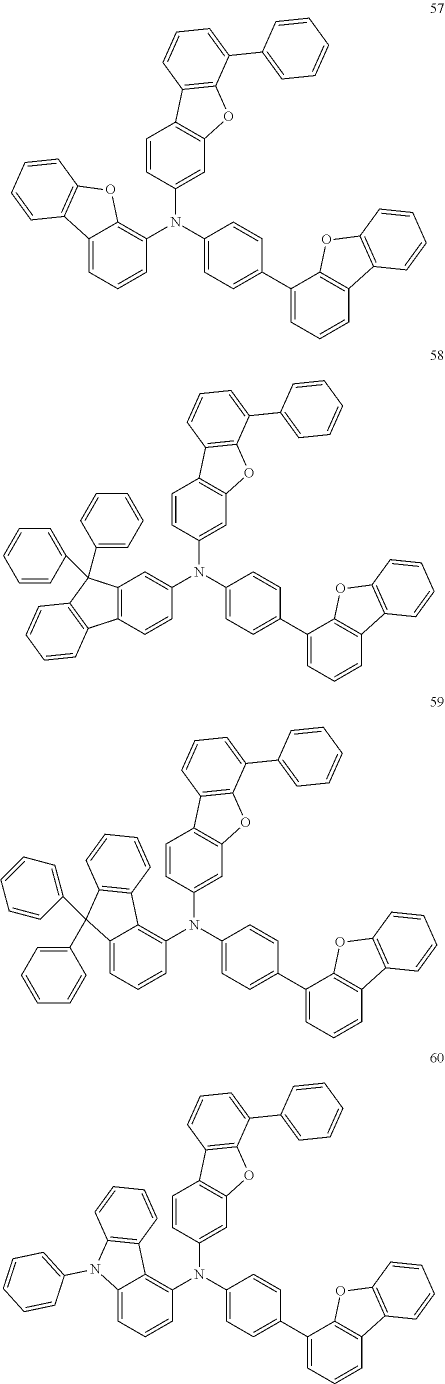

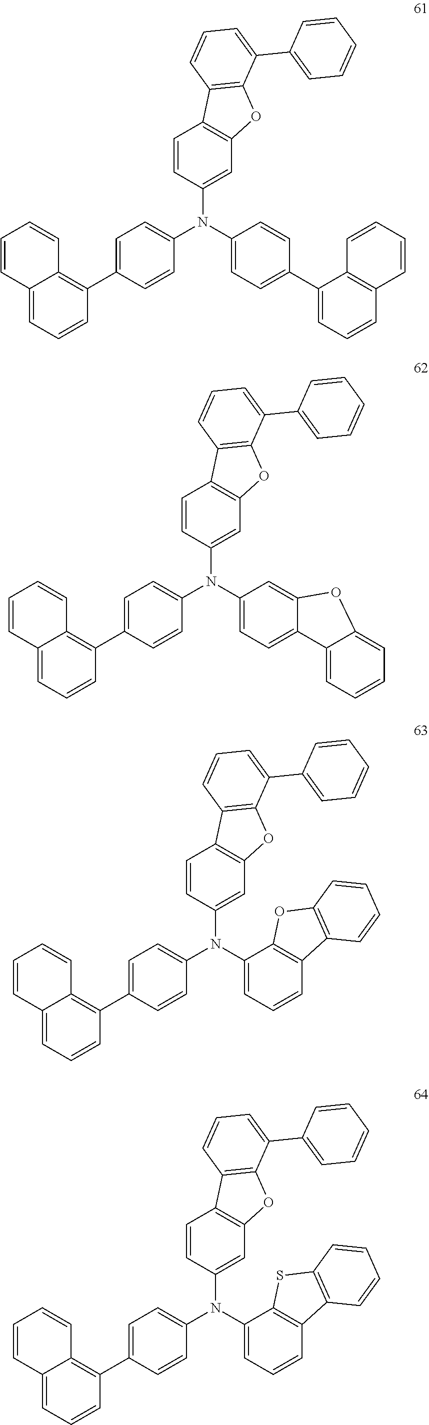

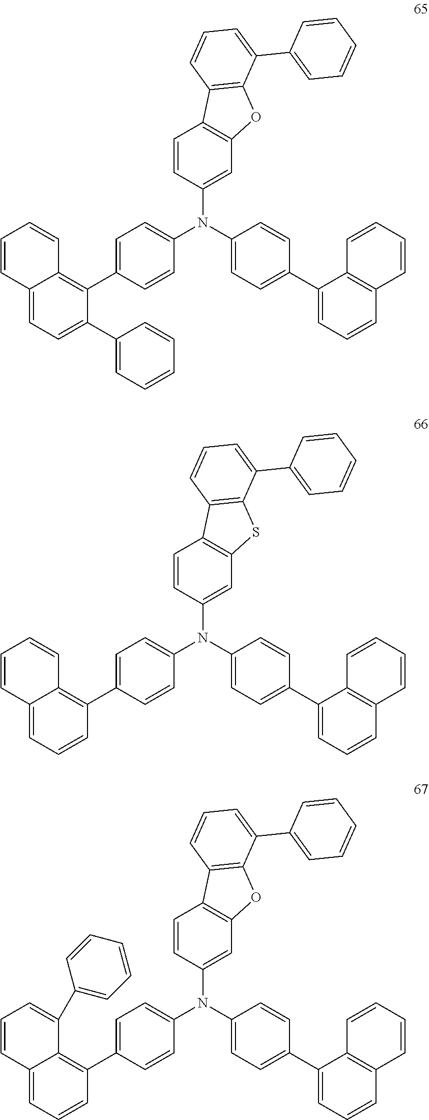

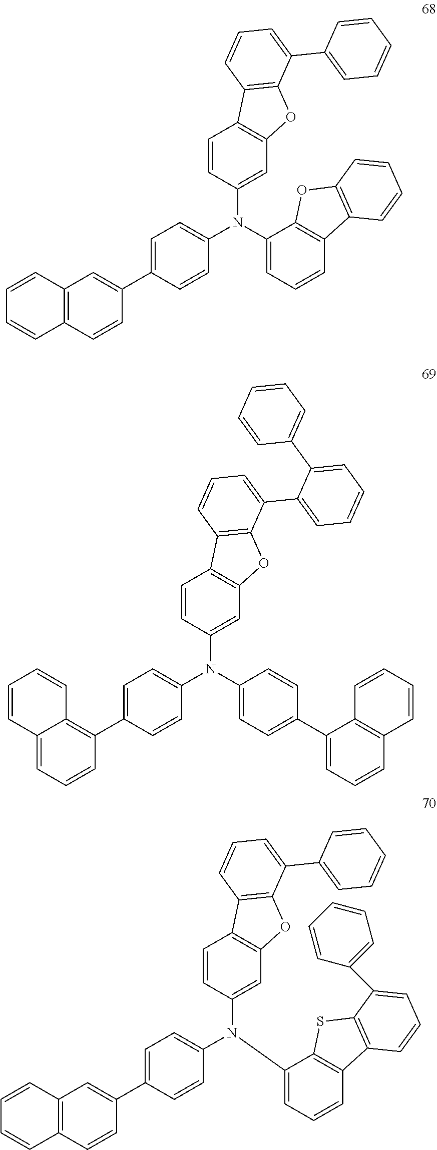

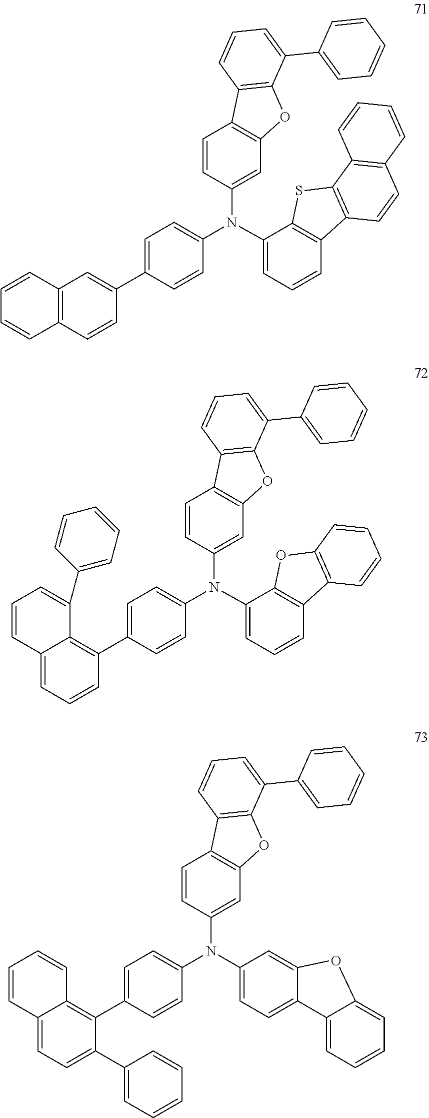 [SMILES: c1ccc(-c2cccc3c2oc2cc(N(c4ccc(-c5ccc6ccccc6c5)cc4)c4cccc5c4sc4c6ccccc6ccc54)ccc23)cc1 ; c1ccc(-c2cccc3c2oc2cc(N(c4ccc(-c5cccc6cccc(-c7ccccc7)c56)cc4)c4cccc5c4oc4ccccc45)ccc23)cc1 ; c1ccc(-c2ccc3ccccc3c2-c2ccc(N(c3ccc4c(c3)oc3ccccc34)c3ccc4c(c3)oc3c(-c5ccccc5)cccc34)cc2)cc1]

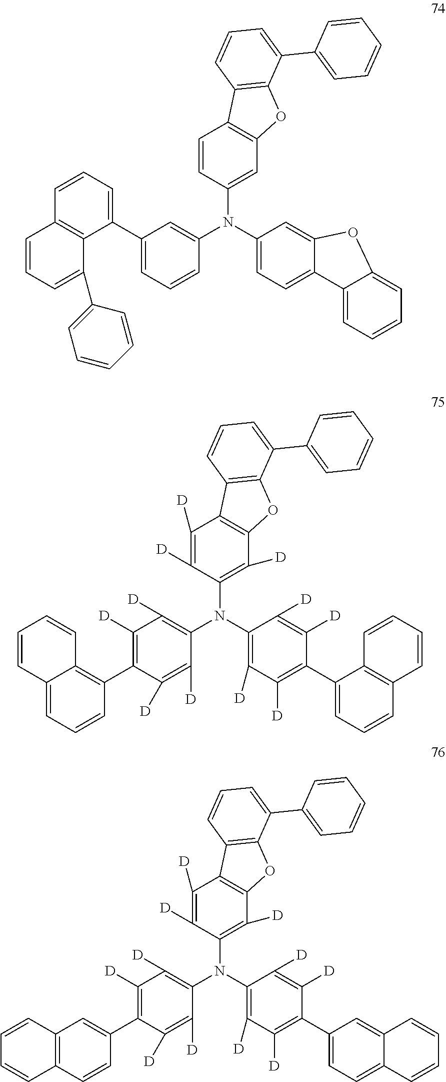 [SMILES: c1ccc(-c2cccc3c2oc2cc(N(c4cccc(-c5cccc6cccc(-c7ccccc7)c56)c4)c4ccc5c(c4)oc4ccccc45)ccc23)cc1 ; [2H]c1c([2H])c(N(c2c([2H])c([2H])c(-c3cccc4ccccc34)c([2H])c2[2H])c2c([2H])c([2H])c3c(oc4c(-c5ccccc5)cccc43)c2[2H])c([2H])c([2H])c1-c1cccc2ccccc12 ; [2H]c1c([2H])c(N(c2c([2H])c([2H])c(-c3ccc4ccccc4c3)c([2H])c2[2H])c2c([2H])c([2H])c3c(oc4c(-c5ccccc5)cccc43)c2[2H])c([2H])c([2H])c1-c1ccc2ccccc2c1]

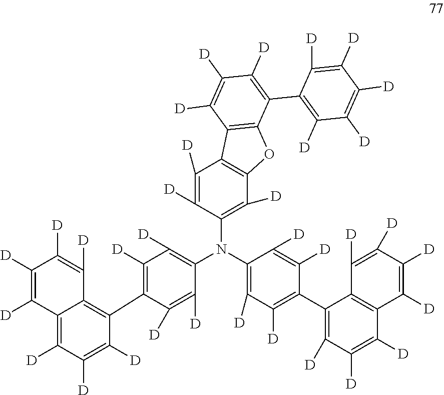 [SMILES: [2H]c1c([2H])c([2H])c(-c2c([2H])c([2H])c([2H])c3c2oc2c([2H])c(N(c4c([2H])c([2H])c(-c5c([2H])c([2H])c([2H])c6c([2H])c([2H])c([2H])c([2H])c56)c([2H])c4[2H])c4c([2H])c([2H])c(-c5c([2H])c([2H])c([2H])c6c([2H])c([2H])c([2H])c([2H])c56)c([2H])c4[2H])c([2H])c([2H])c23)c([2H])c1[2H]]

The amine compound of an embodiment may be a tertiary amine compound. For example, the amine compound of an embodiment may include a first substituent, a second substituent, and a third substituent.

The first substituent may be a substituent having a dibenzoheterole skeleton. For example, the first substituent may include a dibenzofuran group or a dibenzothiophene group, combined with a substituted or unsubstituted phenyl group at position 6, for example, may be characterized in directly bonding to the nitrogen atom of an amine at position 3 of the dibenzofuran group or dibenzothiophene group. The amine compound of an embodiment includes the first substituent, and accordingly, may not induce destabilization by three-dimensional twist around the amine and may have high stability. Accordingly, by suitably controlling a LUMO level, the amine compound may be suitably used as a material of an electron blocking layer EBL.

The second substituent and the third substituent may be each independently a substituted or unsubstituted naphthyl group, or a substituted or unsubstituted heteroaryl group, bonded to the nitrogen atom of an amine via a linker or direct linkage. In the amine compound of an embodiment, at least one among the second substituent and the third substituent may be a substituted or unsubstituted naphthyl group, or a substituted or unsubstituted heteroaryl group, bonded to the nitrogen atom of an amine via a linker in para relation (e.g., at a para bonding position). Accordingly, the amine compound of an embodiment may have improved hole transport properties and stability.

The amine compound of an embodiment may include a first substituent, a second substituent and a third substituent, having high electron tolerance, and may have high electron tolerance. In addition, the first to third substituents are substituents of which thermal decomposition is difficult, and the amine compound of an embodiment may suppress or reduce the excessive increase of the deposition temperature and may suppress or reduce the deterioration of a material by a deposition process. Accordingly, the lifetime of the light emitting element of an embodiment, including the amine compound of an embodiment may be improved.

In the light emitting element ED of an embodiment, the hole transport region HTR may further include a compound represented by Formula H-1 below.

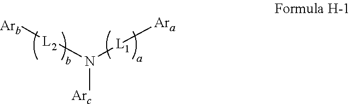

In Formula H-1 above, L₁ and L₂ may be each independently a direct linkage, a substituted or unsubstituted arylene group of 6 to 30 ring-forming carbon atoms, or a substituted or unsubstituted heteroarylene group of 2 to 30 ring-forming carbon atoms. “a” and “b” may be each independently an integer of 0 to 10. If “a” or “b” is an integer of 2 or more, a plurality of L₁ and L₂ may be each independently a substituted or unsubstituted arylene group of 6 to 30 ring-forming carbon atoms, or a substituted or unsubstituted heteroarylene group of 2 to 30 ring-forming carbon atoms.

In Formula H-1, Arₐ and Ar_{b} may be each independently a substituted or unsubstituted aryl group of 6 to 30 ring-forming carbon atoms, or a substituted or unsubstituted heteroaryl group of 2 to 30 ring-forming carbon atoms. In addition, in Formula H-1, Ar_{c} may be a substituted or unsubstituted aryl group of 6 to 30 ring-forming carbon atoms.

The compound represented by Formula H-1 may be a monoamine compound. Otherwise, the compound represented by Formula H-1 may be a diamine compound in which at least one among Arₐ to Ar_{c} includes an amine group as a substituent. In addition, the compound represented by Formula H-1 may be a carbazole-based compound in which at least one among Arₐ and Ar_{b} includes a substituted or unsubstituted carbazole group, or a fluorene-based compound in which at least one among Arₐ and Ar_{b} includes a substituted or unsubstituted fluorene group.

The compound represented by Formula H-1 may be represented by any one among the compounds in Compound Group H below. However, the compounds listed in Compound Group H are only illustrations, and the compound represented by Formula H-1 is not limited to the compounds represented in Compound Group H below.

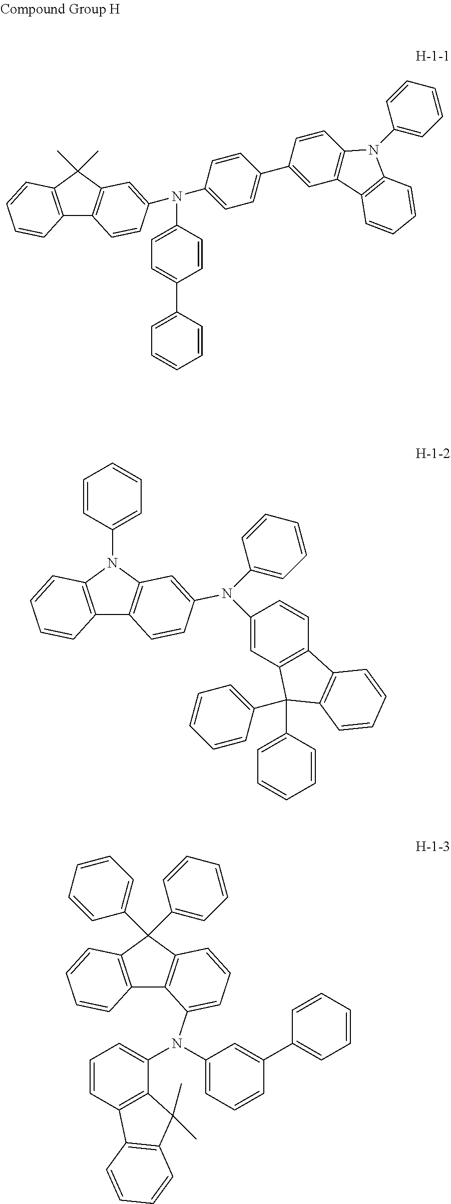 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(N(c2ccc3c(c2)C(c2ccccc2)(c2ccccc2)c2ccccc2-3)c2ccc3c4ccccc4n(-c4ccccc4)c3c2)cc1 ; CC1(C)c2ccccc2-c2cccc(N(c3cccc(-c4ccccc4)c3)c3cccc4c3-c3ccccc3C4(c3ccccc3)c3ccccc3)c21]

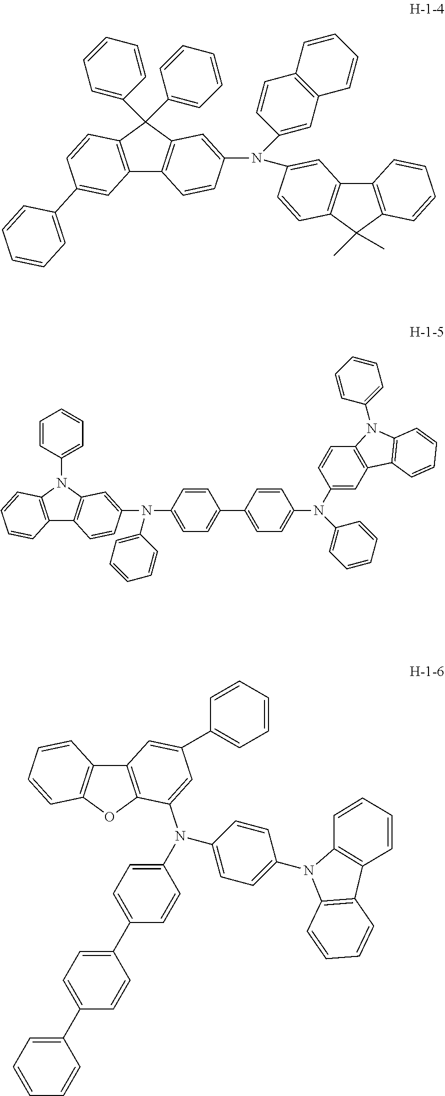 [SMILES: CC1(C)c2ccccc2-c2cc(N(c3ccc4c(c3)C(c3ccccc3)(c3ccccc3)c3ccc(-c5ccccc5)cc3-4)c3ccc4ccccc4c3)ccc21 ; c1ccc(N(c2ccc(-c3ccc(N(c4ccccc4)c4ccc5c6ccccc6n(-c6ccccc6)c5c4)cc3)cc2)c2ccc3c(c2)c2ccccc2n3-c2ccccc2)cc1 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1]

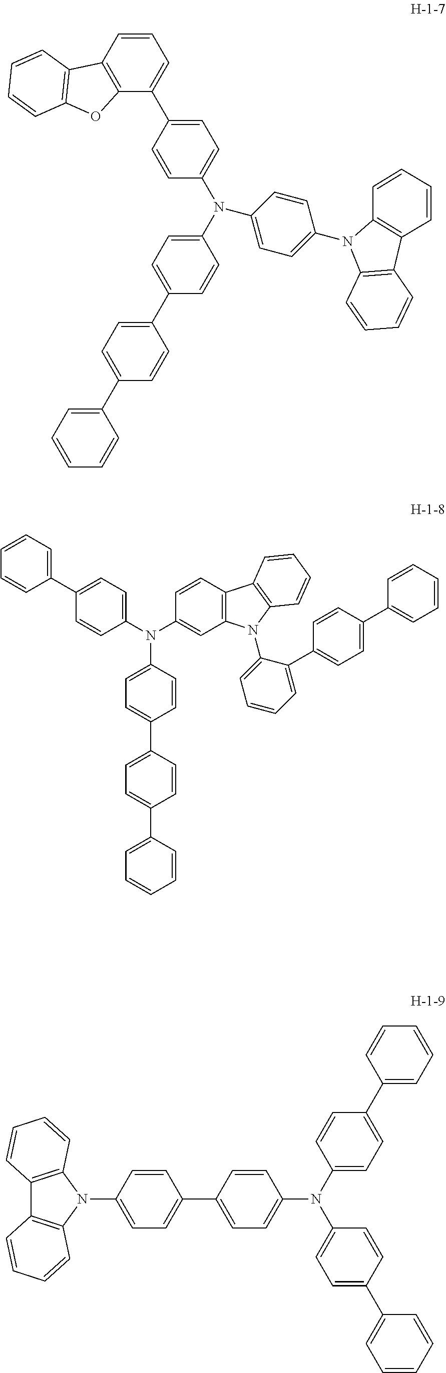 [SMILES: c1ccc(-c2ccc(-c3ccc(N(c4ccc(-c5cccc6c5oc5ccccc56)cc4)c4ccc(-n5c6ccccc6c6ccccc65)cc4)cc3)cc2)cc1 ; c1ccc(-c2ccc(-c3ccc(N(c4ccc(-c5ccccc5)cc4)c4ccc5c6ccccc6n(-c6ccccc6-c6ccc(-c7ccccc7)cc6)c5c4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1]

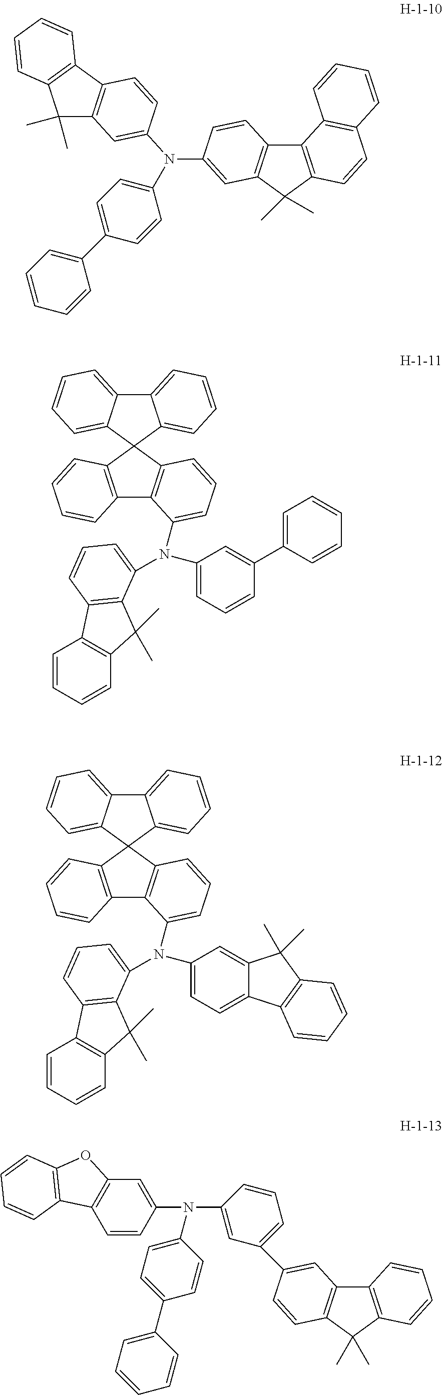 [SMILES: CC1(C)c2ccccc2-c2ccc(N(c3ccc(-c4ccccc4)cc3)c3ccc4c(c3)C(C)(C)c3ccc5ccccc5c3-4)cc21 ; CC1(C)c2ccccc2-c2cccc(N(c3cccc(-c4ccccc4)c3)c3cccc4c3-c3ccccc3C43c4ccccc4-c4ccccc43)c21 ; CC1(C)c2ccccc2-c2ccc(N(c3cccc4c3-c3ccccc3C43c4ccccc4-c4ccccc43)c3cccc4c3C(C)(C)c3ccccc3-4)cc21 ; CC1(C)c2ccccc2-c2cc(-c3cccc(N(c4ccc(-c5ccccc5)cc4)c4ccc5c(c4)oc4ccccc45)c3)ccc21]

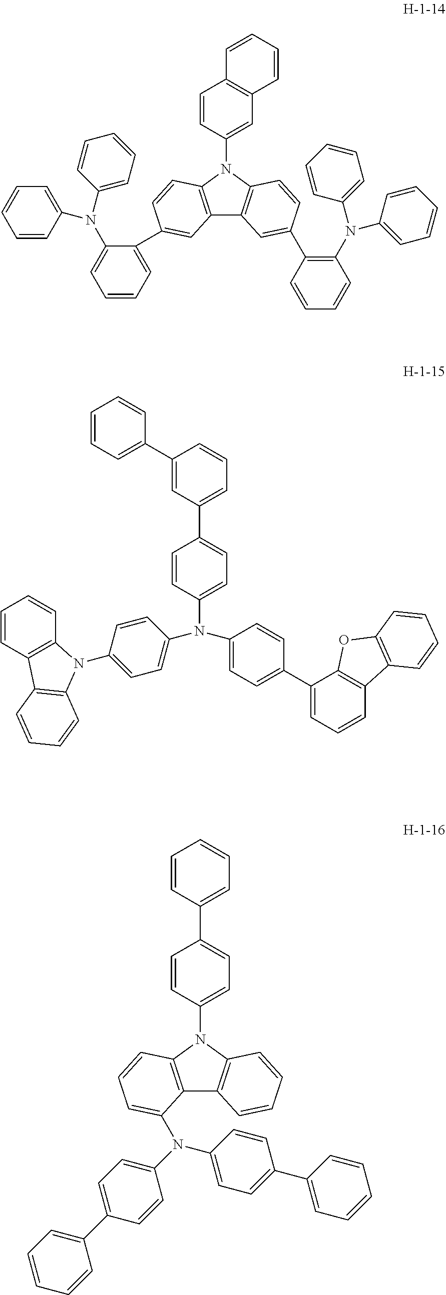 [SMILES: c1ccc(N(c2ccccc2)c2ccccc2-c2ccc3c(c2)c2cc(-c4ccccc4N(c4ccccc4)c4ccccc4)ccc2n3-c2ccc3ccccc3c2)cc1 ; c1ccc(-c2cccc(-c3ccc(N(c4ccc(-c5cccc6c5oc5ccccc56)cc4)c4ccc(-n5c6ccccc6c6ccccc65)cc4)cc3)c2)cc1 ; c1ccc(-c2ccc(N(c3ccc(-c4ccccc4)cc3)c3cccc4c3c3ccccc3n4-c3ccc(-c4ccccc4)cc3)cc2)cc1]

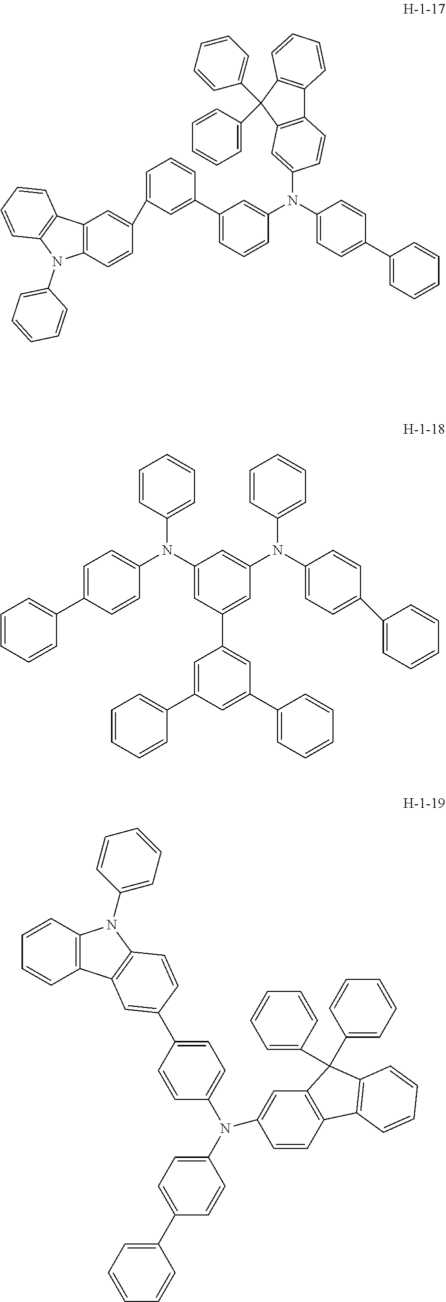 [SMILES: c1ccc(-c2ccc(N(c3cccc(-c4cccc(-c5ccc6c(c5)c5ccccc5n6-c5ccccc5)c4)c3)c3ccc4c(c3)C(c3ccccc3)(c3ccccc3)c3ccccc3-4)cc2)cc1 ; c1ccc(-c2ccc(N(c3ccccc3)c3cc(-c4cc(-c5ccccc5)cc(-c5ccccc5)c4)cc(N(c4ccccc4)c4ccc(-c5ccccc5)cc4)c3)cc2)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

Besides, the hole transport region HTR may further include any suitable hole transport material generally used in the art.

For example, the hole transport region HTR may include a phthalocyanine compound such as copper phthalocyanine, N¹,N^{1′}-([1,1′-biphenyl]-4,4′-diyl)bis(N¹-phenyl-N⁴,N⁴-di-m-tolylbenzene-1,4-diamine) (DNTPD), 4,4′,4″-[tris(3-methylphenyl)phenylamino] triphenylamine (m-MTDATA), 4,4′,4″-tris(N,N-diphenylamino)triphenylamine (TDATA), 4,4′,4″-tris[N(1-naphthyl)-N-phenylamino]-triphenylamine (1-TNATA), 4,4′,4″-tris[N(2-naphthyl)-N-phenylamino]triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonic acid (PANI/CSA), polyaniline/poly(4-styrenesulfonate) (PANI/PSS), N,N′-di(1-naphthalen-1-yl)-N,N′-diphenyl-benzidine (NPB or NPD), triphenylamine-containing polyetherketone (TPAPEK), 4-isopropyl-4′-methyldiphenyliodonium [tetrakis(pentafluorophenyl)borate], and dipyrazino[2,3-f:2′,3′-h] quinoxaline-2,3,6,7,10,11-hexacarbonitrile (HATCN).

The hole transport region HTR may include carbazole derivatives such as N-phenyl carbazole and polyvinyl carbazole, fluorene-based derivatives, triphenylamine-based derivatives such as 4,4′,4″-tris(N-carbazolyl)triphenylamine (TCTA), N,N′-bis(3-methylphenyl)-N,N′-diphenyl-[1,1′-biphenyl]-4,4′-diamine (TPD), N,N′-di(1-naphthalen-1-yl)-N,N′-diphenyl-benzidine (NPB), 4,4′-cyclohexylidene bis[N,N-bis(4-methylphenyl)benzeneamine] (TAPC), 4,4′-bis[N,N′-(3-tolyl)amino]-3,3′-dimethylbiphenyl (HMTPD), 1,3-bis(N-carbazolyl)benzene (mCP), etc.

In addition, the hole transport region HTR may include 9-(4-tert-butylphenyl)-3,6-bis(triphenylsilyl)-9H-carbazole (CzSi), 9-phenyl-9H-3,9′-bicarbazole (CCP), 1,3-bis(1,8-dimethyl-9H-carbazol-9-yl)benzene (mDCP), etc.

The hole transport region HTR may include the compounds of the hole transport region in at least one among the hole injection layer HIL, hole transport layer HTL, and electron blocking layer EBL.

The thickness of the hole transport region HTR may be from about 100 Å to about 10,000 Å, for example, from about 100 Å to about 5,000 Å. In a case where the hole transport region HTR includes a hole injection layer HIL, the thickness of the hole injection region HIL may be, for example, from about 30 Å to about 1,000 Å. In a case where the hole transport region HTR includes a hole transport layer HTL, the thickness of the hole transport layer HTL may be from about 30 Å to about 1,000 Å. For example, in a case where the hole transport region HTR includes an electron blocking layer EBL, the thickness of the electron blocking layer EBL may be from about 10 Å to about 1,000 Å. If the thicknesses of the hole transport region HTR, the hole injection layer HIL, the hole transport layer HTL and the electron blocking layer EBL satisfy the above-described ranges, suitable or satisfactory hole transport properties may be achieved without substantial increase of a driving voltage.

The hole transport region HTR may further include a charge generating material to increase conductivity (e.g., electrical conductivity), in addition to the above-described materials. The charge generating material may be dispersed uniformly or non-uniformly in the hole transport region HTR. The charge generating material may be, for example, a p-dopant. The p-dopant may include at least one of metal halide compounds, quinone derivatives, metal oxides, and/or cyano group-containing compounds, without limitation. For example, the p-dopant may include metal halide compounds such as CuI and/or RbI, quinone derivatives such as tetracyanoquinodimethane (TCNQ) and/or 2,3,5,6-tetrafluoro-7,7,8,8-tetracyanoquinodimethane (F4-TCNQ), metal oxides such as tungsten oxide and/or molybdenum oxide, cyano group-containing compounds such as dipyrazino[2,3-f: 2′,3′-h] quinoxaline-2,3,6,7,10,11-hexacarbonitrile (HATCN) and 4-[[2,3-bis[cyano-(4-cyano-2,3,5,6-tetrafluorophenyl)methylidene]cyclopropylidene]-cyanomethyl]-2,3,5,6-tetrafluorobenzonitrile (NDP9), etc., without limitation.

As described above, the hole transport region HTR may further include a buffer layer) in addition to the hole injection layer HIL, the hole transport layer HTL and the electron blocking layer EBL. The buffer layer may compensate for a resonance distance according to the wavelength of light emitted from an emission layer EML and may increase emission efficiency. As materials included in the buffer layer, materials which may be included in the hole transport region HTR may be used.

The emission layer EML is provided on the hole transport region HTR. The emission layer EML may have a thickness of, for example, about 100 Å to about 1,000 Å or about 100 Å to about 300 Å. The emission layer EML may have a single layer formed using a single material, a single layer formed using a plurality of different materials, or a multilayer structure having a plurality of layers formed using a plurality of different materials.

In the light emitting element ED of an embodiment, the emission layer EML may emit blue light. The light emitting element ED of an embodiment may include the amine compound of an embodiment in a hole transport region HTR and may exhibit high efficiency and long-life characteristics in a blue emission region. However, embodiments of the present disclosure are not limited thereto.

In the light emitting element ED of an embodiment, the emission layer EML may include anthracene derivatives, pyrene derivatives, fluoranthene derivatives, chrysene derivatives, dihydrobenzanthracene derivatives, and/or triphenylene derivatives. In some embodiments, the emission layer EML may include anthracene derivatives and/or pyrene derivatives.

In the light emitting elements ED of embodiments, shown in FIG. 3 to FIG. 6, the emission layer EML may include a host and a dopant, and the emission layer EML may include a compound represented by Formula E-1 below. The compound represented by Formula E-1 below may be used as a fluorescence host material.

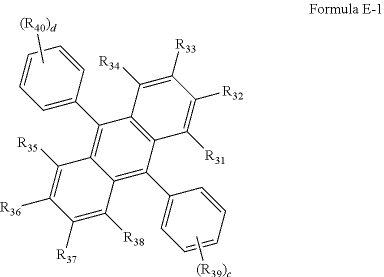

In Formula E-1, R₃₁ to R₄₀ may be each independently a hydrogen atom, a deuterium atom, a halogen atom, a substituted or unsubstituted silyl group, a substituted or unsubstituted thio group, a substituted or unsubstituted oxy group, a substituted or unsubstituted alkyl group of 1 to 10 carbon atoms, a substituted or unsubstituted alkenyl group of 2 to 10 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring. In some embodiments, R₃₁ to R₄₀ may be combined with an adjacent group to form a saturated hydrocarbon ring, an unsaturated hydrocarbon ring, a saturated heterocycle, or an unsaturated heterocycle.

In Formula E-1, “c” and “d” may be each independently an integer of 0 to 5.

Formula E-1 may be represented by any one among Compound E1 to Compound E19 below.

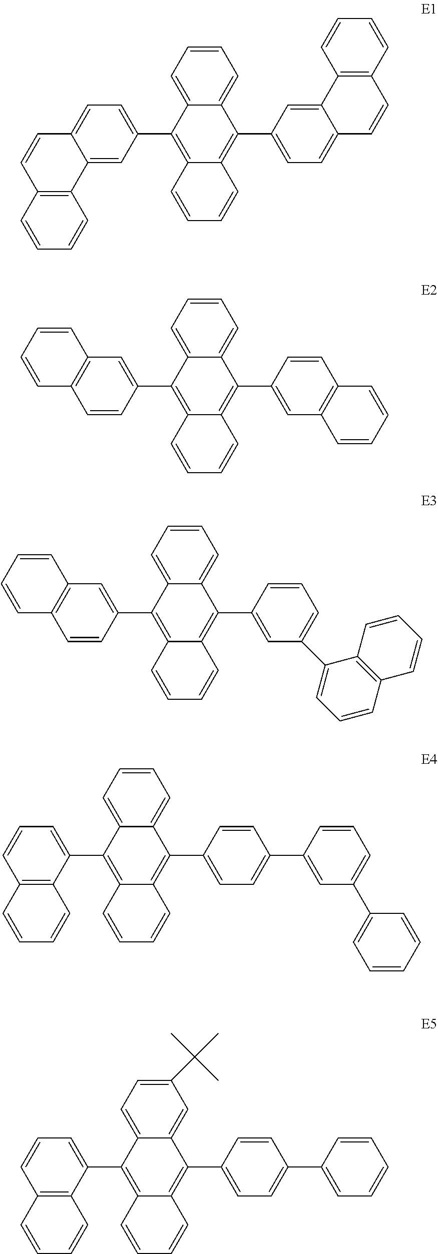

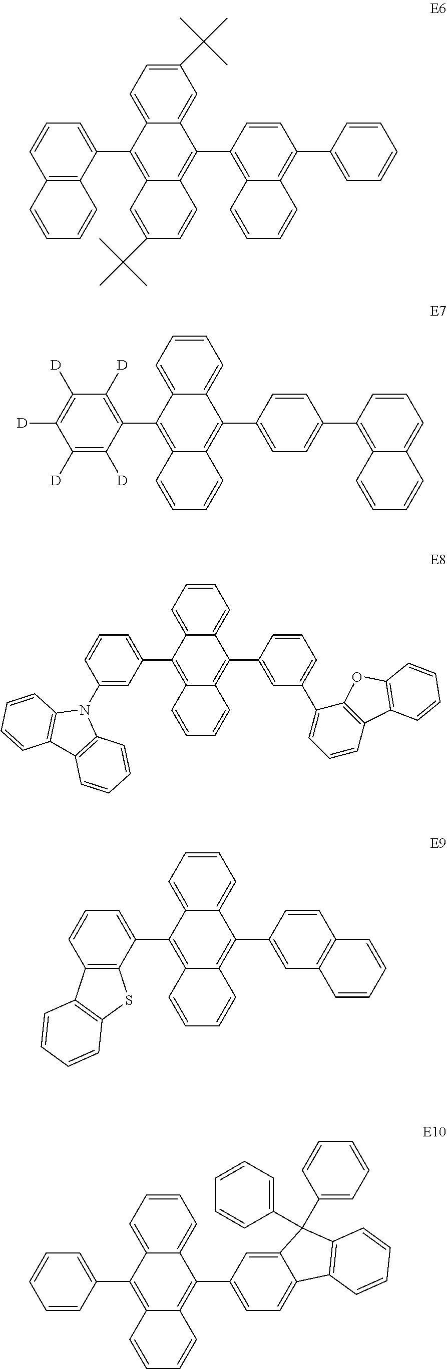

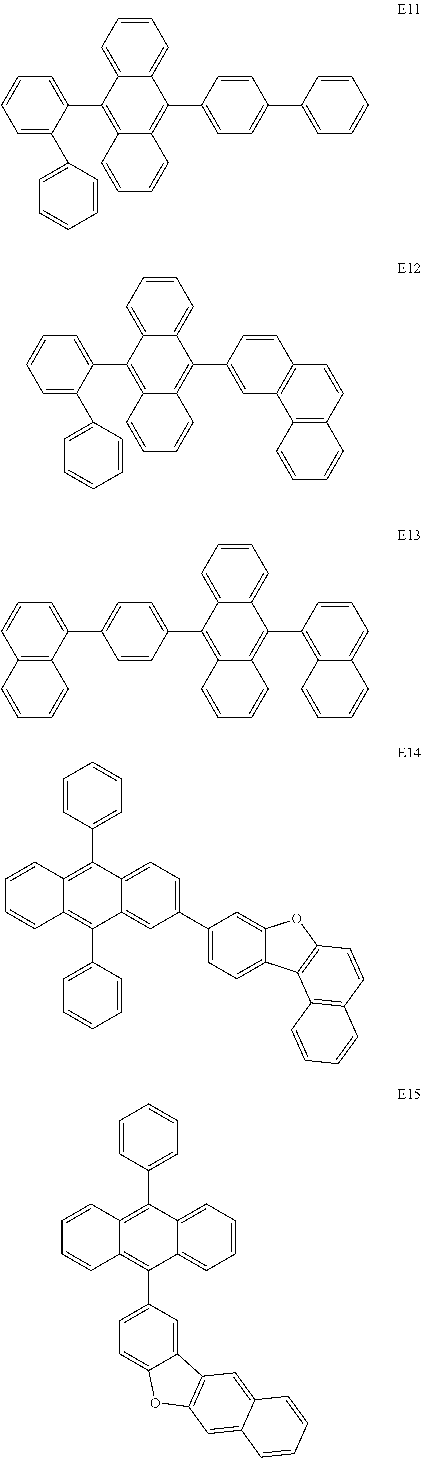

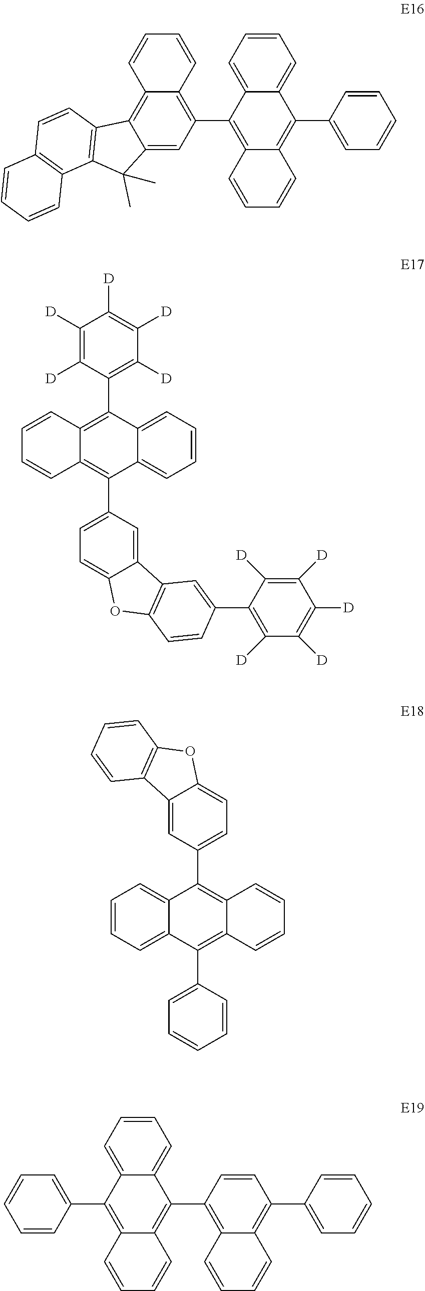

In some embodiments, the emission layer EML may include a compound represented by Formula E-2a or Formula E-2b below. The compound represented by Formula E-2a or Formula E-2b below may be used as a phosphorescence host material.

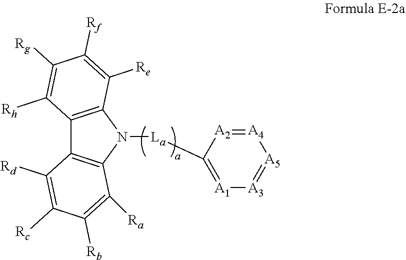

In Formula E-2a, “a” may be an integer of 0 to 10, Lₐ may be a direct linkage, a substituted or unsubstituted arylene group of 6 to 30 ring-forming carbon atoms, or a substituted or unsubstituted heteroarylene group of 2 to 30 ring-forming carbon atoms. If “a” is an integer of 2 or more, a plurality of Lₐ may be each independently a substituted or unsubstituted arylene group of 6 to 30 ring-forming carbon atoms, or a substituted or unsubstituted heteroarylene group of 2 to 30 ring-forming carbon atoms.

In addition, in Formula E-2a, A₁ to A₅ may be each independently N or CRᵢ. Rₐ to Rᵢ may be each independently a hydrogen atom, a deuterium atom, a substituted or unsubstituted amine group, a substituted or unsubstituted thio group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring. Rₐ to Rᵢ may be combined with an adjacent group to form a hydrocarbon ring or a heterocycle including N, O, S, etc. as a ring-forming atom.

In some embodiments, in Formula E-2a, two or three selected from A₁ to A₅ may be N, and the remainder may be CRᵢ.

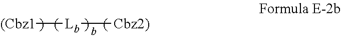

In Formula E-2b, Cbz1 and Cbz2 may be each independently an unsubstituted carbazole group, or a carbazole group substituted with an aryl group of 6 to 30 ring-forming carbon atoms. L_{b} may be a direct linkage, a substituted or unsubstituted arylene group of 6 to 30 ring-forming carbon atoms, or a substituted or unsubstituted heteroarylene group of 2 to 30 ring-forming carbon atoms. “b” is an integer of 0 to 10, and if “b” is an integer of 2 or more, a plurality of L_{b} may be each independently a substituted or unsubstituted arylene group of 6 to 30 ring-forming carbon atoms, or a substituted or unsubstituted heteroarylene group of 2 to 30 ring-forming carbon atoms.

The compound represented by Formula E-2a or Formula E-2b may be represented by any one among the compounds in Compound Group E-2 below. However, the compounds listed in Compound Group E-2 below are only illustrations, and the compound represented by Formula E-2a or Formula E-2b is not limited to the compounds represented in Compound Group E-2 below.

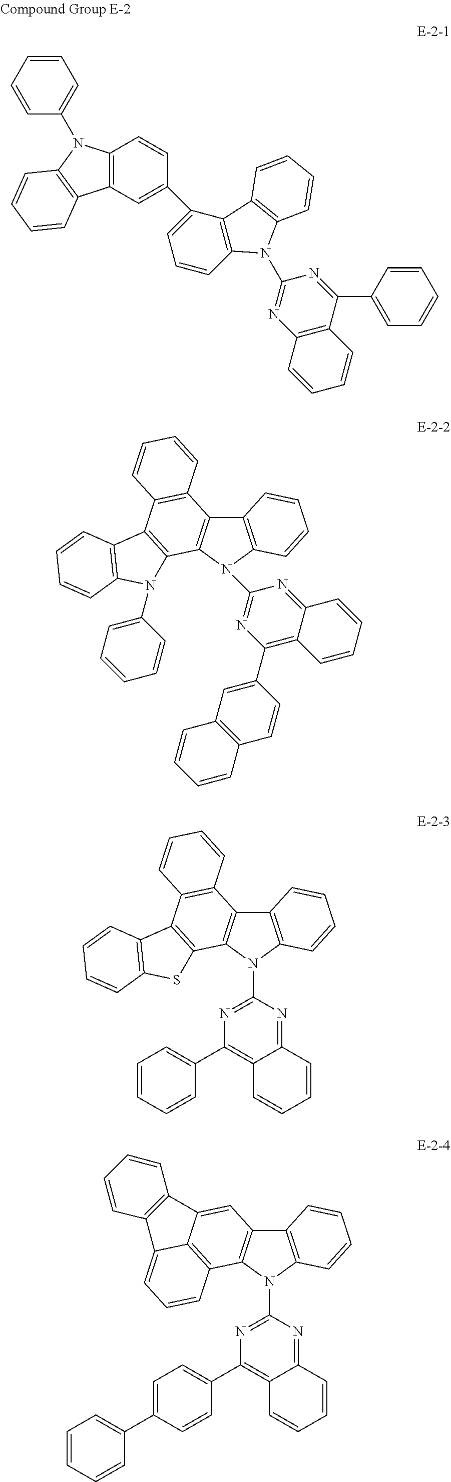

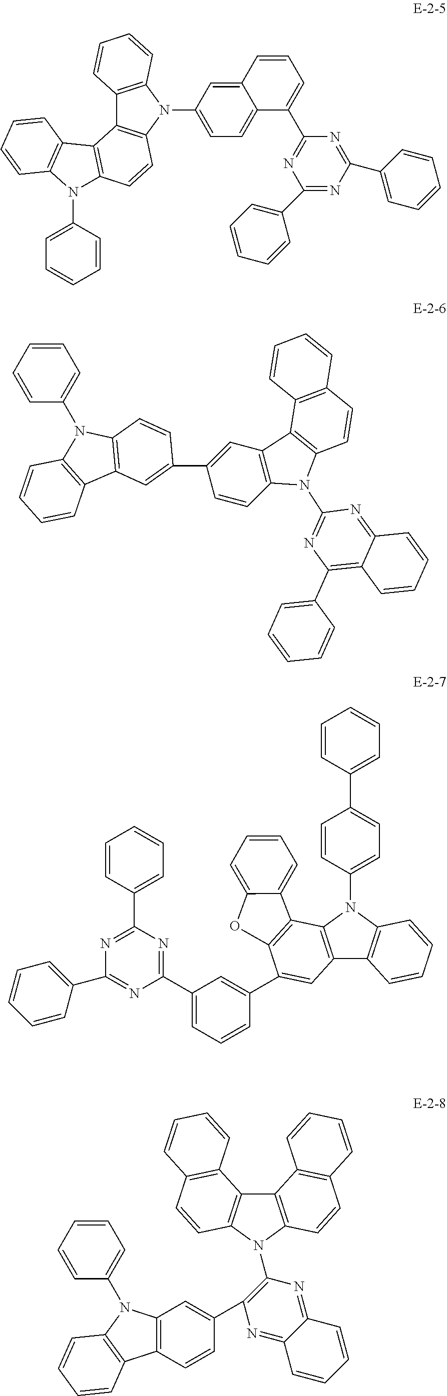 [SMILES: c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1 ; c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1 ; c1ccc(-n2c3ccccc3c3ccc(-c4nc5ccccc5nc4-n4c5ccc6ccccc6c5c5c6ccccc6ccc54)cc32)cc1]

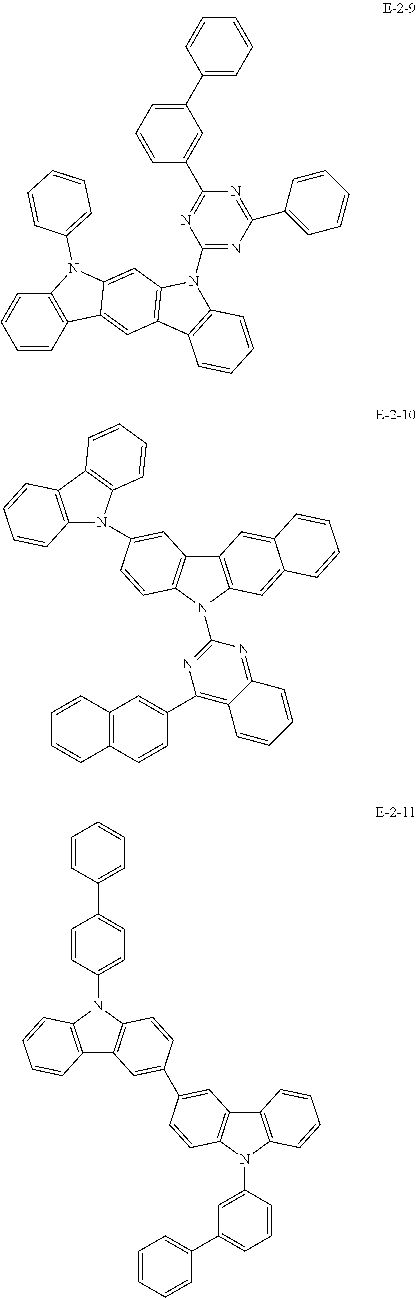 [SMILES: c1ccc(-c2cccc(-c3nc(-c4ccccc4)nc(-n4c5ccccc5c5cc6c7ccccc7n(-c7ccccc7)c6cc54)n3)c2)cc1 ; c1ccc2cc(-c3nc(-n4c5ccc(-n6c7ccccc7c7ccccc76)cc5c5cc6ccccc6cc54)nc4ccccc34)ccc2c1 ; c1ccc(-c2ccc(-n3c4ccccc4c4cc(-c5ccc6c(c5)c5ccccc5n6-c5cccc(-c6ccccc6)c5)ccc43)cc2)cc1]

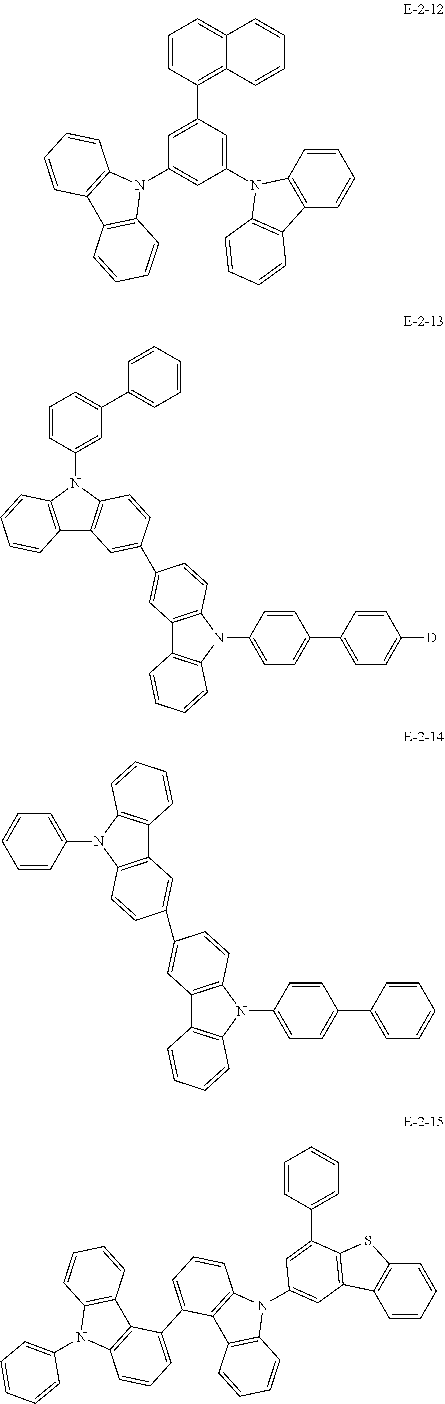 [SMILES: c1ccc2c(-c3cc(-n4c5ccccc5c5ccccc54)cc(-n4c5ccccc5c5ccccc54)c3)cccc2c1 ; [2H]c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cc(-n3c4ccccc4c4c(-c5cccc6c5c5ccccc5n6-c5ccccc5)cccc43)cc3c2sc2ccccc23)cc1]

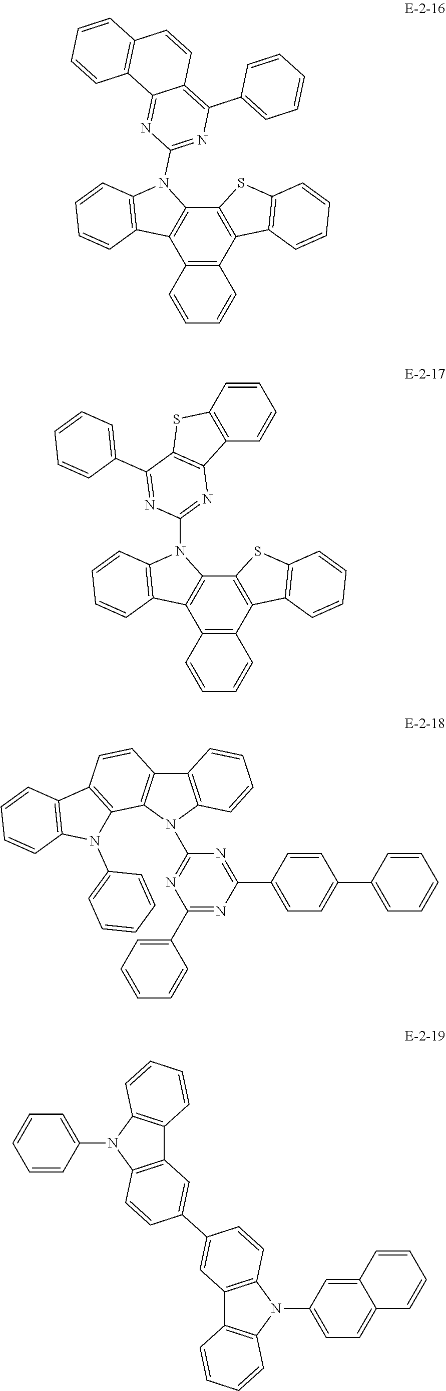 [SMILES: c1ccc(-c2nc(-n3c4ccccc4c4c5ccccc5c5c6ccccc6sc5c43)nc3c2ccc2ccccc23)cc1 ; c1ccc(-c2nc(-n3c4ccccc4c4c5ccccc5c5c6ccccc6sc5c43)nc3c2sc2ccccc23)cc1 ; c1ccc(-c2ccc(-c3nc(-c4ccccc4)nc(-n4c5ccccc5c5ccc6c7ccccc7n(-c7ccccc7)c6c54)n3)cc2)cc1 ; c1ccc(-n2c3ccccc3c3cc(-c4ccc5c(c4)c4ccccc4n5-c4ccc5ccccc5c4)ccc32)cc1]

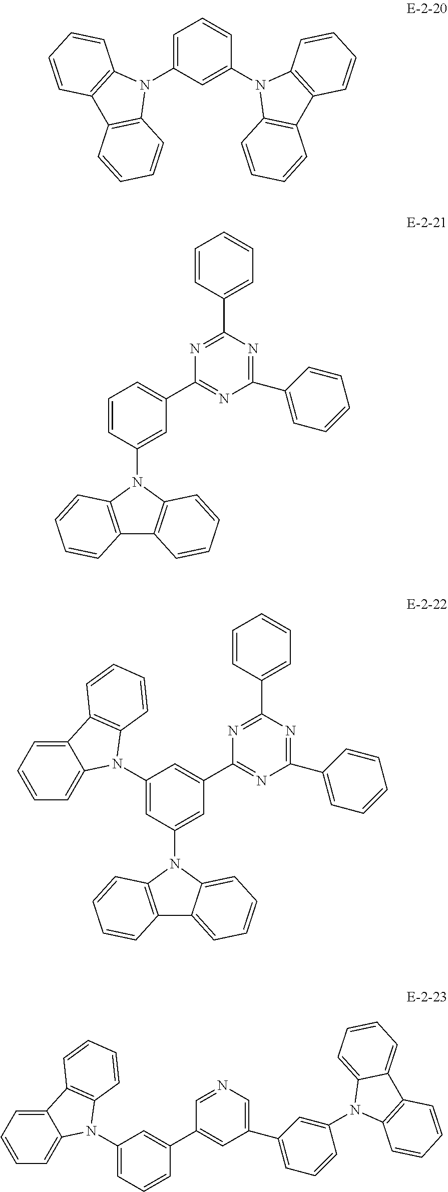 [SMILES: c1cc(-n2c3ccccc3c3ccccc32)cc(-n2c3ccccc3c3ccccc32)c1 ; c1ccc(-c2nc(-c3ccccc3)nc(-c3cccc(-n4c5ccccc5c5ccccc54)c3)n2)cc1 ; c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1]

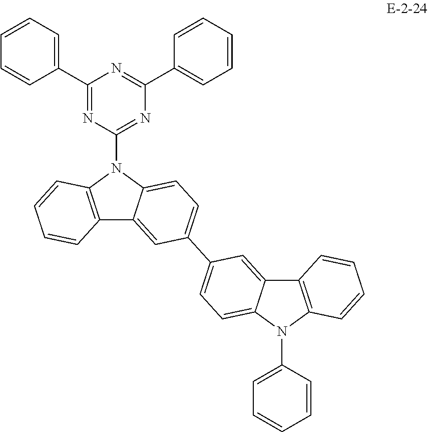 [SMILES: c1ccc(-c2nc(-c3ccccc3)nc(-n3c4ccccc4c4cc(-c5ccc6c(c5)c5ccccc5n6-c5ccccc5)ccc43)n2)cc1]

The emission layer EML may further include any suitable material generally used in the art as a host material. For example, the emission layer EML may include as a host material, at least one of bis (4-(9H-carbazol-9-yl) phenyl) diphenylsilane (BCPDS), (4-(1-(4-(diphenylamino) phenyl) cyclohexyl) phenyl) diphenyl-phosphine oxide (POPCPA), bis[2-(diphenylphosphino)phenyl] ether oxide (DPEPO), 4,4′-bis(N-carbazolyl)-1,1′-biphenyl (CBP), 1,3-bis(carbazol-9-yl)benzene (mCP), 2,8-bis(diphenylphosphoryl)dibenzo[b,d]furan (PPF), 4,4′,4″-tris(carbazol-9-yl)-triphenylamine (TCTA), and/or 1,3,5-tris(1-phenyl-1H-benzo[d]imidazole-2-yl)benzene (TPBi). However, embodiments of the present disclosure are not limited thereto. For example, tris(8-hydroxyquinolinato)aluminum (Alq₃), 9,10-di(naphthalen-2-yl)anthracene (I), 2-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen-2-yl)anthracene (MADN), hexaphenyl cyclotriphosphazene (CP1), 1,4-bis(triphenylsilyl)benzene (UGH2), hexaphenylcyclotrisiloxane (DPSiO₃), octaphenylcyclotetra siloxane (DPSiO₄), etc. may be used as the host material.

The emission layer EML may include a compound represented by Formula M-a or Formula M-b below. The compound represented by Formula M-a or Formula M-b may be used as a phosphorescence dopant material. In addition, in some embodiments, the compound represented by Formula M-a or Formula M-b may be used as an auxiliary dopant material.

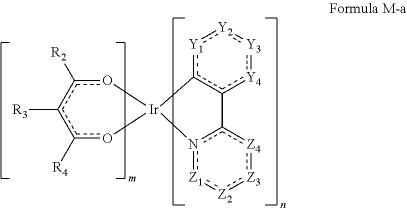

In Formula M-a, Y₁ to Y₄, and Z₁ to Z₄ may be each independently CR₁ or N, and R₁ to R₄ may be each independently a hydrogen atom, a deuterium atom, a substituted or unsubstituted amine group, a substituted or unsubstituted thio group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring. In Formula M-a, “m” is 0 or 1, and “n” is 2 or 3. In Formula M-a, if “m” is 0, “n” is 3, and if “m” is 1, “n” is 2.

The compound represented by Formula M-a may be used as a phosphorescence dopant.

The compound represented by Formula M-a may be represented by any one among Compounds M-a1 to M-a25 below. However, Compounds M-a1 to M-a25 below are illustrations, and the compound represented by Formula M-a is not limited to the compounds represented by Compounds M-a1 to M-a25 below.

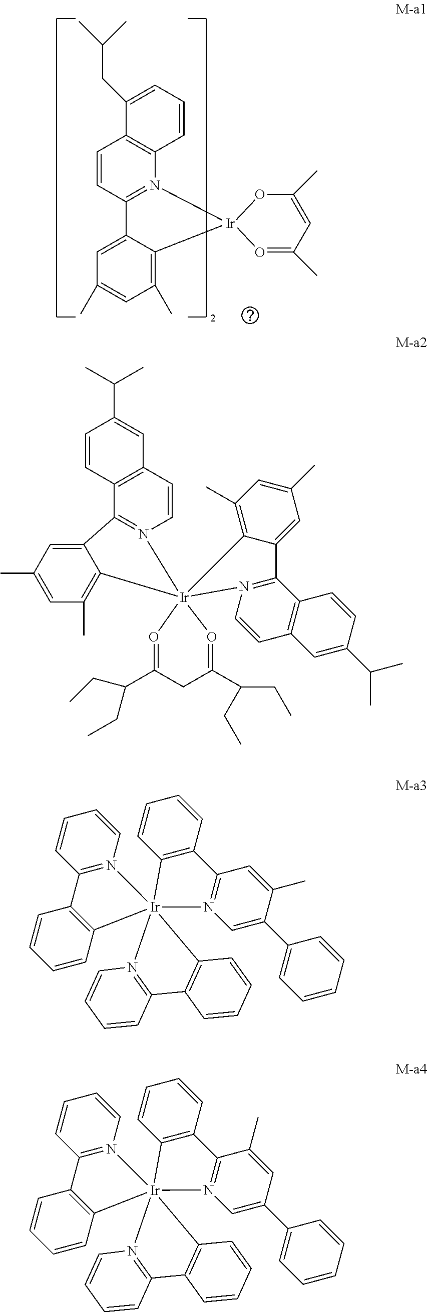

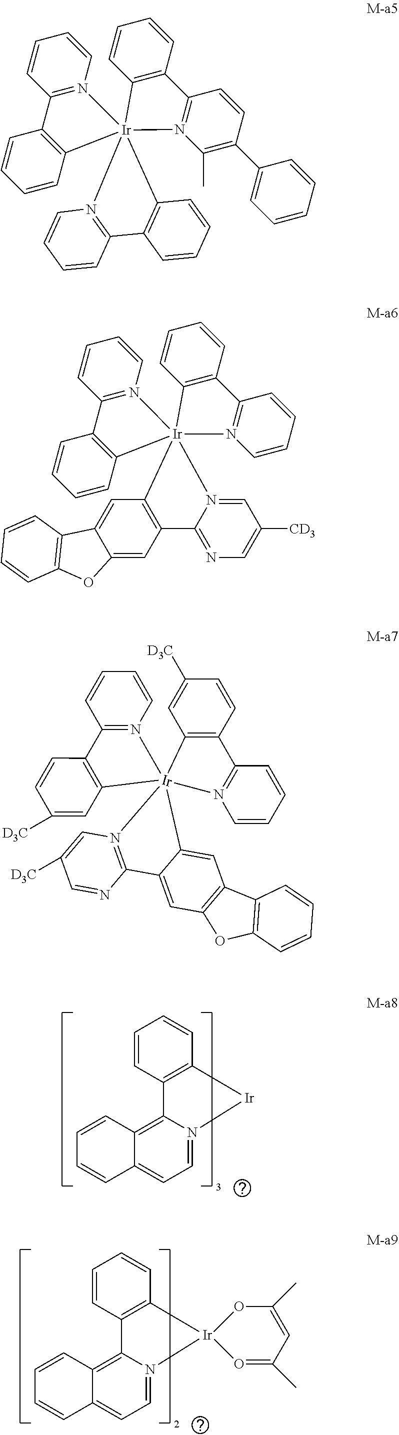

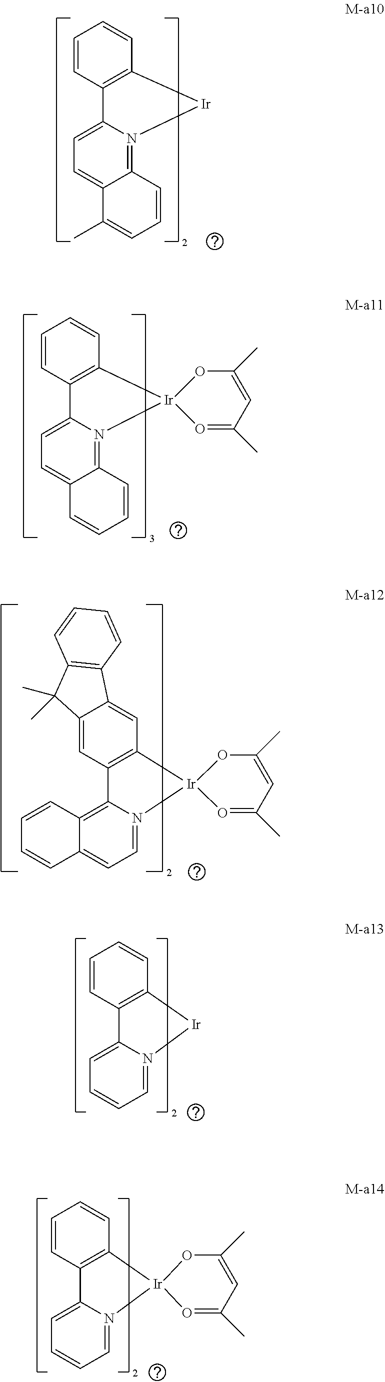

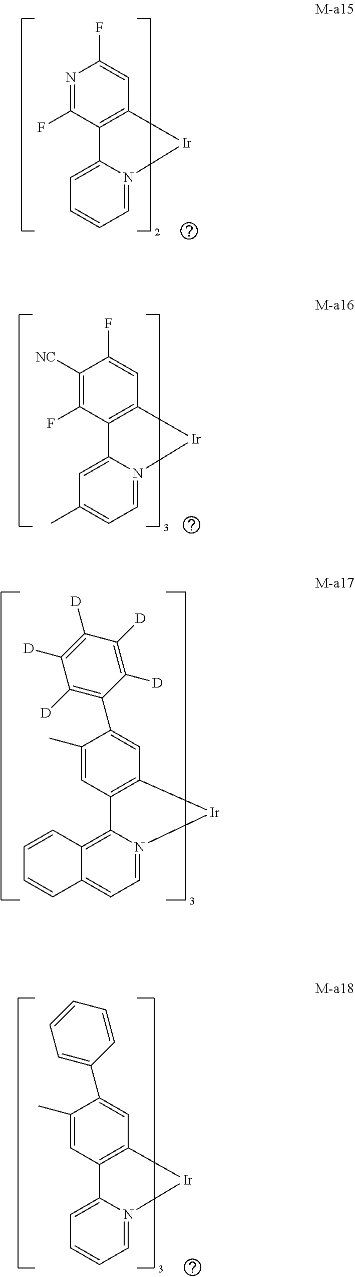

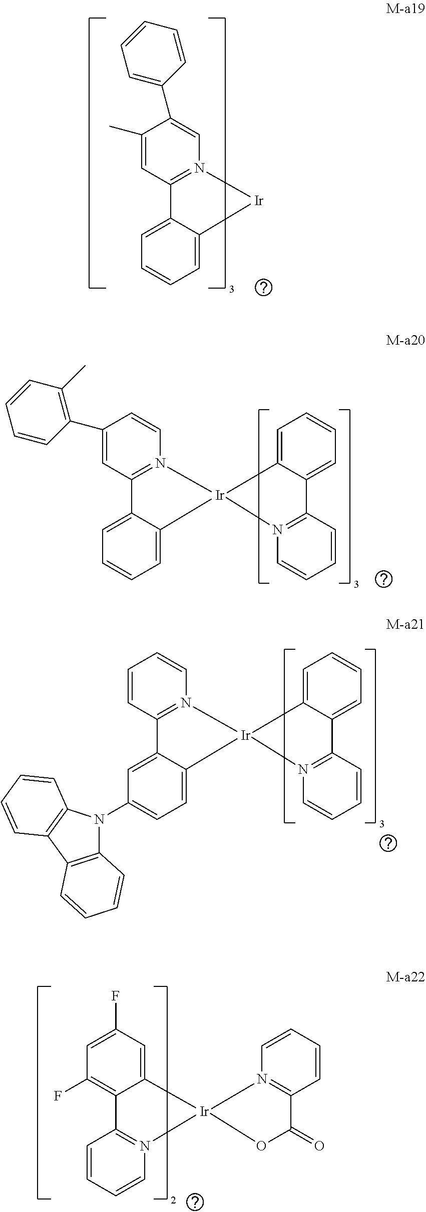

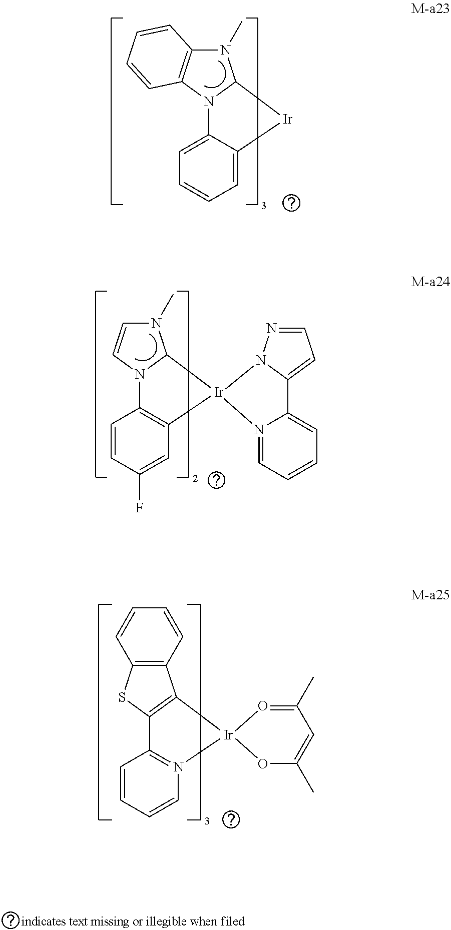

Compound M-a1 and Compound M-a2 may be used as red dopant materials, and Compound M-a3 to Compound M-a7 may be used as green dopant materials.

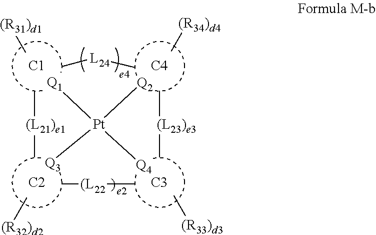

In Formula M-b, Q₁ to Q₄ are each independently C or N, C1 to C4 are each independently a substituted or unsubstituted hydrocarbon ring of 5 to 30 ring-forming carbon atoms, or a substituted or unsubstituted heterocycle of 2 to 30 ring-forming carbon atoms. L₂₁ to L₂₄ are each independently a direct linkage,

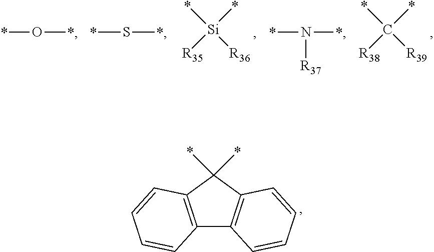

a substituted or unsubstituted divalent alkyl group of 1 to 20 carbon atoms, a substituted or unsubstituted arylene group of 6 to 30 ring-forming carbon atoms, or a substituted or unsubstituted heteroarylene group of 2 to 30 ring-forming carbon atoms, and e1 to e4 are each independently 0 or 1. R₃₁ to R₃₉ are each independently a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or combined with an adjacent group to form a ring, and d1 to d4 are each independently an integer of 0 to 4.

The compound represented by Formula M-b may be used as a blue phosphorescence dopant or a green phosphorescence dopant. In addition, the compound represented by Formula M-b may be an auxiliary dopant according to some embodiments and may be further included in the emission layer EML.

The compound represented by Formula M-b may be represented by any one among the compounds below. However, the compounds below are illustrations, and the compound represented by Formula M-b is not limited to the compounds represented below.

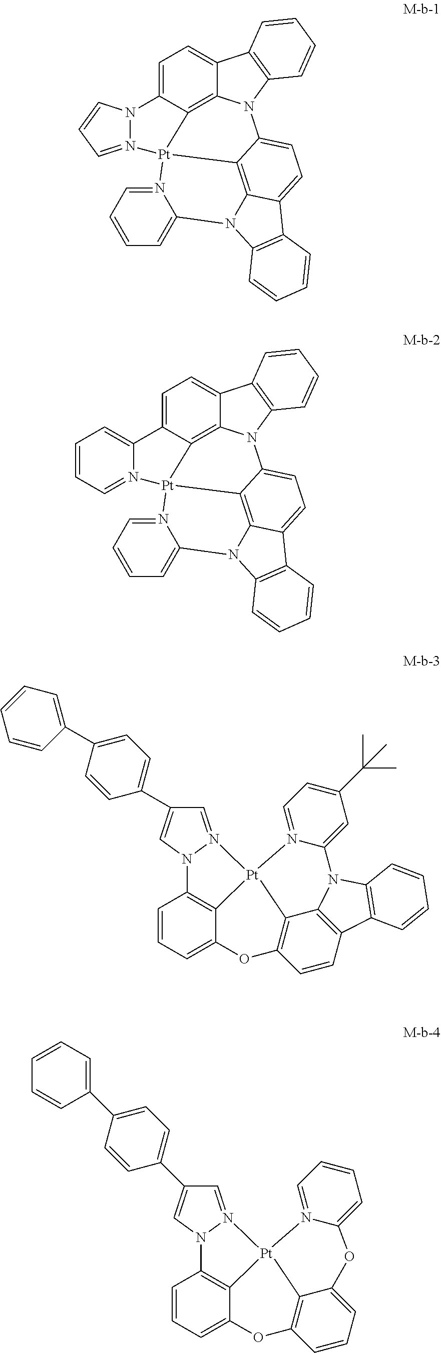

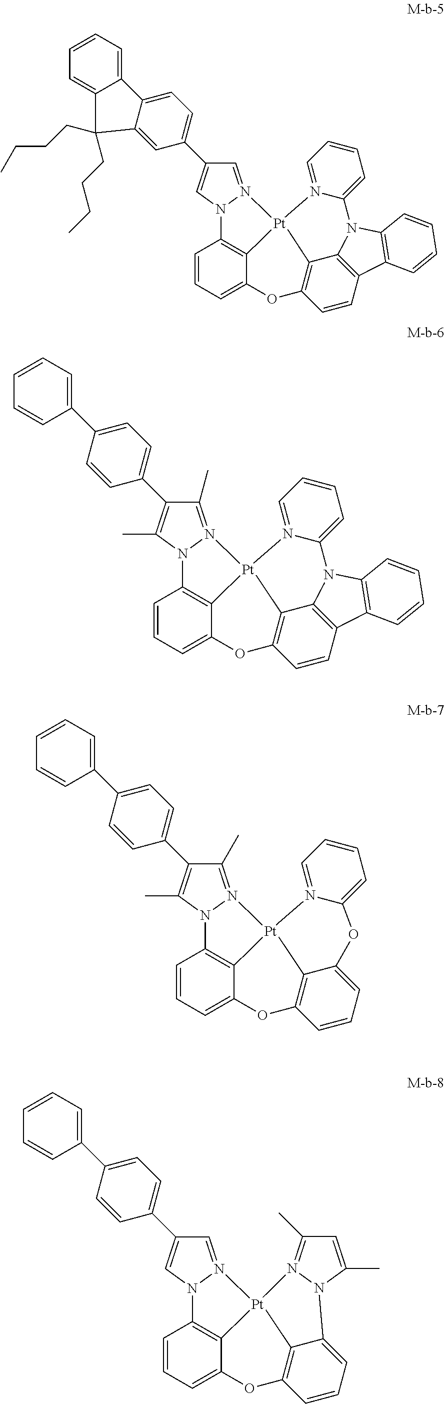

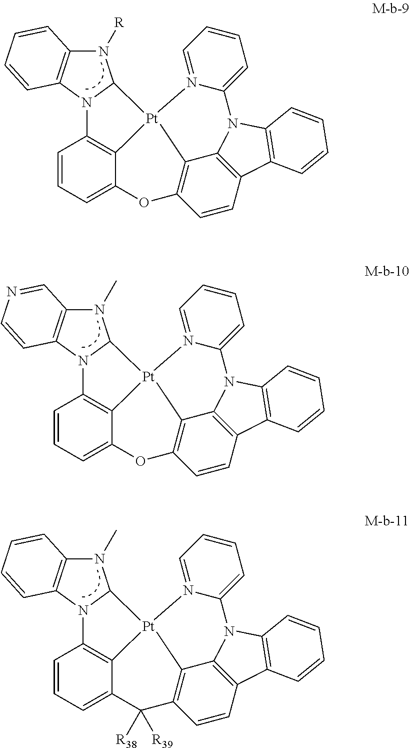

In the compounds above, R, R₃₈, and R₃₉ may be each independently a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

The emission layer EML may further include a compound represented by any one among Formula F-a to Formula F-c below. The compounds represented by Formula F-a to Formula F-c may be used as fluorescence dopant materials.

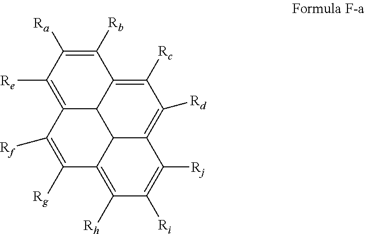

In Formula F-a, two selected from Rₐ to Rⱼ may be each independently substituted with *—NAr₁Ar₂. The remainder not substituted with *—NAr₁Ar₂ among Rₐ to Rⱼ may be each independently a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In *—NAr₁Ar₂, Ar₁ and Ar₂ may be each independently a substituted or unsubstituted aryl group of 6 to 30 ring-forming carbon atoms, or a substituted or unsubstituted heteroaryl group of 2 to 30 ring-forming carbon atoms. For example, at least one among Ar₁ and Ar₂ may be a heteroaryl group including O or S as a ring-forming atom.

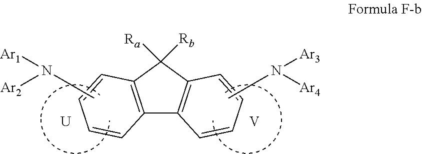

In Formula F-b, Rₐ and R_{b} may be each independently a hydrogen atom, a deuterium atom, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring. Ar₁ to Ar₄ may be each independently a substituted or unsubstituted aryl group of 6 to 30 ring-forming carbon atoms, or a substituted or unsubstituted heteroaryl group of 2 to 30 ring-forming carbon atoms.

In Formula F-b, U and V may be each independently a substituted or unsubstituted hydrocarbon ring of 5 to 30 ring-forming carbon atoms, or a substituted or unsubstituted heterocycle of 2 to 30 ring-forming carbon atoms.

In Formula F-b, the number of rings represented by U and V may be each independently 0 or 1. For example, in Formula F-b, if the number of U or V is 1, one ring forms a fused ring at the designated part by U or V, and if the number of U or V is 0, a ring is not present at the designated part by U or V. In some embodiments, if the number of U is 0, and the number of V is 1, or if the number of U is 1, and the number of V is 0, a fused ring having the fluorene core of Formula F-b may be a ring compound having four rings. In addition, if the number of both U and V is 0, the fused ring of Formula F-b may be a ring compound having three rings. In addition, if the number of both U and V is 1, a fused ring having the fluorene core of Formula F-b may be a ring compound having five rings.

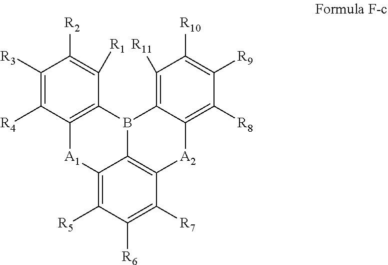

In Formula F-c, A₁ and A₂ may be each independently O, S, Se, or NRₘ, and Rₘ may be a hydrogen atom, a deuterium atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. R₁ to R₁₁ are each independently a hydrogen atom, a deuterium atom, a halogen atom, a cyano group, a substituted or unsubstituted amine group, a substituted or unsubstituted boryl group, a substituted or unsubstituted oxy group, a substituted or unsubstituted thio group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or combined with an adjacent group to form a ring.

In Formula F-c, A₁ and A₂ may be each independently combined with the substituents of an adjacent ring to form a fused ring. For example, if A₁ and A₂ may be each independently NRₘ, A₁ may be combined with R₄ or R₅ to form a ring. In addition, A₂ may be combined with R₇ or R₈ to form a ring.

In some embodiments, the emission layer EML may include as a dopant material, styryl derivatives (for example, 1,4-bis[2-(3-N-ethylcarbazoryl)vinyl]benzene (BCzVB), 4-(di-p-tolylamino)-4′-[(di-p-tolylamino)styryl]stilbene (DPAVB), N-(4-((E)-2-(6-((E)-4-(diphenylamino)styryl)naphthalen-2-yl)vinyl)phenyl)-N-phenylbenzenamine (N-BDAVBi), and 4,4′-bis[2-(4-(N,N-diphenylamino)phenyl)vinyl]biphenyl (DPAVBi)), perylene and the derivatives thereof (for example, 2,5,8,11-tetra-t-butylperylene (TBP)), pyrene and the derivatives thereof (for example, 1,1′-dipyrene, 1,4-dipyrenylbenzene, 1,4-bis(N,N-diphenylamino)pyrene), etc.

In some embodiments, if a plurality of emission layers EML are included, at least one emission layer EML may include any suitable phosphorescence dopant material generally used in the art. For example, the phosphorescence dopant may use a metal complex including iridium (Ir), platinum (Pt), osmium (Os), gold (Au), titanium (Ti), zirconium (Zr), hafnium (Hf), europium (Eu), terbium (Tb) or thulium (Tm). In some embodiments, iridium(III) bis(4,6-difluorophenylpyridinato-N,C2′)picolinate (Flrpic), bis(2,4-difluorophenylpyridinato)-tetrakis(1-pyrazolyl)borate iridium(III) (Fir₆), and/or platinum octaethyl porphyrin (PtOEP) may be used as the phosphorescence dopant. However, embodiments of the present disclosure are not limited thereto.

In some embodiments, the emission layer EML may include a hole transport host and an electron transport host. In addition, the emission layer EML may include an auxiliary dopant and a light emitting dopant. In some embodiments, the auxiliary dopant may include a phosphorescence dopant material and/or a thermally activated delayed fluorescence dopant. For example, in some embodiments, the emission layer EML may include a hole transport host, an electron transport host, an auxiliary dopant, and a light emitting dopant.

In addition, exciplex may be formed by the hole transport host and the electron transport host in the emission layer EML. In this case, the triplet energy of the exciplex formed by the hole transport host and the electron transport host may correspond to T1 which is a gap between the LUMO energy level of the electron transport host and the HOMO energy level of the hole transport host.

In some embodiments, the triplet energy (T1) of the exciplex formed by the hole transport host and the electron transport host may be about 2.4 eV to about 3.0 eV. In addition, the triplet energy of the exciplex may be a value smaller than the energy gap of each host material. Accordingly, the exciplex may have a triplet energy of about 3.0 eV or less, which is the energy gap between the hole transport host and the electron transport host.

In some embodiments, at least one emission layer EML may include a quantum dot material. The core of the quantum dot may be selected from group II-VI compounds, group III-VI compounds, group I-III-VI compounds, group III-V compounds, group III-II-V compounds, group IV-VI compounds, group IV elements, group IV compounds, and combinations thereof.

The group II-VI compound may be selected from the group consisting of: a binary compound selected from the group consisting of CdSe, CdTe, CdS, ZnS, ZnSe, ZnTe, ZnO, HgS, HgSe, HgTe, MgSe, MgS, and mixtures thereof; a ternary compound selected from the group consisting of CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and mixtures thereof; and a quaternary compound selected from the group consisting of CdZnSeS, CdZnSeTe, CdZnSTe, CdHgSeS, CdHgSeTe, CdHgSTe, HgZnSeS, HgZnSeTe, HgZnSTe, and mixtures thereof.

The group III-VI compound may include a binary compound such as In₂S₃, and/or In₂Se₃, a ternary compound such as InGaS₃, and/or InGaSe₃, or optional combinations thereof.

The group I-III-VI compound may be selected from a ternary compound selected from the group consisting of AgInS, AgInS₂, CuInS, CuInS₂, AgGaS₂, CuGaS₂, CuGaO₂, AgGaO₂, AgAlO₂ and mixtures thereof, and/or a quaternary compound such as AgInGaS₂, and/or CuInGaS₂.

The group III-V compound may be selected from the group consisting of a binary compound selected from the group consisting of GaN, GaP, GaAs, GaSb, AlN, AlP, AlAs, AlSb, InN, InP, InAs, InSb, and mixtures thereof, a ternary compound selected from the group consisting of GaNP, GaNAs, GaNSb, GaPAs, GaPSb, AlNP, AlNAs, AlNSb, AlPAs, AlPSb, InGaP, InAlP, InNP, InNAs, InNSb, InPAs, InPSb, and mixtures thereof, and a quaternary compound selected from the group consisting of GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, and mixtures thereof. In some embodiments, the group III-V compound may further include a group II metal. For example, InZnP, etc. may be selected as a group III-II-V compound.

The group IV-VI compound may be selected from the group consisting of a binary compound selected from the group consisting of SnS, SnSe, SnTe, PbS, PbSe, PbTe, and mixtures thereof, a ternary compound selected from the group consisting of SnSeS, SnSeTe, SnSTe, PbSeS, PbSeTe, PbSTe, SnPbS, SnPbSe, SnPbTe, and mixtures thereof, and a quaternary compound selected from the group consisting of SnPbSSe, SnPbSeTe, SnPbSTe, and mixtures thereof. The group IV element may be selected from the group consisting of Si, Ge, and a mixture thereof. The group IV compound may be a binary compound selected from the group consisting of SiC, SiGe, and a mixture thereof.

In this case, the binary compound, the ternary compound and/or the quaternary compound may be present at uniform (e.g., substantially uniform) concentration in a particle or may be present at a partially different concentration distribution state in the same particle. In addition, a core/shell structure in which one quantum dot wraps another quantum dot may be possible. The interface of the core and the shell may have a concentration gradient in which the concentration of an element present in the shell decreases along a direction toward the center.

In some embodiments, the quantum dot may have the above-described core-shell structure including a core including a nanocrystal and a shell wrapping the core. The shell of the quantum dot may play the role of a protection layer for preventing or reducing the chemical deformation of the core to maintain semiconductor properties and/or a charging layer for imparting the quantum dot with electrophoretic properties. The shell may have a single layer or a multilayer. Examples of the shell of the quantum dot may include a metal and/or non-metal oxide, a semiconductor compound, or combinations thereof.

For example, the metal and/or non-metal oxide may include a binary compound such as SiO₂, Al₂O₃, TiO₂, ZnO, MnO, Mn₂O₃, Mn₃O₄, CuO, FeO, Fe₂O₃, Fe₃O₄, CoO, Co₃O₄ and/or NiO, or a ternary compound such as MgAl₂O₄, CoFe₂O₄, NiFe₂O₄ and/or CoMn₂O₄, but embodiments of the present disclosure are not limited thereto.

Also, the semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, etc., but embodiments of the present disclosure are not limited thereto.

The quantum dot may have a full width of half maximum (FWHM) of emission wavelength spectrum of about 45 nm or less, about 40 nm or less, or, for example, about 30 nm or less. Within this range, color purity and/or color reproducibility may be improved. In addition, light emitted via such quantum dot is emitted in all (e.g., substantially all) directions, and light view angle properties may be improved.

In addition, the shape of the quantum dot may be generally used shapes in the art, without specific limitation. In some embodiments, the shape of spherical, pyramidal, multi-arm, and/or cubic nanoparticle, nanotube, nanowire, nanofiber, nanoplate particle, etc. may be used.

The quantum dot may control the color of light emitted according to the particle size, and accordingly, the quantum dot may have various suitable emission colors such as blue, red and green.

In the light emitting elements ED of embodiments, as shown in FIG. 3 to FIG. 6, the electron transport region ETR is provided on the emission layer EML. The electron transport region ETR may include at least one of a hole blocking layer HBL, an electron transport layer ETL or an electron injection layer EIL. However, embodiments of the present disclosure are not limited thereto.

The electron transport region ETR may have a single layer formed using a single material, a single layer formed using a plurality of different materials, or a multilayer structure having a plurality of layers formed using a plurality of different materials.

For example, the electron transport region ETR may have a single layer structure of an electron injection layer EIL or an electron transport layer ETL, or a single layer structure formed using an electron injection material and an electron transport material. Further, the electron transport region ETR may have a single layer structure formed using a plurality of different materials, or a structure stacked from the emission layer EML of electron transport layer ETL/electron injection layer EIL, hole blocking layer HBL/electron transport layer ETL/electron injection layer EIL, electron transport layer ETL/buffer layer/electron injection layer EIL, without limitation. The thickness of the electron transport region ETR may be, for example, from about 1,000 Å to about 1,500 Å.

The electron transport region ETR may be formed using various suitable methods such as a vacuum deposition method, a spin coating method, a cast method, a Langmuir-Blodgett (LB) method, an inkjet printing method, a laser printing method, and/or a laser induced thermal imaging (LITI) method.

The electron transport region ETR may include a compound represented by Formula ET-1 below.

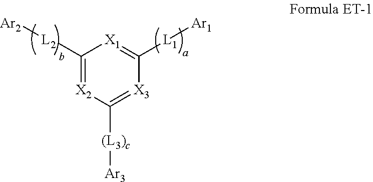

In Formula ET-1, at least one among X₁ to X₃ is N, and the remainder are CRₐ. Rₐ may be a hydrogen atom, a deuterium atom, a substituted or unsubstituted alkyl of 1 to 20 carbon atoms, a substituted or unsubstituted aryl group of 6 to 30 ring-forming carbon atoms, or a substituted or unsubstituted heteroaryl group of 2 to 30 ring-forming carbon atoms. Ar₁ to Ar₃ may be each independently a hydrogen atom, a deuterium atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In Formula ET-1, “a” to “c” may be each independently an integer of 0 to 10. In Formula ET-1, L₁ to L₃ may be each independently a direct linkage, a substituted or unsubstituted arylene group of 6 to 30 ring-forming carbon atoms, or a substituted or unsubstituted heteroarylene group of 2 to 30 ring-forming carbon atoms. If “a” to “c” are integers of 2 or more, L₁ to L₃ may be each independently a substituted or unsubstituted arylene group of 6 to 30 ring-forming carbon atoms, or a substituted or unsubstituted heteroarylene group of 2 to 30 ring-forming carbon atoms.

The electron transport region ETR may include an anthracene-based compound. However, embodiments of the present disclosure are not limited thereto, and the electron transport region ETR may include, for example, tris(8-hydroxyquinolinato)aluminum (Alq₃), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzoimidazolyl-1-yl)phenyl)-9,10-dinaphthylanthracene, 1,3,5-tri(1-phenyl-1H-benzo[d]imidazol-2-yl)benzene (TPBi), 2,9-dimethyl-4,7-diphenyl-1,10-phenanthroline (BCP), 4,7-diphenyl-1,10-phenanthroline (Bphen), 3-(4-biphenylyl)-4-phenyl-5-tert-butylphenyl-1,2,4-triazole (TAZ), 4-(naphthalen-1-yl)-3,5-diphenyl-4H-1,2,4-triazole (NTAZ), 2-(4-biphenylyl)-5-(4-tert-butylphenyl)-1,3,4-oxadiazole (tBu-PBD), bis(2-methyl-8-quinolinolato-N1,O8)-(1,1′-biphenyl-4-olato)aluminum (Balq), berylliumbis(benzoquinolin-10-olate) (Bebq₂), 9,10-di(naphthalene-2-yl)anthracene (ADN), 1,3-bis[3,5-di(pyridin-3-yl)phenyl]benzene (BmPyPhB), diphenyl(4-(triphenylsilyl)phenyl)phosphine oxide (TSPO1), or mixtures thereof, without limitation.

The electron transport region ETR may include at least one among Compounds ET1 to ET36 below.

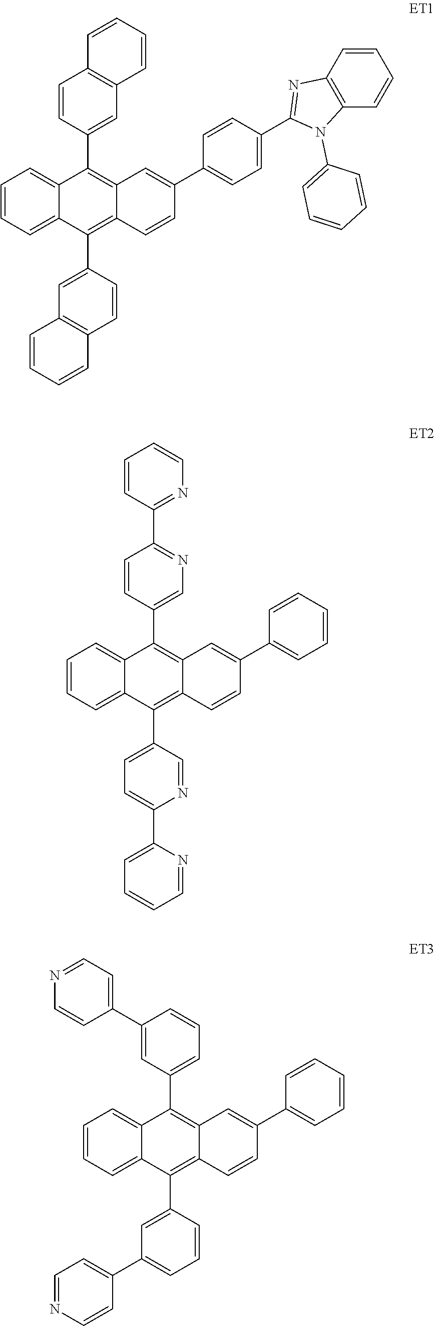 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

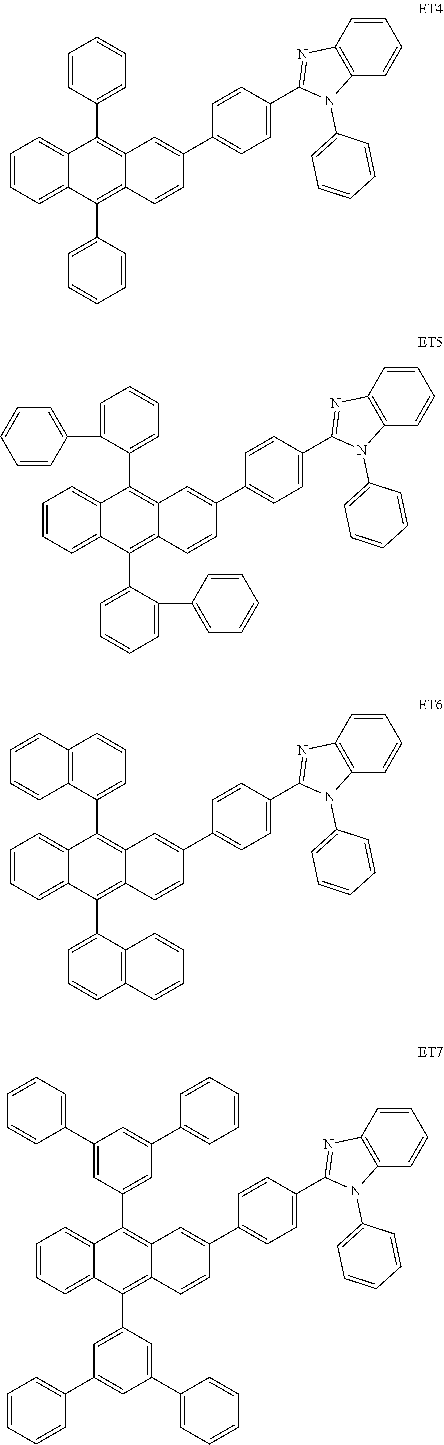 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1 ; c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1]

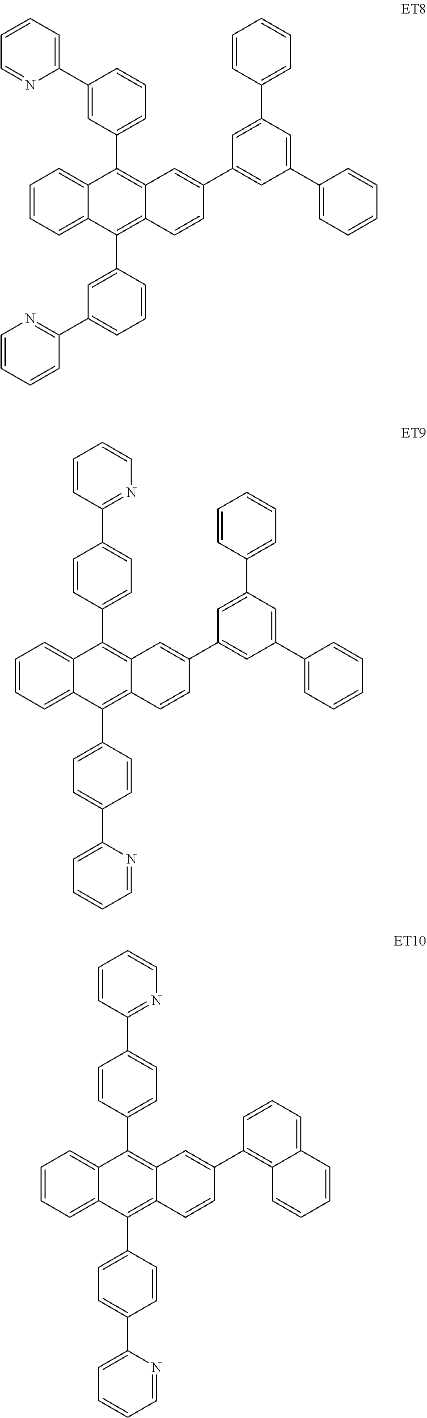 [SMILES: c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1 ; c1ccc(-c2ccc(-c3c4ccccc4c(-c4ccc(-c5ccccn5)cc4)c4cc(-c5cccc6ccccc56)ccc34)cc2)nc1]

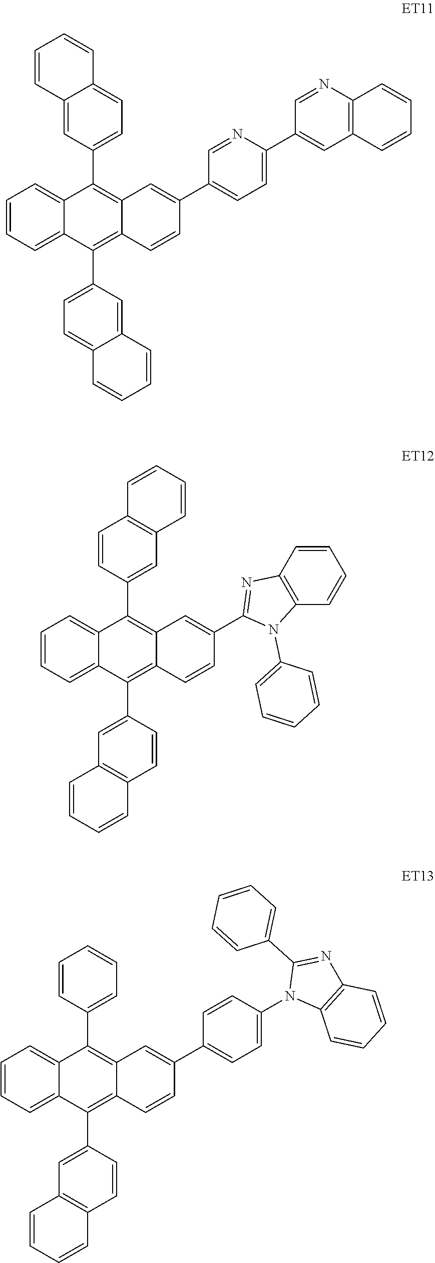 [SMILES: c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1 ; c1ccc(-c2c3ccccc3c(-c3ccc4ccccc4c3)c3ccc(-c4ccc(-n5c(-c6ccccc6)nc6ccccc65)cc4)cc23)cc1]

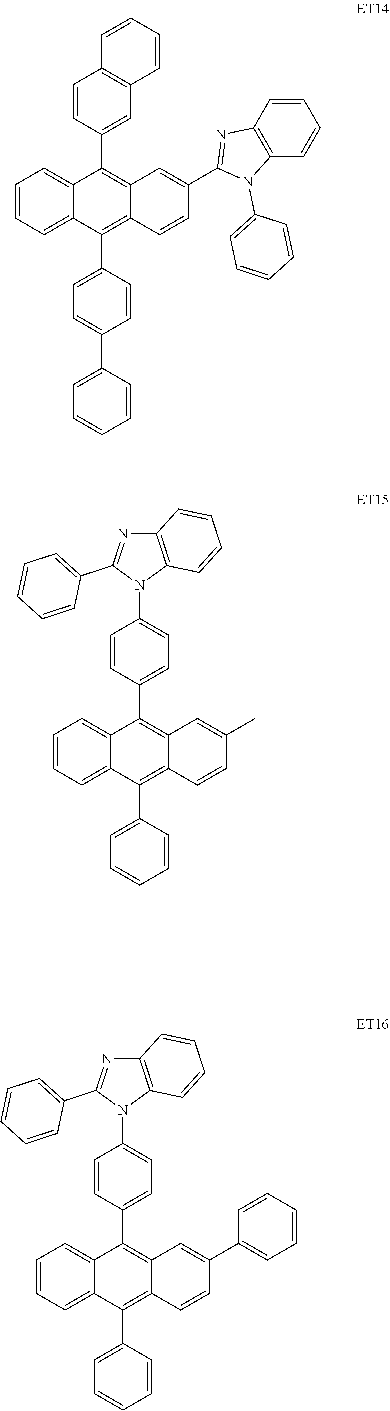 [SMILES: c1ccc(-c2ccc(-c3c4ccccc4c(-c4ccc5ccccc5c4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1 ; c1ccc(-c2ccc3c(-c4ccccc4)c4ccccc4c(-c4ccc(-n5c(-c6ccccc6)nc6ccccc65)cc4)c3c2)cc1]

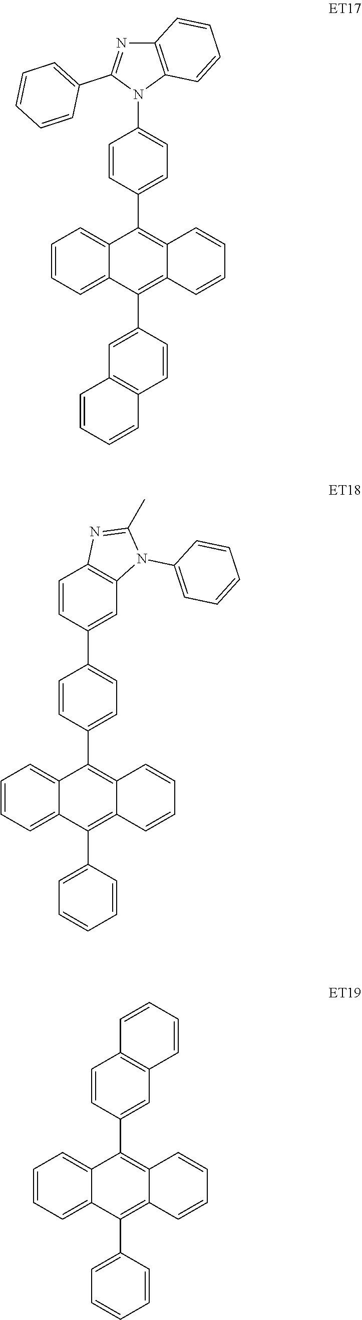

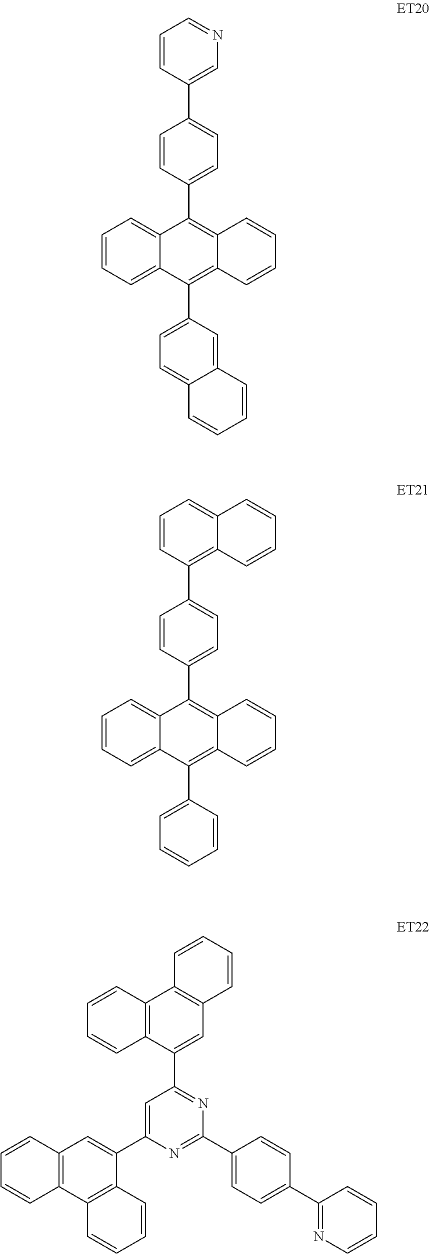

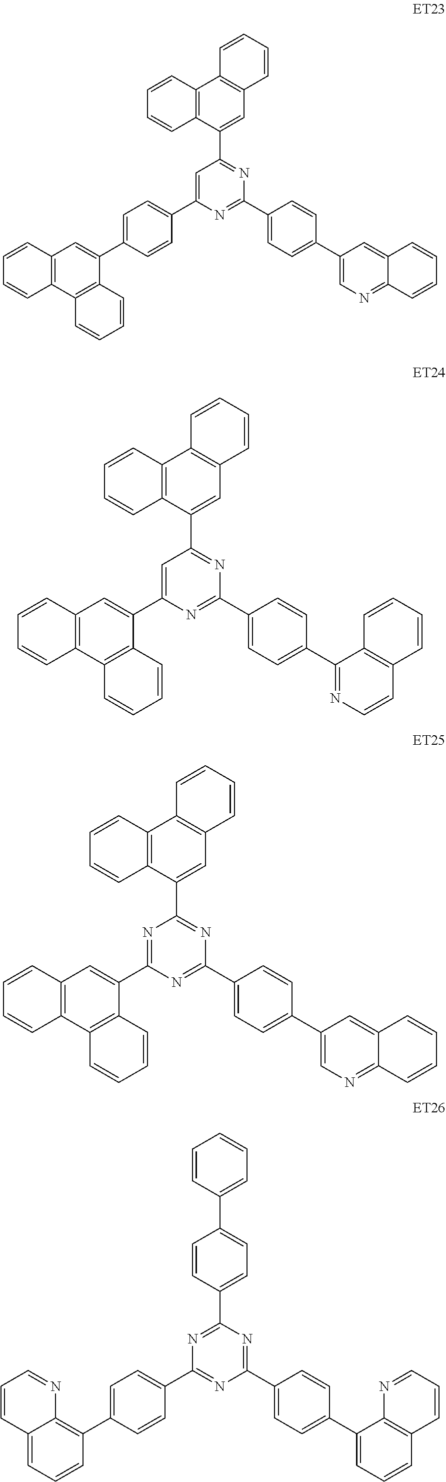 [SMILES: c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1 ; c1ccc2ncc(-c3ccc(-c4nc(-c5cc6ccccc6c6ccccc56)nc(-c5cc6ccccc6c6ccccc56)n4)cc3)cc2c1 ; c1ccc(-c2ccc(-c3nc(-c4ccc(-c5cccc6cccnc56)cc4)nc(-c4ccc(-c5cccc6cccnc56)cc4)n3)cc2)cc1]

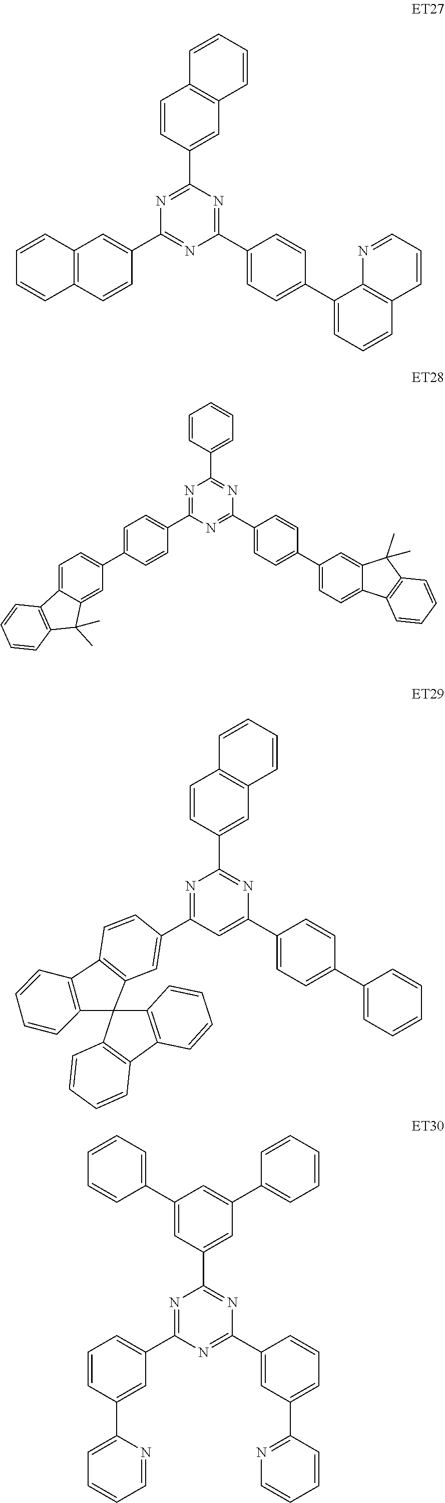

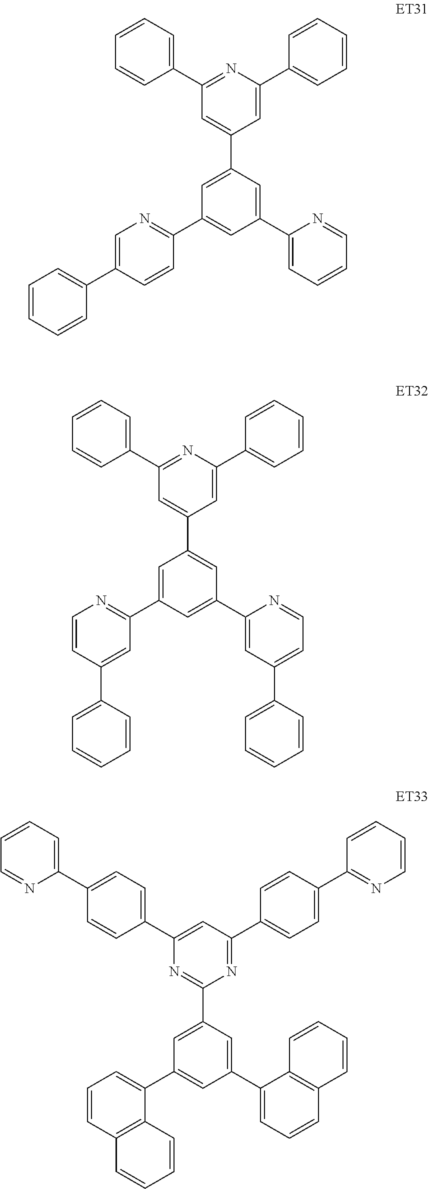 [SMILES: c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1]

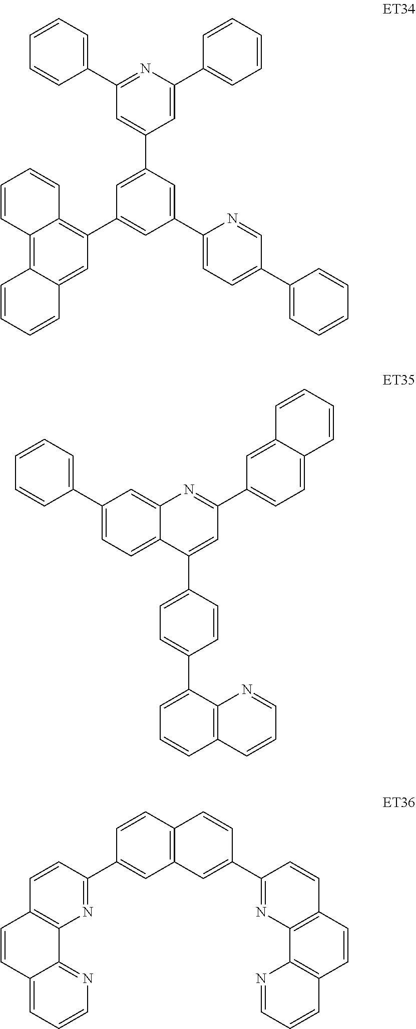 [SMILES: c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12]

In addition, the electron transport region ETR may include a metal halide such as LiF, NaCl, CsF, RbCl, RbI, CuI and/or Kl, a metal of the lanthanoides such as Yb, or a co-deposited material of the metal halide and the metal of the lanthanoides. For example, the electron transport region ETR may include Kl:Yb, RbI:Yb, LiF:Yb, etc., as the co-deposited material. In some embodiments, the electron transport region ETR may use a metal oxide such as Li₂O and BaO, and/or 8-hydroxy-lithium quinolate (Liq). However, embodiments of the present disclosure are not limited thereto. The electron transport region ETR also may be formed using a mixture material of an electron transport material and an insulating organo metal salt. The organo metal salt may be a material having an energy band gap of about 4 eV or more. In some embodiments, the organo metal salt may include, for example, metal acetates, metal benzoates, metal acetoacetates, metal acetylacetonates, and/or metal stearates.

The electron transport region ETR may include at least one of 2,9-dimethyl-4,7-diphenyl-1,10-phenanthroline (BCP), diphenyl(4-(triphenylsilyl)phenyl)phosphine oxide (TSPO1) or 4,7-diphenyl-1,10-phenanthroline (Bphen) in addition to the aforementioned materials. However, embodiments of the present disclosure are not limited thereto.

The electron transport region ETR may include the compounds of the electron transport region in at least one among an electron injection layer EIL, an electron transport layer ETL, and a hole blocking layer HBL.

If the electron transport region ETR includes the electron transport layer ETL, the thickness of the electron transport layer ETL may be from about 100 Å to about 1,000 Å, for example, from about 150 Å to about 500 Å. If the thickness of the electron transport layer ETL satisfies the above-described range, suitable or satisfactory electron transport properties may be obtained without substantial increase of a driving voltage. If the electron transport region ETR includes the electron injection layer EIL, the thickness of the electron injection layer EIL may be from about 1 Å to about 100 Å, and from about 3 Å to about 90 Å. If the thickness of the electron injection layer EIL satisfies the above described range, suitable or satisfactory electron injection properties may be obtained without inducing substantial increase of a driving voltage.

The second electrode EL2 is provided on the electron transport region ETR. The second electrode EL2 may be a common electrode. The second electrode EL2 may be a cathode or an anode, but embodiments of the present disclosure are not limited thereto. For example, if the first electrode EL1 is an anode, the second cathode EL2 may be a cathode, and if the first electrode EL1 is a cathode, the second electrode EL2 may be an anode. The second electrode EL2 may include at least one selected among Ag, Mg, Cu, Al, Pt, Pd, Au, Ni, Nd, Ir, Cr, Li, Ca, LiF, Mo, Ti, W, In, Sn, and Zn, compounds of two or more selected therefrom, mixtures of two or more selected therefrom, and/or oxides thereof.

The second electrode EL2 may be a transmissive electrode, a transflective electrode or a reflective electrode. If the second electrode EL2 is the transmissive electrode, the second electrode EL2 may include a transparent metal oxide, for example, ITO, IZO, ZnO, ITZO, etc.

If the second electrode EL2 is the transflective electrode or the reflective electrode, the second electrode EL2 may include Ag, Mg, Cu, Al, Pt, Pd, Au, Ni, Nd, Ir, Cr, Li, Ca, LiF/Ca (stacked structure of LiF and Ca), LiF/Al (stacked structure of LiF and Al), Mo, Ti, Yb, W, compounds including thereof, or mixtures thereof (for example, AgMg, AgYb, and/or MgYb). Otherwise, the second electrode EL2 may have a multilayered structure including a reflective layer or a transflective layer formed using the above-described materials and a transparent conductive layer formed using ITO, IZO, ZnO, ITZO, etc. For example, the second electrode EL2 may include the aforementioned metal materials, combinations of two or more metal materials selected from the aforementioned metal materials, or oxides of the aforementioned metal materials.

In some embodiments, the second electrode EL2 may be connected with an auxiliary electrode. If the second electrode EL2 is connected with the auxiliary electrode, the resistance of the second electrode EL2 may decrease.

A capping layer CPL may be on the second electrode EL2 in the light emitting element ED of an embodiment. The capping layer CPL may include a multilayer or a single layer.

In some embodiments, the capping layer CPL may be an organic layer and/or an inorganic layer. For example, if the capping layer CPL includes an inorganic material, the inorganic material may include an alkali metal compound such as LiF, an alkaline earth metal compound such as SiON, SiNx, SiOy, etc.

For example, if the capping layer CPL includes an organic material, the organic material may include α-NPD, NPB, TPD, m-MTDATA, Alq₃, CuPc, N4,N4,N4′,N4′-tetra(biphenyl-4-yl) biphenyl-4,4′-diamine (TPD15), 4,4′,4″-tris(carbazol-9-yl) triphenylamine (TCTA), etc., and/or includes an epoxy resin, and/or acrylate such as methacrylate. In addition, a capping layer CPL may include at least one among Compounds P1 to P5 below, but embodiments of the present disclosure are not limited thereto.

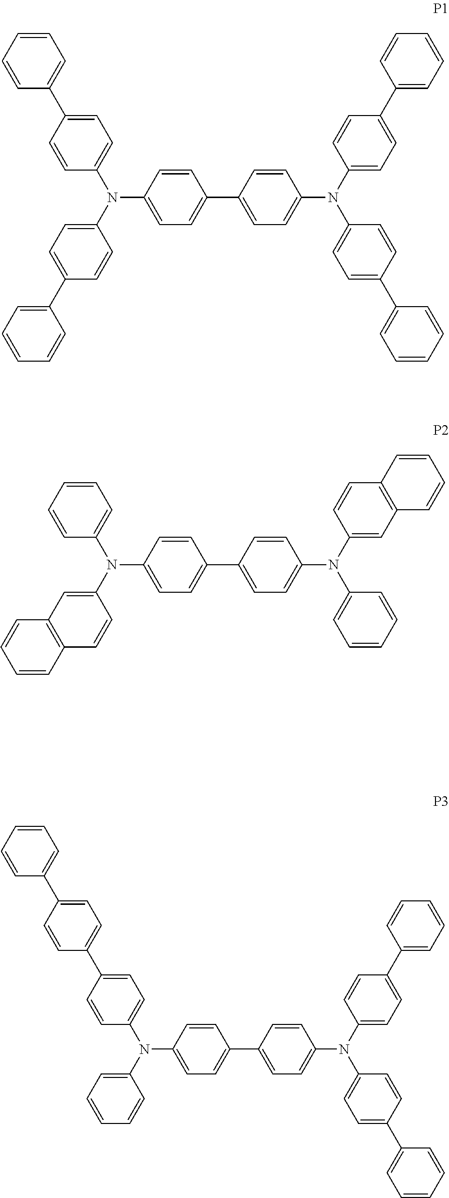 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1]

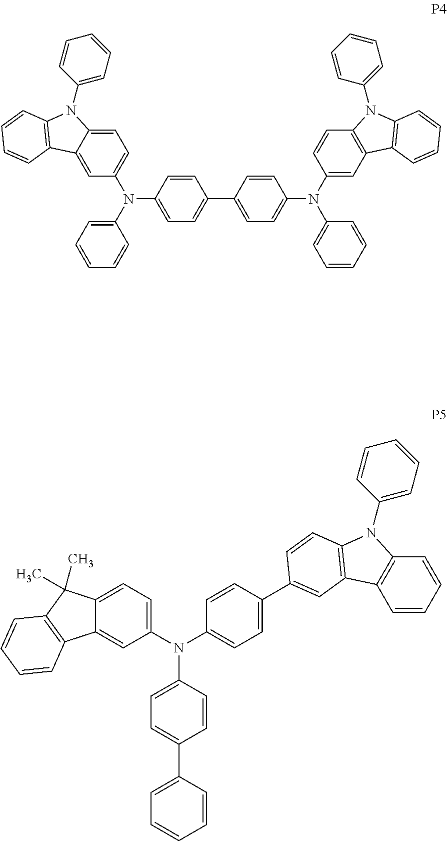 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; CC1(C)c2ccccc2-c2cc(N(c3ccc(-c4ccccc4)cc3)c3ccc(-c4ccc5c(c4)c4ccccc4n5-c4ccccc4)cc3)ccc21]

In some embodiments, the refractive index of the capping layer CPL may be about 1.6 or more. For example, the refractive index of the capping layer CPL with respect to light in a wavelength range of about 550 nm to about 660 nm may be about 1.6 or more.

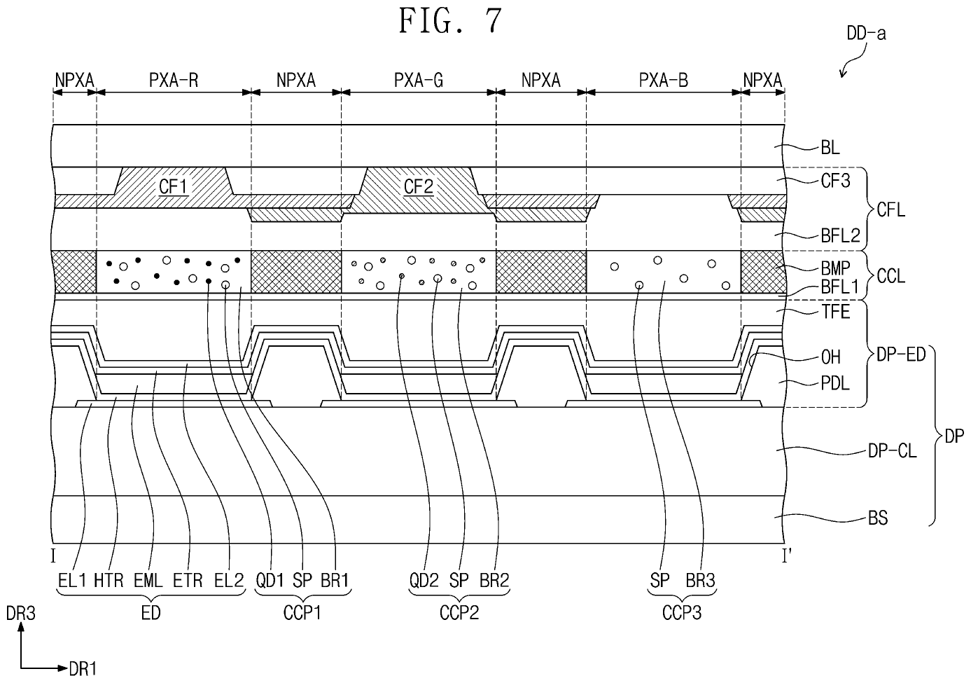

FIG. 7 to FIG. 10 are cross-sectional views of display devices according to embodiments. In the explanation of the display devices of embodiments, referring to FIG. 7 to FIG. 10, the overlapping parts with the explanation of FIG. 1 to FIG. 6 will not be explained again here, and the different features will be explained chiefly.

Referring to FIG. 7, a display device DD-a according to an embodiment may include a display panel DP including a display element layer DP-ED, a light controlling layer CCL on the display panel DP, and a color filter layer CFL.

In some embodiments, as shown in FIG. 7, the display panel DP includes a base layer BS, a circuit layer DP-CL provided on the base layer BS and a display element layer DP-ED, and the display element layer DP-ED may include a light emitting element ED.

The light emitting element ED may include a first electrode EL1, a hole transport region HTR on the first electrode EL1, an emission layer EML on the hole transport region HTR, an electron transport region ETR on the emission layer EML, and a second electrode EL2 on the electron transport region ETR. The structures of the light emitting elements of FIG. 3 to FIG. 6 may be applied to the structure of the light emitting element ED shown in FIG. 7.

The hole transport region HTR of the light emitting element ED included in the display device DD-a according to an embodiment may include the amine compound of an embodiment, described above.

Referring to FIG. 7, the emission layer EML may be in an opening portion OH defined in a pixel definition layer PDL. For example, the emission layer EML divided by the pixel definition layer PDL and correspondingly provided to each of luminous areas PXA-R, PXA-G and PXA-B may emit light in the same wavelength region. In the display device DD-a of an embodiment, the emission layer EML may emit blue light. In some embodiments, different from the drawings, the emission layer EML may be provided as a common layer for all luminous areas PXA-R, PXA-G and PXA-B.

The light controlling layer CCL may be on the display panel DP. The light controlling layer CCL may include a light converter. The light converter may be a quantum dot and/or a phosphor. The light converter may transform the wavelength of light provided and then emit. For example, the light controlling layer CCL may be a layer including a quantum dot and/or a layer including a phosphor.

The light controlling layer CCL may include a plurality of light controlling parts CCP1, CCP2 and CCP3. The light controlling parts CCP1, CCP2 and CCP3 may be separated (e.g., spaced apart) from one another.

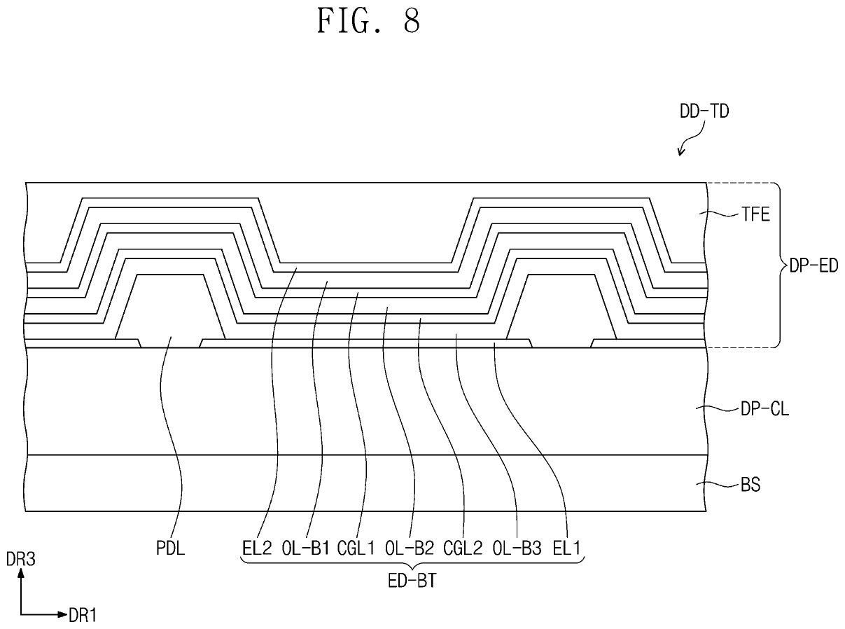

Referring to FIG. 7, a partition pattern BMP may be between the separated light controlling parts CCP1, CCP2 and CCP3, but embodiments of the present disclosure are not limited thereto. In FIG. 8, the partition pattern BMP is shown not to be overlapped with the light controlling parts CCP1, CCP2 and CCP3, but at least a portion of the edge of the light controlling parts CCP1, CCP2 and CCP3 may be overlapped with the partition pattern BMP.

The light controlling layer CCL may include a first light controlling part CCP1 including a first quantum dot QD1 that converts a first color light provided from the light emitting element ED into a second color light, a second light controlling part CCP2 including a second quantum dot QD2 that converts the first color light into a third color light, and a third light controlling part CCP3 that transmits the first color light.

In some embodiments, the first light controlling part CCP1 may provide red light which is the second color light, and the second light controlling part CCP2 may provide green light which is the third color light. The third color controlling part CCP3 may transmit and provide blue light which is the first color light provided from the light emitting element ED. For example, the first quantum dot QD1 may be a red quantum dot, and the second quantum dot QD2 may be a green quantum dot. For the quantum dots QD1 and QD2, the same contents as those described above may be applied.

In addition, the light controlling layer CCL may further include a scatterer SP (e.g., a light scatterer SP). The first light controlling part CCP1 may include the first quantum dot QD1 and the scatterer SP, the second light controlling part CCP2 may include the second quantum dot QD2 and the scatterer SP, and the third light controlling part CCP3 may not include a quantum dot but include the scatterer SP.

The scatterer SP may be an inorganic particle. For example, the scatterer SP may include at least one among TiO₂, ZnO, Al₂O₃, SiO₂, and hollow silica. The scatterer SP may include at least one among TiO₂, ZnO, Al₂O₃, SiO₂, and hollow silica, or may be a mixture of two or more materials selected among TiO₂, ZnO, Al₂O₃, SiO₂, and hollow silica.

Each of the first light controlling part CCP1, the second light controlling part CCP2, and the third light controlling part CCP3 may include base resins BR1, BR2 and BR3 that disperse the quantum dots QD1 and QD2 and the scatterer SP. In some embodiments, the first light controlling part CCP1 may include the first quantum dot QD1 and the scatterer SP dispersed in the first base resin BR1, the second light controlling part CCP2 may include the second quantum dot QD2 and the scatterer SP dispersed in the second base resin BR2, and the third light controlling part CCP3 may include the scatterer particle SP dispersed in the third base resin BR3. The base resins BR1, BR2 and BR3 are mediums in which the quantum dots QD1 and QD2 and the scatterer SP are dispersed, and may be composed of various suitable resin compositions which may be generally referred to as a binder. For example, the base resins BR1, BR2 and BR3 may be acrylic resins, urethane-based resins, silicone-based resins, epoxy-based resins, etc. The base resins BR1, BR2 and BR3 may be transparent resins. In some embodiments, the first base resin BR1, the second base resin BR2 and the third base resin BR3 may be the same or different from each other.

The light controlling layer CCL may include a first barrier layer BFL1. The first barrier layer BFL1 may play the role of blocking the penetration of moisture and/or oxygen (hereinafter, will be referred to as “humidity/oxygen”). The first barrier layer BFL1 may be on the light controlling parts CCP1, CCP2 and CCP3 to block the exposure of the light controlling parts CCP1, CCP2 and CCP3 to humidity/oxygen. In some embodiments, the first barrier layer BFL1 may cover the light controlling parts CCP1, CCP2 and CCP3. In addition, a barrier layer second BFL2 may be provided between the light controlling parts CCP1, CCP2 and CCP3 and filters CF1, CF2 and CF3.

The barrier layers BFL1 and BFL2 may include at least one inorganic layer. For example, the barrier layers BFL1 and BFL2 may be formed by including an inorganic material. For example, the barrier layers BFL1 and BFL2 may be formed by including silicon nitride, aluminum nitride, zirconium nitride, titanium nitride, hafnium nitride, tantalum nitride, silicon oxide, aluminum oxide, titanium oxide, tin oxide, cerium oxide and/or silicon oxynitride and/or a metal thin film securing light transmittance. In some embodiments, the barrier layers BFL1 and BFL2 may further include an organic layer. The barrier layers BFL1 and BFL2 may be composed of a single layer or a plurality of layers.

In the display device DD of an embodiment, the color filter layer CFL may be on the light controlling layer CCL. For example, the color filter layer CFL may be directly on the light controlling layer CCL. In this case, the second barrier layer BFL2 may be omitted.

The color filter layer CFL may include filters CF1, CF2 and CF3. The color filter layer CFL may include a first filter CF1 that transmits the second color light, a second filter CF2 that transmits the third color light, and a third filter CF3 that transmits the first color light. For example, the first filter CF1 may be a red filter, the second filter CF2 may be a green filter, and the third filter CF3 may be a blue filter. Each of the filters CF1, CF2 and CF3 may include a polymer photosensitive resin and a pigment and/or dye. The first filter CF1 may include a red pigment and/or dye, the second filter CF2 may include a green pigment and/or dye, and the third filter CF3 may include a blue pigment and/or dye. Embodiments of the present disclosure are, however, not limited thereto, and the third filter CF3 may not include the pigment or dye. The third filter CF3 may include a polymer photosensitive resin and not include a pigment or dye. The third filter CF3 may be transparent. The third filter CF3 may be formed using a transparent photosensitive resin.

In addition, in some embodiments, the first filter CF1 and the second filter CF2 may be yellow filters. The first filter CF1 and the second filter CF2 may be provided in one body without distinction. Each of the first to third filters CF1, CF2 and CF3 may respectively correspond to a red luminous area PXA-R, green luminous area PXA-G, and blue luminous area PXA-B.

In some embodiments, the color filter layer CFL may include a light blocking part. The color filter layer CFL may include the light blocking part so as to overlap with the boundaries of the neighboring filters CF1, CF2 and CF3. The light blocking part may be a black matrix. The light blocking part may be formed by including an organic light blocking material and/or an inorganic light blocking material, including a black pigment and/or black dye. The light blocking part may divide the boundaries among adjacent filters CF1, CF2 and CF3. In addition, in some embodiments, the light blocking part may be formed as a blue filter.

A base substrate BL may be on the color filter layer CFL. The base substrate BL may be a member providing a base surface that the color filter layer CFL, the light controlling layer CCL, etc. are on. The base substrate BL may be a glass substrate, a metal substrate, a plastic substrate, etc. However, embodiments of the present disclosure are not limited thereto, and the base substrate BL may be an inorganic layer, an organic layer or a composite material layer. In addition, different from the drawing, the base substrate BL may be omitted in some embodiments.

FIG. 8 is a cross-sectional view showing a portion of the display device according to an embodiment. In FIG. 8, the cross-sectional view of a portion corresponding to the display panel DP in FIG. 7 is shown. In a display device DD-TD of an embodiment, the light emitting element ED-BT may include a plurality of light emitting structures OL-B1, OL-B2 and OL-B3. The light emitting element ED-BT may include first electrode EL1 and second electrode EL2 opposite to each other, and the plurality of light emitting structures OL-B1, OL-B2 and OL-B3 stacked in order in a thickness direction and provided between the first electrode EL1 and the second electrode EL2. Each of the light emitting structures OL-B1, OL-B2 and OL-B3 may include an emission layer EML (FIG. 7), a hole transport region HTR and an electron transport region ETR with the emission layer EML (FIG. 7) therebetween.

For example, the light emitting element ED-BT included in the display device DD-TD of an embodiment may be a light emitting element of a tandem structure including a plurality of emission layers.

In some embodiments, as shown in FIG. 8, light emitted from the light emitting structures OL-B1, OL-B2 and OL-B3 may be all blue light. However, embodiments of the present disclosure are not limited thereto, and the wavelength regions of light emitted from the light emitting structures OL-B1, OL-B2 and OL-B3 may be different from each other. For example, the light emitting element ED-BT including the plurality of light emitting structures OL-B1, OL-B2 and OL-B3 emitting light in different wavelength regions may emit white light.

Charge generating layers CGL1 and CGL2 may be between neighboring light emitting structures OL-B1, OL-B2 and OL-B3. The charge generating layers CGL1 and CGL2 may include a p-type charge generating layer and/or an n-type charge generating layer.

In at least one among the light emitting structures OL-B1, OL-B2 and OL-B3, included in the display device DD-TD of an embodiment, the amine compound of an embodiment may be included.

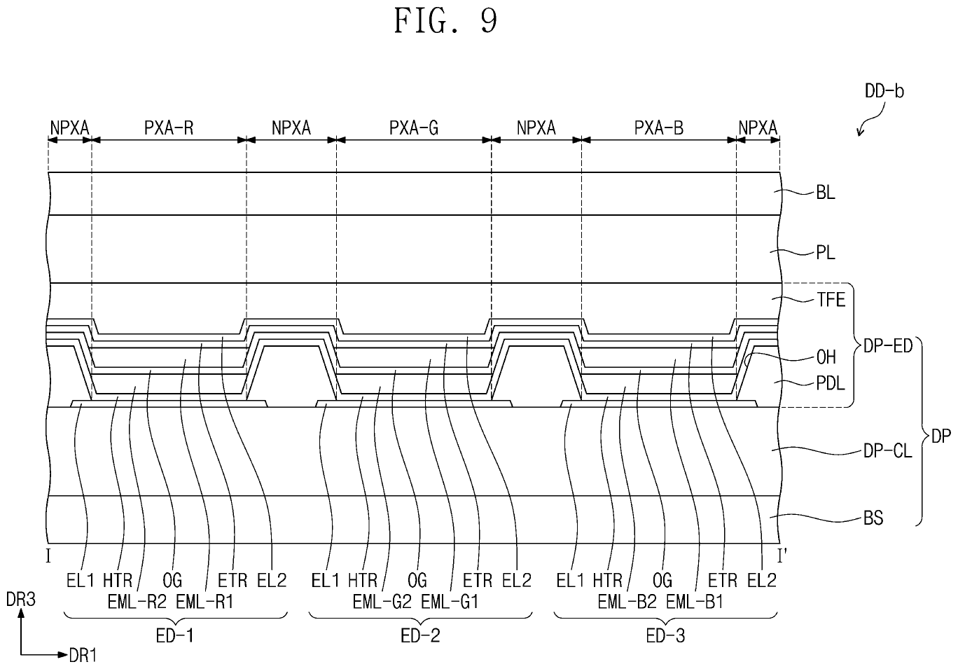

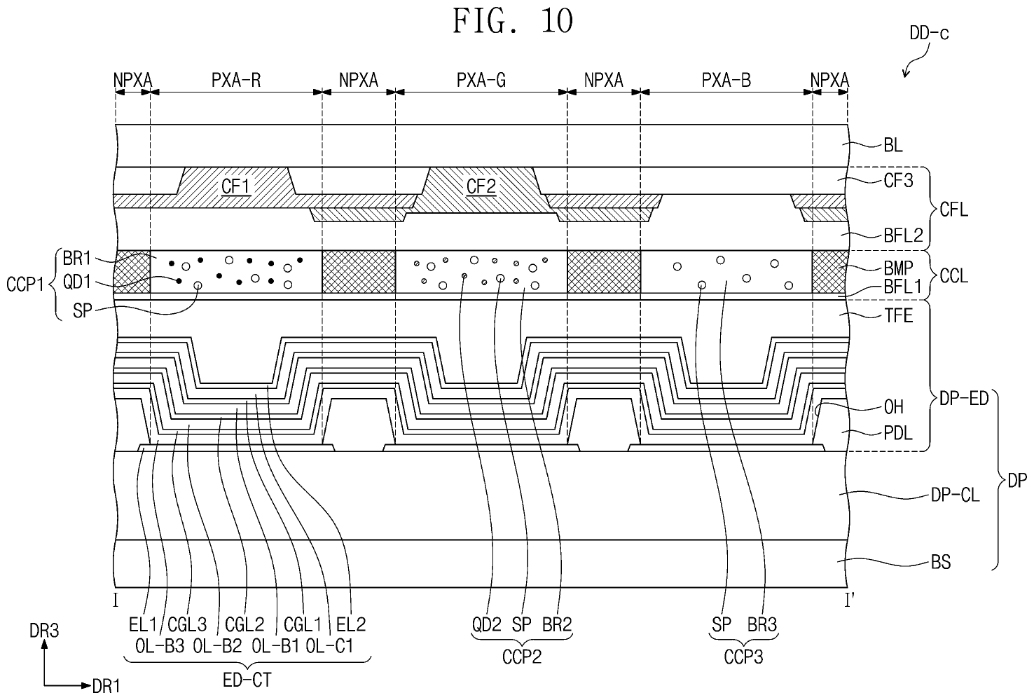

Referring to FIG. 9, a display device DD-b according to an embodiment may include light emitting elements ED-1, ED-2 and ED-3, formed by stacking two emission layers. Compared to the display device DD of an embodiment, shown in FIG. 2, an embodiment shown in FIG. 9 is different in that first to third light emitting elements ED-1, ED-2 and ED-3 include two emission layers stacked in a thickness direction, each. In the first to third light emitting elements ED-1, ED-2 and ED-3, two emission layers may emit light in the same wavelength region.

The first light emitting element ED-1 may include a first red emission layer EML-R1 and a second red emission layer EML-R2. The second light emitting element ED-2 may include a first green emission layer EML-G1 and a second green emission layer EML-G2. In addition, the third light emitting element ED-3 may include a first blue emission layer EML-B1 and a second blue emission layer EML-B2. An emission auxiliary part OG may be between the first red emission layer EML-R1 and the second red emission layer EML-R2, between the first green emission layer EML-G1 and the second green emission layer EML-G2, and between the first blue emission layer EML-B1 and the second blue emission layer EML-B2.

The emission auxiliary part OG may include a single layer or a multilayer. The emission auxiliary part OG may include a charge generating layer. In some embodiments, the emission auxiliary part OG may include an electron transport region, a charge generating layer, and a hole transport region stacked in order. The emission auxiliary part OG may be provided as a common layer in all of the first to third light emitting elements ED-1, ED-2 and ED-3. However, embodiments of the present disclosure are not limited thereto, and the emission auxiliary part OG may be patterned and provided in an opening portion OH defined in a pixel definition layer PDL.

The first red emission layer EML-R1, the first green emission layer EML-G1 and the first blue emission layer EML-B1 may be between the electron transport region ETR and the emission auxiliary part OG. The second red emission layer EML-R2, the second green emission layer EML-G2 and the second blue emission layer EML-B2 may be between the emission auxiliary part OG and the hole transport region HTR.

For example, the first light emitting element ED-1 may include a first electrode EL1, a hole transport region HTR, a second red emission layer EML-R2, an emission auxiliary part OG, a first red emission layer EML-R1, an electron transport region ETR, and a second electrode EL2, stacked in order. The second light emitting element ED-2 may include a first electrode EL1, a hole transport region HTR, a second green emission layer EML-G2, an emission auxiliary part OG, a first green emission layer EML-G1, an electron transport region ETR, and a second electrode EL2, stacked in order. The third light emitting element ED-3 may include a first electrode EL1, a hole transport region HTR, a second blue emission layer EML-B2, an emission auxiliary part OG, a first blue emission layer EML-B1, an electron transport region ETR, and a second electrode EL2, stacked in order.

In some embodiments, an optical auxiliary layer PL may be on a display element layer DP-ED. The optical auxiliary layer PL may include a polarization layer. The optical auxiliary layer PL may be on a display panel DP and may control reflected light at the display panel DP by external light. Different from the drawings, the optical auxiliary layer PL may be omitted from the display device according to an embodiment.

Different from FIG. 8 and FIG. 9, a display device DD-c in FIG. 10 is shown to include four light emitting structures OL-B1, OL-B2, OL-B3 and OL-C1. A light emitting element ED-CT may include first electrode EL1 and second electrode EL2 opposite to each other, and first to fourth light emitting structures OL-B1, OL-B2, OL-B3 and OL-C1 stacked in order in a thickness direction between the first electrode EL1 and the second electrode EL2. Charge generating layers CGL1, CGL2 and CGL3 may be between the first to fourth light emitting structures OL-B1, OL-B2, OL-B3 and OL-C1. Among the four light emitting structures, the first to third light emitting structures OL-B1, OL-B2 and OL-B3 may emit blue light, and the fourth light emitting structure OL-C1 may emit green light. However, embodiments of the present disclosure are not limited thereto, and the first to fourth light emitting structures OL-B1, OL-B2, OL-B3 and OL-C1 may emit different wavelengths of light.

Charge generating layers CGL1, CGL2 and CGL3 located among neighboring light emitting structures OL-B1, OL-B2, OL-B3 and OL-C1 may include a p-type charge generating layer and/or an n-type charge generating layer.

In at least one among the light emitting structures OL-B1, OL-B2, OL-B3 and OL-C1, included in the display device DD-c of an embodiment, the amine compound of an embodiment may be included.

The light emitting element ED according to embodiments of the present disclosure may include the amine compound of an embodiment in at least one functional layer between the first electrode EL1 and the second electrode EL2 to exhibit improved emission efficiency and improved life characteristics. The light emitting element ED according to an embodiment may include the amine compound of an embodiment in at least one among a hole transport region HTR, an emission layer EML, and an electron transport region ETR, between the first electrode EL1 and the second electrode EL2, or in a capping layer CPL. For example, the amine compound according to an embodiment may be included in the hole transport region HTR of the light emitting element ED of an embodiment, and the light emitting element of an embodiment may exhibit long-life characteristics.

The amine compound of an embodiment includes a first to third substituents, and may improve the stability of a material and improve hole transport properties. Accordingly, the lifetime of a light emitting element including the amine compound of an embodiment may increase. In addition, the light emitting element of an embodiment may include the amine compound of an embodiment in an electron blocking layer to exhibit improved life characteristics.

Hereinafter, referring to embodiments and comparative embodiments, the amine compound according to an embodiment and the light emitting element according to embodiments of the present disclosure are will be further explained in more detail. In addition, the embodiments below are illustrations to assist the understanding of embodiments of the present disclosure are, but the scope of the present disclosure is not limited thereto.

### EXAMPLES

### 1. Synthesis of Amine Compounds

First, the synthetic methods of the amine compounds according to embodiments will be further explained by describing the synthetic methods of Compound 1, Compound 3, Compound 4, Compound 5, Compound 6, Compound 7, Compound 8, Compound 9, Compound 10, Compound 11, Compound 12, Compound 13, Compound 14, Compound 18, Compound 28, Compound 61, and Compound 70 shown in Table 1 below. In addition, the synthetic methods of the amine compounds explained hereinafter are embodiments, and the synthetic method of the amine compound according to embodiments of the present disclosure are not limited to the embodiments below.

### Synthetic Method of Compounds

Under an argon (Ar) atmosphere, to a 200 mL, three-neck flask, Intermediate Compound P (10.0 mmol), Intermediate Compound Q (11.0 mmol), Pd(dba)₂ (0.29 g, 0.50 mmol), P(tBu)₃·HBF₄ (0.59 g, 2.0 mmol), and NaOtBu (1.45 g, 15.0 mmol) were added and stirred in 50 mL of a toluene solvent at about 130° C. for about 8 hours. After cooling, the stirred mixture was washed with water, and an organic layer was separated. The separated organic layer was purified by column chromatography (silica gel) to obtain a target compound. The identification of the compound was conducted by measuring FAB-MS and ¹H-NMR (CDCl₃).

In Table 1, the structure and mass added of Intermediate Compound P, the structure and mass added of Intermediate Compound Q, the amount obtained and yield of the target compound produced, and FAB-MS are shown. In Table 2, the ¹H-NMR data of the target compound are shown. Meanwhile, the molecular weight of the Example Compound was obtained by measuring FAB-MS using JMS-700V of JEOL Co. In addition, the ¹H-NMR of the Example Compound was measured using AVAVCE300M of Bruker Biospin K.K.

| | | Amount | Intermediate | Amount |
|---|---|---|---|---|
| Divi- | Intermediate | used | Compound | used |
| sion | Compound P | (mass) | Q | (mass) |
| a01 | 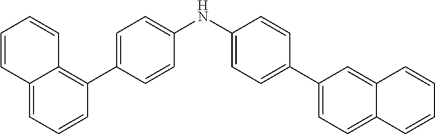 [SMILES: c1ccc2cc(-c3ccc(Nc4ccc(-c5cccc6ccccc56)cc4)cc3)ccc2c1] | 4.22 g | 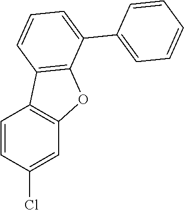 [SMILES: Clc1ccc2c(c1)oc1c(-c3ccccc3)cccc12] | 3.07 g |
| a02 | 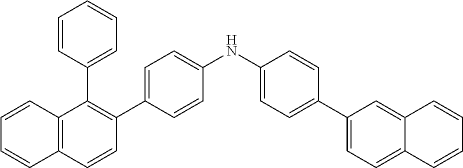 [SMILES: c1ccc(-c2c(-c3ccc(Nc4ccc(-c5ccc6ccccc6c5)cc4)cc3)ccc3ccccc23)cc1] | 4.98 g | 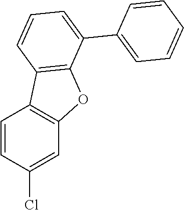 [SMILES: Clc1ccc2c(c1)oc1c(-c3ccccc3)cccc12] | 3.07 g |
| a03 | 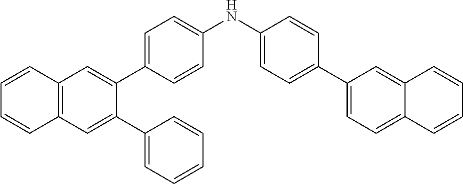 [SMILES: c1ccc(-c2cc3ccccc3cc2-c2ccc(Nc3ccc(-c4ccc5ccccc5c4)cc3)cc2)cc1] | 4.98 g | 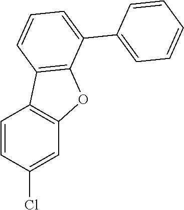 [SMILES: Clc1ccc2c(c1)oc1c(-c3ccccc3)cccc12] | 3.07 g |
| a04 | 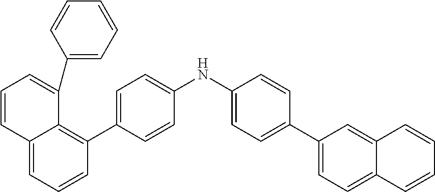 [SMILES: c1ccc(-c2cccc3cccc(-c4ccc(Nc5ccc(-c6ccc7ccccc7c6)cc5)cc4)c23)cc1] | 4.98 g | 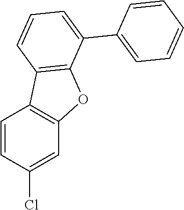 [SMILES: Clc1ccc2c(c1)oc1c(-c3ccccc3)cccc12] | 3.07 g |
| a05 | 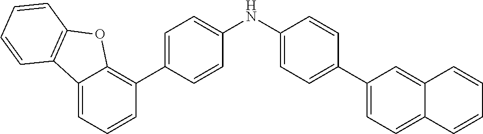 [SMILES: c1ccc2cc(-c3ccc(Nc4ccc(-c5cccc6c5oc5ccccc56)cc4)cc3)ccc2c1] | 4.62 g | 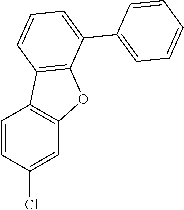 [SMILES: Clc1ccc2c(c1)oc1c(-c3ccccc3)cccc12] | 3.07 g |
| a06 | 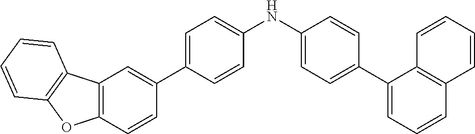 [SMILES: c1ccc2c(-c3ccc(Nc4ccc(-c5ccc6oc7ccccc7c6c5)cc4)cc3)cccc2c1] | 4.62 g | 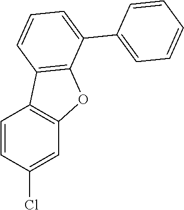 [SMILES: Clc1ccc2c(c1)oc1c(-c3ccccc3)cccc12] | 3.07 g |
| a07 | 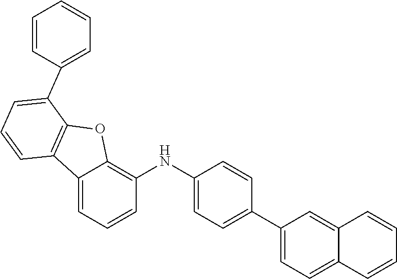 [SMILES: c1ccc(-c2cccc3c2oc2c(Nc4ccc(-c5ccc6ccccc6c5)cc4)cccc23)cc1] | 4.62 g | 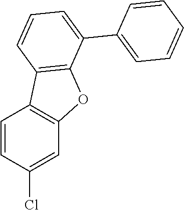 [SMILES: Clc1ccc2c(c1)oc1c(-c3ccccc3)cccc12] | 3.07 g |
| a08 | 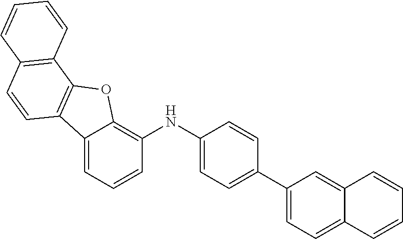 [SMILES: c1ccc2cc(-c3ccc(Nc4cccc5c4oc4c6ccccc6ccc54)cc3)ccc2c1] | 4.36 g | 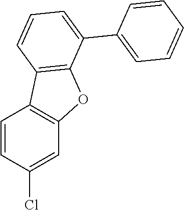 [SMILES: Clc1ccc2c(c1)oc1c(-c3ccccc3)cccc12] | 3.07 g |
| a09 | 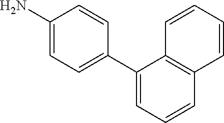 [SMILES: Nc1ccc(-c2cccc3ccccc23)cc1] | 2.19 g | 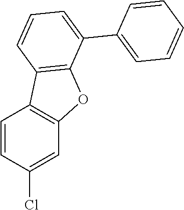 [SMILES: Clc1ccc2c(c1)oc1c(-c3ccccc3)cccc12] | 6.69 g (24 mmol) *NaO^{t} Bu: 2.90 g (30 mmol) used |
| a10 | 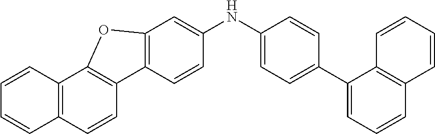 [SMILES: c1ccc2c(-c3ccc(Nc4ccc5c(c4)oc4c6ccccc6ccc54)cc3)cccc2c1] | 4.36 g | 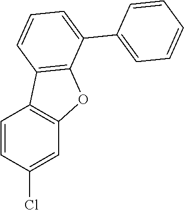 [SMILES: Clc1ccc2c(c1)oc1c(-c3ccccc3)cccc12] | 3.07 g |
| a11 | 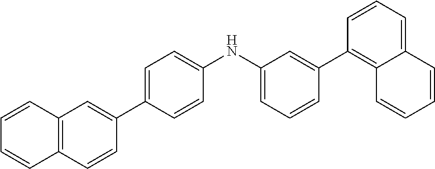 [SMILES: c1cc(Nc2ccc(-c3ccc4ccccc4c3)cc2)cc(-c2cccc3ccccc23)c1] | 4.22 g | 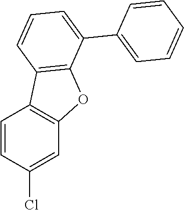 [SMILES: Clc1ccc2c(c1)oc1c(-c3ccccc3)cccc12] | 3.07 g |
| a12 | 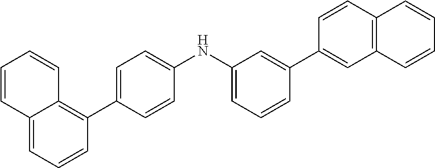 [SMILES: c1cc(Nc2ccc(-c3cccc4ccccc34)cc2)cc(-c2ccc3ccccc3c2)c1] | 4.22 g | 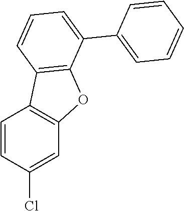 [SMILES: Clc1ccc2c(c1)oc1c(-c3ccccc3)cccc12] | 3.07 g |
| a13 | 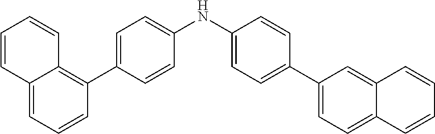 [SMILES: c1ccc2cc(-c3ccc(Nc4ccc(-c5cccc6ccccc56)cc4)cc3)ccc2c1] | 4.22 g | 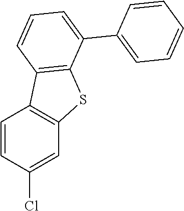 [SMILES: Clc1ccc2c(c1)sc1c(-c3ccccc3)cccc12] | 3.24 g |
| a14 | 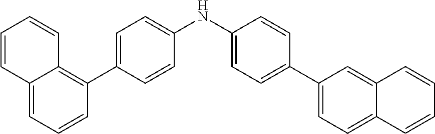 [SMILES: c1ccc2cc(-c3ccc(Nc4ccc(-c5cccc6ccccc56)cc4)cc3)ccc2c1] | 4.22 g | 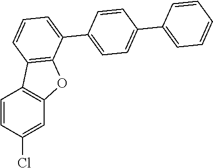 [SMILES: Clc1ccc2c(c1)oc1c(-c3ccc(-c4ccccc4)cc3)cccc12] | 3.55 g |
| a15 | 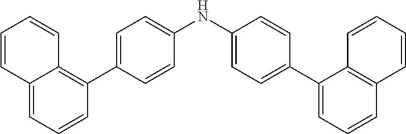 [SMILES: c1ccc2c(-c3ccc(Nc4ccc(-c5cccc6ccccc56)cc4)cc3)cccc2c1] | 4.22 g | 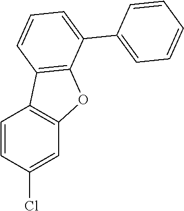 [SMILES: Clc1ccc2c(c1)oc1c(-c3ccccc3)cccc12] | 3.07 g |
| a16 | 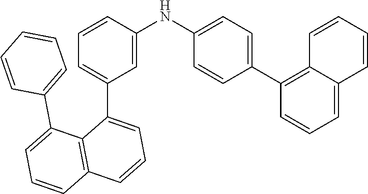 [SMILES: c1ccc(-c2cccc3cccc(-c4cccc(Nc5ccc(-c6cccc7ccccc67)cc5)c4)c23)cc1] | 4.98 g | 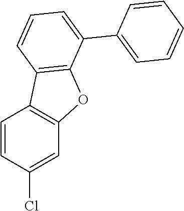 [SMILES: Clc1ccc2c(c1)oc1c(-c3ccccc3)cccc12] | 3.07 g |
| a17 | 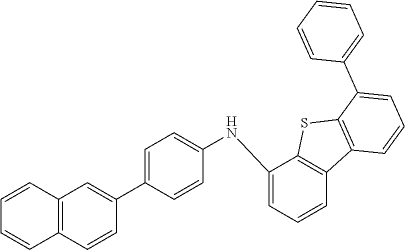 [SMILES: c1ccc(-c2cccc3c2sc2c(Nc4ccc(-c5ccc6ccccc6c5)cc4)cccc23)cc1] | 4.77 g | 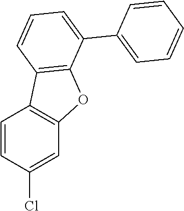 [SMILES: Clc1ccc2c(c1)oc1c(-c3ccccc3)cccc12] | 3.07 g |

| Divi- | Target | Amount | | FAB- |
|---|---|---|---|---|
| sion | compound | obtained | Yield | MS |
| a01 | 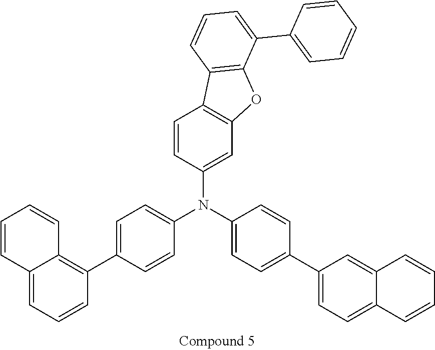 [SMILES: c1ccc(-c2cccc3c2oc2cc(N(c4ccc(-c5ccc6ccccc6c5)cc4)c4ccc(-c5cccc6ccccc56)cc4)ccc23)cc1] | 5.37 g | 81% | 663 |
| a02 | 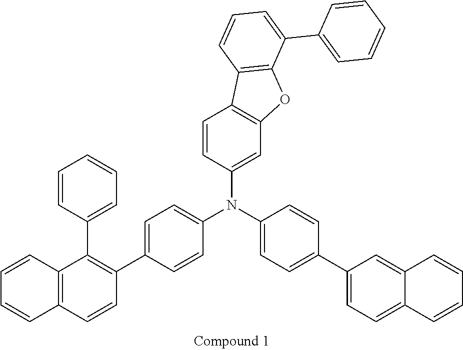 [SMILES: c1ccc(-c2c(-c3ccc(N(c4ccc(-c5ccc6ccccc6c5)cc4)c4ccc5c(c4)oc4c(-c6ccccc6)cccc45)cc3)ccc3ccccc23)cc1] | 5.76 g | 78% | 739 |
| a03 | 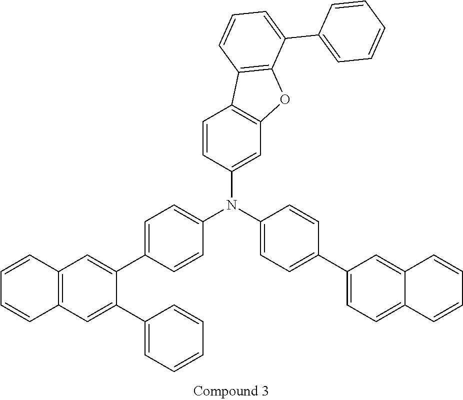 [SMILES: c1ccc(-c2cc3ccccc3cc2-c2ccc(N(c3ccc(-c4ccc5ccccc5c4)cc3)c3ccc4c(c3)oc3c(-c5ccccc5)cccc34)cc2)cc1] | 5.54 g | 75% | 739 |
| a04 | 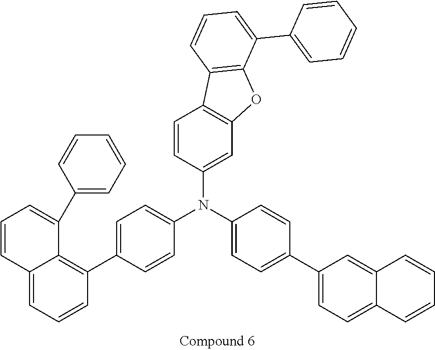 [SMILES: c1ccc(-c2cccc3c2oc2cc(N(c4ccc(-c5ccc6ccccc6c5)cc4)c4ccc(-c5cccc6cccc(-c7ccccc7)c56)cc4)ccc23)cc1] | 5.10 g | 69% | 739 |
| a05 | 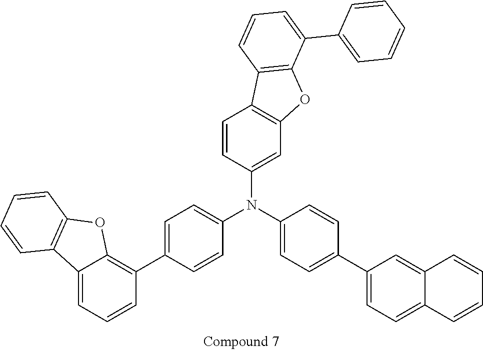 [SMILES: c1ccc(-c2cccc3c2oc2cc(N(c4ccc(-c5ccc6ccccc6c5)cc4)c4ccc(-c5cccc6c5oc5ccccc56)cc4)ccc23)cc1] | 5.76 g | 82% | 703 |
| a06 | 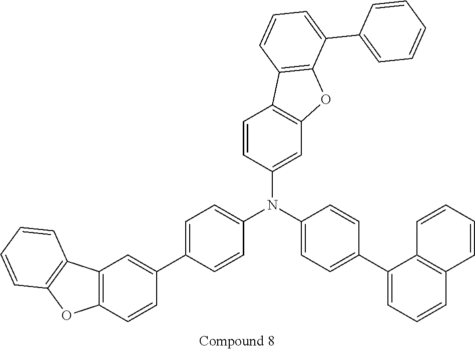 [SMILES: c1ccc(-c2cccc3c2oc2cc(N(c4ccc(-c5ccc6oc7ccccc7c6c5)cc4)c4ccc(-c5cccc6ccccc56)cc4)ccc23)cc1] | 5.98 g | 85% | 703 |
| a07 | 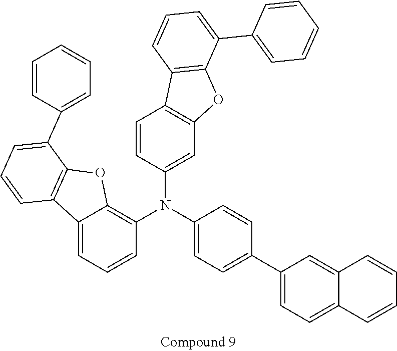 [SMILES: c1ccc(-c2cccc3c2oc2cc(N(c4ccc(-c5ccc6ccccc6c5)cc4)c4cccc5c4oc4c(-c6ccccc6)cccc45)ccc23)cc1] | 5.13 g | 73% | 703 |
| a08 | 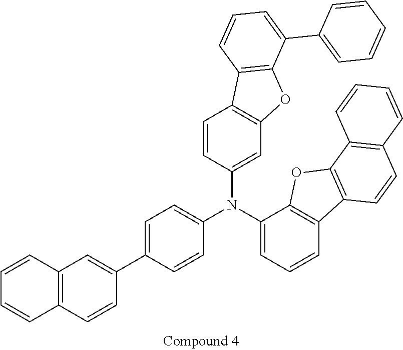 [SMILES: c1ccc(-c2cccc3c2oc2cc(N(c4ccc(-c5ccc6ccccc6c5)cc4)c4cccc5c4oc4c6ccccc6ccc54)ccc23)cc1] | 5.01 g | 74% | 677 |
| a09 | 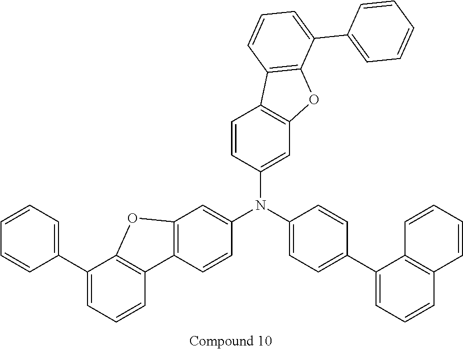 [SMILES: c1ccc(-c2cccc3c2oc2cc(N(c4ccc(-c5cccc6ccccc56)cc4)c4ccc5c(c4)oc4c(-c6ccccc6)cccc45)ccc23)cc1] | 3.16 g | 45% | 703 |
| a10 | 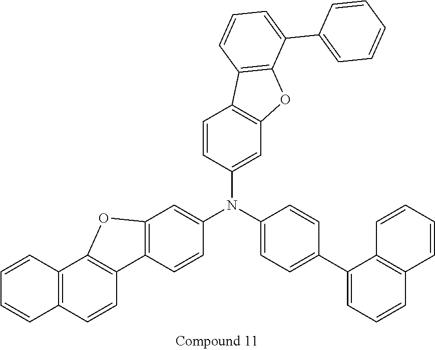 [SMILES: c1ccc(-c2cccc3c2oc2cc(N(c4ccc(-c5cccc6ccccc56)cc4)c4ccc5c(c4)oc4c6ccccc6ccc54)ccc23)cc1] | 5.42 g | 80% | 677 |
| a11 | 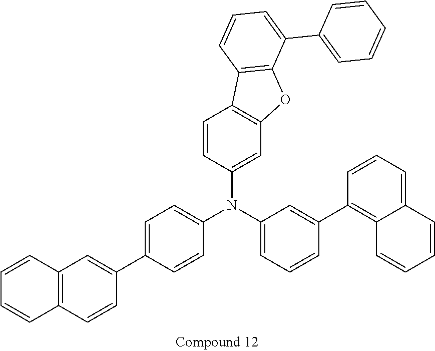 [SMILES: c1ccc(-c2cccc3c2oc2cc(N(c4ccc(-c5ccc6ccccc6c5)cc4)c4cccc(-c5cccc6ccccc56)c4)ccc23)cc1] | 5.03 g | 76% | 663 |
| a12 | 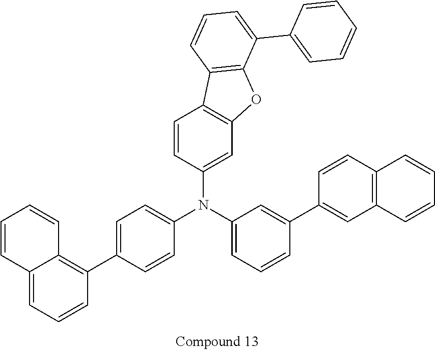 [SMILES: c1ccc(-c2cccc3c2oc2cc(N(c4ccc(-c5cccc6ccccc56)cc4)c4cccc(-c5ccc6ccccc6c5)c4)ccc23)cc1] | 4.71 g | 71% | 663 |
| a13 | 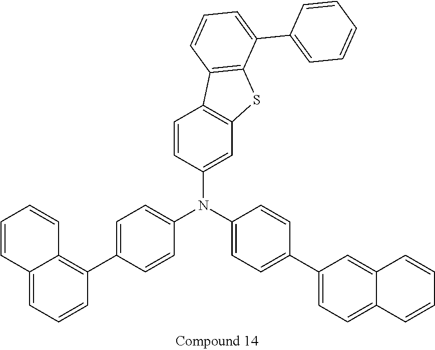 [SMILES: c1ccc(-c2cccc3c2sc2cc(N(c4ccc(-c5ccc6ccccc6c5)cc4)c4ccc(-c5cccc6ccccc56)cc4)ccc23)cc1] | 4.28 g | 63% | 679 |
| a14 | 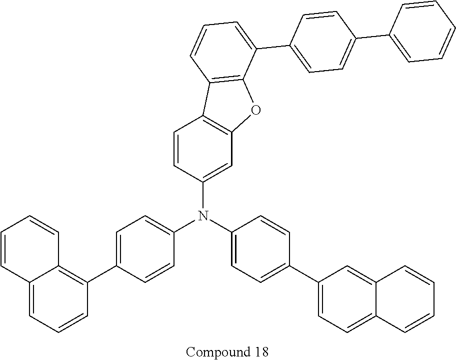 [SMILES: c1ccc(-c2ccc(-c3cccc4c3oc3cc(N(c5ccc(-c6ccc7ccccc7c6)cc5)c5ccc(-c6cccc7ccccc67)cc5)ccc34)cc2)cc1] | 4.80 g | 65% | 739 |
| a15 | 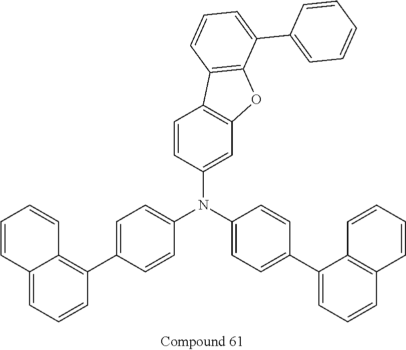 [SMILES: c1ccc(-c2cccc3c2oc2cc(N(c4ccc(-c5cccc6ccccc56)cc4)c4ccc(-c5cccc6ccccc56)cc4)ccc23)cc1] | 5.90 g | 89% | 663 |
| a16 | 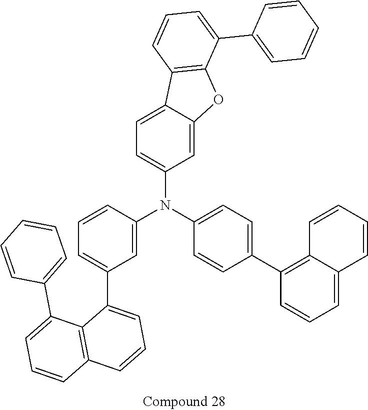 [SMILES: c1ccc(-c2cccc3c2oc2cc(N(c4ccc(-c5cccc6ccccc56)cc4)c4cccc(-c5cccc6cccc(-c7ccccc7)c56)c4)ccc23)cc1] | 6.06 g | 82% | 739 |
| a17 | 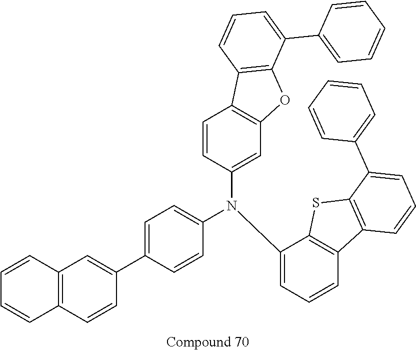 [SMILES: c1ccc(-c2cccc3c2oc2cc(N(c4ccc(-c5ccc6ccccc6c5)cc4)c4cccc5c4sc4c(-c6ccccc6)cccc45)ccc23)cc1] | 4.96 g | 69% | 719 |

TABLE 2

| Division | ¹H-NMR(CDCl3) |
|---|---|
| a01 | 8.09-8.04(2H), 7.97-7.85(10H), 7.77(1H), |
| (Compound 5) | 7.72-7.66(2H), 7.58-7.18(18H), |
| a02 | 8.02(1H), 7.96-7.81 (9H), 7.73(2H), 7.66-7.61(3H), |
| (Compound 1) | 7.56-7.47(6H), 7.43-7.31(6H), 7.26-7.12(6H), |
| | 7.08-6.99(4H) |
| a03 | 8.03(1H), 7.95(1H), 7.92-7.82(10H), 7.74(1H), |
| (Compound 3) | 7.64(2H), 7.56-7.38(9H), 7.33-7.06(13H) |
| a04 | 8.04-8.02(2H), 7.95-7.86(8H), 7.71-7.68(3H), |
| (Compound 6) | 7.55-7.10(24H) |
| a05 | 8.10-7.87(9H), 7.80(1H), 7.74-7.10(23H) |
| (Compound 7) | |
| a06 | 8.05(1H), 7.95(1H), 7.90(1H), 7.85-7.77(5H), |
| (Compound 8) | 7.74-7.66(5H), 7.56-7.10(20H) |
| a07 | 8.10-7.88(8H), 7.80(1H), 7.74-7.26(21H), 7.18(1H), |
| (Compound 9) | 7.08-7.00(2H) |
| a08 | 8.07-7.88(8H), 7.75-7.69(2H), 7.60-7.26(19H), |
| (Compound 4) | 7.08-7.00(2H) |
| a09 | 8.00(2H), 7.91-7.71(8H), 7.61-7.56(3H), |
| (Compound 10) | 7.46-7.16(18H), 7.02-7.00(2H) |
| a10 | 8.05(1H), 7.95(1H), 7.91-7.80(8H), 7.75-7.56(6H), |
| (Compound 11) | 7.46-7.16(13H), 7.02-7.00(2H) |
| a11 | 8.05(1H), 7.95(1H), 7.90(1H), 7.85-7.73(7H), |
| (Compound 12) | 7.66-7.61(2H), 7.54-7.26(17H), 7.22(1H), 7.19-7.10(3H) |
| a12 | 8.05(1H), 7.95(1H), 7.90(1H), 7.85-7.73(7H), |
| (Compound 13) | 7.66-7.61(2H), 7.54-7.26(17H), 7.22(1H), 7.19-7.10(3H) |
| a13 | 8.05(1H), 7.95(1H), 7.90(1H), 7.82(1H), 7.75(1H), |
| (Compound 14) | 7.70-7.58(5H), 7.48-7.16(23H) |
| a14 | 8.05(1H), 7.95(1H), 7.90(1H), 7.85-7.73(7H), |
| (Compound 18) | 7.61-7.54(4H), 7.44-7.01(23H) |
| a15 | 8.08(2H), 7.95-7.85(8H), 7.58-7.32(23H) |
| (Compound 61) | |
| a16 | 8.05(1H), 7.94-7.84(8H), 7.57-7.36(15H), |
| (Compound 28) | 7.32-7.14(8H), 7.00(2H), 6.87(2H), 6.72(1H) |
| a17 | 8.21-8.09(2H), 8.01(1H), 7.92-7.71(9H), 7.65-7.11(21H) |
| (Compound 70) | |

### 2. Manufacture and Evaluation of Light Emitting Elements

### (1) Manufacture of Light Emitting Element

A light emitting element of an embodiment, including the amine compound of an embodiment in an electron blocking layer was manufactured by a method below.

On a glass substrate, ITO with a thickness of about 1500 Å was patterned, washed with pure water and treated with UV ozone for about 10 minutes to form a first electrode. After that, 2-TNATA was deposited to a thickness of about 600 Å to form a hole injection layer. Then, the Example Compound or Comparative Compound was deposited to a thickness of about 100 Å to form an electron blocking layer.

Then, an emission layer having a thickness of about 250 Å was formed using and doped with 3% TBP. Then, Alq₃ was deposited to a thickness of about 250 Å to form an electron transport layer, and LiF was deposited to a thickness of about 10 Å to form an electron injection layer.

After that, a second electrode was formed by providing aluminum (Al) to a thickness of about 1000 Å. In the Example, the hole injection layer, the electron blocking layer, the emission layer, the electron transport layer, the electron injection layer and the second electrode were formed using a vacuum deposition apparatus.

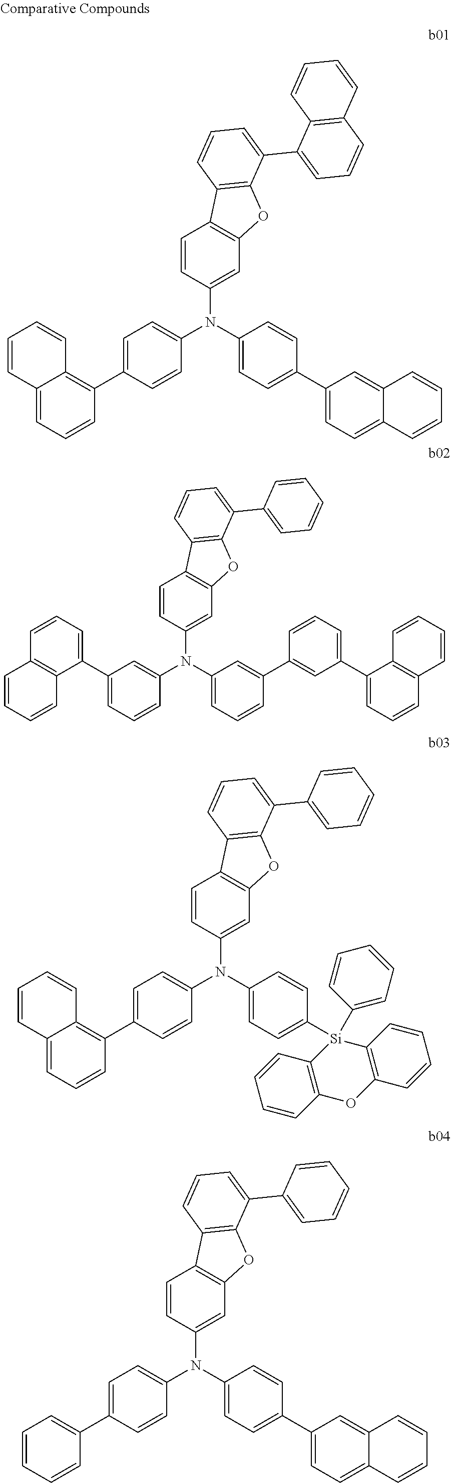 [SMILES: c1ccc2cc(-c3ccc(N(c4ccc(-c5cccc6ccccc56)cc4)c4ccc5c(c4)oc4c(-c6cccc7ccccc67)cccc45)cc3)ccc2c1 ; c1ccc(-c2cccc3c2oc2cc(N(c4cccc(-c5cccc(-c6cccc7ccccc67)c5)c4)c4cccc(-c5cccc6ccccc56)c4)ccc23)cc1 ; c1ccc(-c2cccc3c2oc2cc(N(c4ccc(-c5cccc6ccccc56)cc4)c4ccc([Si]5(c6ccccc6)c6ccccc6Oc6ccccc65)cc4)ccc23)cc1 ; c1ccc(-c2ccc(N(c3ccc(-c4ccc5ccccc5c4)cc3)c3ccc4c(c3)oc3c(-c5ccccc5)cccc34)cc2)cc1]

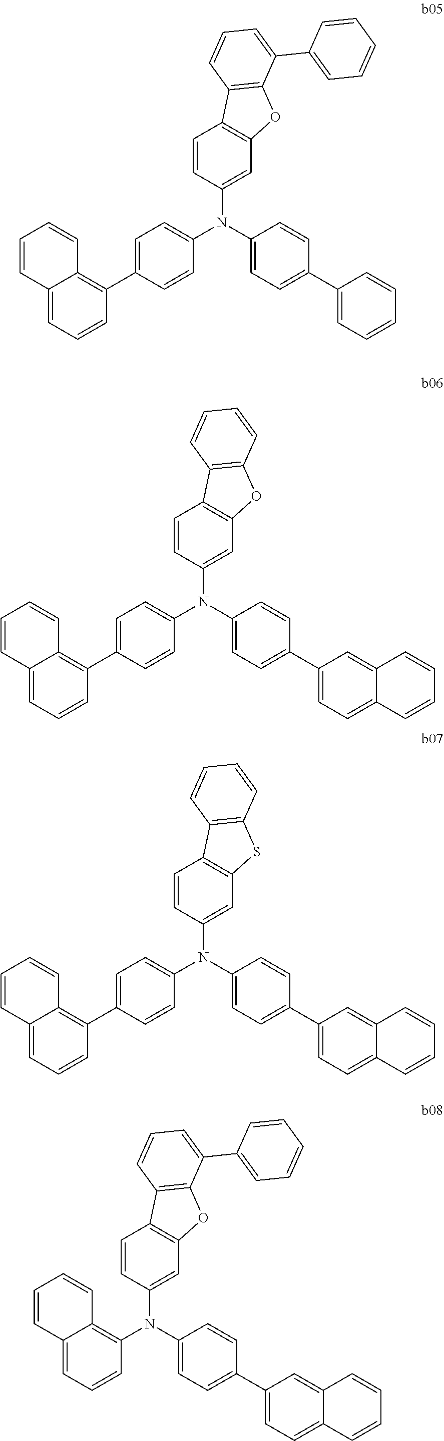 [SMILES: c1ccc(-c2ccc(N(c3ccc(-c4cccc5ccccc45)cc3)c3ccc4c(c3)oc3c(-c5ccccc5)cccc34)cc2)cc1 ; c1ccc2cc(-c3ccc(N(c4ccc(-c5cccc6ccccc56)cc4)c4ccc5c(c4)oc4ccccc45)cc3)ccc2c1 ; c1ccc2cc(-c3ccc(N(c4ccc(-c5cccc6ccccc56)cc4)c4ccc5c(c4)sc4ccccc45)cc3)ccc2c1 ; c1ccc(-c2cccc3c2oc2cc(N(c4ccc(-c5ccc6ccccc6c5)cc4)c4cccc5ccccc45)ccc23)cc1]

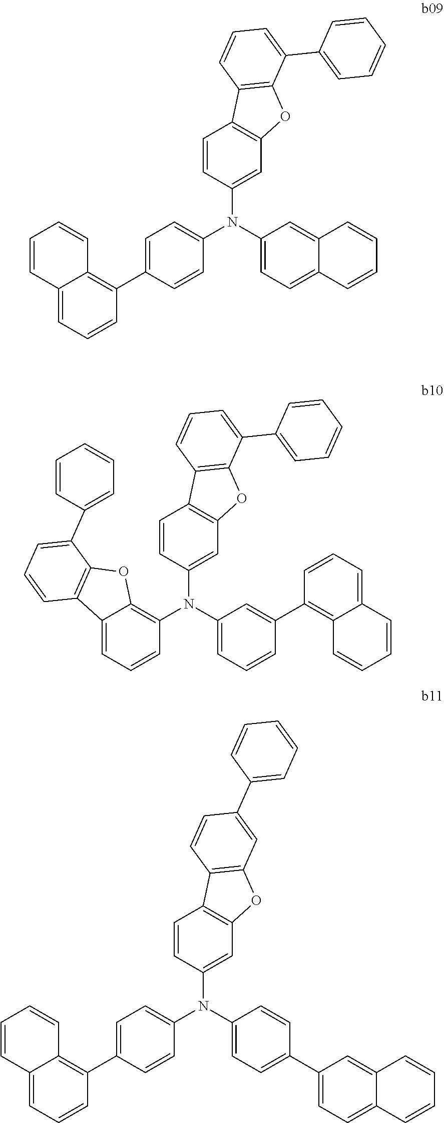 [SMILES: c1ccc(-c2cccc3c2oc2cc(N(c4ccc(-c5cccc6ccccc56)cc4)c4ccc5ccccc5c4)ccc23)cc1 ; c1ccc(-c2cccc3c2oc2cc(N(c4cccc(-c5cccc6ccccc56)c4)c4cccc5c4oc4c(-c6ccccc6)cccc45)ccc23)cc1 ; c1ccc(-c2ccc3c(c2)oc2cc(N(c4ccc(-c5ccc6ccccc6c5)cc4)c4ccc(-c5cccc6ccccc56)cc4)ccc23)cc1]

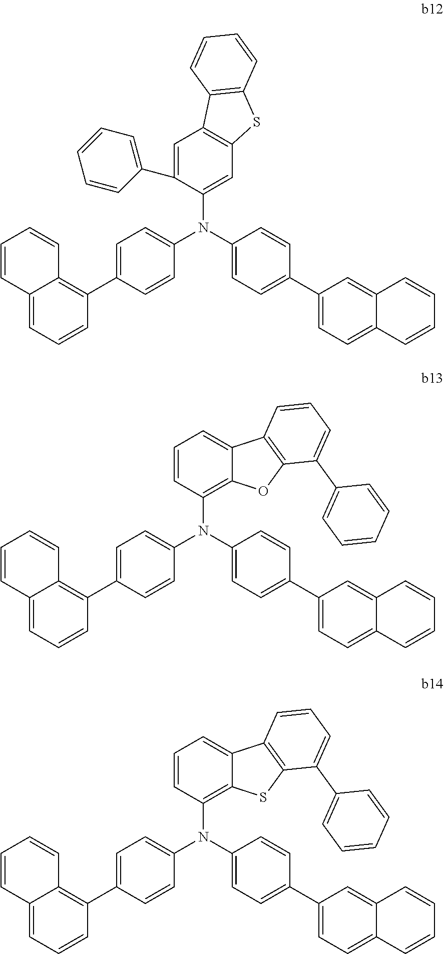 [SMILES: c1ccc(-c2cc3c(cc2N(c2ccc(-c4ccc5ccccc5c4)cc2)c2ccc(-c4cccc5ccccc45)cc2)sc2ccccc23)cc1 ; c1ccc(-c2cccc3c2oc2c(N(c4ccc(-c5ccc6ccccc6c5)cc4)c4ccc(-c5cccc6ccccc56)cc4)cccc23)cc1 ; c1ccc(-c2cccc3c2sc2c(N(c4ccc(-c5ccc6ccccc6c5)cc4)c4ccc(-c5cccc6ccccc56)cc4)cccc23)cc1]

In addition, the compounds of each functional layer used for the manufacture of the light emitting element are as follows.

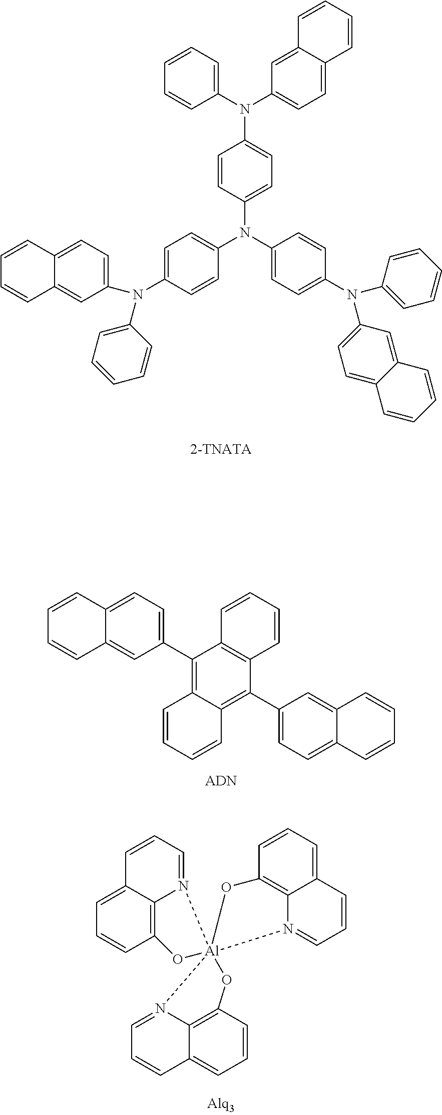

### (2) Evaluation of Light Emitting Element

Table 3 shows evaluation results for the light emitting elements of Examples 1 to 17, and Comparative Examples 1 to 14. In the evaluation results for the properties of the Examples and Comparative Examples, shown in Table 3, the emission efficiency shows an efficiency value at a current density of about 10 mA/cm², and half lifetime shows luminance reduction time to half from about 1000 cd/m². The layer purity shows a measured value by depositing each electron blocking layer material at a rate of about 0.2 nm/s and measuring the purity by HPLC of the electron blocking layer material deposited on a substrate. The purity of all electron blocking layer materials before deposition was about 99.9%. The absorption edge wavelength shows the wavelength at an initiation position at a long wavelength side in the absorption spectrum of a deposition layer of each electron blocking layer material.

TABLE 3

| Element | Electron | | Life- | | Absorption |
|---|---|---|---|---|---|
| manufac- | blocking | Emission | time | | edge |
| turing | layer (EBL) | efficiency | LT50 | Purity | wavelength |
| example | material | (cd/A) | (h) | of EBL | (nm) |
| Example 1 | a01 | 8.0 | 1800 | 99.9% | 390 |
| | (Compound 5) | | | | |
| Example 2 | a02 | 8.0 | 1900 | 99.9% | 397 |
| | (Compound 1) | | | | |
| Example 3 | a03 | 8.0 | 1900 | 99.9% | 398 |
| | (Compound 3) | | | | |
| Example 4 | a04 | 7.9 | 1800 | 99.9% | 400 |
| | (Compound 6) | | | | |
| Example 5 | a05 | 7.9 | 2000 | 99.9% | 395 |
| | (Compound 7) | | | | |
| Example 6 | a06 | 7.9 | 1800 | 99.9% | 386 |
| | (Compound 8) | | | | |
| Example 7 | a07 | 7.8 | 2000 | 99.9% | 388 |
| | (Compound 9) | | | | |
| Example 8 | a08 | 8.1 | 1800 | 99.9% | 391 |
| | (Compound 4) | | | | |
| Example 9 | a09 | 7.9 | 2100 | 99.9% | 394 |
| | (Compound 10) | | | | |
| Example 10 | a10 | 8.0 | 1900 | 99.9% | 396 |
| | (Compound 11) | | | | |
| Example 11 | a11 | 8.0 | 1800 | 99.9% | 384 |
| | (Compound 12) | | | | |
| Example 12 | a12 | 8.0 | 1800 | 99.9% | 380 |
| | (Compound 13) | | | | |
| Example 13 | a13 | 8.1 | 1800 | 99.9% | 395 |
| | (Compound 14) | | | | |
| Example 14 | a14 | 7.9 | 1900 | 99.9% | 394 |
| | (Compound 18) | | | | |
| Example 15 | a15 | 8.0 | 2300 | 99.9% | 385 |
| | (Compound 61) | | | | |
| Example 16 | a15 | 8.0 | 1800 | 99.9% | 393 |
| | (Compound 28) | | | | |
| Example 17 | a14 | 8.1 | 2200 | 99.9% | 386 |
| | (Compound 70) | | | | |
| Comparative | b01 | 7.5 | 700 | 99.8% | 408 |
| Example 1 | | | | | |
| Comparative | b02 | 7.8 | 800 | 99.9% | 370 |
| Example 2 | | | | | |
| Comparative | b03 | 7.7 | 500 | 99.8% | 381 |
| Example 3 | | | | | |
| Comparative | b04 | 7.6 | 1600 | 99.9% | 386 |
| Example 4 | | | | | |
| Comparative | b05 | 7.7 | 1300 | 99.9% | 383 |
| Example 5 | | | | | |
| Comparative | b06 | 7.7 | 1600 | 99.9% | 399 |
| Example 6 | | | | | |
| Comparative | b07 | 7.7 | 1100 | 99.9% | 402 |
| Example 7 | | | | | |
| Comparative | b08 | 7.7 | 1200 | 99.7% | 396 |
| Example 8 | | | | | |
| Comparative | b09 | 7.6 | 900 | 99.9% | 409 |
| Example 9 | | | | | |
| Comparative | b10 | 7.6 | 1000 | 99.9% | 373 |
| Example 10 | | | | | |
| Comparative | b11 | 7.5 | 1500 | 99.9% | 408 |
| Example 11 | | | | | |
| Comparative | b12 | 7.7 | 1000 | 99.7% | 392 |
| Example 12 | | | | | |
| Comparative | b13 | 7.7 | 1600 | 99.9% | 384 |
| Example 13 | | | | | |
| Comparative | b14 | 7.8 | 1400 | 99.9% | 387 |
| Example 14 | | | | | |

Referring to the results of Table 3, it could be found that the Examples of the light emitting elements using the amine compound of an embodiment according to the present disclosure as an electron blocking layer material, exhibited long-life characteristics.

The amine compound of an embodiment, including first to third substituents having high electron tolerance, has high electron tolerance. In addition, the amine compound of an embodiment may suppress or reduce excessive increase of the deposition temperature and may suppress or reduce the deterioration of materials by a deposition process. Accordingly, the lifetime of the light emitting elements of the Examples using the amine compound of an embodiment as an electron blocking layer material, were excellent.

For example, in some embodiments, the first substituent includes a dibenzofuran group or a dibenzothiophene group combined with an aryl group at position 6, and is directly bonded to the nitrogen atom of an amine at position 3 of the dibenzofuran group and the dibenzothiophene group. The amine compound of an embodiment includes the first substituent, and destabilization due to three-dimensional twist around the amine may not occur, thereby providing high stability.

In the amine compound of an embodiment, at least one among the second substituent and the third substituent is a substituted or unsubstituted naphthyl group, or a substituted or unsubstituted heteroaryl group, bonded to the nitrogen atom of an amine via a linker in para relation (e.g., at a para bonding position). The amine compound of an embodiment includes the second substituent and the third substituent, and may have even further improved hole transport properties and stability.

In the Comparative Compound used in Comparative Example 1, when compared to the compound used in Example 1, a naphthyl group was substituted at position 6 of dibenzofuran, and the light emitting element of Comparative Example 1 exhibited low efficiency/short lifetime. While the present application is not limited by any particular mechanism or theory, it is thought that the absorption wavelength of a molecule of Comparative Example 1 was excessively increased, a portion of light emitted from the emission layer was absorbed, and hole transport layer itself was excited.

The Comparative Compound used in Comparative Example 2, when compared to the Example Compounds used in Examples 1-4, 11 and 12, included two phenyl groups having substituents at meta positions among the groups bonded to an amine. Accordingly, the light emitting element of Comparative Example 2 exhibited low efficiency and short lifetime. While the present application is not limited by any particular mechanism or theory, it is thought that both substituents other than 3-dibenzofuran were not at para positions among the substituents bonded to the amine, and thus, the stability to holes was degraded.

In the Comparative Compound used in Comparative Example 3, when compared to the Example Compound used in Example 1, one among the groups bonded to an amine was a phenoxasilin group-substituted phenyl group at a para position, and the light emitting element of Comparative Example 3 exhibited low efficiency and short lifetime. While the present application is not limited by any particular mechanism or theory, it is thought that the stability of the phenoxasilin group against holes/electrons was low.

In the Comparative Compounds used in Comparative Example 4 and Comparative Example 5, when compared to the compounds used in Examples 1-6, 9 and 10, one among the groups bonded to an amine is a biphenyl group, and the results of somewhat degraded lifetime of an element manufactured were observed. While the present application is not limited by any particular mechanism or theory, it is thought that, when compared to the Example Compounds including all three groups bonded to the amine of a naphthalene moiety or a heteroaryl group having high electron tolerance, electron tolerance was degraded due to the biphenyl group by such a degree (e.g., due to the presence and position of the biphenyl group).

In the Compounds used in Comparative Example 6 and Comparative Example 7, when compared to the compounds used in Example 1 and Example 13, position 6 of dibenzofuran/dibenzothiophene was unsubstituted, and the light emitting elements of Comparative Example 6 and Comparative Example 7 exhibited short lifetime. While the present application is not limited by any particular mechanism or theory, it is thought that the surroundings of 0 and S atoms of the 3-dibenzofuran group/3-dibenzothiophene group directly bonded to an amine were not three-dimensionally protected, and thus, stability was degraded.

In the Comparative Compound used in Comparative Example 8, when compared to the Example Compounds used in Examples 1-5, 7 and 8, one among the groups bonded to an amine was a directly bonded α-naphthyl group, and the light emitting element of Comparative Example 8 exhibited degraded results of lifetime. While the present application is not limited by any particular mechanism or theory, it is thought that the stability of the directly bonded α-naphthyl group to the amine was low.

In the Comparative Compound used in Comparative Example 9, when compared to the Example Compounds used in Examples 1-6, 9 and 10, one among the groups bonded to an amine was a directly bonded β-naphthyl group, and the light emitting element of Comparative Example 9 exhibited low efficiency and short lifetime. While the present application is not limited by any particular mechanism or theory, it is thought that the absorption wavelength of a molecule of Comparative Example 9 increased too excessively, a portion of light emitted from an emission layer was absorbed, and thus, the transport layer itself was excited.

In the Comparative Compound used in Comparative Example 10, when compared to the Example Compounds used in Example 7 and Example 10, one among the groups bonded to an amine was a 4-dibenzofuran group, and another one was a phenyl group having a substituent at a meta position. Accordingly, the light emitting element of Comparative Example 10 exhibited low efficiency and short lifetime. While the present application is not limited by any particular mechanism or theory, it is thought that both other than the 3-dibenzofuran group, bonded to the amine had no substituent at a para position, and thus, stability against holes was degraded.

In the Comparative Compound used in Comparative Example 11, when compared to the Example Compound used in Example 1, a phenyl group was substituted not at position 6 but at position 7 of dibenzofuran, and the light emitting element of Comparative Example 11 exhibited low efficiency and short lifetime. While the present application is not limited by any particular mechanism or theory, it is thought that the absorption wavelength of a molecule of Comparative Example 11 was increased excessively, a portion of light emitted from an emission layer was absorbed, and thus, the transport layer itself was excited, and, in addition, the surrounding of the O atom of the 3-dibenzofuran group directly bonded to the amine was not three-dimensionally protected, and thus, stability was degraded.

In the Comparative Compound used in Comparative Example 12, when compared to the Example Compound used in Example 13, a phenyl group was substituted not at position 6 but at position 2 of dibenzothiophene, and the light emitting element of Comparative Example 12 exhibited low efficiency and short lifetime. While the present application is not limited by any particular mechanism or theory, it is thought that the surrounding of the S atom of the 3-dibenzothiophene group directly bonded to the amine was not three-dimensionally protected, and thus, the bond between dibenzothiophene and nitrogen was twisted and became unstable, to exhibit short lifetime.

In the Comparative Compounds used in Comparative Example 13 and Comparative Example 14, when compared to the Example Compounds used in Example 1 and Example 13, position 4 of dibenzofuran/dibenzothiophene was bonded to nitrogen, and the light emitting elements of Comparative Example 13 and Comparative Example 14 exhibited somewhat degraded results of lifetime in contrast to the light emitting elements of the Examples. While the present application is not limited by any particular mechanism or theory, it is thought that the unit of dibenzofuran/dibenzothiophene had a substitution mode without a substituent at para position to an amine, and thus, hole transportation became difficult.

The light emitting element of an embodiment includes an amine compound of an embodiment and may exhibit long-life characteristics.

The amine compound of an embodiment may be used as a material for accomplishing improved properties of a light emitting element with long lifetime.

Although example embodiments of the present disclosure have been described, it is to be understood that the present disclosure should not be limited to these embodiments, but various changes and modifications can be made by one ordinary skilled in the art within the spirit and scope of the present disclosure as hereinafter claimed in the appended claims, and equivalents thereof.

What is claimed is:

1. A light emitting element, comprising:
a first electrode;
a second electrode on the first electrode; and
at least one functional layer between the first electrode and the second electrode, and comprising an amine compound represented by the following Formula 1: 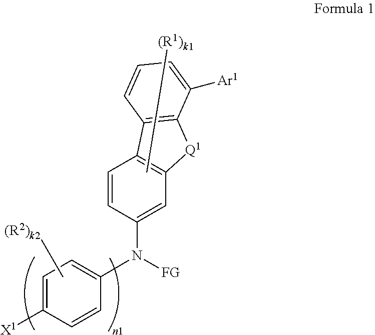
in Formula 1,
Q¹ is O or S,
Ar¹ is a substituted or unsubstituted phenyl group,
R¹ and R² are each independently a hydrogen atom, a deuterium atom, a halogen atom, a substituted or unsubstituted silyl group, a substituted or unsubstituted aryl group of 6 to 30 ring-forming carbon atoms, or a substituted or unsubstituted heteroaryl group of 2 to 30 ring-forming carbon atoms,
X¹ is a substituted or unsubstituted naphthyl group, or a substituted or unsubstituted heteroaryl group of 2 to 30 ring-forming carbon atoms,
n1 is an integer of 1 to 3, k1 is an integer of 0 to 6, k2 is an integer of 0 to 4, and
FG is represented by the following Formula 2-1 or Formula 2-2: 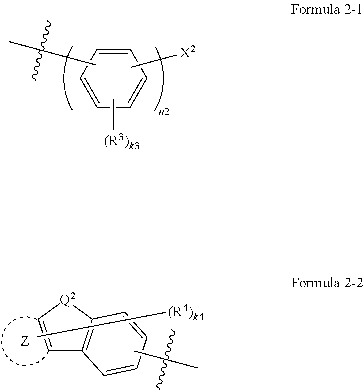
in Formula 2-1 and Formula 2-2,
X² is a substituted or unsubstituted naphthyl group, or a substituted or unsubstituted heteroaryl group of 2 to 30 ring-forming carbon atoms,
R³ and R⁴ are each independently a hydrogen atom, a deuterium atom, a halogen atom, a substituted or unsubstituted silyl group, a substituted or unsubstituted aryl group of 6 to 30 ring-forming carbon atoms, or a substituted or unsubstituted heteroaryl group of 2 to 30 ring-forming carbon atoms,
Q² is O, S, NR⁵, or CR⁶R⁷,
R⁵ to R⁷ are each independently a substituted or unsubstituted alkyl group of 1 to 20 carbon atoms, a substituted or unsubstituted aryl group of 6 to 50 ring-forming carbon atoms, or a substituted or unsubstituted heteroaryl group of 2 to 50 ring-forming carbon atoms,
Z is a substituted or unsubstituted aromatic hydrocarbon ring of 6 to 30 ring-forming carbon atoms, or a substituted or unsubstituted aromatic heterocycle of 2 to 30 ring-forming carbon atoms,
k3 is an integer of 0 to 4, k4 is an integer of 0 to 7, and n2 is an integer of 1 to 3, and
a case where X¹ and X² are 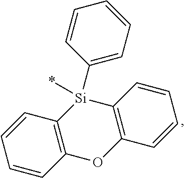 [SMILES: *[Si]1(c2ccccc2)c2ccccc2Oc2ccccc21]
is excluded.

2. The light emitting element of claim 1, wherein the at least one functional layer comprises an emission layer, a hole transport region between the first electrode and the emission layer, and an electron transport region between the emission layer and the second electrode, and
the hole transport region comprises the amine compound represented by Formula 1.

3. The light emitting element of claim 2, wherein the hole transport region comprises a hole injection layer on the first electrode, and an electron blocking layer on the hole injection layer, and
the electron blocking layer comprises the amine compound represented by Formula 1.

4. The light emitting element of claim 1, wherein Formula 2-1 is represented by the following Formula 2-1a or Formula 2-1b: 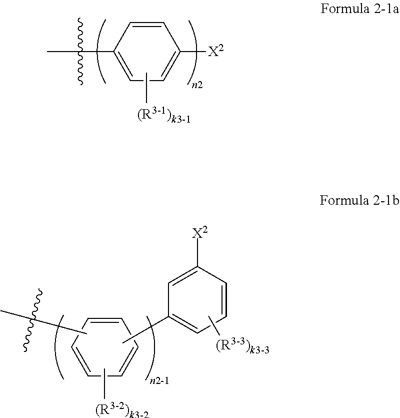
in Formula 2-1a and Formula 2-1b,
R³⁻¹ to R³⁻³ are each independently a hydrogen atom, a deuterium atom, a halogen atom, a substituted or unsubstituted aryl group or 6 to 15 ring-forming carbon atoms, or a substituted or unsubstituted heteroaryl group of 2 to 15 ring-forming carbon atoms,
k3-1 to k3-3 are each independently an integer of 0 to 4,
n2-1 is an integer of 0 to 2, and
X² and n2 are the same as defined with respect to Formula 2-1 and Formula 2-2.

5. The light emitting element of claim 1, wherein Formula 2-2 is represented by any one among the following Formula 2-2a to Formula 2-2c: 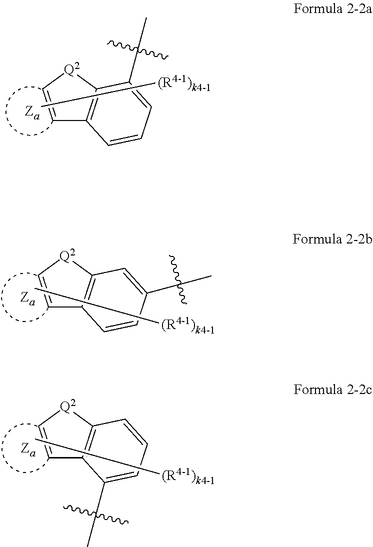
in Formula 2-2a to Formula 2-2c,
Zₐ is a substituted or unsubstituted aromatic hydrocarbon ring of 6 to 10 ring-forming carbon atoms,
R⁴⁻¹ is a hydrogen atom, a deuterium atom, a halogen atom, or a substituted or unsubstituted aryl group of 6 to 15 ring-forming carbon atoms,
k4-1 is an integer of 0 to 7, and
Q² is the same as defined with respect to Formula 2-2.

6. The light emitting element of claim 1, wherein FG is represented by any one among the following Formula FG-1 to Formula FG-6: 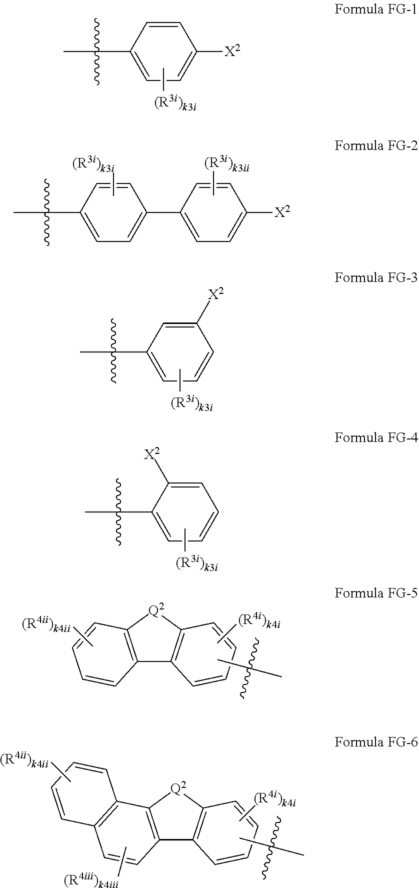
in Formula FG-1 to Formula FG-6,
R³ⁱ, R³ⁱⁱ, and R⁴ⁱ to R⁴ⁱⁱⁱ are each independently a hydrogen atom, a deuterium atom, a halogen atom, or a substituted or unsubstituted phenyl group,
k3i, k3ii, and k4ii are each independently an integer of 0 to 4,
k4i is an integer of 0 to 3,
k4iii is an integer of 0 to 2, and
X² and Q² are the same as defined with respect to Formula 2-1 and Formula 2-2.

7. The light emitting element of claim 1, wherein Formula 1 is represented by the following Formula 3-1 or Formula 3-2: 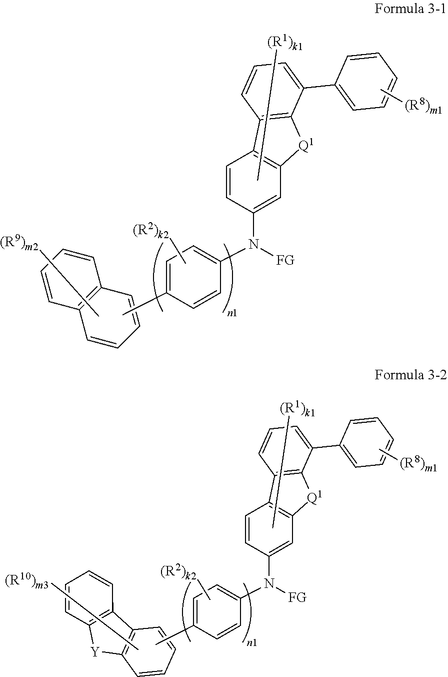
in Formula 3-1 and Formula 3-2,
Y is O or NR¹¹,
R⁸ to R¹¹ are each independently a hydrogen atom, a deuterium atom, a halogen atom, or a substituted or unsubstituted aryl group of 6 to 20 ring-forming carbon atoms,
m1 is an integer of 0 to 5, m2 and m3 are each independently an integer of 0 to 7, and
Q¹, R¹, R², n1, k1, k2, and FG are the same as defined with respect to Formula 1.

8. The light emitting element of claim 1, wherein Formula 1 is represented by the following Formula 4-1 or Formula 4-2: 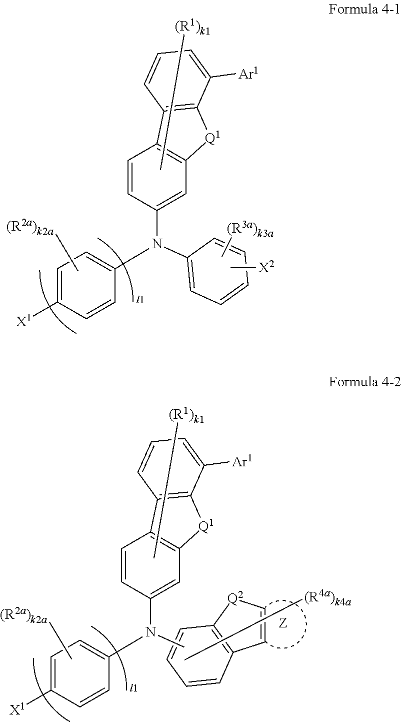
in Formula 4-1 and Formula 4-2,
R^{2a} to R^{4a} are each independently a hydrogen atom, a deuterium atom, a halogen atom, or a substituted or unsubstituted phenyl group,
k2a and k3a are each independently an integer of 0 to 4, k4a is an integer of 0 to 7,
l1 is 1 or 2, and
Ar¹, R¹, X¹, X², Q¹, Q², Z, and k1 are the same as defined with respect to Formula 1, Formula 2-1 and Formula 2-2.

9. The light emitting element of claim 1, wherein,
if FG is represented by Formula 2-1, at least one among X¹ and X² is a substituted or unsubstituted naphthyl group, and
if FG is represented by Formula 2-2, X¹ is a substituted or unsubstituted naphthyl group.

10. The light emitting element of claim 1, wherein X¹ and X² are each independently represented by any one among the following XS-1 to XS-6: 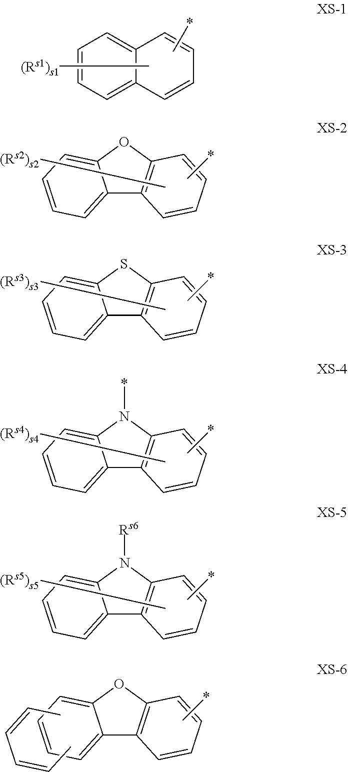
in XS-1 to XS-6,
R^{s1} to R^{s6} are each independently a hydrogen atom, a deuterium atom, a halogen atom, or a substituted or unsubstituted aryl group of 6 to 20 ring-forming carbon atoms,
s1 to s3, and s5 are each independently an integer of 0 to 7, and
s4 is an integer of 0 to 8.

11. The light emitting element of claim 2, wherein the emission layer comprises a compound represented by the following Formula E-1: 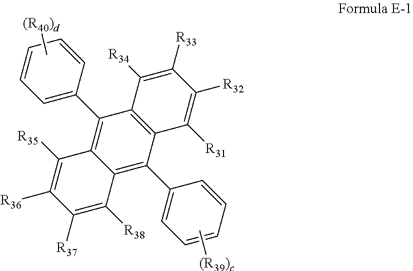
in Formula E-1,
R₃₁ to R₄₀ are each independently a hydrogen atom, a deuterium atom, a halogen atom, a substituted or unsubstituted silyl group, a substituted or unsubstituted thio group, a substituted or unsubstituted oxy group, a substituted or unsubstituted alkyl group of 1 to 10 carbon atoms, a substituted or unsubstituted alkenyl group of 2 to 10 carbon atoms, a substituted or unsubstituted aryl group of 6 to 30 ring-forming carbon atoms, or a substituted or unsubstituted heteroaryl group of 2 to 30 ring-forming carbon atoms, or combined with an adjacent group to form a ring, and
“c” and “d” are each independently an integer of 0 to 5.

12. The light emitting element of claim 1, wherein the amine compound is represented by any one among compounds in the following Compound Group 1:

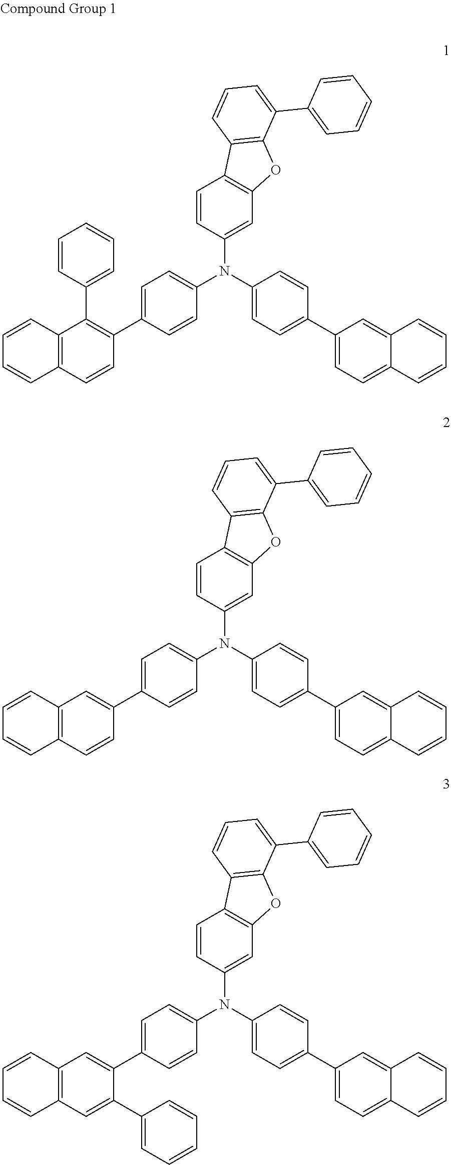 [SMILES: c1ccc(-c2c(-c3ccc(N(c4ccc(-c5ccc6ccccc6c5)cc4)c4ccc5c(c4)oc4c(-c6ccccc6)cccc45)cc3)ccc3ccccc23)cc1 ; c1ccc(-c2cccc3c2oc2cc(N(c4ccc(-c5ccc6ccccc6c5)cc4)c4ccc(-c5ccc6ccccc6c5)cc4)ccc23)cc1 ; c1ccc(-c2cc3ccccc3cc2-c2ccc(N(c3ccc(-c4ccc5ccccc5c4)cc3)c3ccc4c(c3)oc3c(-c5ccccc5)cccc34)cc2)cc1]

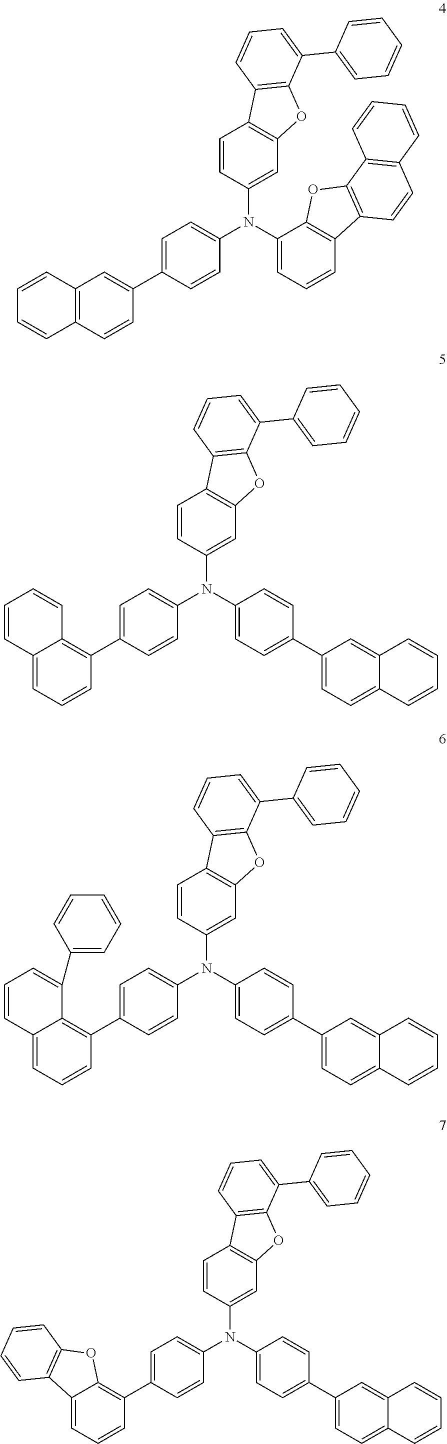 [SMILES: c1ccc(-c2cccc3c2oc2cc(N(c4ccc(-c5ccc6ccccc6c5)cc4)c4cccc5c4oc4c6ccccc6ccc54)ccc23)cc1 ; c1ccc(-c2cccc3c2oc2cc(N(c4ccc(-c5ccc6ccccc6c5)cc4)c4ccc(-c5cccc6ccccc56)cc4)ccc23)cc1 ; c1ccc(-c2cccc3c2oc2cc(N(c4ccc(-c5ccc6ccccc6c5)cc4)c4ccc(-c5cccc6cccc(-c7ccccc7)c56)cc4)ccc23)cc1 ; c1ccc(-c2cccc3c2oc2cc(N(c4ccc(-c5ccc6ccccc6c5)cc4)c4ccc(-c5cccc6c5oc5ccccc56)cc4)ccc23)cc1]

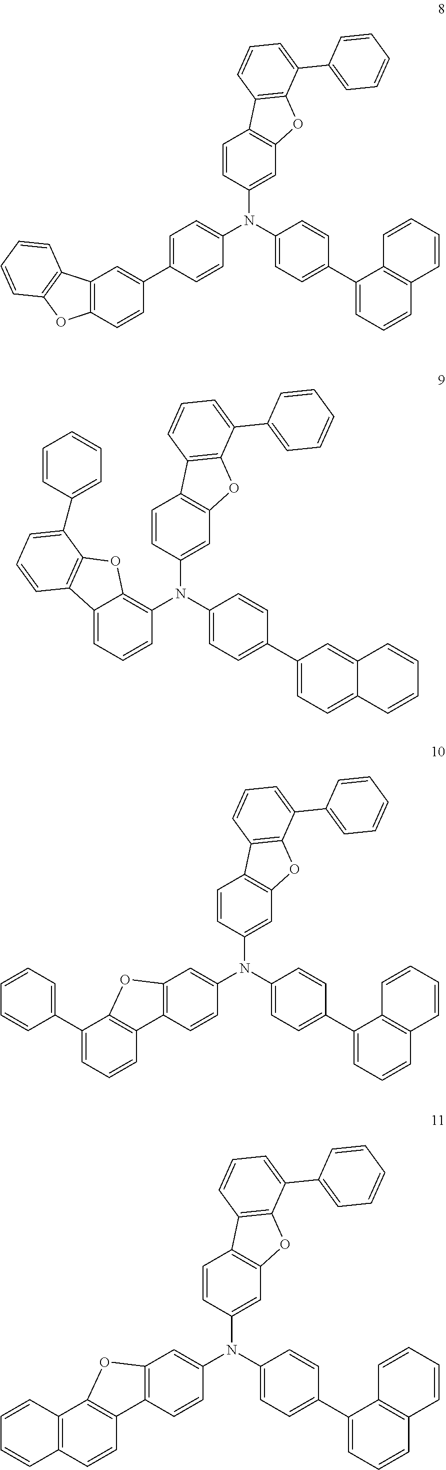 [SMILES: c1ccc(-c2cccc3c2oc2cc(N(c4ccc(-c5ccc6oc7ccccc7c6c5)cc4)c4ccc(-c5cccc6ccccc56)cc4)ccc23)cc1 ; c1ccc(-c2cccc3c2oc2cc(N(c4ccc(-c5ccc6ccccc6c5)cc4)c4cccc5c4oc4c(-c6ccccc6)cccc45)ccc23)cc1 ; c1ccc(-c2cccc3c2oc2cc(N(c4ccc(-c5cccc6ccccc56)cc4)c4ccc5c(c4)oc4c(-c6ccccc6)cccc45)ccc23)cc1 ; c1ccc(-c2cccc3c2oc2cc(N(c4ccc(-c5cccc6ccccc56)cc4)c4ccc5c(c4)oc4c6ccccc6ccc54)ccc23)cc1]

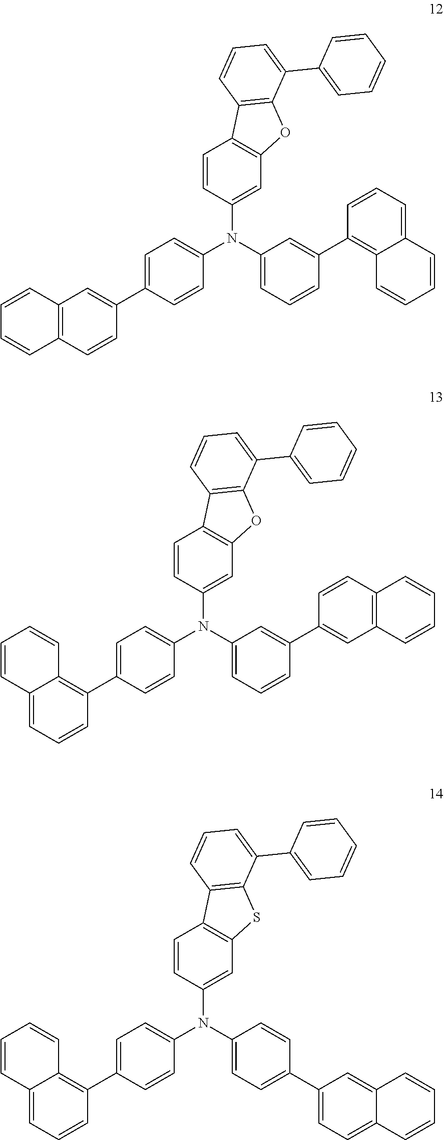 [SMILES: c1ccc(-c2cccc3c2oc2cc(N(c4ccc(-c5ccc6ccccc6c5)cc4)c4cccc(-c5cccc6ccccc56)c4)ccc23)cc1 ; c1ccc(-c2cccc3c2oc2cc(N(c4ccc(-c5cccc6ccccc56)cc4)c4cccc(-c5ccc6ccccc6c5)c4)ccc23)cc1 ; c1ccc(-c2cccc3c2sc2cc(N(c4ccc(-c5ccc6ccccc6c5)cc4)c4ccc(-c5cccc6ccccc56)cc4)ccc23)cc1]

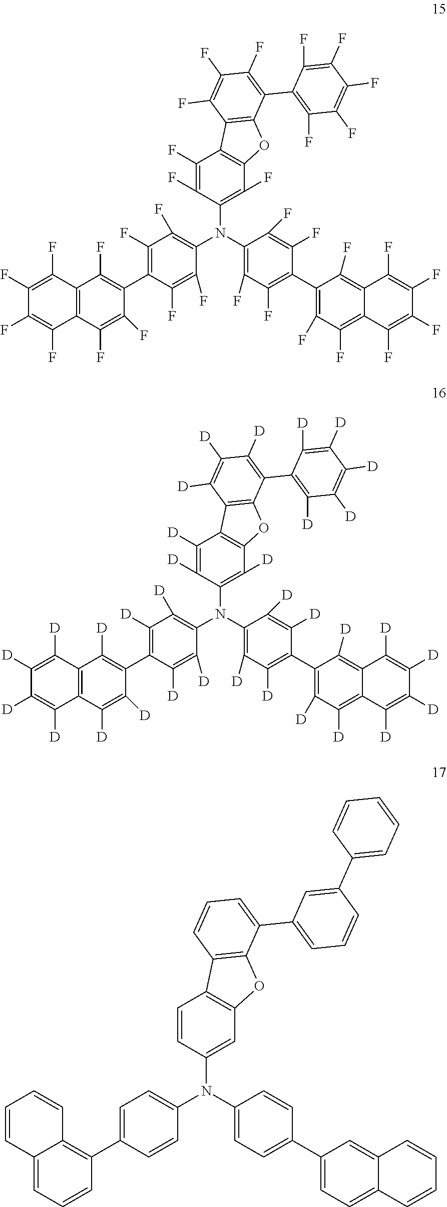

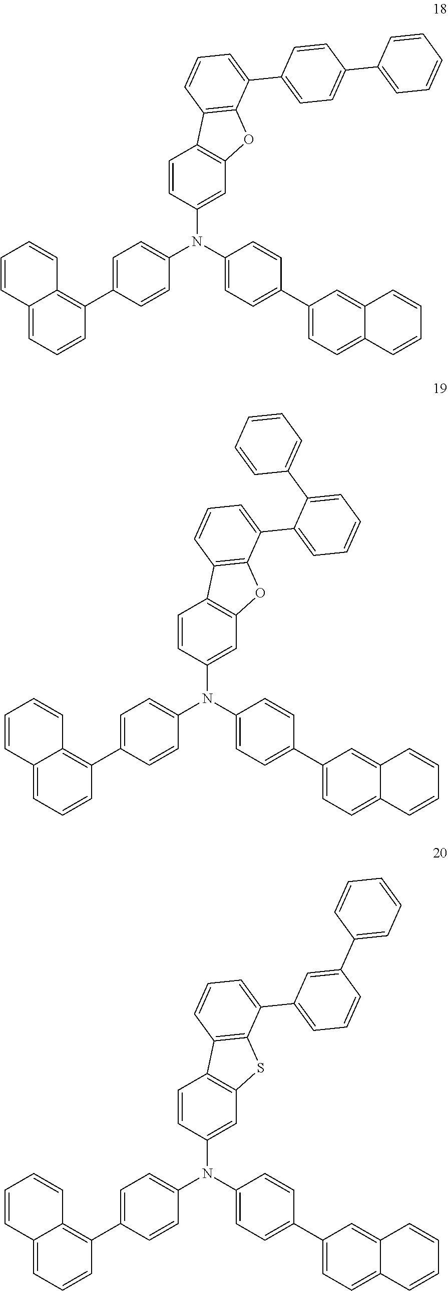 [SMILES: c1ccc(-c2ccc(-c3cccc4c3oc3cc(N(c5ccc(-c6ccc7ccccc7c6)cc5)c5ccc(-c6cccc7ccccc67)cc5)ccc34)cc2)cc1 ; c1ccc(-c2ccccc2-c2cccc3c2oc2cc(N(c4ccc(-c5ccc6ccccc6c5)cc4)c4ccc(-c5cccc6ccccc56)cc4)ccc23)cc1 ; c1ccc(-c2cccc(-c3cccc4c3sc3cc(N(c5ccc(-c6ccc7ccccc7c6)cc5)c5ccc(-c6cccc7ccccc67)cc5)ccc34)c2)cc1]

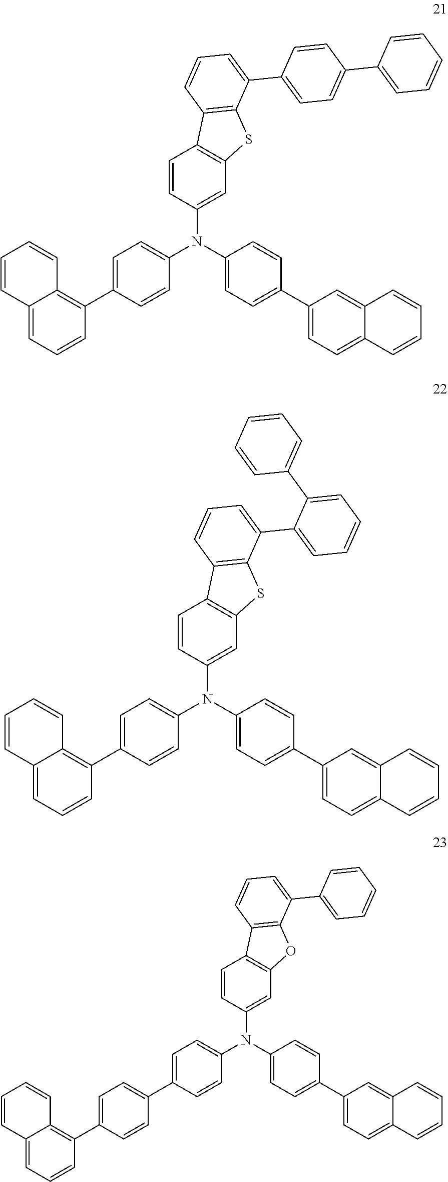 [SMILES: c1ccc(-c2ccc(-c3cccc4c3sc3cc(N(c5ccc(-c6ccc7ccccc7c6)cc5)c5ccc(-c6cccc7ccccc67)cc5)ccc34)cc2)cc1 ; c1ccc(-c2ccccc2-c2cccc3c2sc2cc(N(c4ccc(-c5ccc6ccccc6c5)cc4)c4ccc(-c5cccc6ccccc56)cc4)ccc23)cc1 ; c1ccc(-c2cccc3c2oc2cc(N(c4ccc(-c5ccc(-c6cccc7ccccc67)cc5)cc4)c4ccc(-c5ccc6ccccc6c5)cc4)ccc23)cc1]

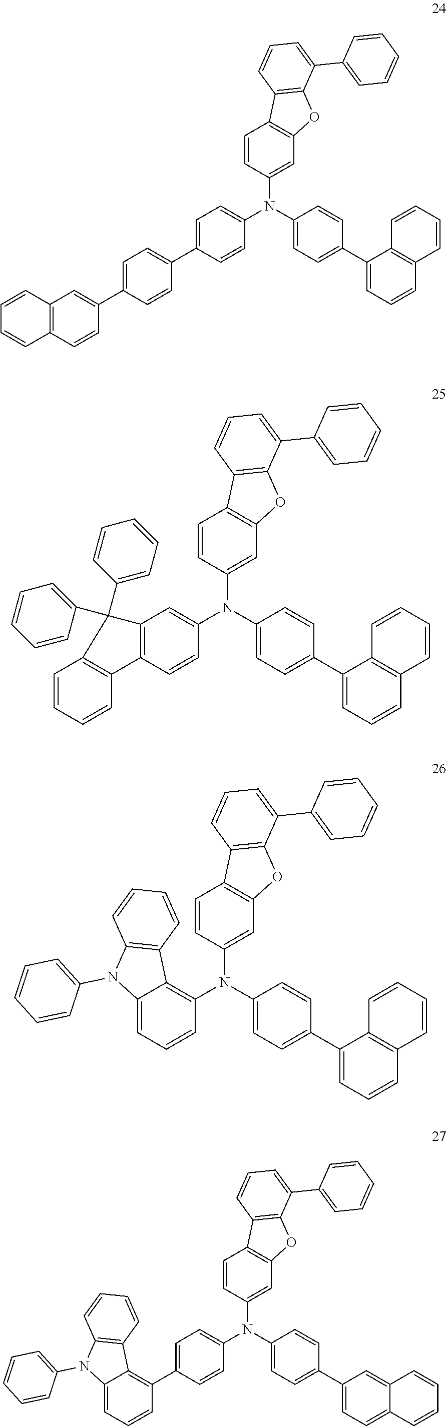 [SMILES: c1ccc(-c2cccc3c2oc2cc(N(c4ccc(-c5ccc(-c6ccc7ccccc7c6)cc5)cc4)c4ccc(-c5cccc6ccccc56)cc4)ccc23)cc1 ; c1ccc(-c2cccc3c2oc2cc(N(c4ccc(-c5cccc6ccccc56)cc4)c4ccc5c(c4)C(c4ccccc4)(c4ccccc4)c4ccccc4-5)ccc23)cc1 ; c1ccc(-c2cccc3c2oc2cc(N(c4ccc(-c5cccc6ccccc56)cc4)c4cccc5c4c4ccccc4n5-c4ccccc4)ccc23)cc1 ; c1ccc(-c2cccc3c2oc2cc(N(c4ccc(-c5ccc6ccccc6c5)cc4)c4ccc(-c5cccc6c5c5ccccc5n6-c5ccccc5)cc4)ccc23)cc1]

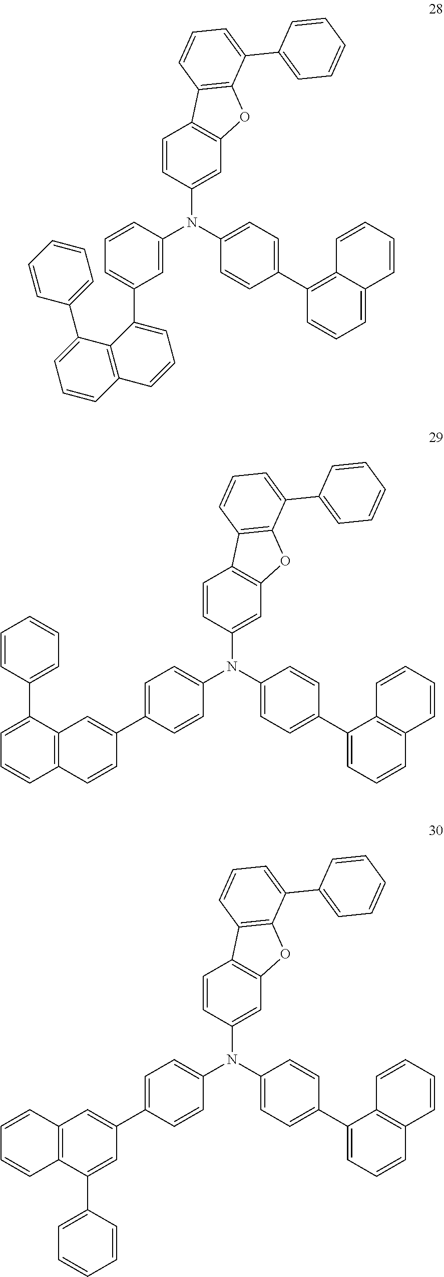 [SMILES: c1ccc(-c2cccc3c2oc2cc(N(c4ccc(-c5cccc6ccccc56)cc4)c4cccc(-c5cccc6cccc(-c7ccccc7)c56)c4)ccc23)cc1 ; c1ccc(-c2cccc3ccc(-c4ccc(N(c5ccc(-c6cccc7ccccc67)cc5)c5ccc6c(c5)oc5c(-c7ccccc7)cccc56)cc4)cc23)cc1 ; c1ccc(-c2cc(-c3ccc(N(c4ccc(-c5cccc6ccccc56)cc4)c4ccc5c(c4)oc4c(-c6ccccc6)cccc45)cc3)cc3ccccc23)cc1]

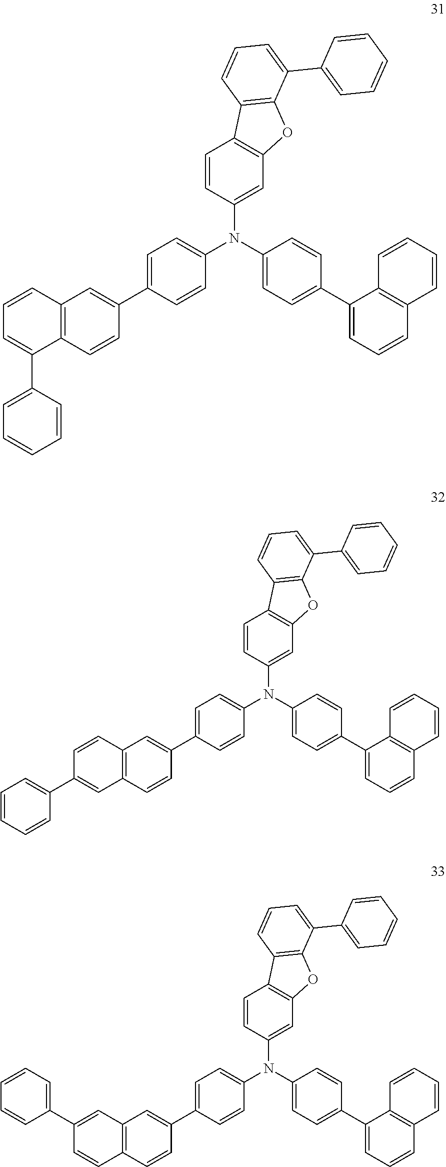 [SMILES: c1ccc(-c2cccc3cc(-c4ccc(N(c5ccc(-c6cccc7ccccc67)cc5)c5ccc6c(c5)oc5c(-c7ccccc7)cccc56)cc4)ccc23)cc1 ; c1ccc(-c2ccc3cc(-c4ccc(N(c5ccc(-c6cccc7ccccc67)cc5)c5ccc6c(c5)oc5c(-c7ccccc7)cccc56)cc4)ccc3c2)cc1 ; c1ccc(-c2ccc3ccc(-c4ccc(N(c5ccc(-c6cccc7ccccc67)cc5)c5ccc6c(c5)oc5c(-c7ccccc7)cccc56)cc4)cc3c2)cc1]

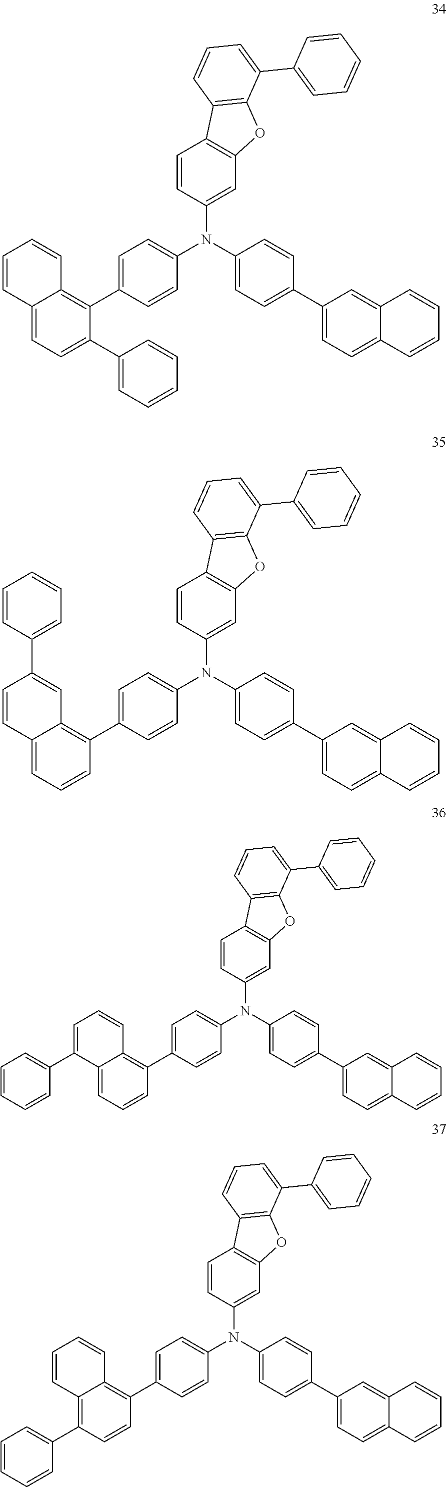 [SMILES: c1ccc(-c2ccc3ccccc3c2-c2ccc(N(c3ccc(-c4ccc5ccccc5c4)cc3)c3ccc4c(c3)oc3c(-c5ccccc5)cccc34)cc2)cc1 ; c1ccc(-c2ccc3cccc(-c4ccc(N(c5ccc(-c6ccc7ccccc7c6)cc5)c5ccc6c(c5)oc5c(-c7ccccc7)cccc56)cc4)c3c2)cc1 ; c1ccc(-c2cccc3c(-c4ccc(N(c5ccc(-c6ccc7ccccc7c6)cc5)c5ccc6c(c5)oc5c(-c7ccccc7)cccc56)cc4)cccc23)cc1 ; c1ccc(-c2ccc(-c3ccc(N(c4ccc(-c5ccc6ccccc6c5)cc4)c4ccc5c(c4)oc4c(-c6ccccc6)cccc45)cc3)c3ccccc23)cc1]

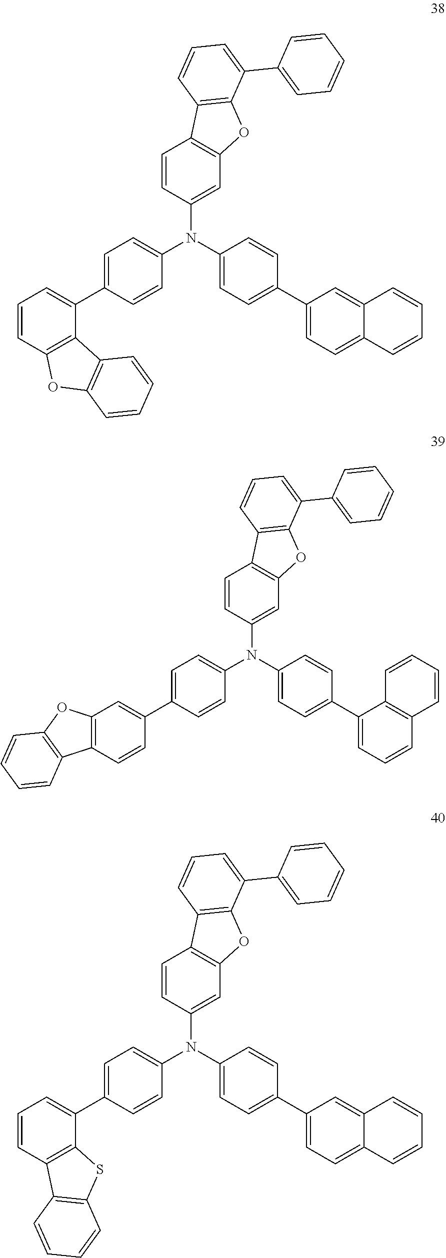 [SMILES: c1ccc(-c2cccc3c2oc2cc(N(c4ccc(-c5ccc6ccccc6c5)cc4)c4ccc(-c5cccc6oc7ccccc7c56)cc4)ccc23)cc1 ; c1ccc(-c2cccc3c2oc2cc(N(c4ccc(-c5ccc6c(c5)oc5ccccc56)cc4)c4ccc(-c5cccc6ccccc56)cc4)ccc23)cc1 ; c1ccc(-c2cccc3c2oc2cc(N(c4ccc(-c5ccc6ccccc6c5)cc4)c4ccc(-c5cccc6c5sc5ccccc56)cc4)ccc23)cc1]

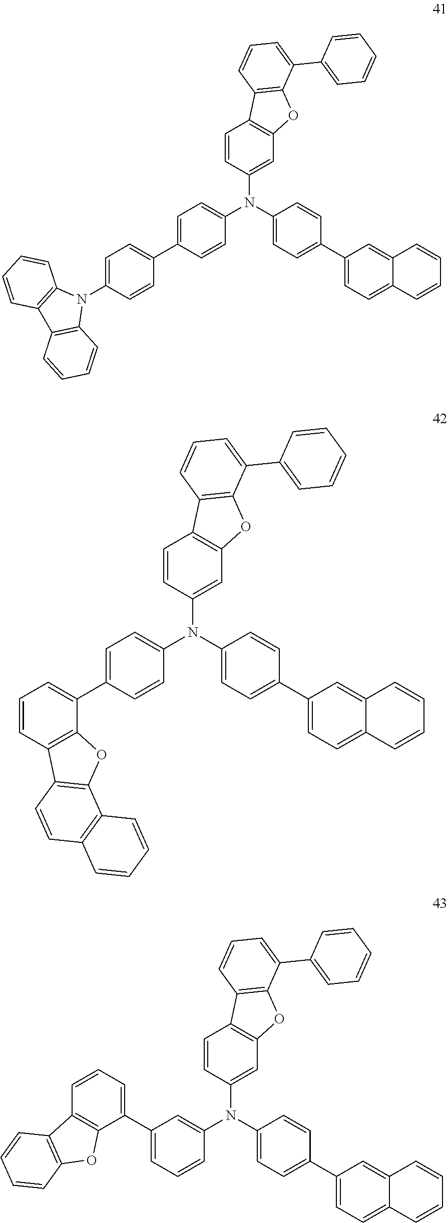 [SMILES: c1ccc(-c2cccc3c2oc2cc(N(c4ccc(-c5ccc(-n6c7ccccc7c7ccccc76)cc5)cc4)c4ccc(-c5ccc6ccccc6c5)cc4)ccc23)cc1 ; c1ccc(-c2cccc3c2oc2cc(N(c4ccc(-c5ccc6ccccc6c5)cc4)c4ccc(-c5cccc6c5oc5c7ccccc7ccc65)cc4)ccc23)cc1 ; c1ccc(-c2cccc3c2oc2cc(N(c4ccc(-c5ccc6ccccc6c5)cc4)c4cccc(-c5cccc6c5oc5ccccc56)c4)ccc23)cc1]

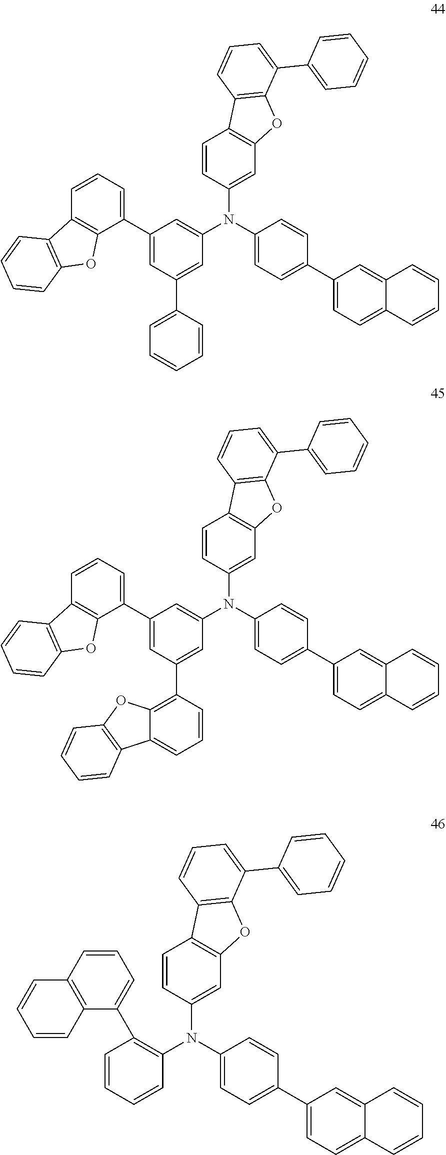 [SMILES: c1ccc(-c2cc(-c3cccc4c3oc3ccccc34)cc(N(c3ccc(-c4ccc5ccccc5c4)cc3)c3ccc4c(c3)oc3c(-c5ccccc5)cccc34)c2)cc1 ; c1ccc(-c2cccc3c2oc2cc(N(c4ccc(-c5ccc6ccccc6c5)cc4)c4cc(-c5cccc6c5oc5ccccc56)cc(-c5cccc6c5oc5ccccc56)c4)ccc23)cc1 ; c1ccc(-c2cccc3c2oc2cc(N(c4ccc(-c5ccc6ccccc6c5)cc4)c4ccccc4-c4cccc5ccccc45)ccc23)cc1]

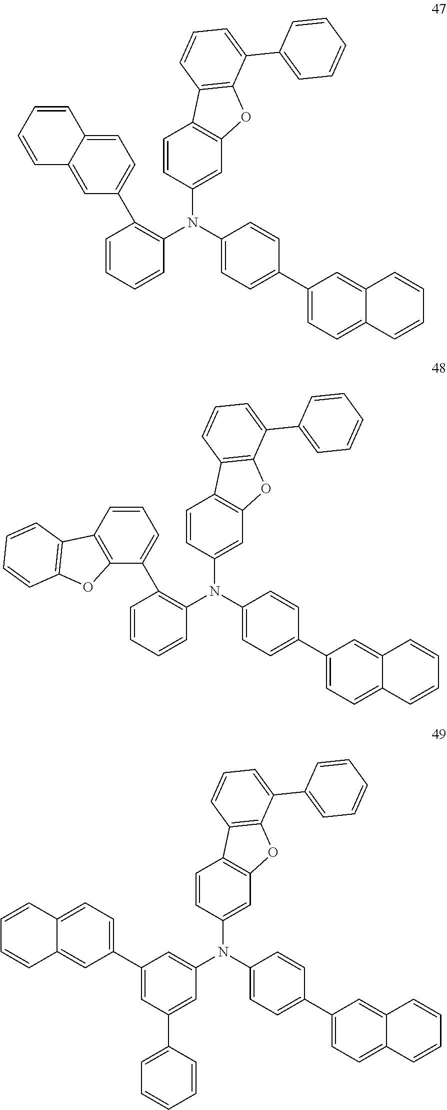 [SMILES: c1ccc(-c2cccc3c2oc2cc(N(c4ccc(-c5ccc6ccccc6c5)cc4)c4ccccc4-c4ccc5ccccc5c4)ccc23)cc1 ; c1ccc(-c2cccc3c2oc2cc(N(c4ccc(-c5ccc6ccccc6c5)cc4)c4ccccc4-c4cccc5c4oc4ccccc45)ccc23)cc1 ; c1ccc(-c2cc(-c3ccc4ccccc4c3)cc(N(c3ccc(-c4ccc5ccccc5c4)cc3)c3ccc4c(c3)oc3c(-c5ccccc5)cccc34)c2)cc1]

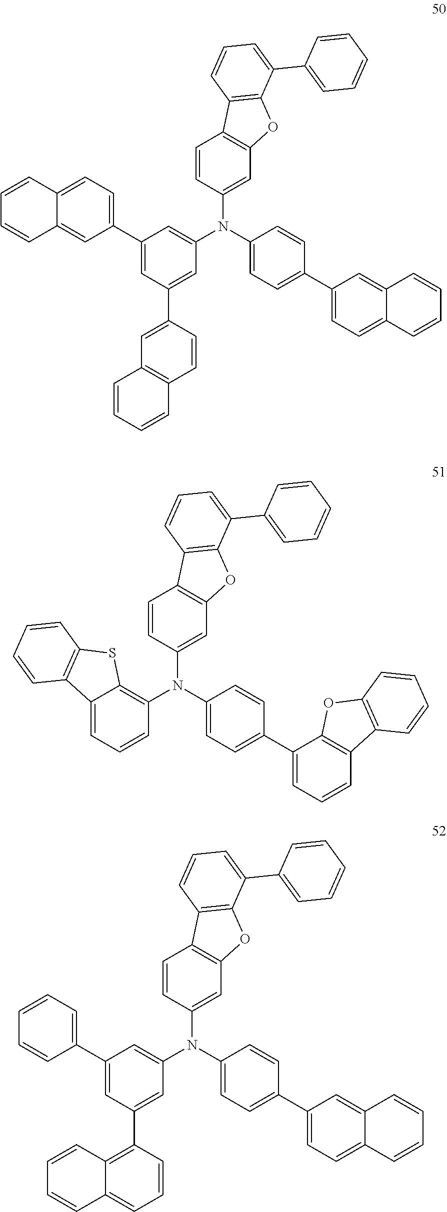

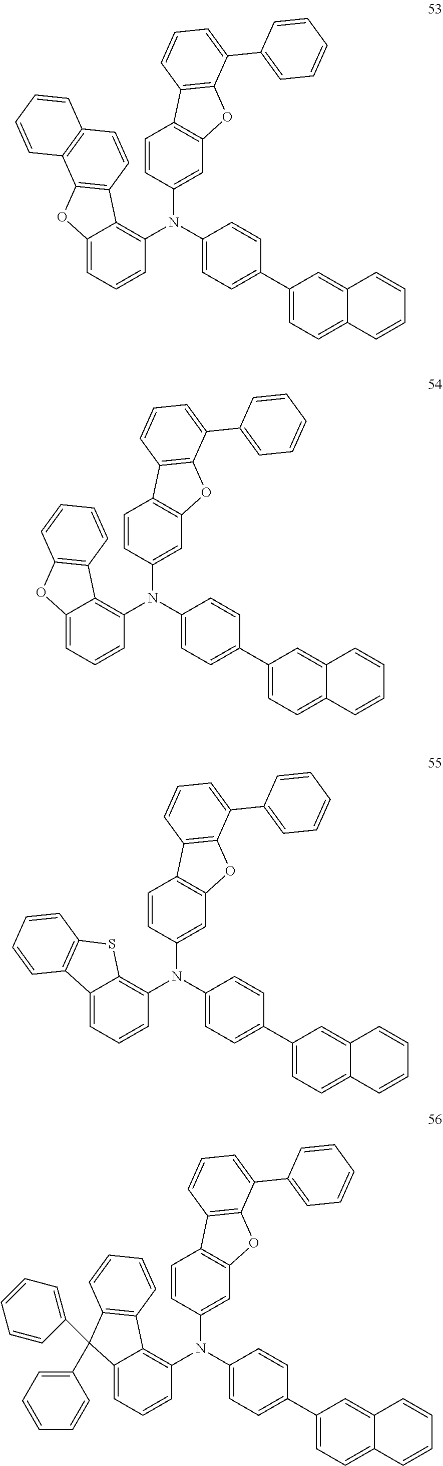 [SMILES: c1ccc(-c2cccc3c2oc2cc(N(c4ccc(-c5ccc6ccccc6c5)cc4)c4cccc5oc6c7ccccc7ccc6c45)ccc23)cc1 ; c1ccc(-c2cccc3c2oc2cc(N(c4ccc(-c5ccc6ccccc6c5)cc4)c4cccc5oc6ccccc6c45)ccc23)cc1 ; c1ccc(-c2cccc3c2oc2cc(N(c4ccc(-c5ccc6ccccc6c5)cc4)c4cccc5c4sc4ccccc45)ccc23)cc1 ; c1ccc(-c2cccc3c2oc2cc(N(c4ccc(-c5ccc6ccccc6c5)cc4)c4cccc5c4-c4ccccc4C5(c4ccccc4)c4ccccc4)ccc23)cc1]

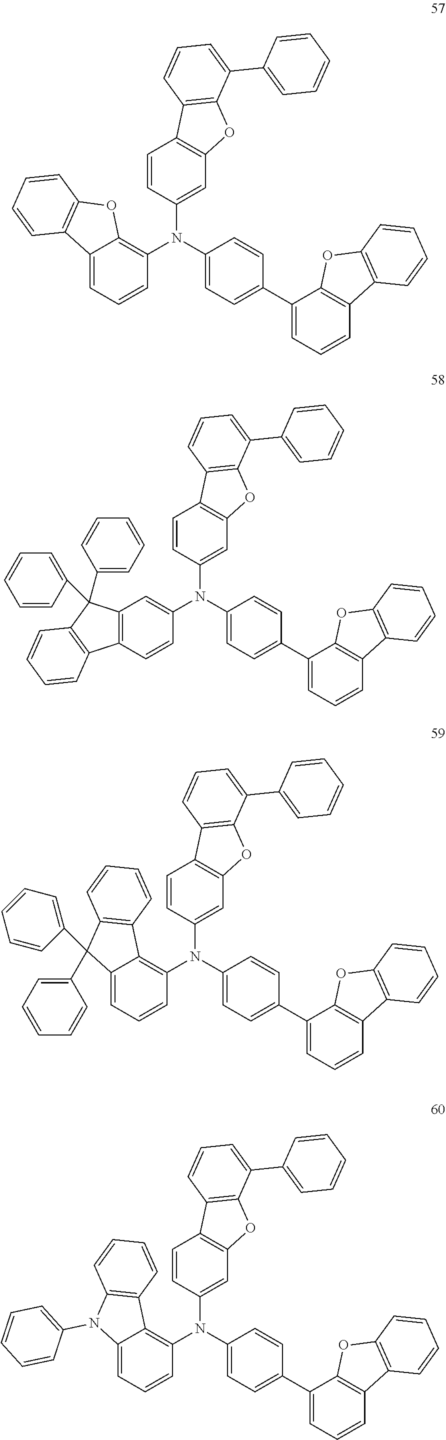 [SMILES: c1ccc(-c2cccc3c2oc2cc(N(c4ccc(-c5cccc6c5oc5ccccc56)cc4)c4cccc5c4oc4ccccc45)ccc23)cc1 ; c1ccc(-c2cccc3c2oc2cc(N(c4ccc(-c5cccc6c5oc5ccccc56)cc4)c4ccc5c(c4)C(c4ccccc4)(c4ccccc4)c4ccccc4-5)ccc23)cc1 ; c1ccc(-c2cccc3c2oc2cc(N(c4ccc(-c5cccc6c5oc5ccccc56)cc4)c4cccc5c4-c4ccccc4C5(c4ccccc4)c4ccccc4)ccc23)cc1 ; c1ccc(-c2cccc3c2oc2cc(N(c4ccc(-c5cccc6c5oc5ccccc56)cc4)c4cccc5c4c4ccccc4n5-c4ccccc4)ccc23)cc1]

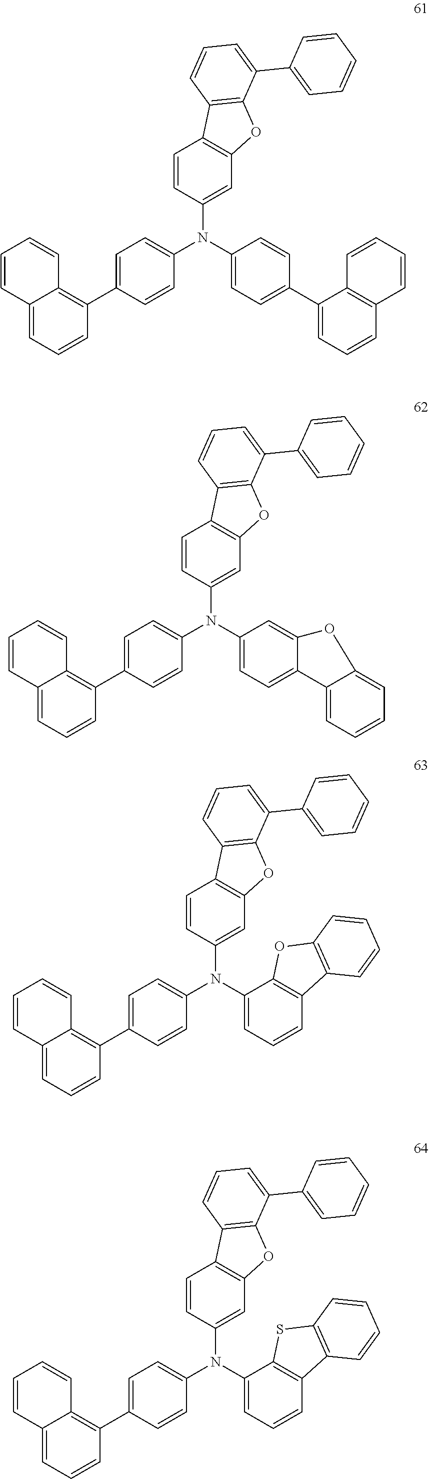

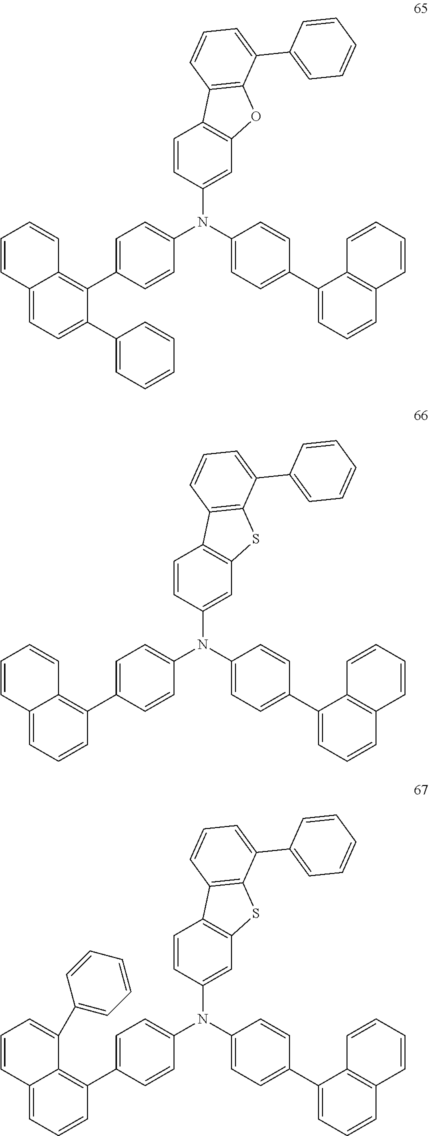 [SMILES: c1ccc(-c2ccc3ccccc3c2-c2ccc(N(c3ccc(-c4cccc5ccccc45)cc3)c3ccc4c(c3)oc3c(-c5ccccc5)cccc34)cc2)cc1 ; c1ccc(-c2cccc3c2sc2cc(N(c4ccc(-c5cccc6ccccc56)cc4)c4ccc(-c5cccc6ccccc56)cc4)ccc23)cc1 ; c1ccc(-c2cccc3c2sc2cc(N(c4ccc(-c5cccc6ccccc56)cc4)c4ccc(-c5cccc6cccc(-c7ccccc7)c56)cc4)ccc23)cc1]

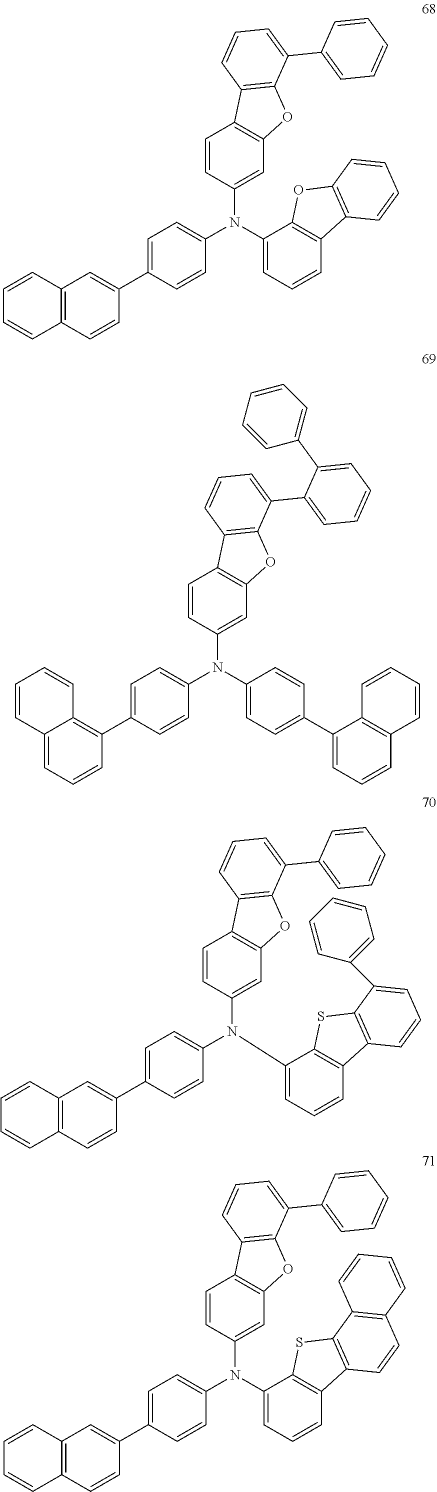

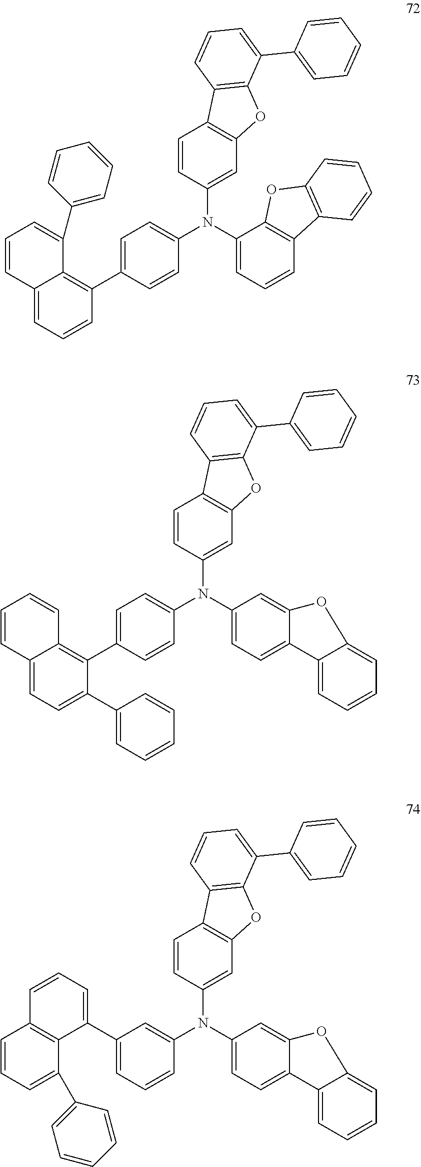 [SMILES: c1ccc(-c2cccc3c2oc2cc(N(c4ccc(-c5cccc6cccc(-c7ccccc7)c56)cc4)c4cccc5c4oc4ccccc45)ccc23)cc1 ; c1ccc(-c2ccc3ccccc3c2-c2ccc(N(c3ccc4c(c3)oc3ccccc34)c3ccc4c(c3)oc3c(-c5ccccc5)cccc34)cc2)cc1 ; c1ccc(-c2cccc3c2oc2cc(N(c4cccc(-c5cccc6cccc(-c7ccccc7)c56)c4)c4ccc5c(c4)oc4ccccc45)ccc23)cc1]

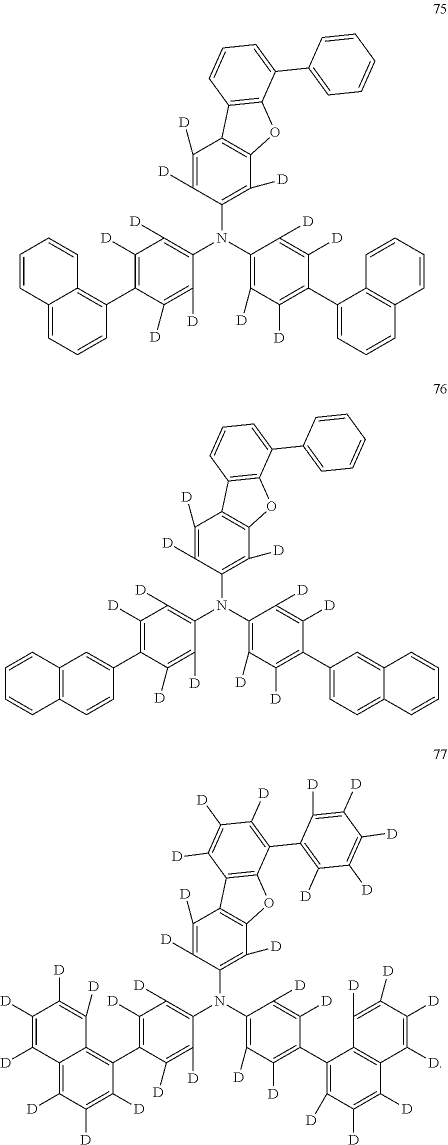

13. An amine compound represented by the following Formula 1: 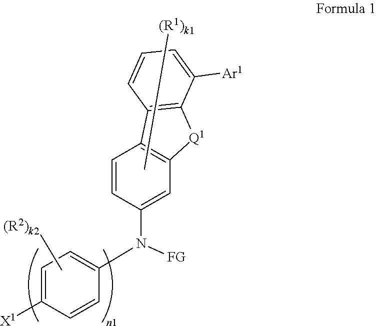
in Formula 1,
Q¹ is O or S,
Ar¹ is a substituted or unsubstituted phenyl group,
R¹ and R² are each independently a hydrogen atom, a deuterium atom, a halogen atom, a substituted or unsubstituted silyl group, a substituted or unsubstituted aryl group of 6 to 30 ring-forming carbon atoms, or a substituted or unsubstituted heteroaryl group of 2 to 30 ring-forming carbon atoms,
X¹ is a substituted or unsubstituted naphthyl group, or a substituted or unsubstituted heteroaryl group of 2 to 30 ring-forming carbon atoms,
n1 is an integer of 1 to 3, k1 is an integer of 0 to 6, k2 is an integer of 0 to 4, and
FG is represented by the following Formula 2-1 or Formula 2-2: 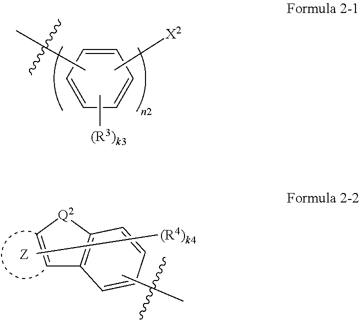
in Formula 2-1 and Formula 2-2,
X² is a substituted or unsubstituted naphthyl group, or a substituted or unsubstituted heteroaryl group of 2 to 30 ring-forming carbon atoms,
R³ and R⁴ are each independently a hydrogen atom, a deuterium atom, a halogen atom, a substituted or unsubstituted silyl group, a substituted or unsubstituted aryl group of 6 to 30 ring-forming carbon atoms, or a substituted or unsubstituted heteroaryl group of 2 to 30 ring-forming carbon atoms,
Q² is O, S, NR⁵, or CR⁶R⁷,
R⁵ to R⁷ are each independently a substituted or unsubstituted alkyl group of 1 to 20 carbon atoms, a substituted or unsubstituted aryl group of 6 to 50 ring-forming carbon atoms, or a substituted or unsubstituted heteroaryl group of 2 to 50 ring-forming carbon atoms,
Z is a substituted or unsubstituted aromatic hydrocarbon ring of 6 to 30 ring-forming carbon atoms, or a substituted or unsubstituted aromatic heterocycle of 2 to 30 ring-forming carbon atoms,
k3 is an integer of 0 to 4, k4 is an integer of 0 to 7, and n2 is an integer of 1 to 3, and
a case where X¹ and X² are 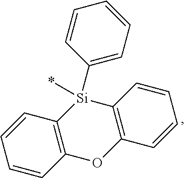 [SMILES: *[Si]1(c2ccccc2)c2ccccc2Oc2ccccc21]
is excluded.

14. The amine compound of claim 13, wherein Formula 2-1 is represented by the following Formula 2-1a or Formula 2-1b: 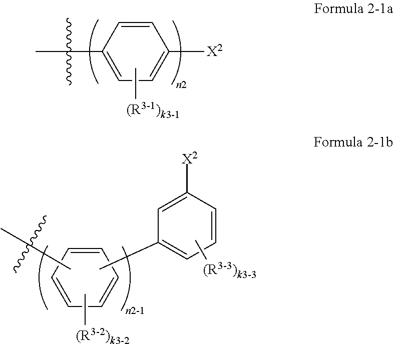
in Formula 2-1a and Formula 2-1b,
R³⁻¹ to R³⁻³ are each independently a hydrogen atom, a deuterium atom, a halogen atom, a substituted or unsubstituted aryl group or 6 to 15 ring-forming carbon atoms, or a substituted or unsubstituted heteroaryl group of 2 to 15 ring-forming carbon atoms,
k3-1 to k3-3 are each independently an integer of 0 to 4,
n2-1 is an integer of 0 to 2, and
X² and n2 are the same as defined with respect to Formula 2-1 and Formula 2-2.

15. The amine compound of claim 13, wherein Formula 2-2 is represented by any one among the following Formula 2-2a to Formula 2-2c: 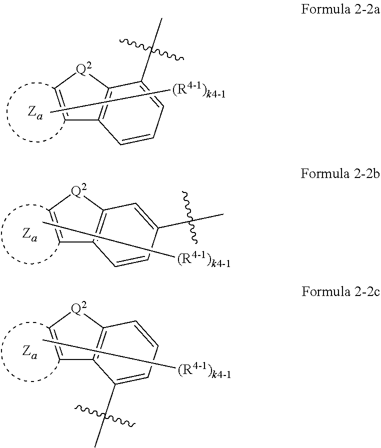
in Formula 2-2a to Formula 2-2c,
Zₐ is a substituted or unsubstituted aromatic hydrocarbon ring of 6 to 10 ring-forming carbon atoms,
R⁴⁻¹ is a hydrogen atom, a deuterium atom, a halogen atom, or a substituted or unsubstituted aryl group of 6 to 15 ring-forming carbon atoms,
k4-1 is an integer of 0 to 7, and
Q² is the same as defined with respect to Formula 2-2.

16. The amine compound of claim 13, wherein FG is represented by any one among the following Formula FG-1 to Formula FG-6: 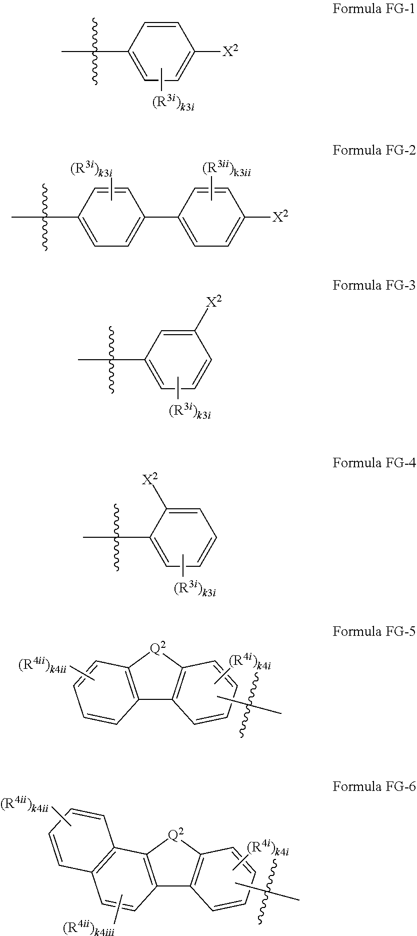
in Formula FG-1 to Formula FG-6,
R³ⁱ, R³ⁱⁱ and R⁴ⁱ to R⁴ⁱⁱⁱ are each independently a hydrogen atom, a deuterium atom, a halogen atom, or a substituted or unsubstituted phenyl group,
k3i, k3ii, and k4ii are each independently an integer of 0 to 4,
k4i is an integer of 0 to 3,
k4iii is an integer of 0 to 2, and
X² and Q² are the same as defined with respect to Formula 2-1 and Formula 2-2.

17. The amine compound of claim 13, wherein Formula 1 is represented by the following Formula 3-1 or Formula 3-2: 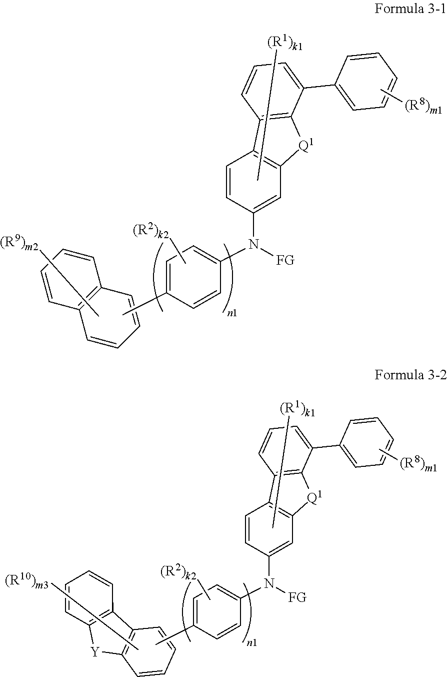
in Formula 3-1 and Formula 3-2,
Y is O or NR¹¹,
R⁸ to R¹¹ are each independently a hydrogen atom, a deuterium atom, a halogen atom, or a substituted or unsubstituted aryl group of 6 to 20 ring-forming carbon atoms,
m1 is an integer of 0 to 5, m2 and m3 are each independently an integer of 0 to 7, and
Q¹, R¹, R², n1, k1, k2, and FG are the same as defined with respect to Formula 1.

18. The amine compound of claim 13, wherein Formula 1 is represented by the following Formula 4-1 or Formula 4-2: 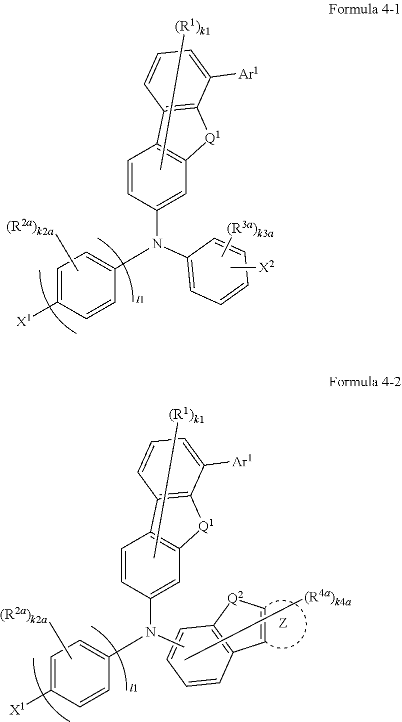
in Formula 4-1 and Formula 4-2,
R^{2a} to R^{4a} are each independently a hydrogen atom, a deuterium atom, a halogen atom, or a substituted or unsubstituted phenyl group,
k2a and k3a are each independently an integer of 0 to 4, k4a is an integer of 0 to 7,
l1 is 1 or 2, and
Ar¹, R¹, X¹, X², Q¹, Q², Z, and k1 are the same as defined with respect to Formula 1, Formula 2-1 and Formula 2-2.

19. The amine compound of claim 13, wherein
if FG is represented by Formula 2-1, at least one among X¹ and X² is a substituted or unsubstituted naphthyl group, and
if FG is represented by Formula 2-2, X¹ is a substituted or unsubstituted naphthyl group.

20. The amine compound of claim 13, wherein the amine compound represented by Formula 1 is represented by any one among compounds in the following Compound Group 1:

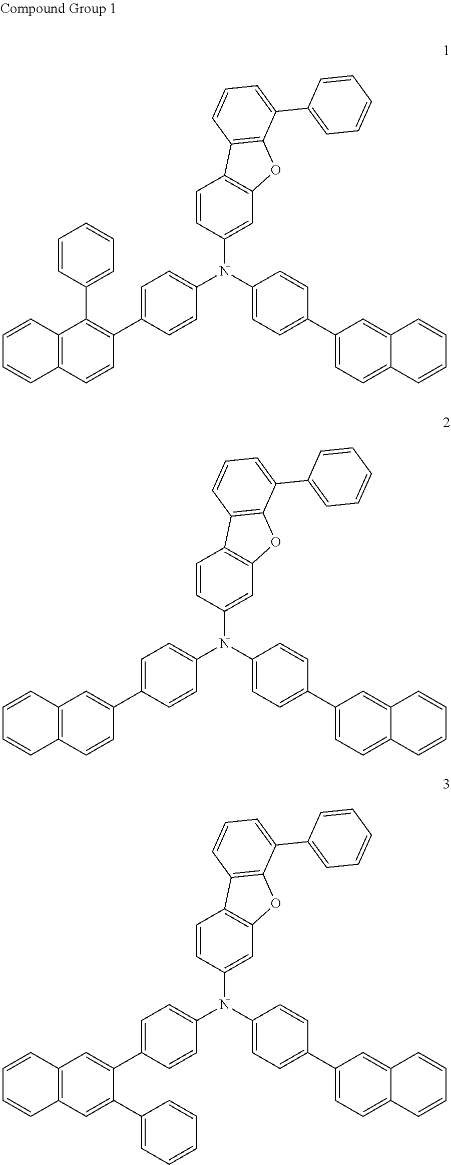 [SMILES: c1ccc(-c2c(-c3ccc(N(c4ccc(-c5ccc6ccccc6c5)cc4)c4ccc5c(c4)oc4c(-c6ccccc6)cccc45)cc3)ccc3ccccc23)cc1 ; c1ccc(-c2cccc3c2oc2cc(N(c4ccc(-c5ccc6ccccc6c5)cc4)c4ccc(-c5ccc6ccccc6c5)cc4)ccc23)cc1 ; c1ccc(-c2cc3ccccc3cc2-c2ccc(N(c3ccc(-c4ccc5ccccc5c4)cc3)c3ccc4c(c3)oc3c(-c5ccccc5)cccc34)cc2)cc1]

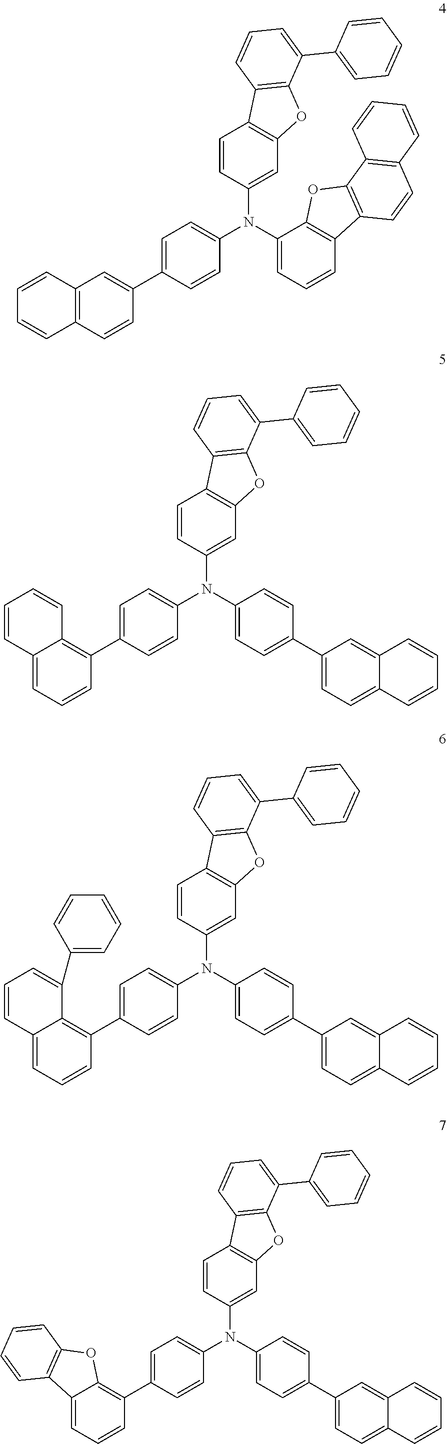 [SMILES: c1ccc(-c2cccc3c2oc2cc(N(c4ccc(-c5ccc6ccccc6c5)cc4)c4cccc5c4oc4c6ccccc6ccc54)ccc23)cc1 ; c1ccc(-c2cccc3c2oc2cc(N(c4ccc(-c5ccc6ccccc6c5)cc4)c4ccc(-c5cccc6ccccc56)cc4)ccc23)cc1 ; c1ccc(-c2cccc3c2oc2cc(N(c4ccc(-c5ccc6ccccc6c5)cc4)c4ccc(-c5cccc6cccc(-c7ccccc7)c56)cc4)ccc23)cc1 ; c1ccc(-c2cccc3c2oc2cc(N(c4ccc(-c5ccc6ccccc6c5)cc4)c4ccc(-c5cccc6c5oc5ccccc56)cc4)ccc23)cc1]

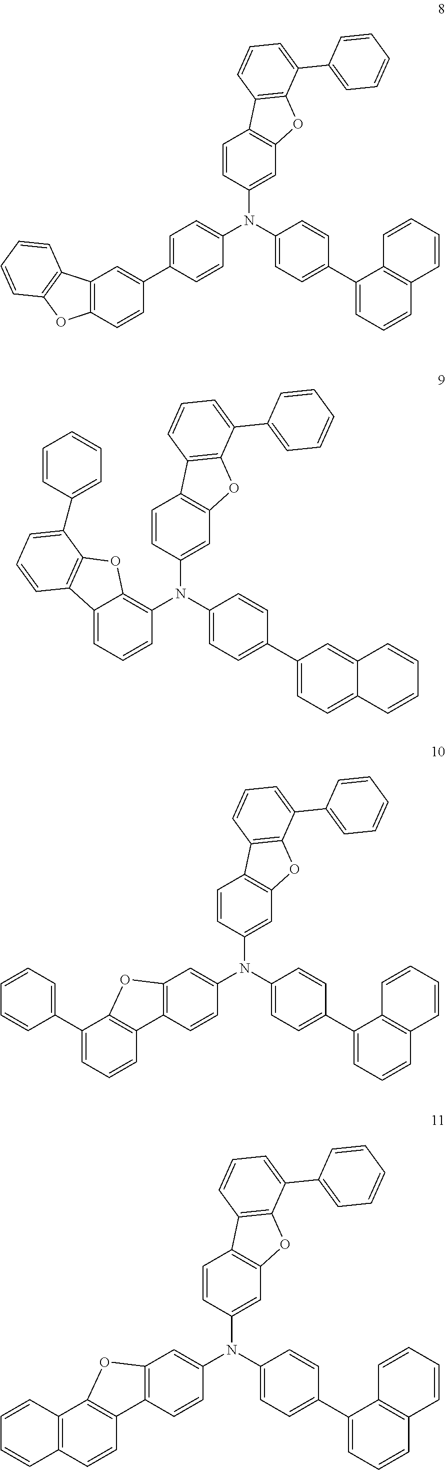 [SMILES: c1ccc(-c2cccc3c2oc2cc(N(c4ccc(-c5ccc6oc7ccccc7c6c5)cc4)c4ccc(-c5cccc6ccccc56)cc4)ccc23)cc1 ; c1ccc(-c2cccc3c2oc2cc(N(c4ccc(-c5ccc6ccccc6c5)cc4)c4cccc5c4oc4c(-c6ccccc6)cccc45)ccc23)cc1 ; c1ccc(-c2cccc3c2oc2cc(N(c4ccc(-c5cccc6ccccc56)cc4)c4ccc5c(c4)oc4c(-c6ccccc6)cccc45)ccc23)cc1 ; c1ccc(-c2cccc3c2oc2cc(N(c4ccc(-c5cccc6ccccc56)cc4)c4ccc5c(c4)oc4c6ccccc6ccc54)ccc23)cc1]

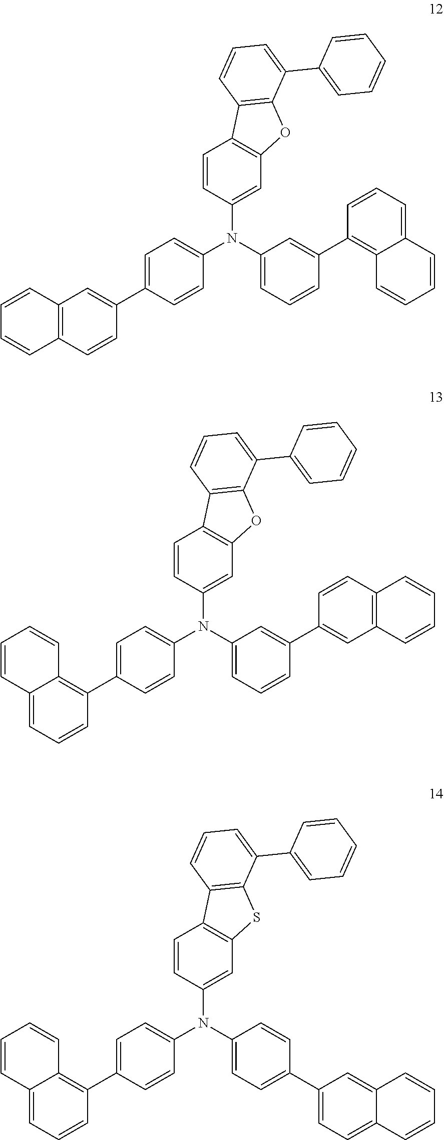 [SMILES: c1ccc(-c2cccc3c2oc2cc(N(c4ccc(-c5ccc6ccccc6c5)cc4)c4cccc(-c5cccc6ccccc56)c4)ccc23)cc1 ; c1ccc(-c2cccc3c2oc2cc(N(c4ccc(-c5cccc6ccccc56)cc4)c4cccc(-c5ccc6ccccc6c5)c4)ccc23)cc1 ; c1ccc(-c2cccc3c2sc2cc(N(c4ccc(-c5ccc6ccccc6c5)cc4)c4ccc(-c5cccc6ccccc56)cc4)ccc23)cc1]

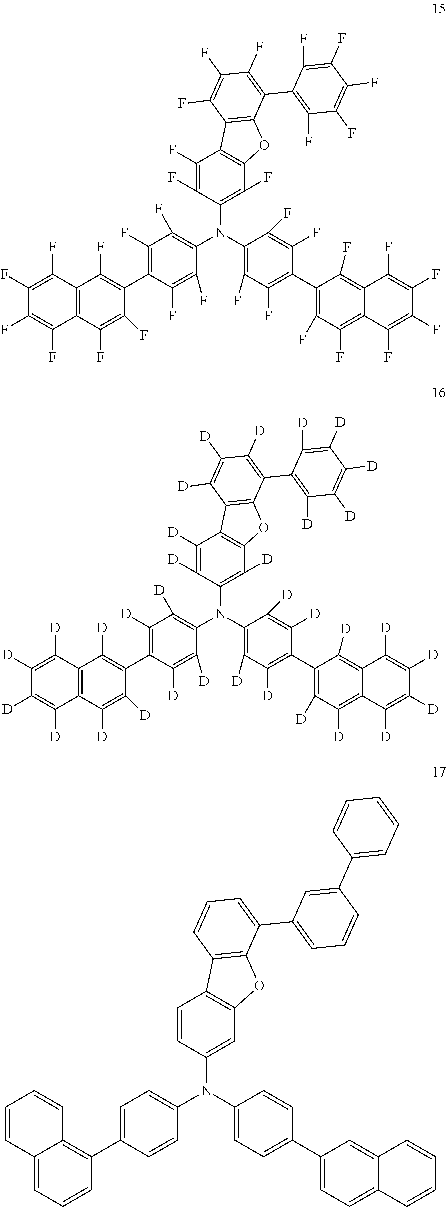

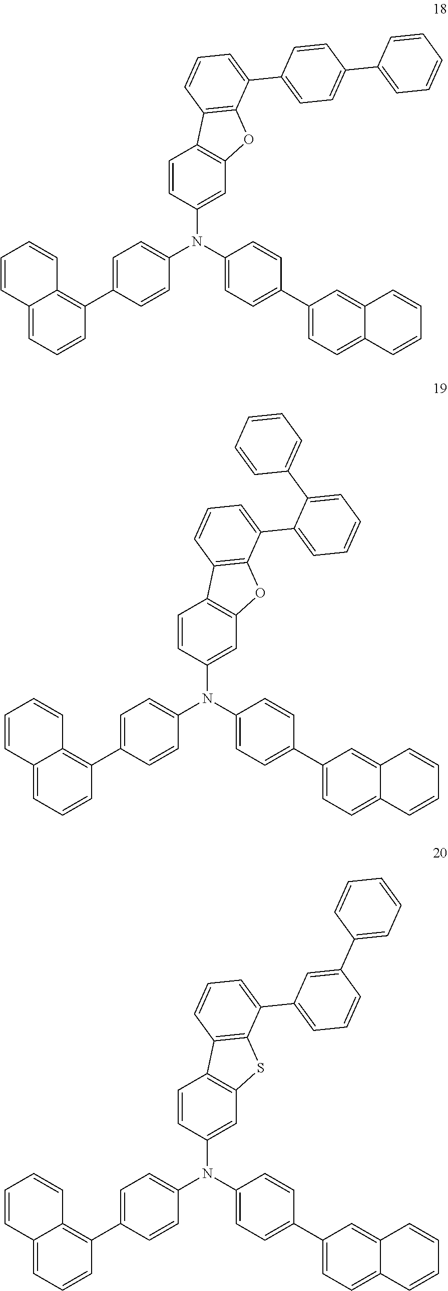 [SMILES: c1ccc(-c2ccc(-c3cccc4c3oc3cc(N(c5ccc(-c6ccc7ccccc7c6)cc5)c5ccc(-c6cccc7ccccc67)cc5)ccc34)cc2)cc1 ; c1ccc(-c2ccccc2-c2cccc3c2oc2cc(N(c4ccc(-c5ccc6ccccc6c5)cc4)c4ccc(-c5cccc6ccccc56)cc4)ccc23)cc1 ; c1ccc(-c2cccc(-c3cccc4c3sc3cc(N(c5ccc(-c6ccc7ccccc7c6)cc5)c5ccc(-c6cccc7ccccc67)cc5)ccc34)c2)cc1]

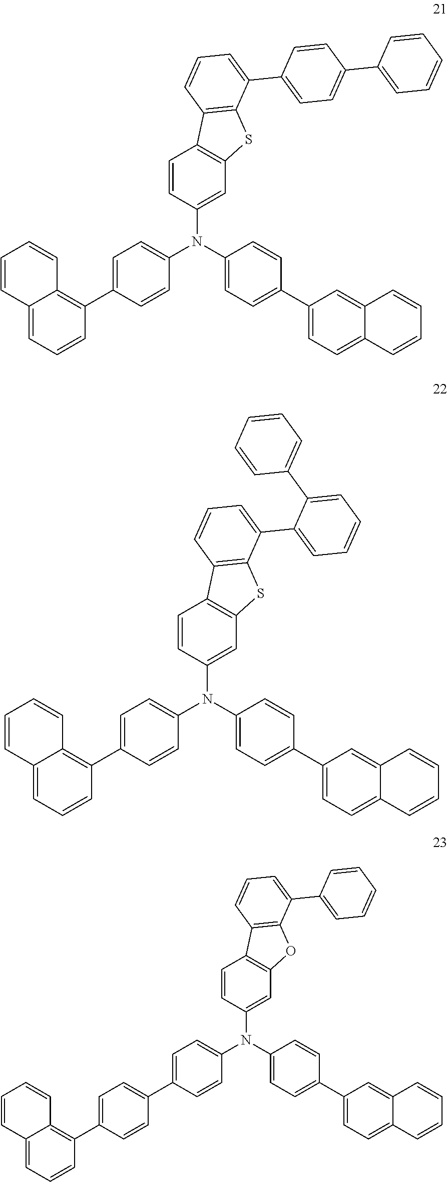 [SMILES: c1ccc(-c2ccc(-c3cccc4c3sc3cc(N(c5ccc(-c6ccc7ccccc7c6)cc5)c5ccc(-c6cccc7ccccc67)cc5)ccc34)cc2)cc1 ; c1ccc(-c2ccccc2-c2cccc3c2sc2cc(N(c4ccc(-c5ccc6ccccc6c5)cc4)c4ccc(-c5cccc6ccccc56)cc4)ccc23)cc1 ; c1ccc(-c2cccc3c2oc2cc(N(c4ccc(-c5ccc(-c6cccc7ccccc67)cc5)cc4)c4ccc(-c5ccc6ccccc6c5)cc4)ccc23)cc1]

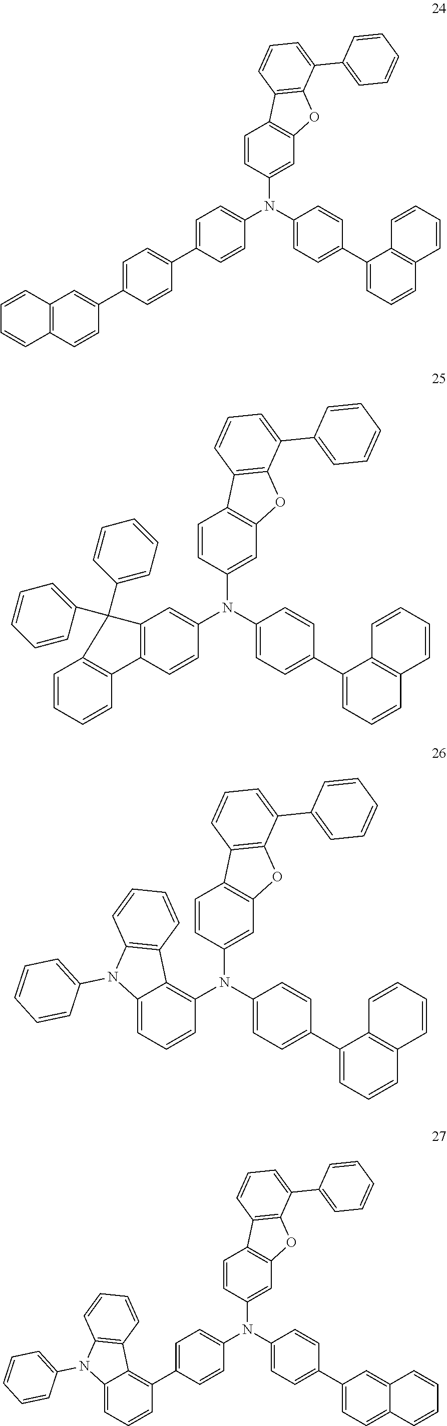 [SMILES: c1ccc(-c2cccc3c2oc2cc(N(c4ccc(-c5ccc(-c6ccc7ccccc7c6)cc5)cc4)c4ccc(-c5cccc6ccccc56)cc4)ccc23)cc1 ; c1ccc(-c2cccc3c2oc2cc(N(c4ccc(-c5cccc6ccccc56)cc4)c4ccc5c(c4)C(c4ccccc4)(c4ccccc4)c4ccccc4-5)ccc23)cc1 ; c1ccc(-c2cccc3c2oc2cc(N(c4ccc(-c5cccc6ccccc56)cc4)c4cccc5c4c4ccccc4n5-c4ccccc4)ccc23)cc1 ; c1ccc(-c2cccc3c2oc2cc(N(c4ccc(-c5ccc6ccccc6c5)cc4)c4ccc(-c5cccc6c5c5ccccc5n6-c5ccccc5)cc4)ccc23)cc1]

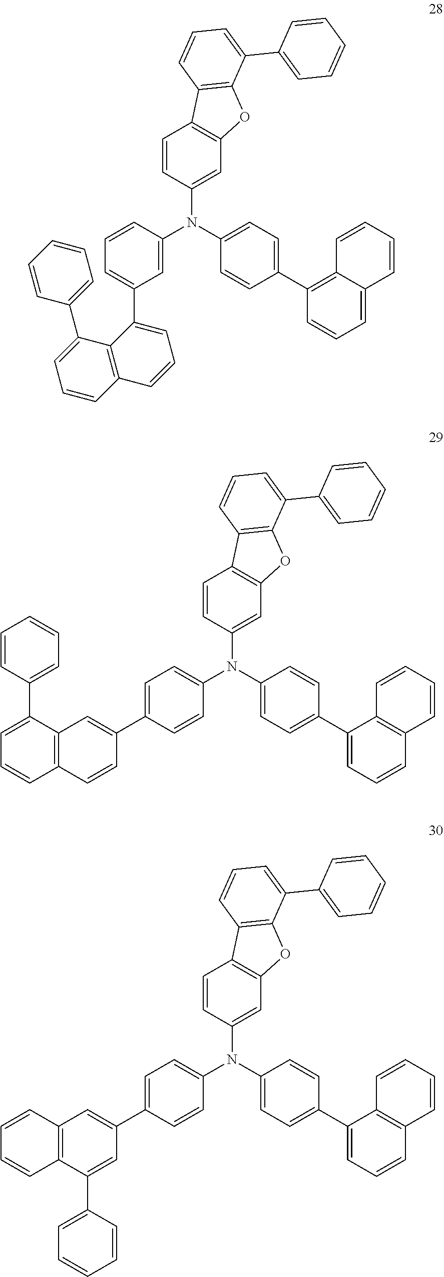 [SMILES: c1ccc(-c2cccc3c2oc2cc(N(c4ccc(-c5cccc6ccccc56)cc4)c4cccc(-c5cccc6cccc(-c7ccccc7)c56)c4)ccc23)cc1 ; c1ccc(-c2cccc3ccc(-c4ccc(N(c5ccc(-c6cccc7ccccc67)cc5)c5ccc6c(c5)oc5c(-c7ccccc7)cccc56)cc4)cc23)cc1 ; c1ccc(-c2cc(-c3ccc(N(c4ccc(-c5cccc6ccccc56)cc4)c4ccc5c(c4)oc4c(-c6ccccc6)cccc45)cc3)cc3ccccc23)cc1]

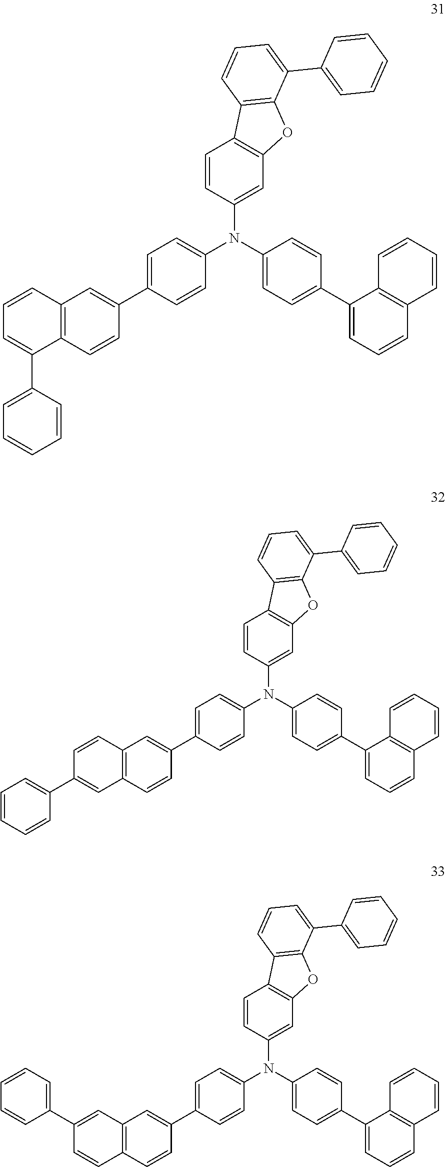 [SMILES: c1ccc(-c2cccc3cc(-c4ccc(N(c5ccc(-c6cccc7ccccc67)cc5)c5ccc6c(c5)oc5c(-c7ccccc7)cccc56)cc4)ccc23)cc1 ; c1ccc(-c2ccc3cc(-c4ccc(N(c5ccc(-c6cccc7ccccc67)cc5)c5ccc6c(c5)oc5c(-c7ccccc7)cccc56)cc4)ccc3c2)cc1 ; c1ccc(-c2ccc3ccc(-c4ccc(N(c5ccc(-c6cccc7ccccc67)cc5)c5ccc6c(c5)oc5c(-c7ccccc7)cccc56)cc4)cc3c2)cc1]

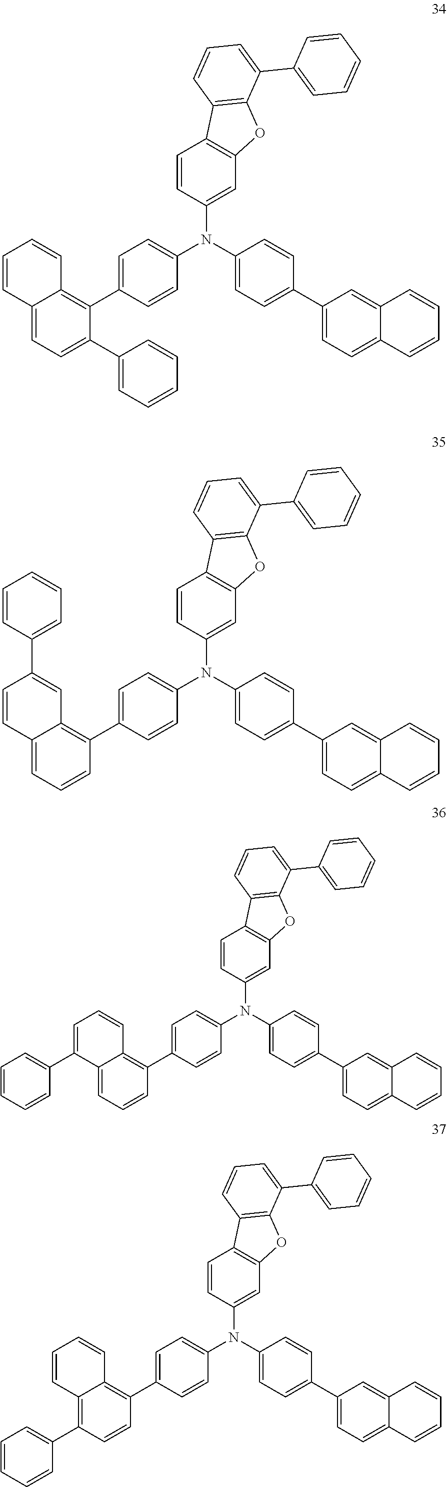 [SMILES: c1ccc(-c2ccc3ccccc3c2-c2ccc(N(c3ccc(-c4ccc5ccccc5c4)cc3)c3ccc4c(c3)oc3c(-c5ccccc5)cccc34)cc2)cc1 ; c1ccc(-c2ccc3cccc(-c4ccc(N(c5ccc(-c6ccc7ccccc7c6)cc5)c5ccc6c(c5)oc5c(-c7ccccc7)cccc56)cc4)c3c2)cc1 ; c1ccc(-c2cccc3c(-c4ccc(N(c5ccc(-c6ccc7ccccc7c6)cc5)c5ccc6c(c5)oc5c(-c7ccccc7)cccc56)cc4)cccc23)cc1 ; c1ccc(-c2ccc(-c3ccc(N(c4ccc(-c5ccc6ccccc6c5)cc4)c4ccc5c(c4)oc4c(-c6ccccc6)cccc45)cc3)c3ccccc23)cc1]

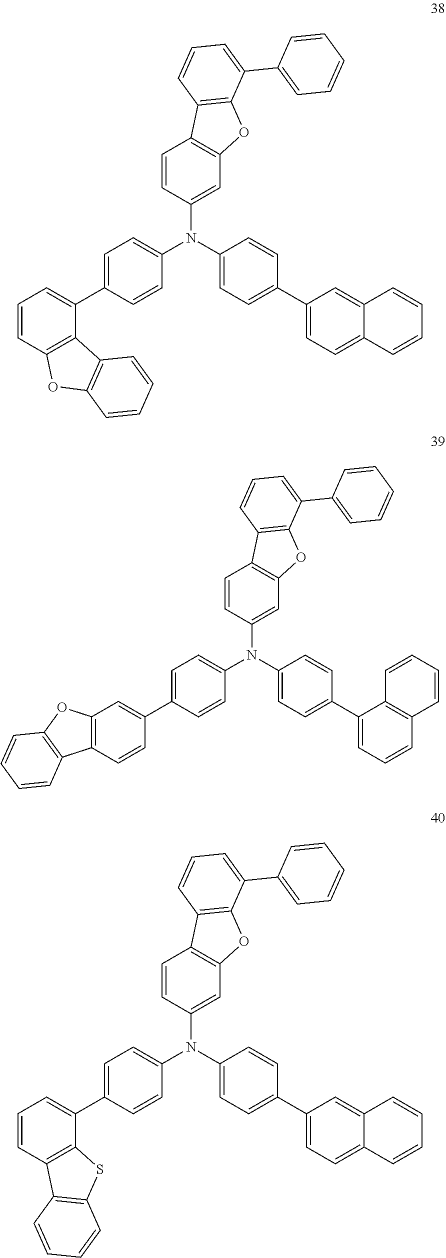 [SMILES: c1ccc(-c2cccc3c2oc2cc(N(c4ccc(-c5ccc6ccccc6c5)cc4)c4ccc(-c5cccc6oc7ccccc7c56)cc4)ccc23)cc1 ; c1ccc(-c2cccc3c2oc2cc(N(c4ccc(-c5ccc6c(c5)oc5ccccc56)cc4)c4ccc(-c5cccc6ccccc56)cc4)ccc23)cc1 ; c1ccc(-c2cccc3c2oc2cc(N(c4ccc(-c5ccc6ccccc6c5)cc4)c4ccc(-c5cccc6c5sc5ccccc56)cc4)ccc23)cc1]

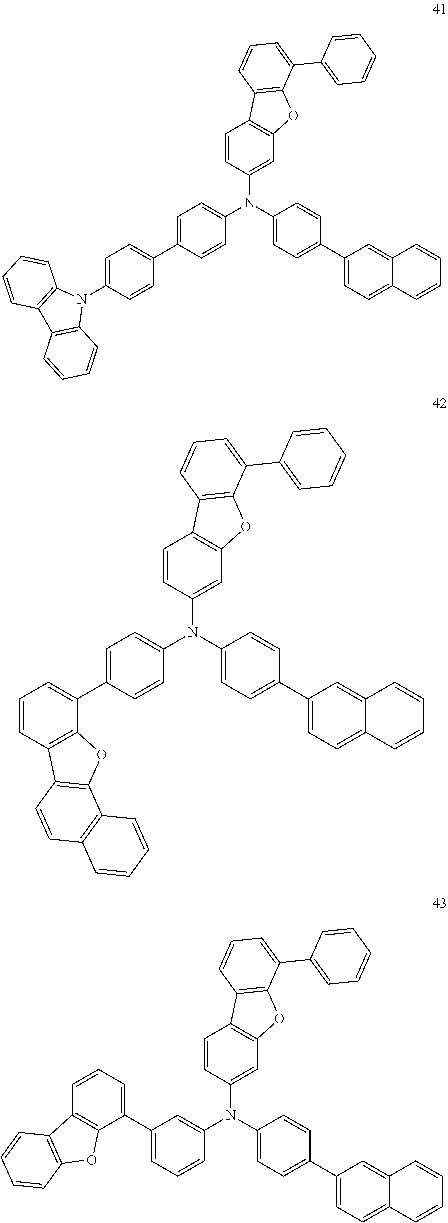 [SMILES: c1ccc(-c2cccc3c2oc2cc(N(c4ccc(-c5ccc(-n6c7ccccc7c7ccccc76)cc5)cc4)c4ccc(-c5ccc6ccccc6c5)cc4)ccc23)cc1 ; c1ccc(-c2cccc3c2oc2cc(N(c4ccc(-c5ccc6ccccc6c5)cc4)c4ccc(-c5cccc6c5oc5c7ccccc7ccc65)cc4)ccc23)cc1 ; c1ccc(-c2cccc3c2oc2cc(N(c4ccc(-c5ccc6ccccc6c5)cc4)c4cccc(-c5cccc6c5oc5ccccc56)c4)ccc23)cc1]

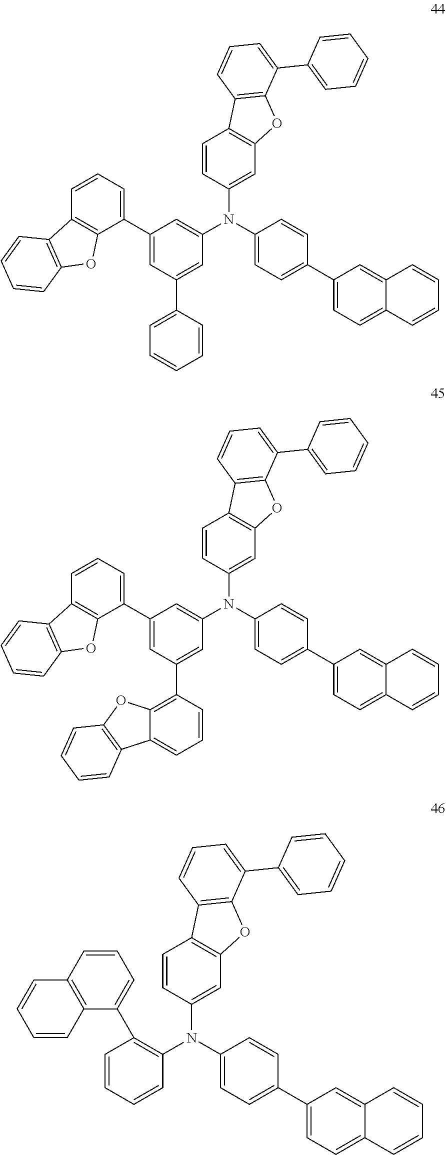 [SMILES: c1ccc(-c2cc(-c3cccc4c3oc3ccccc34)cc(N(c3ccc(-c4ccc5ccccc5c4)cc3)c3ccc4c(c3)oc3c(-c5ccccc5)cccc34)c2)cc1 ; c1ccc(-c2cccc3c2oc2cc(N(c4ccc(-c5ccc6ccccc6c5)cc4)c4cc(-c5cccc6c5oc5ccccc56)cc(-c5cccc6c5oc5ccccc56)c4)ccc23)cc1 ; c1ccc(-c2cccc3c2oc2cc(N(c4ccc(-c5ccc6ccccc6c5)cc4)c4ccccc4-c4cccc5ccccc45)ccc23)cc1]

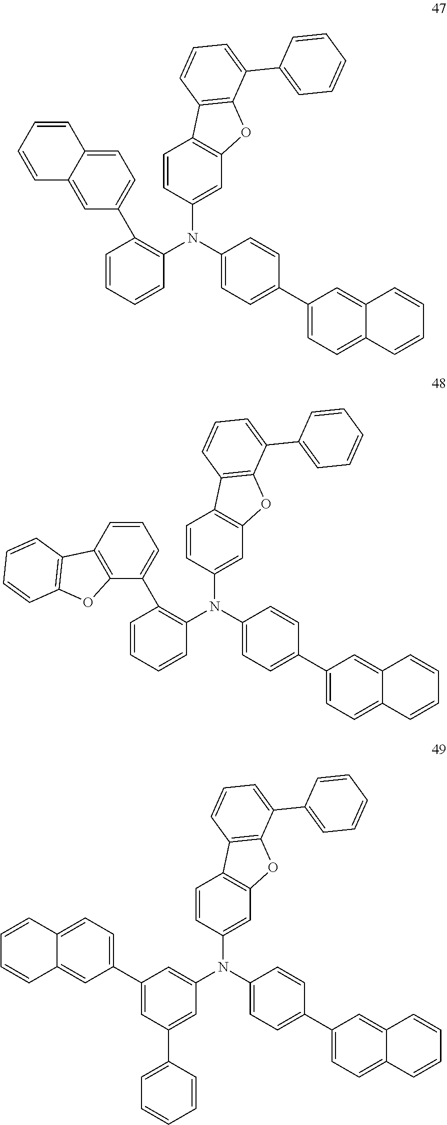 [SMILES: c1ccc(-c2cccc3c2oc2cc(N(c4ccc(-c5ccc6ccccc6c5)cc4)c4ccccc4-c4ccc5ccccc5c4)ccc23)cc1 ; c1ccc(-c2cccc3c2oc2cc(N(c4ccc(-c5ccc6ccccc6c5)cc4)c4ccccc4-c4cccc5c4oc4ccccc45)ccc23)cc1 ; c1ccc(-c2cc(-c3ccc4ccccc4c3)cc(N(c3ccc(-c4ccc5ccccc5c4)cc3)c3ccc4c(c3)oc3c(-c5ccccc5)cccc34)c2)cc1]

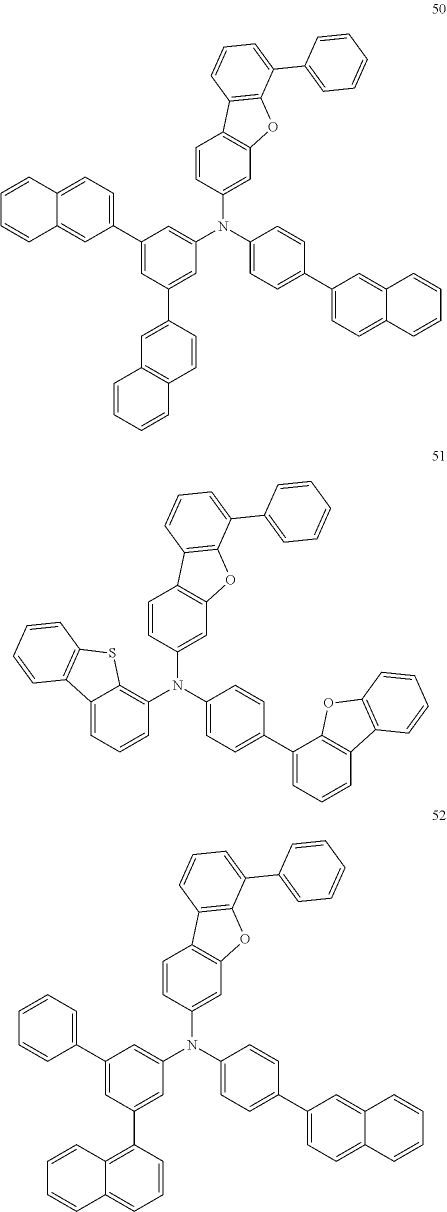

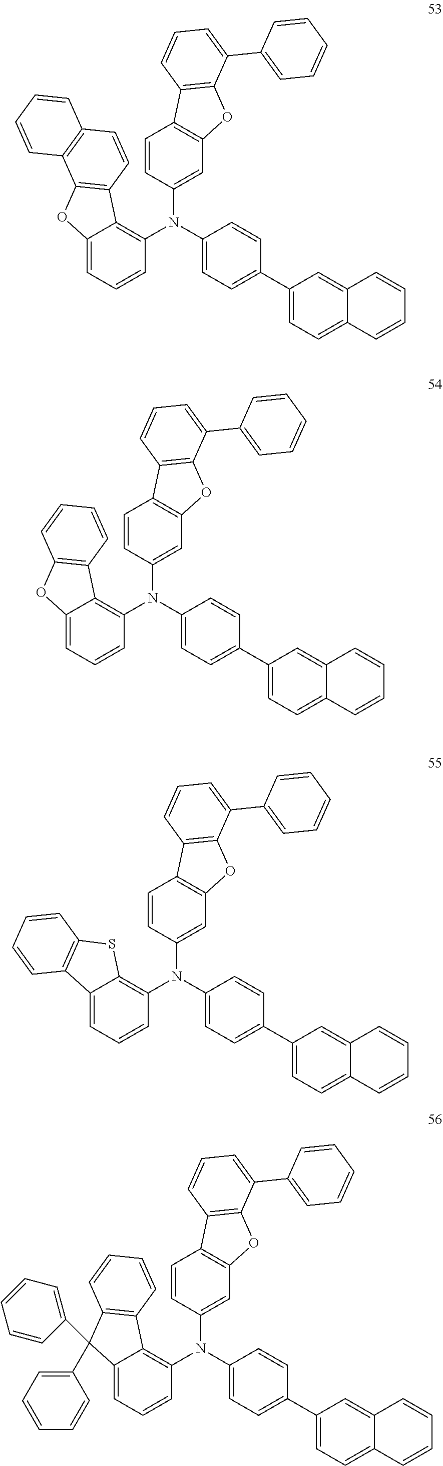 [SMILES: c1ccc(-c2cccc3c2oc2cc(N(c4ccc(-c5ccc6ccccc6c5)cc4)c4cccc5oc6c7ccccc7ccc6c45)ccc23)cc1 ; c1ccc(-c2cccc3c2oc2cc(N(c4ccc(-c5ccc6ccccc6c5)cc4)c4cccc5oc6ccccc6c45)ccc23)cc1 ; c1ccc(-c2cccc3c2oc2cc(N(c4ccc(-c5ccc6ccccc6c5)cc4)c4cccc5c4sc4ccccc45)ccc23)cc1 ; c1ccc(-c2cccc3c2oc2cc(N(c4ccc(-c5ccc6ccccc6c5)cc4)c4cccc5c4-c4ccccc4C5(c4ccccc4)c4ccccc4)ccc23)cc1]

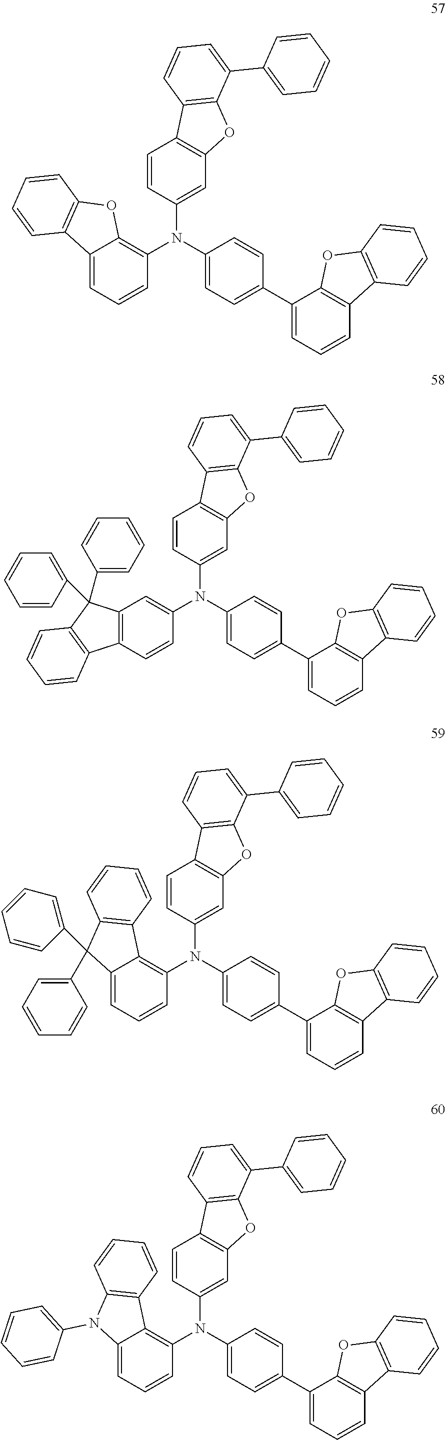 [SMILES: c1ccc(-c2cccc3c2oc2cc(N(c4ccc(-c5cccc6c5oc5ccccc56)cc4)c4cccc5c4oc4ccccc45)ccc23)cc1 ; c1ccc(-c2cccc3c2oc2cc(N(c4ccc(-c5cccc6c5oc5ccccc56)cc4)c4ccc5c(c4)C(c4ccccc4)(c4ccccc4)c4ccccc4-5)ccc23)cc1 ; c1ccc(-c2cccc3c2oc2cc(N(c4ccc(-c5cccc6c5oc5ccccc56)cc4)c4cccc5c4-c4ccccc4C5(c4ccccc4)c4ccccc4)ccc23)cc1 ; c1ccc(-c2cccc3c2oc2cc(N(c4ccc(-c5cccc6c5oc5ccccc56)cc4)c4cccc5c4c4ccccc4n5-c4ccccc4)ccc23)cc1]

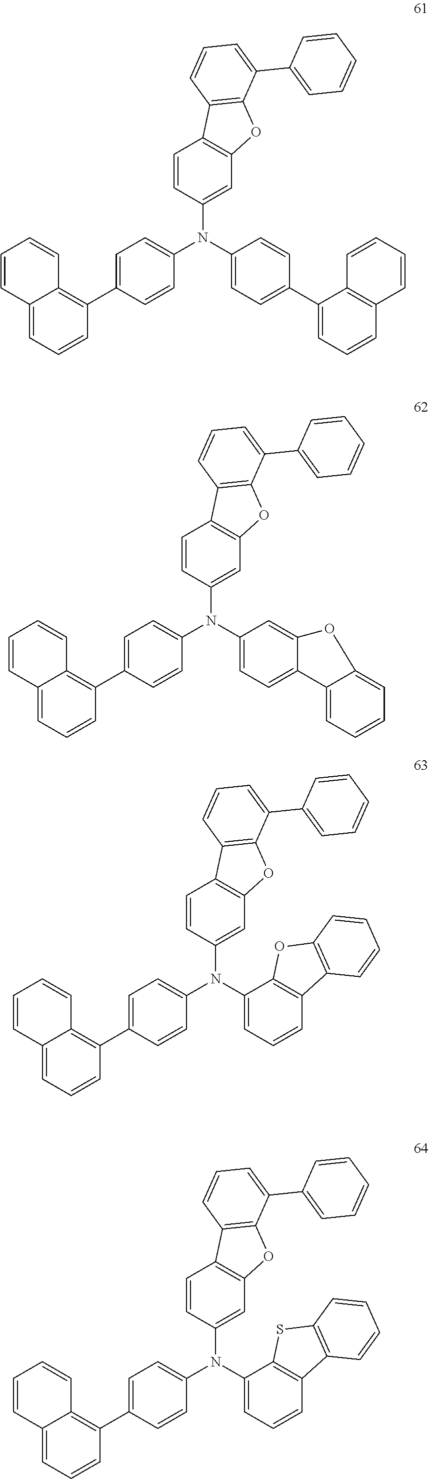

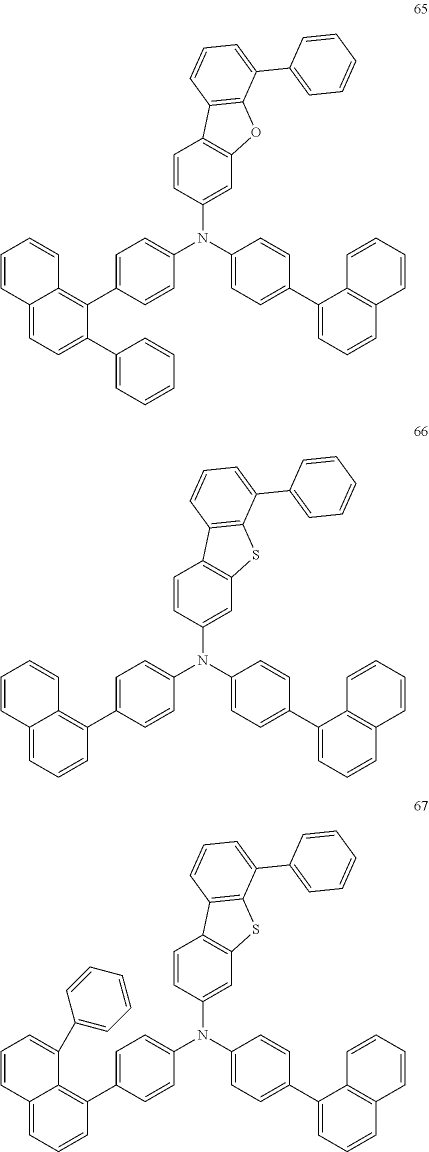 [SMILES: c1ccc(-c2ccc3ccccc3c2-c2ccc(N(c3ccc(-c4cccc5ccccc45)cc3)c3ccc4c(c3)oc3c(-c5ccccc5)cccc34)cc2)cc1 ; c1ccc(-c2cccc3c2sc2cc(N(c4ccc(-c5cccc6ccccc56)cc4)c4ccc(-c5cccc6ccccc56)cc4)ccc23)cc1 ; c1ccc(-c2cccc3c2sc2cc(N(c4ccc(-c5cccc6ccccc56)cc4)c4ccc(-c5cccc6cccc(-c7ccccc7)c56)cc4)ccc23)cc1]

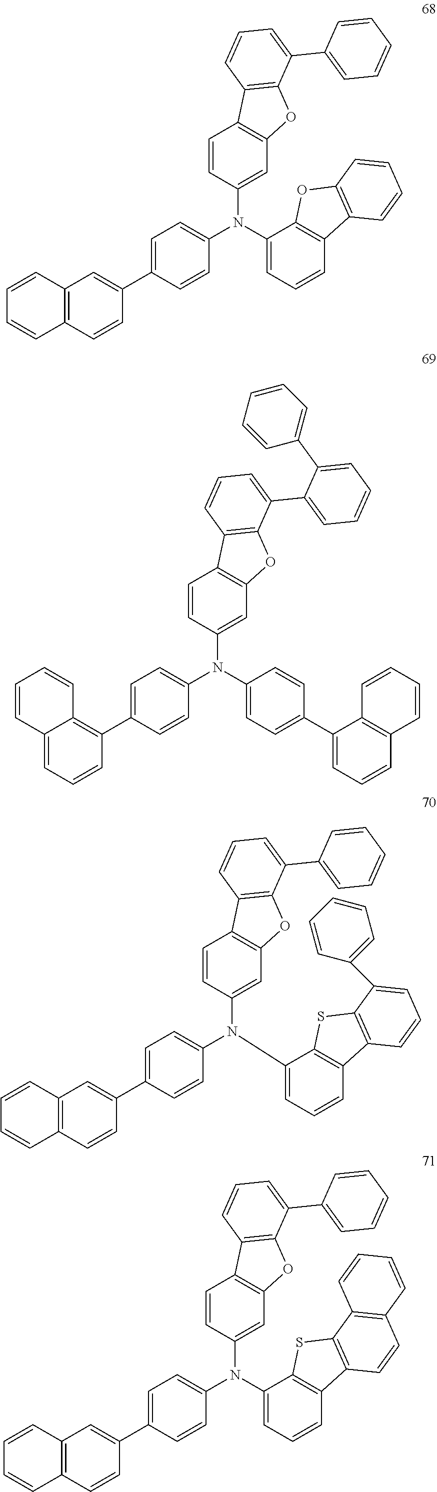

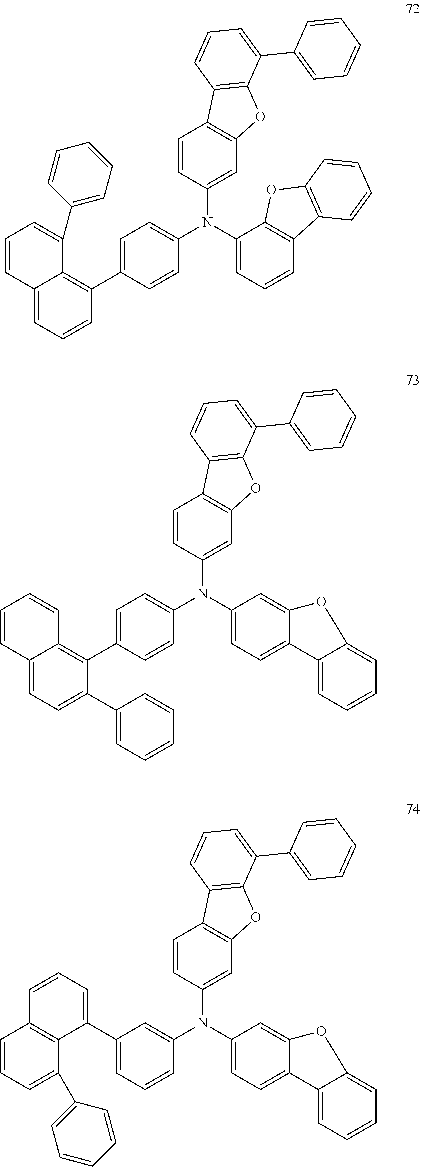 [SMILES: c1ccc(-c2cccc3c2oc2cc(N(c4ccc(-c5cccc6cccc(-c7ccccc7)c56)cc4)c4cccc5c4oc4ccccc45)ccc23)cc1 ; c1ccc(-c2ccc3ccccc3c2-c2ccc(N(c3ccc4c(c3)oc3ccccc34)c3ccc4c(c3)oc3c(-c5ccccc5)cccc34)cc2)cc1 ; c1ccc(-c2cccc3c2oc2cc(N(c4cccc(-c5cccc6cccc(-c7ccccc7)c56)c4)c4ccc5c(c4)oc4ccccc45)ccc23)cc1]

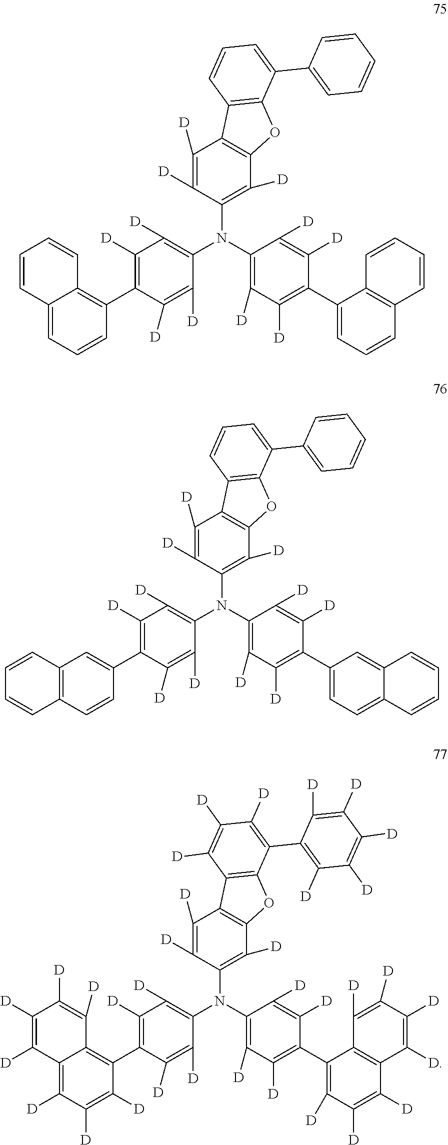

21. A display device, comprising:
a base layer;
a circuit layer on the base layer; and
a display element layer on circuit layer, and comprising a light emitting element, the light emitting element comprises:
a first electrode;
a hole transport region on the first electrode;
an emission layer on the hole transport region;
an electron transport region on the emission layer; and
a second electrode on the electron transport region, wherein
the hole transport region comprises an amine compound represented by the following Formula 1: 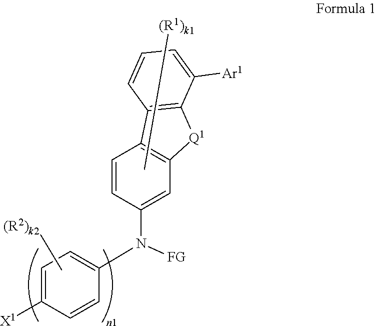
in Formula 1,
Q¹ is O or S,
Ar¹ is a substituted or unsubstituted phenyl group,
R¹ and R² are each independently a hydrogen atom, a deuterium atom, a halogen atom, a substituted or unsubstituted silyl group, a substituted or unsubstituted aryl group of 6 to 30 ring-forming carbon atoms, or a substituted or unsubstituted heteroaryl group of 2 to 30 ring-forming carbon atoms,
X¹ is a substituted or unsubstituted naphthyl group, or a substituted or unsubstituted heteroaryl group of 2 to 30 ring-forming carbon atoms,
n1 is an integer of 1 to 3, k1 is an integer of 0 to 6, k2 is an integer of 0 to 4, and
FG is represented by the following Formula 2-1 or Formula 2-2: 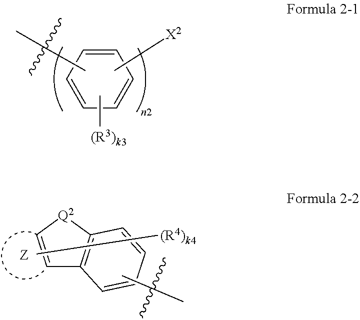
in Formula 2-1 and Formula 2-2,
X² is a substituted or unsubstituted naphthyl group, or a substituted or unsubstituted heteroaryl group of 2 to 30 ring-forming carbon atoms,
R³ and R⁴ are each independently a hydrogen atom, a deuterium atom, a halogen atom, a substituted or unsubstituted silyl group, a substituted or unsubstituted aryl group of 6 to 30 ring-forming carbon atoms, or a substituted or unsubstituted heteroaryl group of 2 to 30 ring-forming carbon atoms,
Q² is O, S, NR⁵, or CR⁶R⁷,
R⁵ to R⁷ are each independently a substituted or unsubstituted alkyl group of 1 to 20 carbon atoms, a substituted or unsubstituted aryl group of 6 to 50 ring-forming carbon atoms, or a substituted or unsubstituted heteroaryl group of 2 to 50 ring-forming carbon atoms,
Z is a substituted or unsubstituted aromatic hydrocarbon ring of 6 to 30 ring-forming carbon atoms, or a substituted or unsubstituted aromatic heterocycle of 2 to 30 ring-forming carbon atoms,
k3 is an integer of 0 to 4, k4 is an integer of 0 to 7, and n2 is an integer of 1 to 3, and
a case where X¹ and X² are 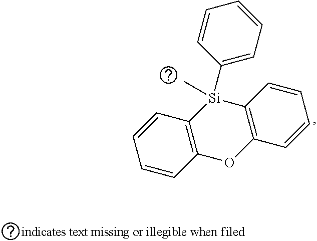 [SMILES: C[Si]1(c2ccccc2)c2ccccc2Oc2ccccc21]
is excluded.

22. The display device of claim 21, further comprising a light controlling layer on the display element layer, and including a quantum dot.
